(19) United States
(12) Patent Application Publication
(10) Pub. No.: US 2024/0043904 A1
(43) Pub. Date: Feb. 8, 2024

(54) MORPHOLOGICAL MARKER STAINING

(71) Applicant: Ventana Medical Systems, Inc., Tucson, AZ (US)

(72) Inventors: Daniel R. BAUER, Tucson, AZ (US); William DAY, Tucson, AZ (US); Mark R. LEFEVER, Quepos (CR); Larry MORRISON, Oro Valley, AZ (US)

(21) Appl. No.: 18/480,970

(22) Filed: Oct. 4, 2023

Related U.S. Application Data
(63) Continuation of application No. PCT/EP2021/073733, filed on Aug. 27, 2021.
(60) Provisional application No. 63/176,326, filed on Apr. 18, 2021.

(51) Int. Cl. C12Q 1/6804; G01N 33/68

(52) U.S. Cl. CPC C12Q 1/6804; G01N 33/6875; G01N 2474/20; C12Q 2600/16

(57) ABSTRACT

Disclosed are systems and methods for labelling one or more morphological markers in a biological sample that are characteristic of one or more molecular features. In particular, system and methods are described for labelling one or more morphological markers in a biological sample with covalently deposited narrow band detectable moieties. Narrow band detectable moiety labelling of the one or more morphological markers permits higher order multiplexed assays due to conservation of available spectral bandwidth. Furthermore, as compared to conventional counterstaining methods, covalent deposition of one or more detectable moieties can provide flexibility and robustness with regard to the order in which biomarkers and morphological markers are labeled in a given staining protocol.

### CROSS REFERENCE TO RELATED APPLICATIONS

The present disclosure is a continuation of International Application No. PCT/EP2021/073733 filed on Aug. 27, 2021, which application claims the benefit of the filing date of U.S. Provisional Patent Application No. 63/176,326 filed on Apr. 18, 2021, the disclosure of which is hereby incorporated by reference herein in its entirety.

### FIELD OF THE DISCLOSURE

The present disclosure is directed to labeling one or more morphological markers and/or one or more biomarkers with different detectable moieties.

### BACKGROUND OF THE DISCLOSURE

Immunohistochemistry (IHC) refers to processes of detecting, localizing, and/or quantifying an antigen, such as a protein, in a biological sample, using antibodies specific to the antigen. In a cellular sample, such as a tissue sample, IHC provides the substantial advantage of providing information regarding where a particular protein is located within the biological sample. In situ hybridization (ISH) refers to the process of using nucleic acid probes for detecting, localizing, and/or quantifying specific nucleic acid sequences within the DNA and RNA that may be present in the sample. Both IHC and ISH can be performed on various biological samples, for example, tissue samples (e.g. fresh frozen or formalin fixed, paraffin embedded (FFPE)) and cytological samples, and can be used to detect a wide variety of specific antigen and sequence targets. Recognition of targets within a sample by antibodies and nucleic acid probes can be detected, such as visualized, using various labels (e.g., chromogenic, fluorescent, luminescent, radiometric). Amplification of the recognition event is desirable as is the ability to confidently detect cellular markers of low abundance. For example, depositing at the marker's site hundreds or thousands of label molecules in response to a single antigen detection event enhances, through amplification, the ability to detect that recognition event.

Adverse events often accompany amplification, such as non-specific signals that are apparent as an increased background signal. An increased background signal interferes with the clinical analysis by obscuring faint signals that may be associated with low, but clinically significant, expressions. Accordingly, while amplification of recognition events is desirable, amplification methods that minimize background signals are highly desirable.

Even with precise localization of nucleic acids and protein targets within a sample, additional diagnostic information may also be available from the locations of these targets relative to specific morphological structures with cells and tissues. Conventional bright field stains for visualization of morphological structures are typically broadly absorbing dyes that can present a challenge to combining IHC and ISH detection with morphologic staining on a single sample, particularly when there is a need or desire for multiplexed detection of multiple targets in their morphological context within a single sample. For example, the broad absorption of the hematoxylin and eosin (H&E) stain contributes strong absorption across the visible spectrum that obscures other chromogenic compounds that otherwise might be visible through a microscope. The H&E absorption complicates quantification of target molecules by image analysis techniques that rely on un-mixing of the spectral contributions of the visible chromogens used to detect target molecules and the morphological dye. As a result, it is more common to stain a first tissue section for the target(s) and a second tissue section for the morphological stain, in so-called “serial slides.” Alternatively, it is possible to first stain a tissue for either of the target(s) or the morphological stain, to de-stain the tissue section, and then to stain for the other of the target(s) or the morphological stain. Another possibility is to reduce the intensity of the conventional morphological stain through dilution of the stain or reducing the time the sample is in contact with the stain so as to not obscure target IHC and ISH signals. Each of these alternatives have disadvantages.

In serial slices cut from the tissue with the microtome, a new set of cells is sliced through in each successive cut, and therefore, serial slices from a tissue block do not always match up with each other morphologically. Staining, destaining and restaining can damage tissue structure and morphology, especially if it must be repeated to achieve higher orders of multiplexing. If a reduction in the stain intensity of the conventional morphological stain is utilized, fine morphological features can become indiscernible due to inadequate staining, and yet, regardless of the lower levels of hematoxylin staining, large portions of the available detection spectrum are less useful for detecting biomarkers because the broad hematoxylin absorption must be unmixed from any biomarker signals with which it overlaps. Therefore, it is desirable to provide improved methods for establishing morphological context for one or more biomarker signals while still permitting detection of the biomarker signal(s) themselves.

### BRIEF SUMMARY OF THE DISCLOSURE

In one aspect, the present disclosure is directed to methods of labeling one or more morphological markers in a biological sample to provide morphological context within the sample during manual or automated microscopic analysis. In some embodiments, the disclosed morphological marker labelling methods are combined with biomarker detection methods to provide morphological context to the locations of one or more biomarkers detected within the biological sample. For example, staining a combination of one or more morphological markers and one or more medically relevant biomarkers can be used to help determine one or more locations of the one or more medically relevant biomarkers relative to morphological features (e.g. cellular components, the nucleus, etc.) visualized through the staining of the one or more morphological markers. In some embodiments, the one or more morphological markers may be representative or characteristic of the same morphological feature. In other embodiments, the one or more morphological markers may be representative or characteristic of different marker morphological features. Biomarker presence along with biomarker location relative to morphological features in a sample is often indicative of a particular medical condition, and can be determinative in qualifying a patient for targeted treatment with a particular class of therapeutics. Biomarker presence and biomarker location can also serve as quality control for the staining process itself, allowing detection of aberrant staining patterns that could signal a variety of process step failures, for example, failure properly to dispense a reagent to the sample. In some embodiments, the one or more morphological markers and/or the one or more biomarkers are stained with detectable moieties, such as those detectable moieties including coumarin core, a phenoxazinone core, a 4-Hydroxy-3-phenoxazinone core, a 7-amino-4-Hydroxy-3-phenoxazinone core, a thioninium core, a phenoxazine core, a phenoxathiin-3-one core, a xanthene core, a heptamethine cyanine core, and a croconate core.

In another aspect, the disclosed methods of morphological marker labelling free-up spectral wavelength ranges for detection of one or more biomarkers in a single sample, particularly for brightfield multiplexing where biomarker signals would otherwise become masked using a conventional chemical counterstain. Thus, in some embodiments, a detectable moiety with a narrow first absorption band is deposited on, across or in proximity to at least a part of one or more morphological features within a cell or tissue sample, thereby preserving available detection spectrum for the detection of one or more biomarkers in the same sample, even if they are present in low abundance. In particular embodiments, a detectable moiety with a narrow first absorption band in the UV portion (such as the UVA portion) of the electromagnetic spectrum is utilized. In other particular embodiments, a detectable moiety with a narrow first absorption band in the near-IR (NIR) portion of the electromagnetic spectrum is utilized. In some embodiments, detectable moieties include those having a coumarin core, a phenoxazinone core, a 4-Hydroxy-3-phenoxazinone core, a 7-amino-4-Hydroxy-3-phenoxazinone core, a thioninium core, a phenoxazine core, a phenoxathiin-3-one core, a xanthene core, a heptamethine cyanine core, and a croconate core.

By spreading the ranges of wavelengths available into the UV and NIR and using detectable moieties having narrow first absorption bands, it is possible to perform highly multiplexed assays. Thus, for example, in some embodiments, at least one morphological marker and 5 or more, such as 7 or more, 9 or more, 10 or more, or 11 or more biomarkers can be detected in a single sample in spatial relationship to the at least one morphological feature. In other embodiments, two or more morphological markers and at least 4 or more, such as 6 or more, 8 or more, or 10 or more biomarkers in spatial relationship to the two or more morphological markers. For example, FIG. 13 shows a particular spectral palette of disclosed detectable moieties that can be selected and deposited for detection of a morphological marker and a biomarker, it is clear that very high order multiplexes of multiple biomarker signals and morphological marker signals are possible.

In some embodiments, the detectable moieties are covalently deposited onto the biological sample yielding a sample that can be processed flexibly with regard to the order in which particular parts (such as one or more morphological features and one or more biomarkers) of the biological sample are stained. For example, one or more morphological features can first be detected, followed by detection of the one or more biomarkers. Alternatively, the detection of the one or more biomarkers can be performed first, followed by staining of the one or more morphological features. Overall, any particular order of biomarker staining and morphological staining is possible.

In some embodiments, covalent deposition of a chromophore or detectable moiety is accomplished using Tyramide Signal Amplification (TSA), which has also been referred to as catalyzed reporter deposition (CARD). U.S. Pat. No. 5,583,001 discloses a method for detecting and/or quantitating an analyte using an analyte-dependent enzyme activation system that relies on catalyzed reporter deposition to amplify the detectable label signal. Catalysis of an enzyme in a CARD or TSA method is enhanced by reacting a labeled phenol molecule with an enzyme. Modern methods utilizing TSA effectively increase the signals obtained from IHC and ISH assays while not producing significant background signal amplification (see, for example, U.S. application publication No. 2012/0171668 which is hereby incorporated by reference in its entirety for disclosure related to tyramide amplification reagents). Reagents for these amplification approaches are being applied to clinically important targets to provide robust diagnostic capabilities previously unattainable (VENTANA OptiView Amplification Kit, Ventana Medical Systems, Tucson AZ, Catalog No. 760-099).

TSA takes advantage of a reaction catalyzed by horseradish peroxidase (HRP) acting on tyramide. In the presence of H₂O₂, tyramide is converted to a highly-reactive and short-lived radical intermediate that reacts preferentially with electron-rich amino acid residues on proteins. Covalently-bound detectable moieties can then be detected by variety of chromogenic visualization techniques and/or by fluorescence microscopy. In IHC and ISH, where spatial and morphological context is highly valued, the short lifetime of the radical intermediate results in covalent binding of the tyramide to on the tissue in close proximity to the site of generation, thereby giving discrete and specific signals at the locations of proteins and nucleic acid targets.

In other embodiments, covalent deposition of a chromophore or detectable moiety is performed using quinone methide chemistry. U.S. Pat. No. 10,168,336, entitled “Quinone Methide Analog Signal Amplification,” granted on Jan. 1, 2019, describes a technique (“QMSA”) that, like TSA, may be used to increase signal amplification without significantly increasing background signals. In particular, U.S. Pat. No. 10,168,336 describes novel quinone methide analog precursors and methods of using the quinone methide analog precursors to detect one or more targets in a biological sample. In a particular embodiment, the method of detection includes contacting the sample with a detection antibody or probe, then contacting the sample with a labeling conjugate that comprises an alkaline phosphatase (AP) enzyme and a binding moiety, where the binding moiety recognizes the antibody or probe (for example, by binding to a hapten or a species specific antibody epitope, or a combination thereof). The alkaline phosphatase enzyme of the labeling conjugate interacts with a quinone methide analog precursor comprising the detectable moiety, thereby forming a reactive quinone methide analog, which binds covalently to the biological sample proximally to or directly on the target. The detectable label is then detected, such as visually or through imaging techniques. U.S. Pat. No. 10,168,336 is incorporated by reference herein in its entirety.

Another technique for depositing detectable moieties employs “click” chemistry to form a covalent bond between a detectable moiety and a morphological marker or a biomarker in a sample. “Click chemistry” is a chemical philosophy, independently defined by the groups of Sharpless and Meldal, that describes chemistry tailored to generate substances quickly and reliably by joining small units together. “Click chemistry” has been applied to a collection of reliable and self-directed organic reactions (Kolb, H. C.; Finn, M. G.; Sharpless, K. B. Angew. Chen. Int. Ed. 2001, 40, 2004-2021). In the context of covalently depositing detectable labels onto a biological sample, a click chemistry technique is described in US2019/0204330, which incorporated by reference herein. In this technique, either tyramide deposition as described above or quinone methide deposition also described above, is used to covalently anchor a first reactive group capable of participating in a click chemistry reaction to the biological sample. A second component of the detection system having a corresponding second reactive group capable of participating in a click chemistry reaction is then reacted with the first reactive group to covalently bind the second component to the biological sample. In a particular embodiment, the technique described includes contacting the biological sample with a first detection probe specific to a first target. The first detection probe may be a primary antibody or a nucleic acid probe. Subsequently, the sample is contacted with a first labeling conjugate, the first labeling conjugate comprising a first enzyme. In some embodiments, the first labeling conjugate is a secondary antibody specific for either the primary antibody (such as the species from which the antibody was obtained) or to a label (such as a hapten) conjugated to the nucleic acid probe. Next, the biological sample is contacted with a first member of a pair of click conjugates. The first enzyme cleaves the first member of the pair of click conjugates having a tyramide or quinone methide precursor, thereby converting the first member into a reactive intermediate which covalently binds to the biological sample proximally to or directly on the first target. Next, a second member of the pair of click conjugates is contacted with the biological sample, the second member of the pair of click conjugates comprising a first reporter moiety (e.g. a chromophore) and a second reactive functional group, where the second reactive functional group of the second member of the first pair of click conjugates is capable of reacting with the first reactive functional group of the first member of the pair of click conjugates. Finally, signals from the first reporter moiety are detected.

In one embodiment, a method is disclosed for detecting a biomarker in morphological context within a biological sample, where the method includes labeling at least a portion of a first morphological feature of the biological sample with a first detectable moiety, wherein labeling the first morphological feature (e.g. a nucleus or a portion thereof) comprises contacting morphological marker (e.g. DNA or a histone marker) characteristic of the first morphological feature with a first detection probe that binds to the morphological marker. The method further includes covalently depositing the first detectable moiety at or near where the first detection probe is bound to the morphological marker characteristic of the morphological feature. Labeling of a first biomarker in the biological sample with a second detectable moiety is also part of the method, where the second detectable moiety is different from the first detectable moiety, and where the labeling of the first biomarker includes contacting the first biomarker with a second detection probe that binds to the first biomarker. The method further includes covalently depositing the second detectable moiety at or near where the second antibody is bound to the first biomarker. In some embodiments, the first and second detectable markers include those having a coumarin core, a phenoxazinone core, a 4-Hydroxy-3-phenoxazinone core, a 7-amino-4-Hydroxy-3-phenoxazinone core, a thioninium core, a phenoxazine core, a phenoxathiin-3-one core, a xanthene core, a heptamethine cyanine core, and a croconate core.

In a more particular embodiment, the morphological feature is a nucleus or a component of a nucleus within a cell and the morphological marker is present in the nucleus or a component of the nucleus in the cell. In an even more particular embodiment the first detection probe is an antibody against a component of a nucleus (e.g. DNA, histone proteins, etc.), and one or more biomarkers in a biological sample. Examples of other suitable morphological features and morphological markers and biomarkers are described herein.

In some embodiments, the labeling of the one or more morphological markers provides positional context to the one or more labeled biomarkers. In some embodiments, the labeling of the one or more morphological markers allows for cell and/or tissue morphology to be detected and/or visualized concurrently with one or more labeled biomarkers. In some embodiments, the labeling of one or more morphological markers serves as a surrogate for a special stain. In other embodiments, the labeling of the one or more morphological markers services as a substitute for a counterstain, e.g., hematoxylin. These and other advantageous uses of staining a sample in accordance with the presently disclosed methods are described herein.

Another aspect of the present disclosure is a method of detecting one or more targets within a biological sample, comprising: labeling a first morphological marker with a first detectable moiety, wherein the first detectable moiety has a first absorbance peak with FWHM of less than about 200 nm and an absorbance maximum (λₘₐₓ) between 330 nm+/−10 and 950 nm+/−10; and labeling a first biomarker with a second detectable moiety, wherein the second detectable moiety is different than the first detectable moiety, and wherein the second detectable moiety has a first absorbance peak with FWHM of less than about 200 nm and an absorbance maximum (λₘₐₓ) between 330 nm+/−10 and 950 nm+/−10. In some embodiments, the first and second detectable moieties have a first absorbance peak with FWHM of less than 160 nm. In some embodiments, the method further comprises labeling a second morphological marker with a third detectable moiety where the third detectable moiety is different than either the first or second detectable moieties. In some embodiments, the first and second morphological markers are both characteristic of a same morphological feature.

In some embodiments, the first absorbance peak with FWHM of the first and/or second detectable moieties is less than about 130 nm. In some embodiments, the first absorbance peak with FWHM of the first and/or second detectable moieties is less than about 100 nm. In some embodiments, the first absorbance peak with FWHM of the first and/or second detectable moieties is less than about 80 nm. In some embodiments, the first absorbance peak with FWHM of the first and/or second detectable moieties is less than about 60 nm.

In some embodiments, the absorbance maximum (λₘₐₓ) of the first detectable moiety and the absorbance maximum (λₘₐₓ) of the second detectable moiety are separated by at least about 20 nm. In some embodiments, the absorbance maximum (λₘₐₓ) of the first detectable moiety and the absorbance maximum (λₘₐₓ) of the second detectable moiety are separated by at least about 30 nm. In some embodiments, the absorbance maximum (λₘₐₓ) of the first detectable moiety and the absorbance maximum (λₘₐₓ) of the second detectable moiety are separated by at least about 40 nm. In some embodiments, the absorbance maximum (λₘₐₓ) of the first detectable moiety and the absorbance maximum (λₘₐₓ) of the second detectable moiety are separated by at least about 50 nm.

In some embodiments, the first morphological marker comprises DNA. In some embodiments, the labeling of the DNA with the first detectable moiety comprises: (a) contacting the biological sample with an anti-DNA primary antibody; (b) contacting the biological sample with an anti-species secondary antibody specific to the anti-DNA primary antibody, wherein the anti-species antibody is conjugated directly or indirectly to at least one enzyme; and (c) contacting the biological sample with a first detectable conjugate comprising (i) the first detectable moiety, and (ii) a tyramide moiety, a quinone methide precursor moiety, or a derivative or analog of a tyramide moiety or quinone methide precursor moiety. In some embodiments, the labeling of the DNA with the first detectable moiety comprises: (a) contacting the biological sample with an anti-DNA primary antibody; (b) contacting the biological sample with an anti-specifies secondary antibody specific to the anti-DNA antibody, wherein the anti-species antibody is conjugated directly or indirectly to at least one enzyme; (c) contacting the biological sample with a first tissue reactive conjugate comprising: (i) a first member of a pair of reactive functional groups capable of participating in a click chemistry reaction, and (ii) a tyramide moiety, a quinone methide precursor moiety, or a derivative or analog of a tyramide moiety or quinone methide precursor moiety; and (d) contacting the biological sample with a detectable conjugate comprising (i) the first detectable moiety, and (ii) a second member of the pair of reactive functional groups. In alternative embodiments, the primary antibody is labeled with a hapten and the secondary antibody specifically binds to the hapten conjugated to the primary antibody.

In some embodiments, the first morphological marker comprises a histone protein. In some embodiments, the labeling of the histone proteins with the first detectable moiety comprises: (a) contacting the biological sample with an anti-histone primary antibody; (b) contacting the biological sample with an anti-specifies secondary antibody specific to the anti-histone primary antibody, wherein the anti-species antibody is conjugated directly or indirectly to at least one enzyme; and (c) contacting the biological sample with a first detectable conjugate comprising (i) the first detectable moiety, and (ii) a tyramide moiety, a quinone methide precursor moiety, or a derivative or analog of a tyramide moiety or quinone methide precursor moiety. In some embodiments, the labeling of the histone proteins with the first detectable moiety comprises: (a) contacting the biological sample with an anti-histone primary antibody; (b) contacting the biological sample with an anti-species secondary antibody specific to the anti-histone antibody, wherein the anti-species antibody is conjugated directly or indirectly to at least one enzyme; (c) contacting the biological sample with a first tissue reactive conjugate comprising: (i) a first member of a pair of reactive functional groups capable of participating in a click chemistry reaction, and (ii) a tyramide moiety, a quinone methide precursor moiety, or a derivative or analog of a tyramide moiety or quinone methide precursor moiety; and (d) contacting the biological sample with a detectable conjugate comprising (i) the first detectable moiety, and (ii) a second member of the pair of reactive functional groups. In alternative embodiments, the primary antibody is labeled with a hapten and the secondary antibody specifically binds to the hapten conjugated to the primary antibody.

In some embodiments, the first morphological marker is selected from the group consisting of a marker for cytosol, a nuclear marker, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers. Suitable morphological markers, antibodies and sources of antibodies are provided below in Tables 4 through 10.

In some embodiments, the first biomarker is a protein biomarker. In some embodiments, the first biomarker is selected from the group consisting of PD-L1, Ki-67, CD3, CD8, CD4, CD20, CD68, p40, p63, TTF-1, ERG, ERBB2 (HER2), alpha-methylacyl-CoA racemase (AMACR), and synaptophysin. In some embodiments, the first biomarker is a nucleic acid biomarker, selected from the group consisting of ERBB2, EGFR, PTEN, p63, TOP2A, CCND1, RREB1, CKS1B, CDKN2C, MCL1, NTRK1, PBX1, ALK, N-MYC, BCL6, PIK3CA, RPN1, TERC, IGH, FGFR3, PDGFRA, EGR1, PDGFRB, and NSD1.

In some embodiments, the first detectable moiety comprises a coumarin core. In some embodiments, the second detectable moiety is within the visible spectrum or within the infrared spectrum. In some embodiments, the second detectable moiety is within the ultraviolet spectrum. In some embodiments, the first and second detectable moieties have absorbance maximums (λₘₐₓ) that are separated by at least 20 nm.

In some embodiments, the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core. In some embodiments, the second detectable moiety is within the ultraviolet spectrum or within the infrared spectrum. In some embodiments, the second detectable moiety is within the visible spectrum. In some embodiments, the first and second detectable moieties have absorbance maximums (λₘₐₓ) that are separated by at least 20 nm.

In some embodiments, the first detectable moiety comprises a heptamethine cyanine core or a croconate core. In some embodiments, the second detectable moiety is within the visible spectrum or within the ultraviolet spectrum. In some embodiments, the second detectable moiety is within the infrared spectrum. In some embodiments, the first and second detectable moieties have absorbance maximums (λₘₐₓ) that are separated by at least 20 nm.

A further aspect of the present disclosure is a method of detecting one or more targets within a biological sample, comprising: labeling a first morphological marker with a first detectable moiety comprising a core selected from the group consisting of a coumarin core, a phenoxazinone core, a 4-Hydroxy-3-phenoxazinone core, a 7-amino-4-Hydroxy-3-phenoxazinone core, a thioninium core, a phenoxazine core, a phenoxathiin-3-one core, a xanthene core, a heptamethine cyanine core and a croconate core; labeling a first biomarker with a second detectable moiety comprising a core selected from the group consisting of a coumarin core, a phenoxazinone core, a 4-Hydroxy-3-phenoxazinone core, a 7-amino-4-Hydroxy-3-phenoxazinone core, a thioninium core, a phenoxazine core, a phenoxathiin-3-one core, a xanthene core, a heptamethine cyanine core and a croconate core; wherein the first and second detectable moieties are different and have absorbance maximums (λₘₐₓ) which differ by at least 10 nm.

In some embodiments, the absorbance maximums of the first and second detectable moieties (λₘₐₓ) differ by at least 20 nm. In some embodiments, the absorbance maximums (λₘₐₓ) of the first and second detectable moieties differ by at least 30 nm. In some embodiments, the absorbance maximums (λₘₐₓ) of the first and second detectable moieties differ by at least 40 nm. In some embodiments, the absorbance maximums (λₘₐₓ) of the first and second detectable moieties differ by at least 50 nm. In some embodiments, the absorbance maximums (λₘₐₓ) of the first and second detectable moieties differ by at least 60 nm. In some embodiments, the absorbance maximums (λₘₐₓ) of the first and second detectable moieties differ by at least 70 nm. In some embodiments, the absorbance maximums (λₘₐₓ) of the first and second detectable moieties differ by at least 80 nm. In some embodiments, the absorbance maximums (λₘₐₓ) of the first and second detectable moieties differ by at least 90 nm.

In some embodiments, the first biomarker is a cancer biomarker. Suitable cancer biomarkers include Ki-67, PD-L1, ER, PR, ERBB2 (HER2), EGFR, AMACR, CD8, CD3, or ERG. In some embodiments, the first morphological marker comprises DNA. In some embodiments, the first morphological marker comprises a histone protein. In some embodiments, the first morphological marker is selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers.

In some embodiments, the method further comprises labeling a second biomarker with a third detectable moiety, wherein the third detectable moiety is different than the first and second detectable moieties, and wherein the first, second, and third detectable moieties have absorbance maximums (λₘₐₓ) which differ by at least 10 nm. In some embodiments, the absorbance maximums (λₘₐₓ) of the first, second, and third detectable moieties differ by at least 20 nm. In some embodiments, the absorbance maximums (λₘₐₓ) of the first, second, and third detectable moieties differ by at least 30 nm. In some embodiments, the absorbance maximums (λₘₐₓ) of the first, second, and third detectable moieties differ by at least 40 nm. In some embodiments, the absorbance maximums (λₘₐₓ) of the first, second, and third detectable moieties differ by at least 30 nm. In some embodiments, the absorbance maximums (λₘₐₓ) of the first, second, and third detectable moieties differ by at least 50 nm. In some embodiments, the absorbance maximums (λₘₐₓ) of the first, second, and third detectable moieties differ by at least 60 nm. In some embodiments, the absorbance maximums (λₘₐₓ) of the first, second, and third detectable moieties differ by at least 70 nm. In some embodiments, the absorbance maximums (λₘₐₓ) of the first, second, and third detectable moieties differ by at least 80 nm. In some embodiments, the absorbance maximums (λₘₐₓ) of the first, second, and third detectable moieties differ by at least 90 nm.

In some embodiments, the first and second detectable moieties are selected from the group consisting of:

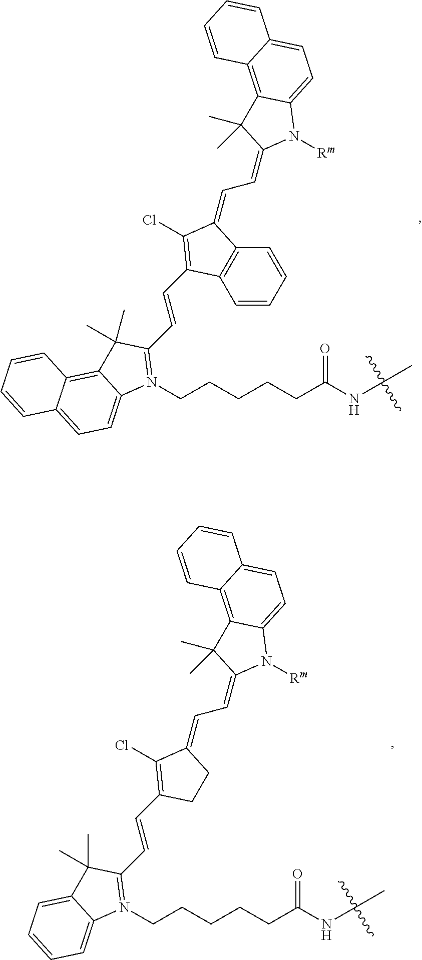

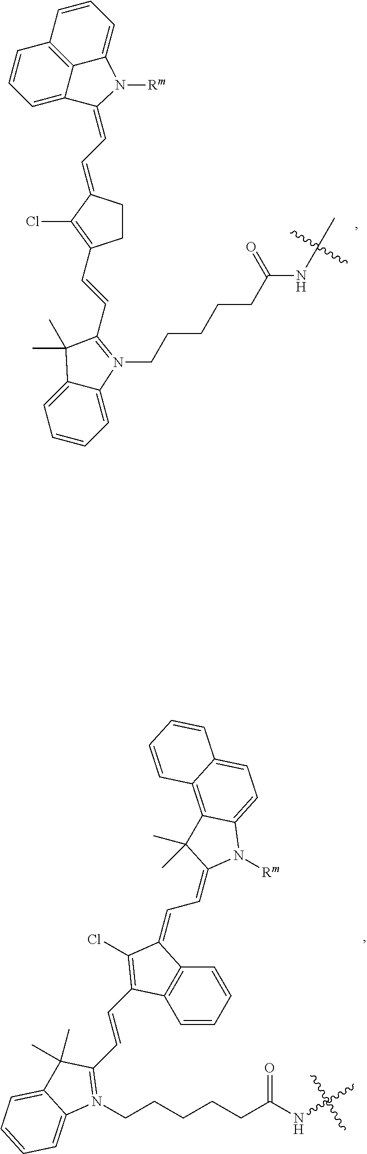

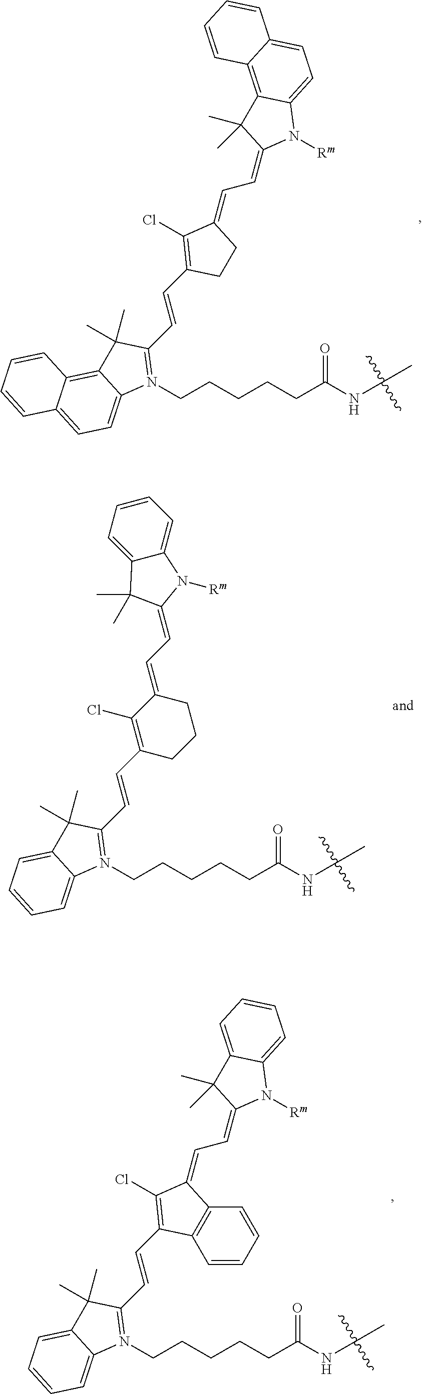

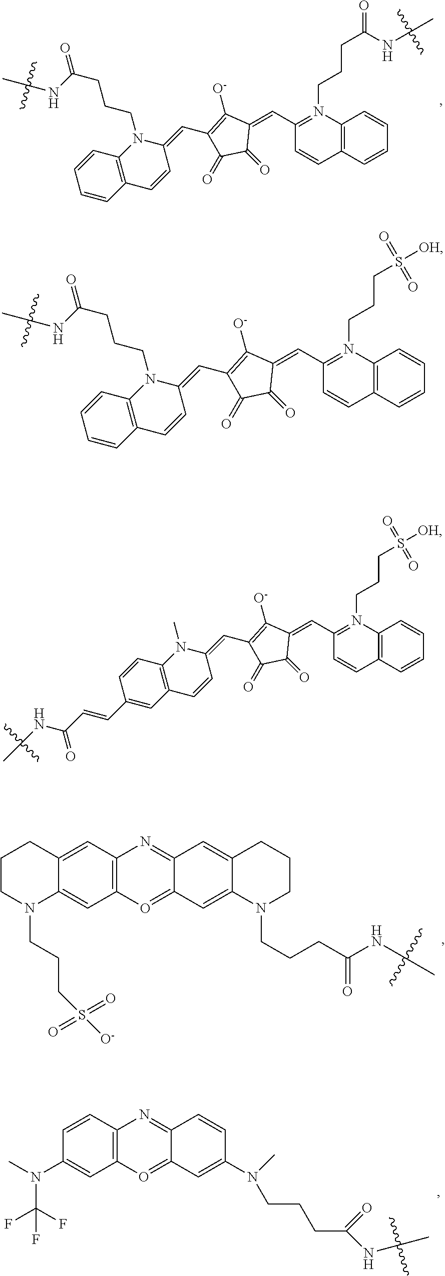

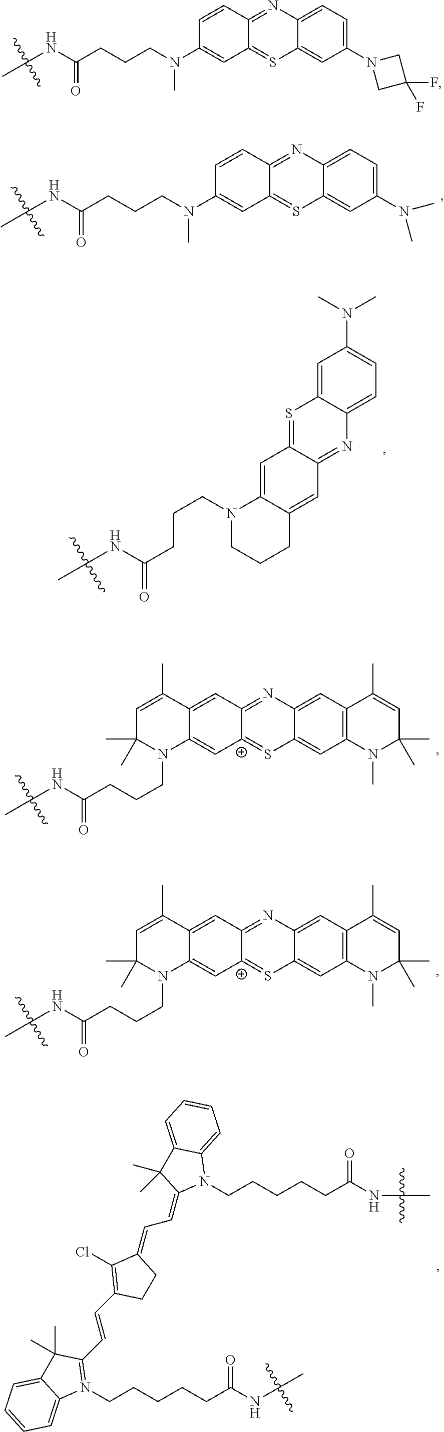

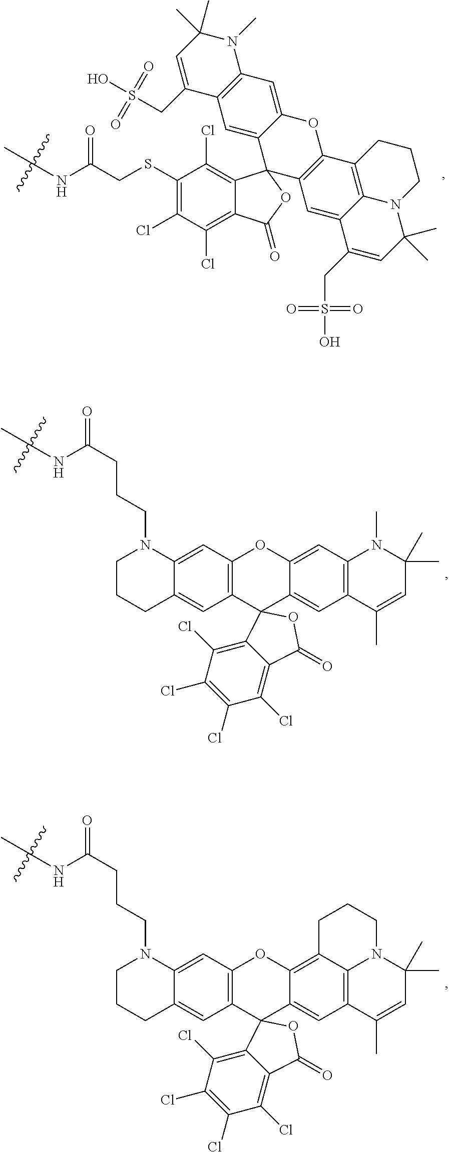 [SMILES: *NC(=O)CSc1c(Cl)c(Cl)c2c(c1Cl)C1(OC2=O)c2cc3c(cc2Oc2c1cc1c4c2CCCN4C(C)(C)C=C1CS(=O)(=O)O)N(C)C(C)(C)C=C3CS(=O)(=O)O ; *NC(=O)CCCN1CCCc2cc3c(cc21)Oc1cc2c(cc1C31OC(=O)c3c(Cl)c(Cl)c(Cl)c(Cl)c31)C(C)=CC(C)(C)N2C ; *NC(=O)CCCN1CCCc2cc3c(cc21)Oc1c(cc2c4c1CCCN4C(C)(C)C=C2C)C31OC(=O)c2c(Cl)c(Cl)c(Cl)c(Cl)c21]

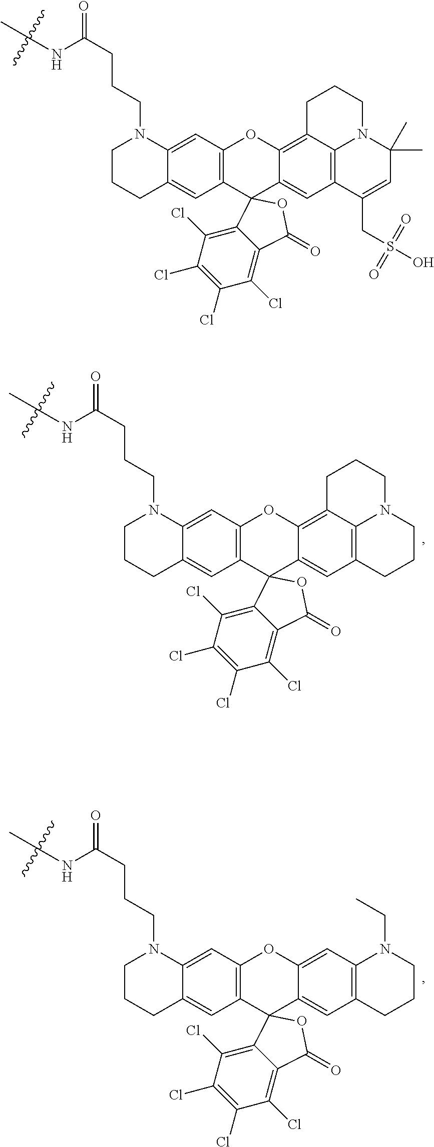 [SMILES: *NC(=O)CCCN1CCCc2cc3c(cc21)Oc1c(cc2c4c1CCCN4C(C)(C)C=C2CS(=O)(=O)O)C31OC(=O)c2c(Cl)c(Cl)c(Cl)c(Cl)c21 ; *NC(=O)CCCN1CCCc2cc3c(cc21)Oc1c(cc2c4c1CCCN4CCC2)C31OC(=O)c2c(Cl)c(Cl)c(Cl)c(Cl)c21 ; *NC(=O)CCCN1CCCc2cc3c(cc21)Oc1cc2c(cc1C31OC(=O)c3c(Cl)c(Cl)c(Cl)c(Cl)c31)CCCN2CC]

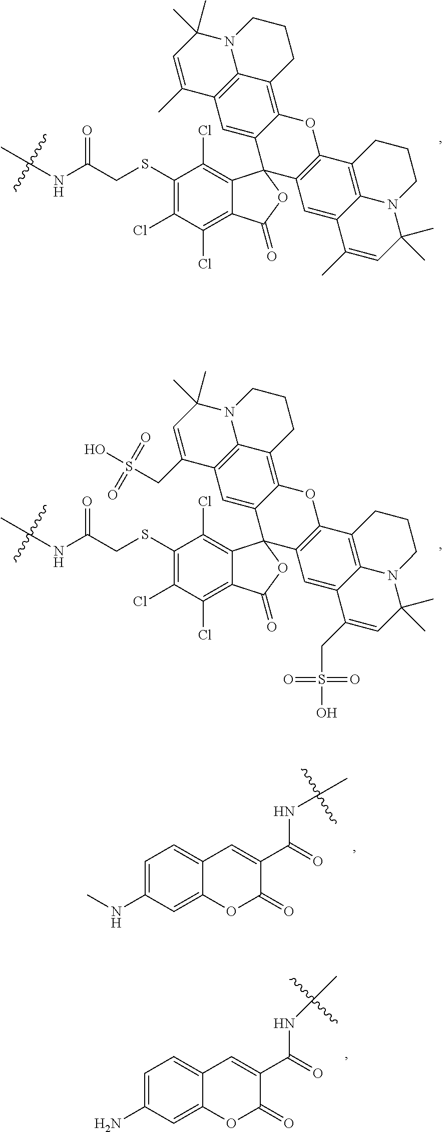

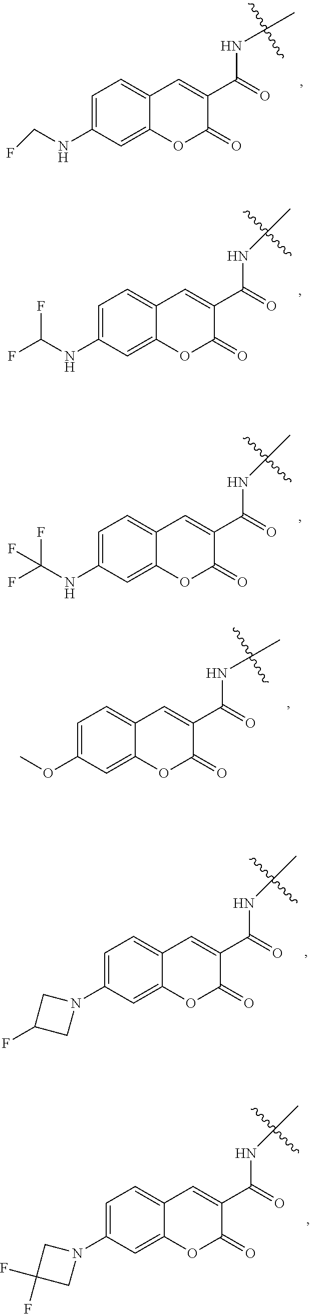 [SMILES: *NC(=O)c1cc2ccc(NCF)cc2oc1=O ; *NC(=O)c1cc2ccc(NC(F)F)cc2oc1=O ; *NC(=O)c1cc2ccc(NC(F)(F)F)cc2oc1=O ; *NC(=O)c1cc2ccc(OC)cc2oc1=O ; *NC(=O)c1cc2ccc(N3CC(F)C3)cc2oc1=O ; *NC(=O)c1cc2ccc(N3CC(F)(F)C3)cc2oc1=O]

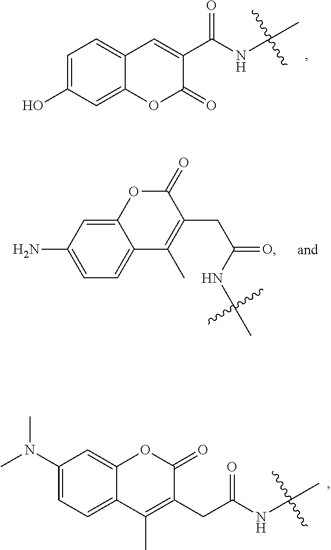 [SMILES: *NC(=O)c1cc2ccc(O)cc2oc1=O ; *NC(=O)Cc1c(C)c2ccc(N)cc2oc1=O ; *NC(=O)Cc1c(C)c2ccc(N(C)C)cc2oc1=O]

where the symbol “ [symbol] ” refers to the site in which the detectable moiety is conjugated to another moiety of a detectable conjugate.

Another aspect of the present disclosure is a biological sample comprising: (a) a first morphological marker labeled with a first detectable moiety; and (b) a first biomarker labeled with a second detectable moiety; wherein the first and second detectable moieties each have a first absorbance peak with FWHM of less than about 200 nm and an absorbance maximum (λₘₐₓ) between 330 nm+/−10 and 950 nm+/−10; and wherein an absorbance maximum (λₘₐₓ) of the first detectable moiety and an absorbance maximum (λₘₐₓ) of the second detectable moiety are separated by at least 20 nm. In some embodiments, the separation between the absorbance maximums (λₘₐₓ) of the first and second detectable moieties is at least 30 nm. In some embodiments, the separation between the absorbance maximums (λₘₐₓ) of the first and second detectable moieties is at least 45 nm. In some embodiments, the separation between the absorbance maximums (λₘₐₓ) of the first and second detectable moieties is at least 60 nm. In some embodiments, the separation between the absorbance maximums (λₘₐₓ) of the first and second detectable moieties is at least 75 nm. In some embodiments, the separation between the absorbance maximums (λₘₐₓ) of the first and second detectable moieties is at least 90 nm.

In some embodiments, the first morphological marker is selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers. In some embodiments, the first morphological marker is DNA. In some embodiments, the first morphological marker is a histone protein.

In some embodiments, the first detectable moiety comprises a coumarin core. In some embodiments, the second detectable moiety is within the visible spectrum or within the infrared spectrum. In some embodiments, the second detectable moiety is within the ultraviolet spectrum. In some embodiments, the first and second detectable moieties have absorbance maximums (λₘₐₓ) that are separated by at least 20 nm.

In some embodiments, the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core. In some embodiments, the second detectable moiety is within the ultraviolet spectrum or within the infrared spectrum. In some embodiments, the second detectable moiety is within the visible spectrum. In some embodiments, the first and second detectable moieties have absorbance maximums (λₘₐₓ) that are separated by at least 20 nm.

In some embodiments, the first detectable moiety comprises a heptamethine cyanine core or a croconate core. In some embodiments, the second detectable moiety is within the visible spectrum or within the ultraviolet spectrum. In some embodiments, the second detectable moiety is within the infrared spectrum. In some embodiments, the first and second detectable moieties have absorbance maximums (λₘₐₓ) that are separated by at least 20 nm.

A further aspect of the present disclosure is a biological sample comprising: (a) first biomarker labeled with a first detectable moiety; and (b) one of DNA or histone proteins labeled with a second detectable moiety; wherein the first and second detectable moieties each have a first absorbance peak with FWHM of less than about 200 nm and an absorbance maximum (λₘₐₓ) between 330 nm+/−10 and 950 nm+/−10; and wherein an absorbance maximum (λₘₐₓ) of the first detectable moiety and an absorbance maximum (λₘₐₓ) of the second detectable moiety are separated by at least 20 nm. In some embodiments, the separation between the absorbance maximums (λₘₐₓ) of the first and second detectable moieties is at least 30 nm. In some embodiments, the separation between the absorbance maximums (λₘₐₓ) of the first and second detectable moieties is at least 45 nm. In some embodiments, the separation between the absorbance maximums (λₘₐₓ) of the first and second detectable moieties is at least 60 nm.

In some embodiments, the biological sample further comprises a second biomarker labeled with a third detectable moiety, where the first, second, and third detectable moieties have absorbance maximums (λₘₐₓ) which differ by at least 10 nm. In some embodiments, the first, second, and third detectable moieties have absorbance maximums (λₘₐₓ) which differ by at least 20 nm. In some embodiments, the first, second, and third detectable moieties have absorbance maximums (λₘₐₓ) which differ by at least 30 nm. In some embodiments, the first, second, and third detectable moieties have absorbance maximums (λₘₐₓ) which differ by at least 40 nm. In some embodiments, the first, second, and third detectable moieties have absorbance maximums (λₘₐₓ) which differ by at least 50 nm. In some embodiments, the first, second, and third detectable moieties have absorbance maximums (λₘₐₓ) which differ by at least 60 nm. In some embodiments, the first, second, and third detectable moieties have absorbance maximums (λₘₐₓ) which differ by at least 200 nm. In some embodiments, the first, second, and third detectable moieties have absorbance maximums (λₘₐₓ) which differ by at least 80 nm. In some embodiments, the first, second, and third detectable moieties have absorbance maximums (λₘₐₓ) which differ by at least 90 nm.

In some embodiments, the biological sample further comprises a third biomarker labeled with a fourth detectable moiety, where the first, second, third and fourth detectable moieties have absorbance maximums (λₘₐₓ) which differ by at least 10 nm. In some embodiments, the first, second, third and fourth detectable moieties have absorbance maximums (λₘₐₓ) which differ by at least 20 nm. In some embodiments, the first, second, third and fourth detectable moieties have absorbance maximums (λₘₐₓ) which differ by at least 30 nm. In some embodiments, the first, second, third and fourth detectable moieties have absorbance maximums (λₘₐₓ) which differ by at least 40 nm. In some embodiments, the first, second, third and fourth detectable moieties have absorbance maximums (λₘₐₓ) which differ by at least 50 nm. In some embodiments, the first, second, third and fourth detectable moieties have absorbance maximums (λₘₐₓ) which differ by at least 60 nm. In some embodiments, the first, second, third and fourth detectable moieties have absorbance maximums (λₘₐₓ) which differ by at least 70 nm. In some embodiments, the first, second, third and fourth detectable moieties have absorbance maximums (λₘₐₓ) which differ by at least 80 nm. In some embodiments, the first, second, third and fourth detectable moieties have absorbance maximums (λₘₐₓ) which differ by at least 90 nm.

In some embodiments, the first and second detectable moieties are selected from the group consisting of:

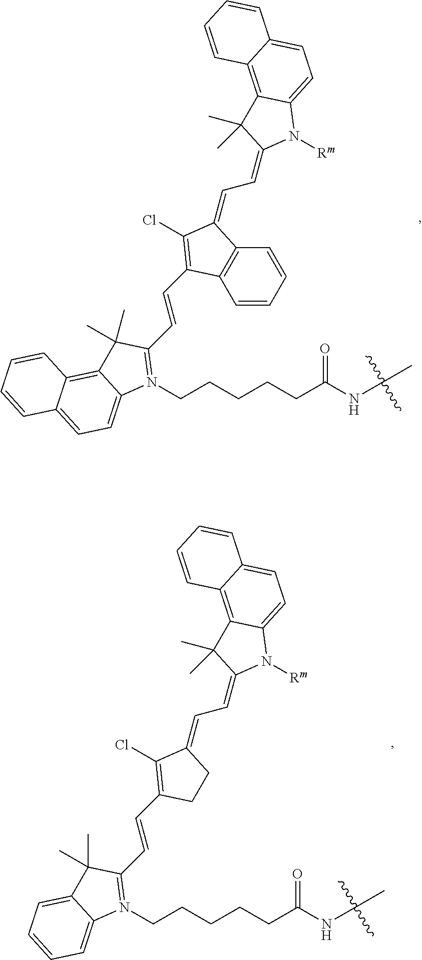

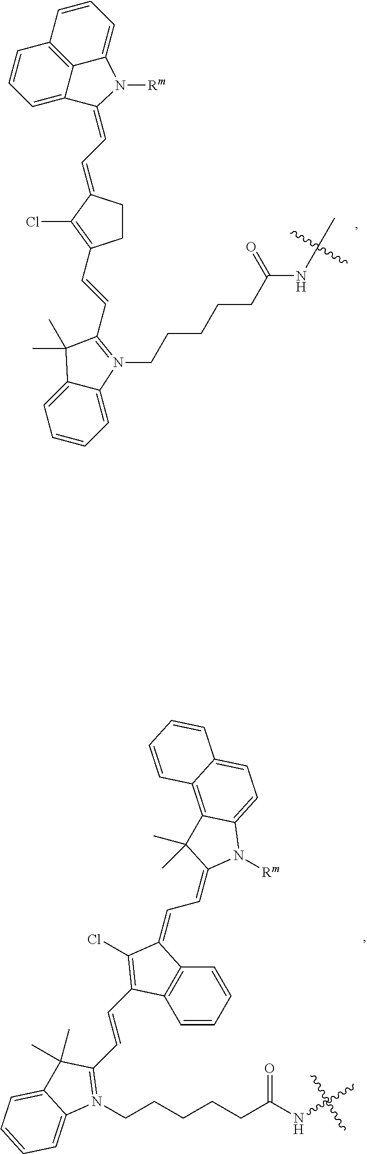

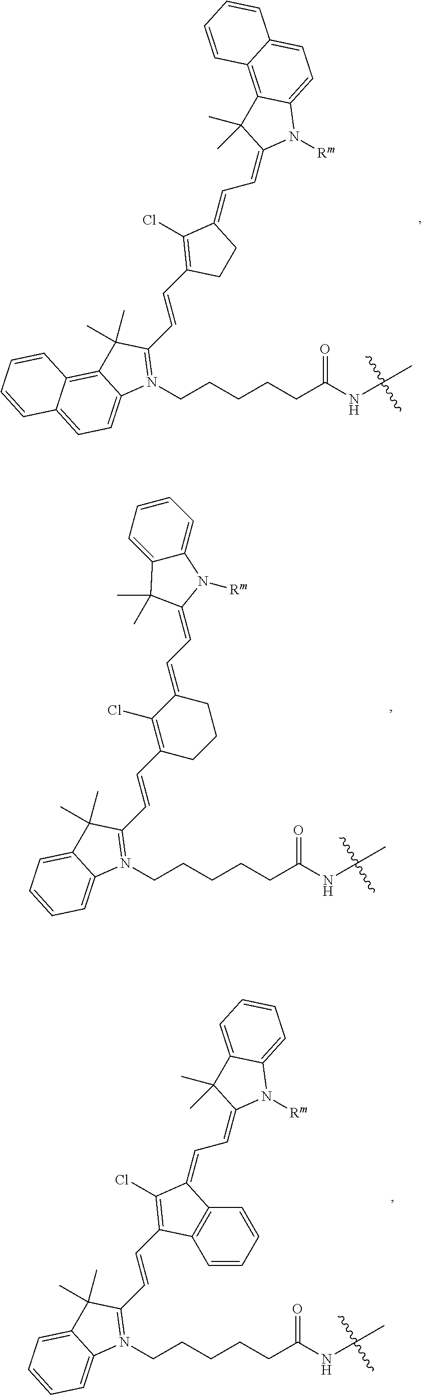

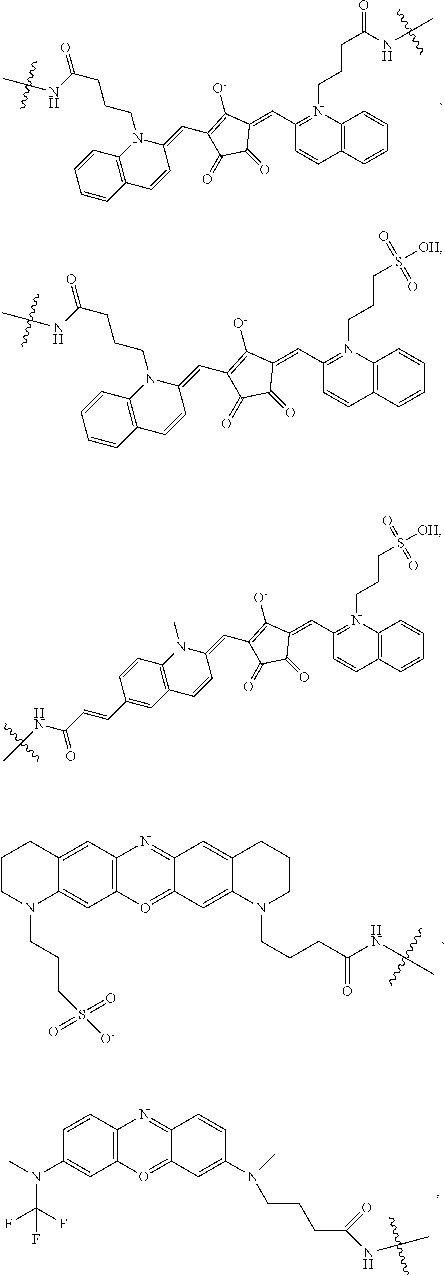

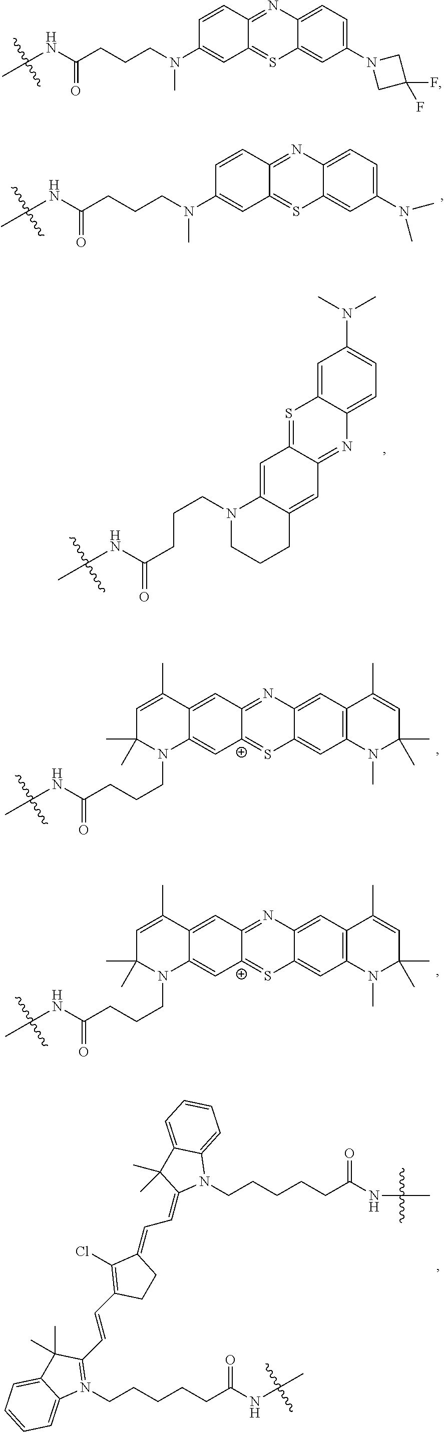

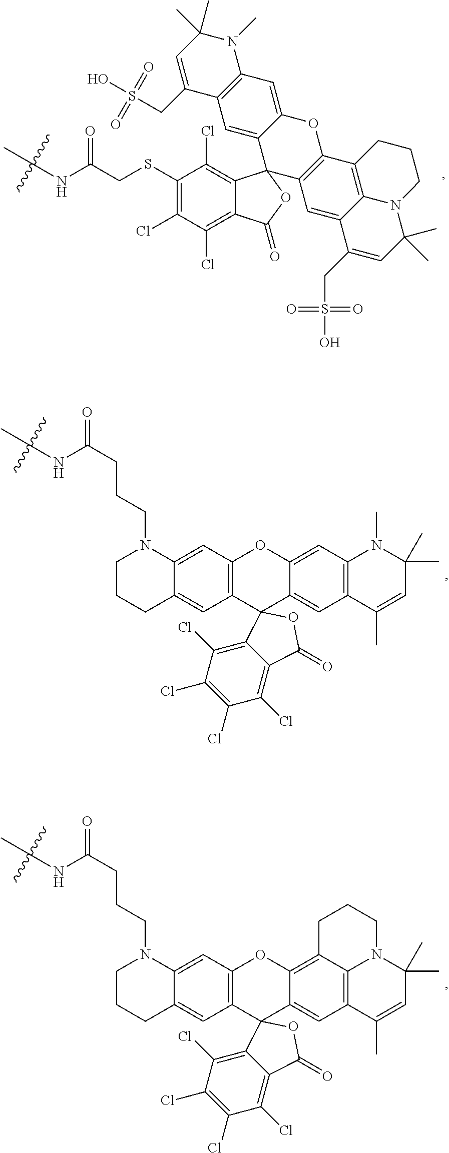 [SMILES: *NC(=O)CSc1c(Cl)c(Cl)c2c(c1Cl)C1(OC2=O)c2cc3c(cc2Oc2c1cc1c4c2CCCN4C(C)(C)C=C1CS(=O)(=O)O)N(C)C(C)(C)C=C3CS(=O)(=O)O ; *NC(=O)CCCN1CCCc2cc3c(cc21)Oc1cc2c(cc1C31OC(=O)c3c(Cl)c(Cl)c(Cl)c(Cl)c31)C(C)=CC(C)(C)N2C ; *NC(=O)CCCN1CCCc2cc3c(cc21)Oc1c(cc2c4c1CCCN4C(C)(C)C=C2C)C31OC(=O)c2c(Cl)c(Cl)c(Cl)c(Cl)c21]

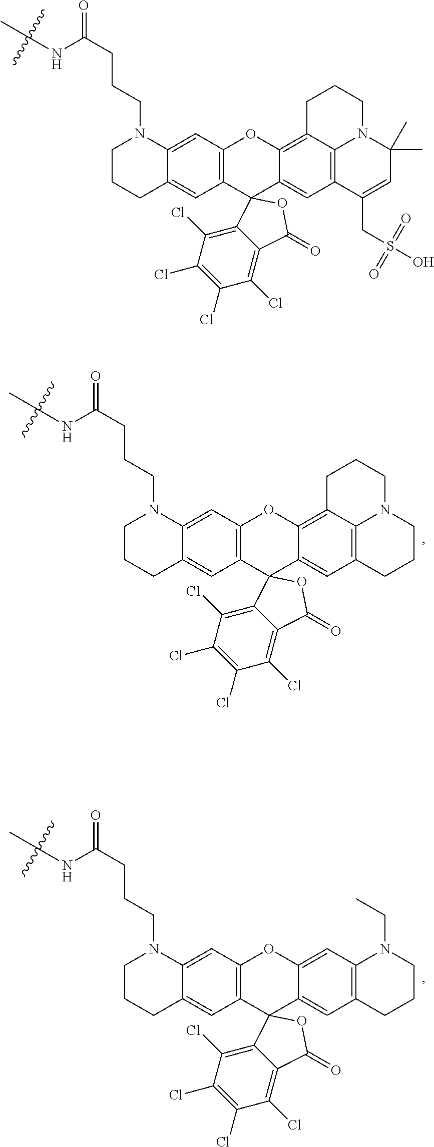 [SMILES: *NC(=O)CCCN1CCCc2cc3c(cc21)Oc1c(cc2c4c1CCCN4C(C)(C)C=C2CS(=O)(=O)O)C31OC(=O)c2c(Cl)c(Cl)c(Cl)c(Cl)c21 ; *NC(=O)CCCN1CCCc2cc3c(cc21)Oc1c(cc2c4c1CCCN4CCC2)C31OC(=O)c2c(Cl)c(Cl)c(Cl)c(Cl)c21 ; *NC(=O)CCCN1CCCc2cc3c(cc21)Oc1cc2c(cc1C31OC(=O)c3c(Cl)c(Cl)c(Cl)c(Cl)c31)CCCN2CC]

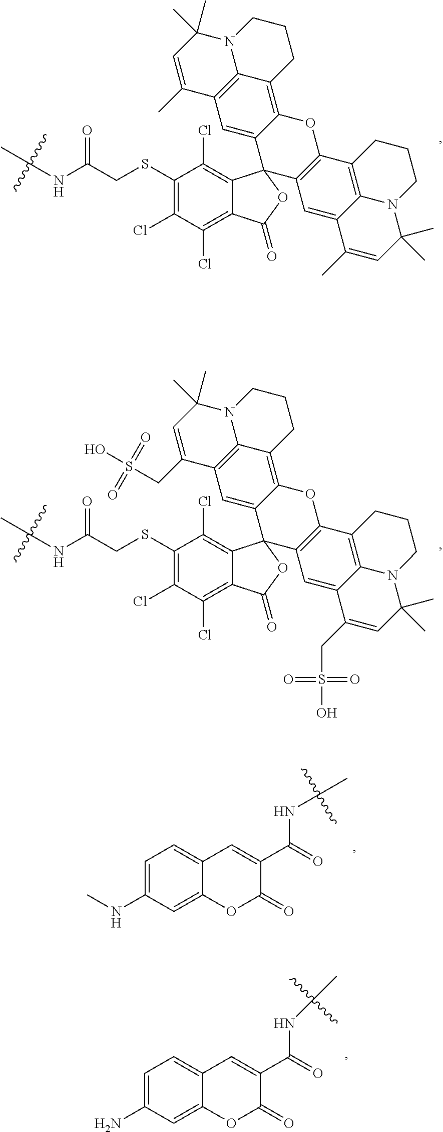

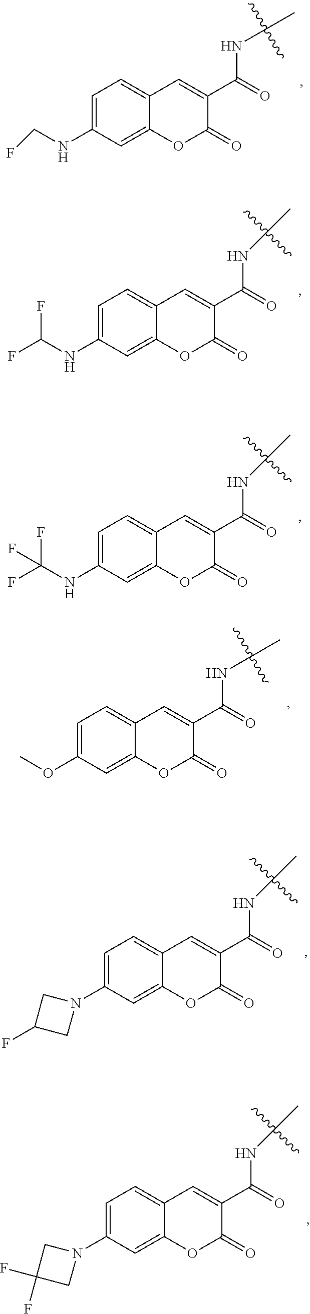 [SMILES: *NC(=O)c1cc2ccc(NCF)cc2oc1=O ; *NC(=O)c1cc2ccc(NC(F)F)cc2oc1=O ; *NC(=O)c1cc2ccc(NC(F)(F)F)cc2oc1=O ; *NC(=O)c1cc2ccc(OC)cc2oc1=O ; *NC(=O)c1cc2ccc(N3CC(F)C3)cc2oc1=O ; *NC(=O)c1cc2ccc(N3CC(F)(F)C3)cc2oc1=O]

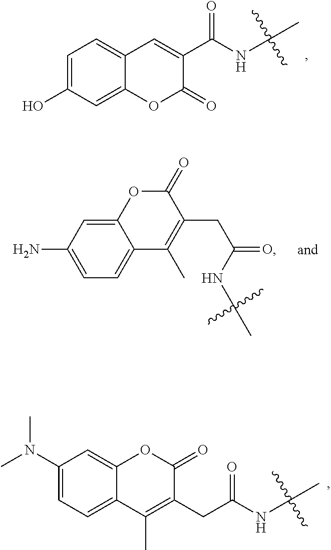 [SMILES: *NC(=O)c1cc2ccc(O)cc2oc1=O ; *NC(=O)Cc1c(C)c2ccc(N)cc2oc1=O ; *NC(=O)Cc1c(C)c2ccc(N(C)C)cc2oc1=O]

where the symbol “ [symbol] ” refers to the site in which the detectable moiety is conjugated to another moiety of a detectable conjugate.

A further aspect of the present disclosure is a biological sample comprising: (a) a first morphological marker labeled with a first detectable moiety; and (b) a first biomarker labeled with a second detectable moiety; wherein the first and second detectable moieties each have a first absorbance peak with FWHM of less than about 200 nm and an absorbance maximum (λₘₐₓ) between 330 nm+/−10 and 950 nm+/−10; and wherein an absorbance maximum (λₘₐₓ) of the first detectable moiety and an absorbance maximum (λₘₐₓ) of the second detectable moiety are separated by at least 20 nm; wherein the biological sample is prepared by: contacting the biological sample with a first primary antibody specific to the first morphological marker; contacting the biological sample with a first secondary antibody specific to the first primary antibody, wherein the first secondary antibody is conjugated to an enzyme; contacting the biological sample with a first detectable conjugate comprising (a) a tyramide moiety, a quinone methide precursor moiety, or a derivative or analog of a tyramide moiety or quinone methide precursor moiety; and (b) the first detectable moiety; contacting the biological sample with a second primary antibody specific to the first biomarker; contacting the biological sample with a second secondary antibody specific to the second primary antibody, wherein the second secondary antibody is conjugated to an enzyme; and contacting the biological sample with a second detectable conjugate comprising (a) a tyramide moiety, a quinone methide precursor moiety, or a derivative or analog of a tyramide moiety or quinone methide precursor moiety; and (b) the second detectable moiety. In some embodiments, the biological sample is free of hematoxylin. In some embodiments, the biological sample is free of a special stain.

In some embodiments, the separation between the absorbance maximums (λₘₐₓ) of the first and second detectable moieties is at least 30 nm. In some embodiments, the separation between the absorbance maximums (λₘₐₓ) of the first and second detectable moieties is at least 45 nm. In some embodiments, the separation between the absorbance maximums (λₘₐₓ) of the first and second detectable moieties is at least 60 nm. In some embodiments, the separation between the absorbance maximums (λₘₐₓ) of the first and second detectable moieties is at least 75 nm. In some embodiments, the separation between the absorbance maximums (λₘₐₓ) of the first and second detectable moieties is at least 90 nm.

In some embodiments, the first morphological marker is selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers. In some embodiments, the first morphological marker is selected DNA and/or histone proteins.

In some embodiments, the first detectable moiety comprises a coumarin core. In some embodiments, the second detectable moiety is within the visible spectrum or within the infrared spectrum. In some embodiments, the second detectable moiety is within the ultraviolet spectrum. In some embodiments, first and second detectable moieties have absorbance maximums (λₘₐₓ) that are separated by at least 20 nm.

In some embodiments, the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core. In some embodiments, the second detectable moiety is within the ultraviolet spectrum or within the infrared spectrum. In some embodiments, second detectable moiety is within the visible spectrum. In some embodiments, the first and second detectable moieties have absorbance maximums (λₘₐₓ) that are separated by at least 20 nm.

In some embodiments, the first detectable moiety comprises a heptamethine cyanine core or a croconate core. In some embodiments, the second detectable moiety is within the visible spectrum or within the ultraviolet spectrum. In some embodiments, the second detectable moiety is within the infrared spectrum. In some embodiments, the first and second detectable moieties have absorbance maximums (λₘₐₓ) that are separated by at least 20 nm.

Another aspect of the present disclosure is a biological sample comprising: (a) a first morphological marker labeled with a first detectable moiety; and (b) a first biomarker labeled with a second detectable moiety; wherein the first and second detectable moieties each have a first absorbance peak with FWHM of less than about 200 nm and an absorbance maximum (λₘₐₓ) between 330 nm+/−10 and 950 nm+/−10; and wherein an absorbance maximum (λₘₐₓ) of the first detectable moiety and an absorbance maximum (λₘₐₓ) of the second detectable moiety are separated by at least 20 nm; wherein the biological sample is prepared by: contacting the biological sample with a first primary antibody specific to the first morphological marker; contacting the biological sample with a first secondary antibody specific to the first primary antibody, wherein the first secondary antibody is conjugated to an enzyme; contacting the biological sample with a first tissue reactive moiety comprising (a) a tyramide moiety, a quinone methide precursor moiety, or a derivative or analog of a tyramide moiety or quinone methide precursor moiety; and (b) a first reactive functional group capable of participating in a click chemistry reaction; contacting the biological sample with a first detectable conjugate comprising: (a) the first detectable moiety; and (b) a second reactive functional group; contacting the biological sample with a second primary antibody specific to the first biomarker; contacting the biological sample with a second secondary antibody specific to the second primary antibody, wherein the second secondary antibody is conjugated to an enzyme; contacting the biological sample with a second tissue reactive moiety comprising (a) a tyramide moiety, a quinone methide precursor moiety, or a derivative or analog of a tyramide moiety or quinone methide precursor moiety; and (b) a first reactive functional group capable of participating in a click chemistry reaction; contacting the biological sample with a second detectable conjugate comprising: (a) the second detectable moiety; and (b) a second reactive functional group. In some embodiments, the biological sample is free of hematoxylin. In some embodiments, the biological sample is free of a special stain.

In some embodiments, the separation between the absorbance maximums (λₘₐₓ) of the first and second detectable moieties is at least 30 nm. In some embodiments, the separation between the absorbance maximums (λₘₐₓ) of the first and second detectable moieties is at least 45 nm. In some embodiments, the separation between the absorbance maximums (λₘₐₓ) of the first and second detectable moieties is at least 60 nm. In some embodiments, the separation between the absorbance maximums (λₘₐₓ) of the first and second detectable moieties is at least 75 nm. In some embodiments, the separation between the absorbance maximums (λₘₐₓ) of the first and second detectable moieties is at least 90 nm.

In some embodiments, the first morphological marker is selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers. In some embodiments, the first morphological marker is selected from the group consisting of DNA and histone proteins.

In some embodiments, the first detectable moiety comprises a coumarin core. In some embodiments, the second detectable moiety is within the visible spectrum or within the infrared spectrum. In some embodiments, the second detectable moiety is within the ultraviolet spectrum. In some embodiments, the first and second detectable moieties have absorbance maximums (λₘₐₓ) that are separated by at least 20 nm.

In some embodiments, the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core. In some embodiments, the second detectable moiety is within the ultraviolet spectrum or within the infrared spectrum. In some embodiments, the second detectable moiety is within the visible spectrum. In some embodiments, the first and second detectable moieties have absorbance maximums (λₘₐₓ) that are separated by at least 20 nm.

In some embodiments, the first detectable moiety comprises a heptamethine cyanine core or a croconate core. In some embodiments, the second detectable moiety is within the visible spectrum or within the ultraviolet spectrum. In some embodiments, the second detectable moiety is within the infrared spectrum. In some embodiments, the first and second detectable moieties have absorbance maximums (λₘₐₓ) that are separated by at least 20 nm.

A still further aspect of the present disclosure is a biological sample comprising: (a) a first morphological marker labeled with a first detectable moiety; and (b) a first biomarker labeled with a second detectable moiety; wherein the first and second detectable moieties each have a first absorbance peak with FWHM of less than about 200 nm and an absorbance maximum (λₘₐₓ) between 330 nm+/−10 and 950 nm+/−10; and wherein an absorbance maximum (λₘₐₓ) of the first detectable moiety and an absorbance maximum (λₘₐₓ) of the second detectable moiety are separated by at least 20 nm; wherein the biological sample is prepared by: contacting the biological sample with a first primary antibody specific to the first morphological marker; contacting the biological sample with a first secondary antibody specific to the first primary antibody, wherein the first secondary antibody is conjugated to an enzyme; contacting the biological sample with a first detectable conjugate comprising (a) a tyramide moiety, a quinone methide precursor moiety, or a derivative or analog of a tyramide moiety or quinone methide precursor moiety; and (b) the first detectable moiety; contacting the biological sample with a second primary antibody specific to the first biomarker; contacting the biological sample with a second secondary antibody specific to the second primary antibody, wherein the second secondary antibody is conjugated to an enzyme; contacting the biological sample with a first tissue reactive moiety comprising (a) a tyramide moiety, a quinone methide precursor moiety, or a derivative or analog of a tyramide moiety or quinone methide precursor moiety; and (b) a first reactive functional group capable of participating in a click chemistry reaction; contacting the biological sample with a second detectable conjugate comprising: (a) the second detectable moiety; and (b) a second reactive functional group. In some embodiments, the biological sample is free of hematoxylin. In some embodiments, the biological sample is free of a special stain.

In some embodiments, the biological sample is further prepared by contacting the biological sample with a third primary antibody specific to a second biomarker. In some embodiments, the first and second detectable conjugates are selected from the group consisting of:

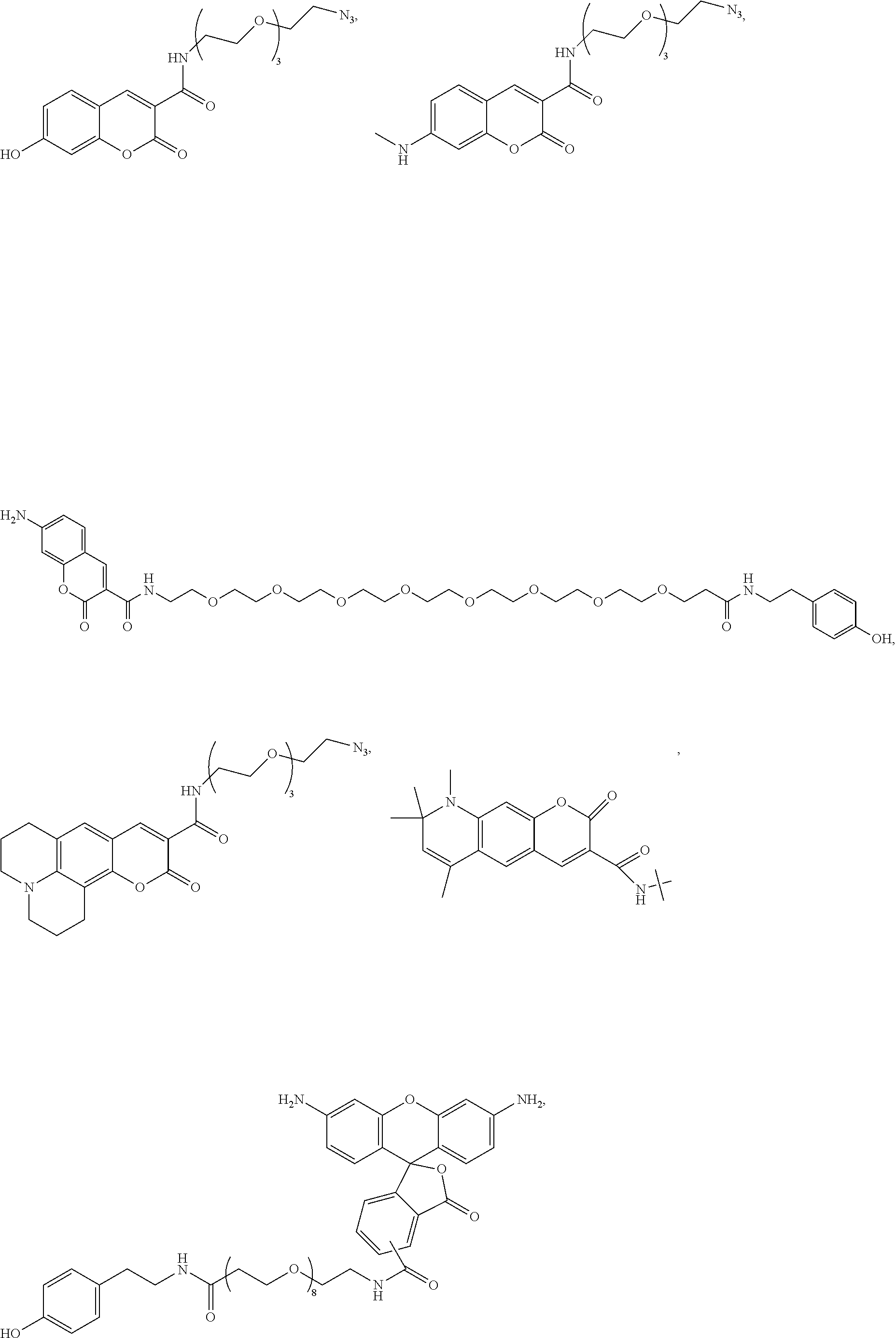

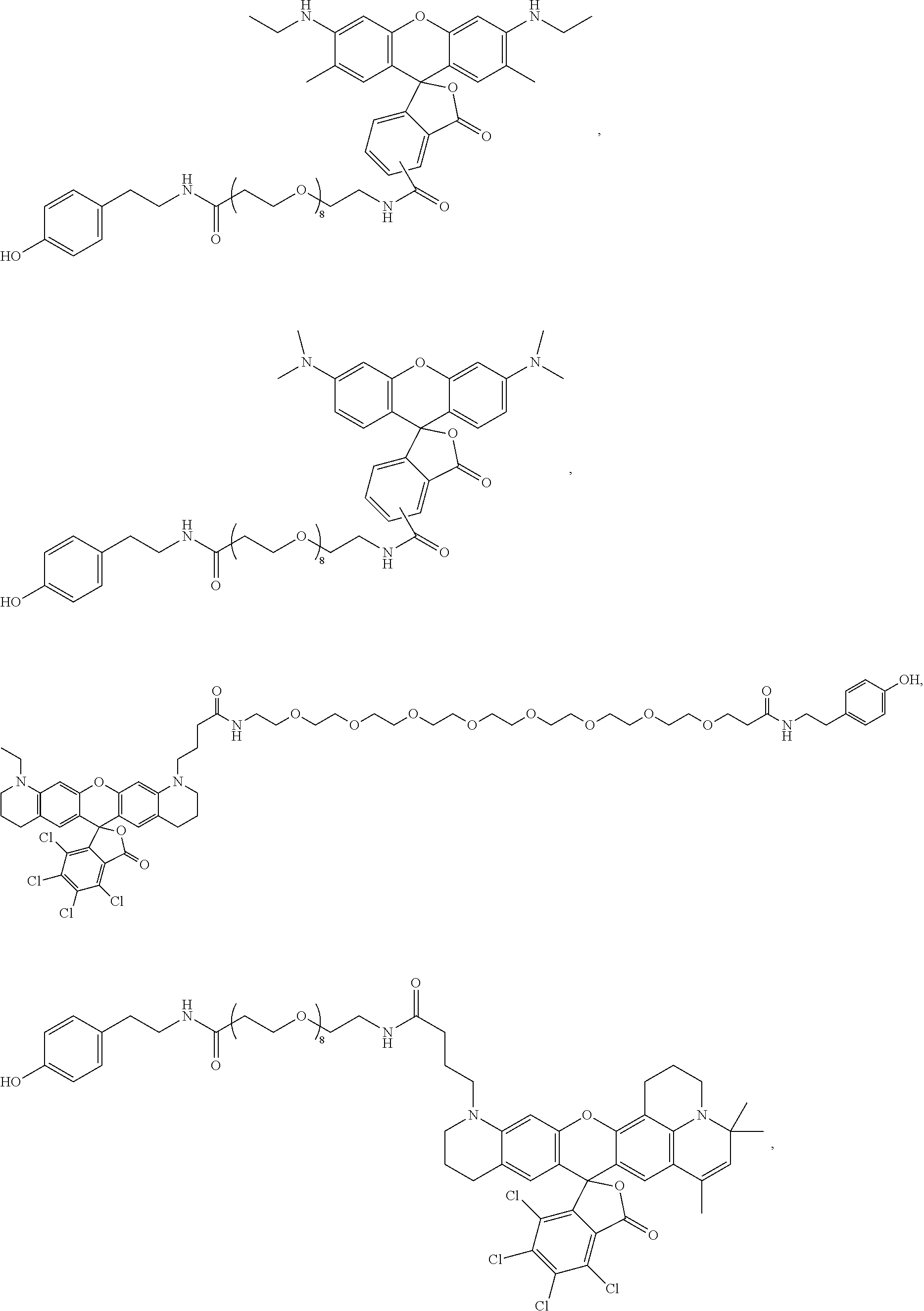

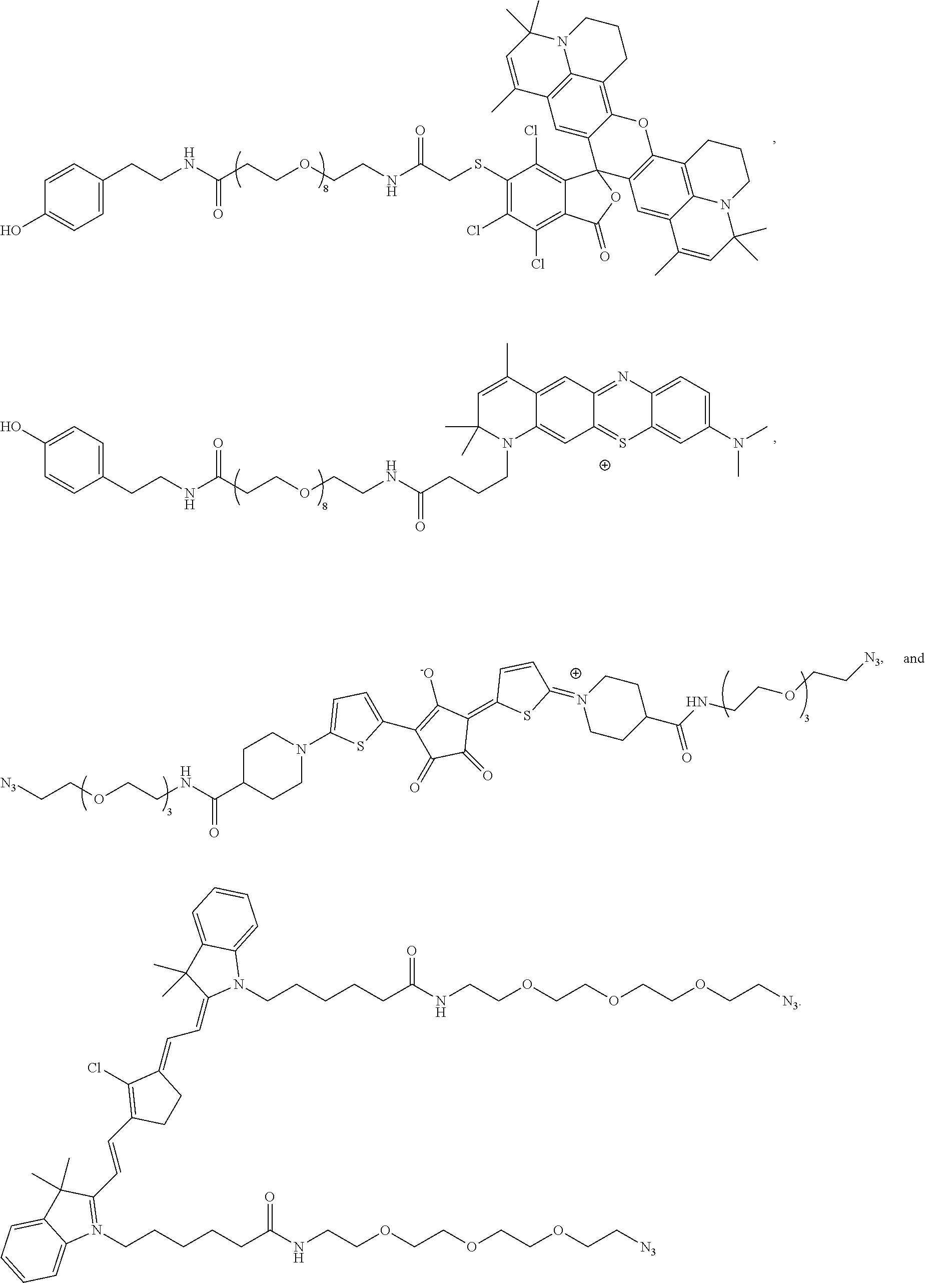

Another aspect of the present disclosure is a biological sample comprising: (a) a first morphological marker labeled with a first detectable moiety; and (b) a first biomarker labeled with a second detectable moiety; wherein the first and second detectable moieties each have a first absorbance peak with FWHM of less than about 200 nm and an absorbance maximum (λₘₐₓ) between 330 nm+/−10 and 950 nm+/−10; and wherein an absorbance maximum (λₘₐₓ) of the first detectable moiety and an absorbance maximum (λₘₐₓ) of the second detectable moiety are separated by at least 20 nm; wherein the biological sample is prepared by: contacting the biological sample with a first primary antibody specific to the first morphological marker; contacting the biological sample with a first secondary antibody specific to the first primary antibody, wherein the first secondary antibody is conjugated to an enzyme; contacting the biological sample with a first tissue reactive moiety comprising (a) a tyramide moiety, a quinone methide precursor moiety, or a derivative or analog of a tyramide moiety or quinone methide precursor moiety; and (b) a first reactive functional group capable of participating in a click chemistry reaction; contacting the biological sample with a first detectable conjugate comprising: (a) the first detectable moiety; and (b) a second reactive functional group; contacting the biological sample with a second primary antibody specific to the first biomarker; contacting the biological sample with a second secondary antibody specific to the second primary antibody, wherein the second secondary antibody is conjugated to an enzyme; and contacting the biological sample with a second detectable conjugate comprising (a) a tyramide moiety, a quinone methide precursor moiety, or a derivative or analog of a tyramide moiety or quinone methide precursor moiety; and (b) the second detectable moiety. In some embodiments, the biological sample is free of hematoxylin. In some embodiments, the biological sample is free of a special stain.

In some embodiments, the process of preparing the biological sample further comprises contacting the biological sample with a third primary antibody specific to a second biomarker.

In some embodiments, the first and second detectable moieties are selected from the group consisting of:

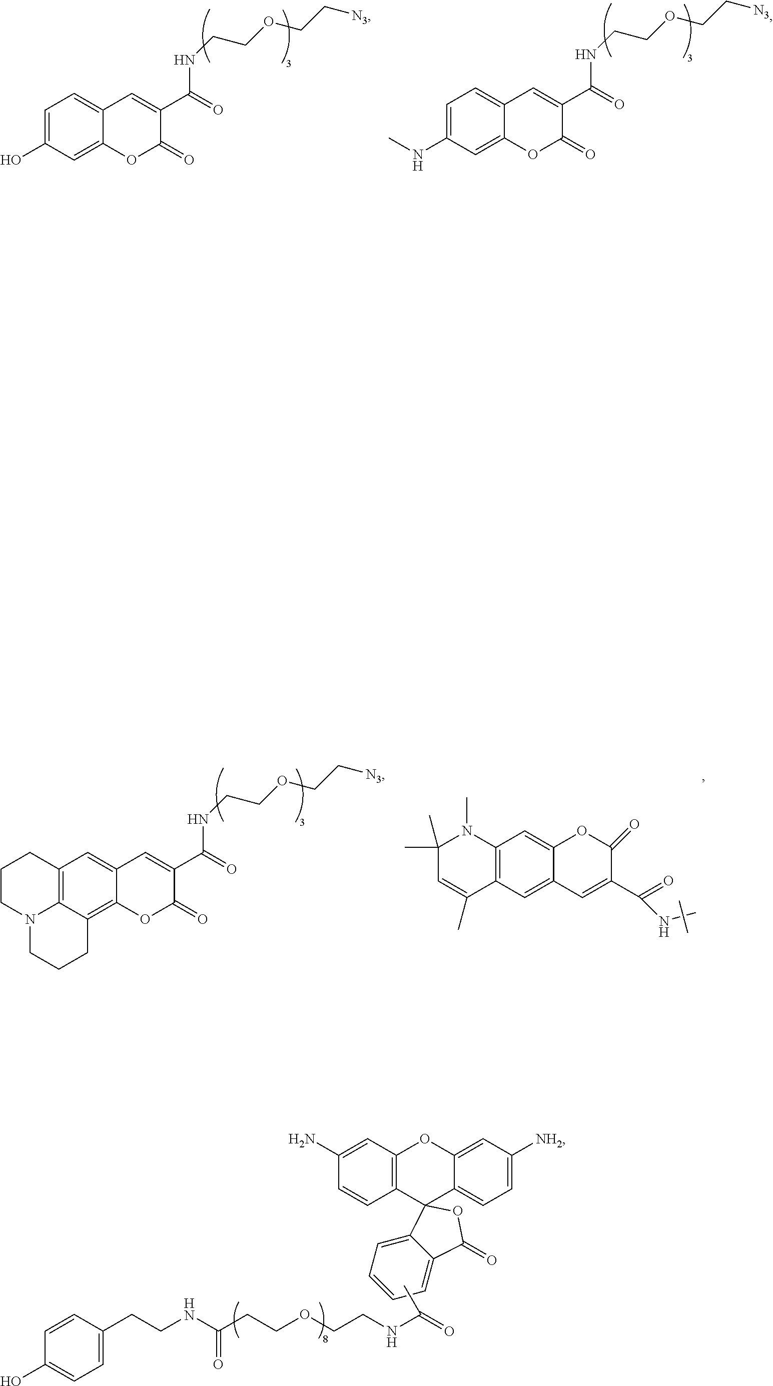

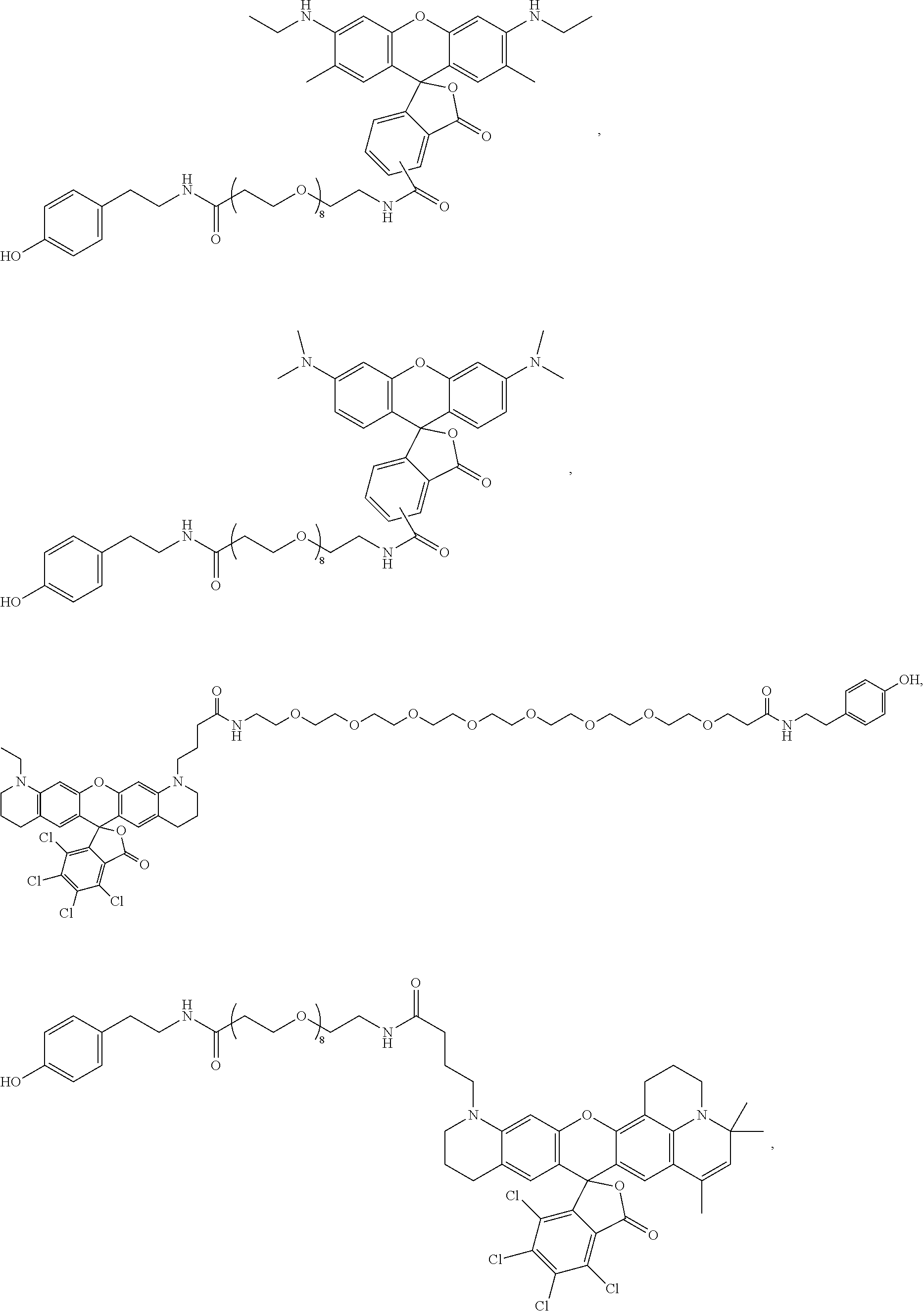

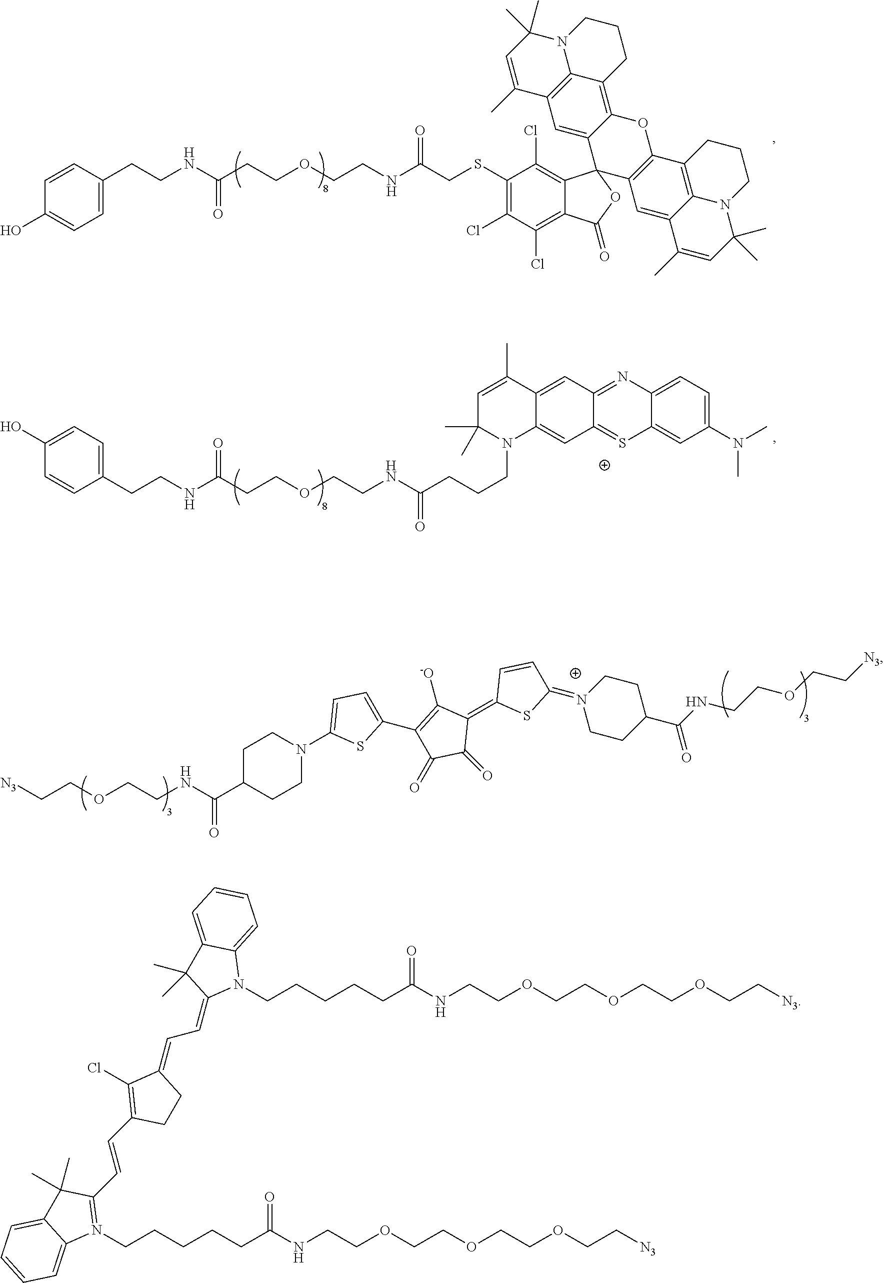

A further aspect of the present disclosure is a kit comprising: (a) a primary antibody specific to a first morphological marker; (b) a primary antibody specific to a first biomarker; and (c) at least two detection conjugates, wherein the at least two detection conjugates each include a different detectable moiety, wherein each detectable moiety has a first absorbance peak with FWHM of less than about 200 nm and an absorbance maximum (λₘₐₓ) between 330 nm+/−10 and 950 nm+/−10; and wherein an absorbance maximum (λₘₐₓ) of a first detectable moiety and an absorbance maximum (λₘₐₓ) of a second detectable moiety are separated by at least 20 nm.

In some embodiments, the at least two detection conjugates are selected from the group consisting of:

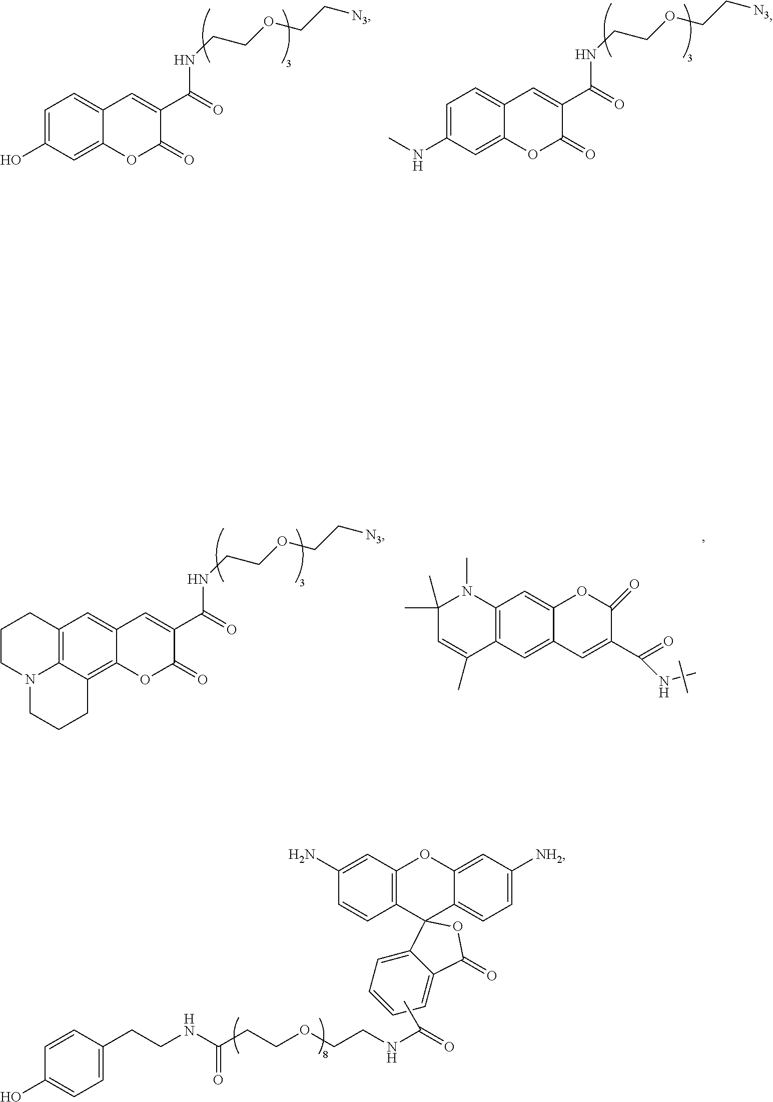

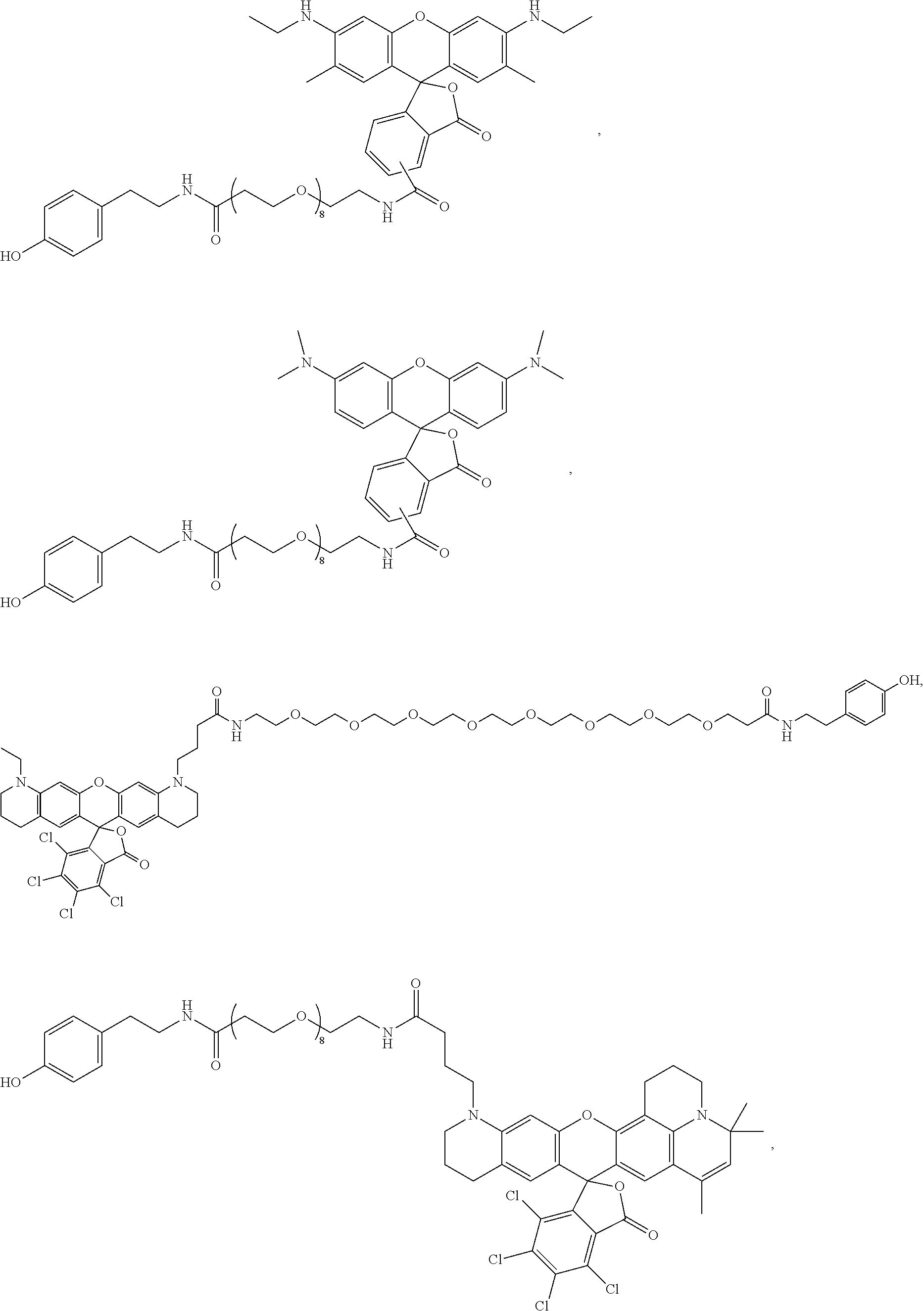

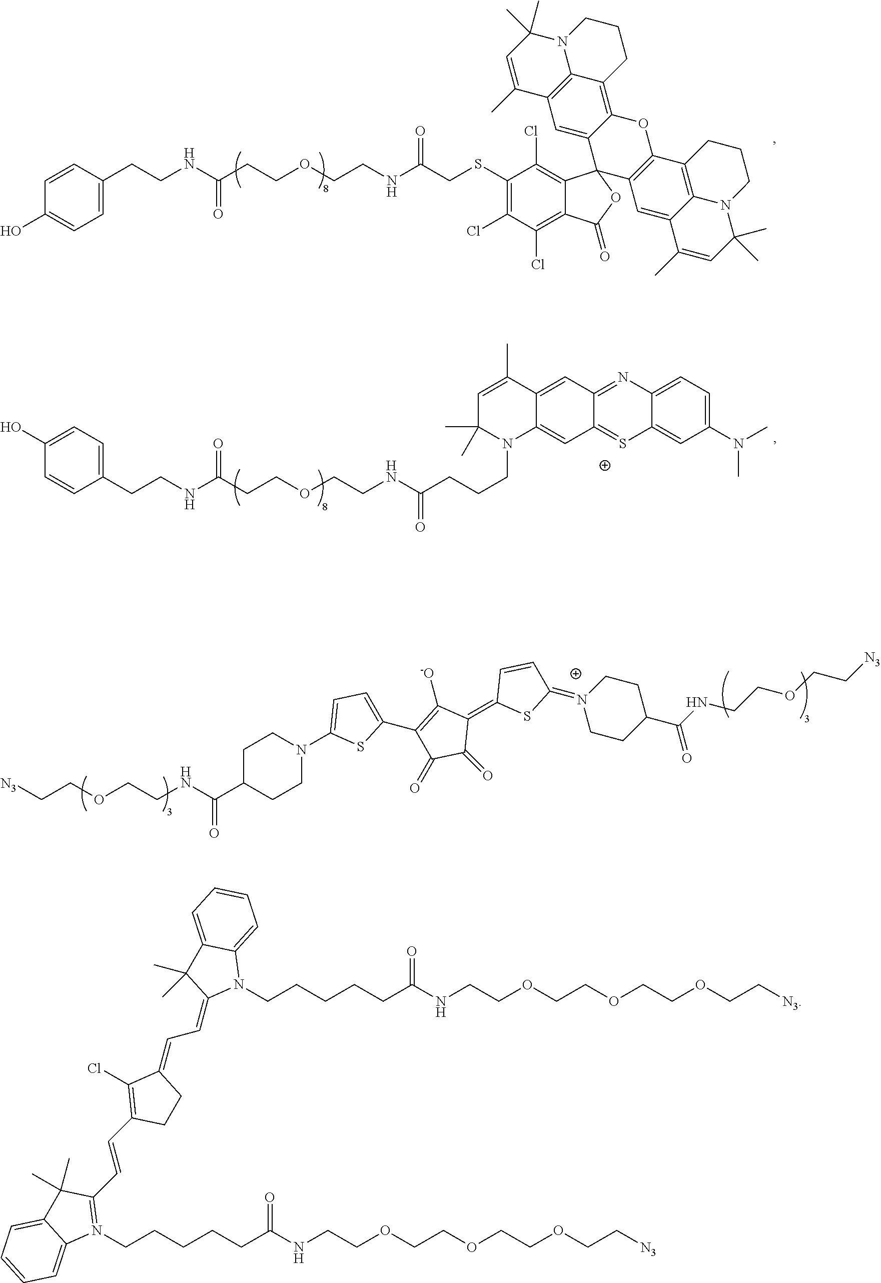

### BRIEF DESCRIPTION OF THE FIGURES

The patent or application file contains at least one drawing executed in color. Copies of this patent or patent application publication with color drawings will be provided to the Office upon request and the payment of the necessary fee.

FIG. 1 illustrates a method of detecting signals corresponding to one or more morphological markers and one or more biomarkers in a biological sample in accordance with one embodiment of the present disclosure.

FIG. 2A illustrates methods of labeling one or more morphological markers and/or one or more biomarkers with a detectable moiety in accordance with one embodiment of the present disclosure.

FIG. 2B illustrates methods of labeling one or more morphological markers and/or one or more biomarkers with a detectable moiety in accordance with one embodiment of the present disclosure.

FIG. 2C illustrates methods of labeling one or more morphological markers and/or one or more biomarkers with a detectable moiety in accordance with one embodiment of the present disclosure.

FIG. 2D illustrates methods of labeling one or more morphological markers and/or one or more biomarkers with a detectable moiety in accordance with one embodiment of the present disclosure.

FIG. 3 illustrates a method of detecting signals corresponding to one or more morphological markers and one or more biomarkers in a biological sample, where the method utilizes detectable conjugates including (i) a detectable moiety, and (ii) a tyramide moiety, a derivative of a tyramide moiety, a quinone methide precursor moiety, or a derivative of a quinone methide precursor moiety, in accordance with one embodiment of the present disclosure.

FIG. 4 illustrates the deposition of a conjugate including a quinone methide precursor moiety in accordance with one embodiment of the present disclosure.

FIG. 5 illustrates the deposition of a conjugate including a tyramide moiety in accordance with one embodiment of the present disclosure.

FIG. 6 illustrates a method of detecting signals corresponding to one or more morphological markers and one or more biomarkers in a biological sample in accordance with one embodiment of the present disclosure.

FIG. 7 illustrates a method of detecting signals corresponding to one or more morphological markers and one or more biomarkers in a biological sample, where the method utilizes detectable conjugates including (i) a detectable moiety, and (ii) reactive functional groups capable of participating in a click chemistry reaction, in accordance with one embodiment of the present disclosure.

FIG. 8 illustrates the deposition of a conjugate including a quinone methide precursor moiety in accordance with one embodiment of the present disclosure.

FIG. 9 illustrates the deposition of a conjugate including a tyramide moiety in accordance with one embodiment of the present disclosure.

FIG. 10 provides a plot of several conventional chromogens and the conventional chemical dye hematoxylin, all having broad spectral absorbances.

FIG. 11 provides a comparison of the broad spectral absorbance of the conventional dye hematoxylin to several different detectable moieties according to the disclosure having relatively narrower spectral absorbances.

FIG. 12 illustrates brightfield microscope images of a formalin-fixed paraffin-embedded (FFPE) tonsil tissue specimen, stained with both hematoxylin and anti-ds DNA IHC using the Cy7 covalently deposited chromophore (CDC). The images were recorded at 20× magnification with a monochrome CMOS camera using illumination from a 770 nm light emitting diode (LED) on the left side of the figure, and with a color (RGB) CMOS camera using white light illumination from a tungsten halogen lamp on the right side of the figure. At 770 nm Cy7 absorbs strongly and hematoxylin has negligible absorbance, so the image on the left represent staining by the anti-ds DNA IHC. Cy7 absorbs considerably less visible light while hematoxylin absorbs broadly in the visible range such that the image on the right reflects hematoxylin absorbance. Comparison of these two images of the same microscope field shows that anti-ds DNA IHC provides specific nuclear staining of all cells in the same manner as hematoxylin, and that anti-ds DNA can therefore replace hematoxylin as an effective nuclear counterstain.

FIG. 13 illustrates brightfield microscope images of an FFPE tonsil tissue specimen, stained with both hematoxylin and anti-histone IHC using the Cy7 CDC. The images were recorded at 20× magnification with a monochrome CMOS camera using illumination from a 770 nm LED on the left side of the figure, and with a color (RGB) CMOS camera using white light illumination from a tungsten halogen lamp on the right side of the figure. At 770 nm Cy7 absorbs strongly and hematoxylin has negligible absorbance, so the image on the left represents staining by the anti-histone IHC. Cy7 absorbs considerably less visible light while hematoxylin absorbs broadly in the visible range such that the image on the right reflects hematoxylin absorbance. Comparison of these two images of the same microscope field shows that anti-histone IHC provides specific nuclear staining of all cells in the same manner as hematoxylin, and that anti-histone can therefore replace hematoxylin as an effective nuclear counterstain.

FIG. 14 illustrates brightfield microscope images of the same FFPE tonsil tissue specimen, stained with both hematoxylin and anti-ds DNA IHC using the Cy7 CDC, as shown in FIG. 12. The images were recorded at 20× magnification with a monochrome CMOS camera using illumination from a 770 nm LED on the left side of the figure, and using illumination from a 595 nm LED on the right side of the figure. Hematoxylin strongly absorbs at 595 nm while Cy7 has minimal absorbance such that the image on the right reflects hematoxylin absorbance and the image on the right side reflects the anti-ds DNA IHC staining with the Cy7 CDC, as in FIG. 12. Presenting both the hematoxylin and anti-ds DNA IHC staining in monochrome provides a better comparison of staining intensities across the microscope field. The staining patterns for antibody and HTX look similar, as in FIG. 12, but the antibody staining for the anti-ds IHC appears to provide a more uniform level of nuclear staining across the field. Since the counterstain purpose is often to identify all cell nuclei without respect to cell type, uniform staining is a desirable property, providing an unexpected advantage of the IHC-based counterstain over the conventional hematoxylin counterstain.

FIG. 15 illustrates brightfield microscope images of the same FFPE tonsil tissue specimen, stained with both hematoxylin and anti-histone IHC using the Cy7 CDC, as shown in FIG. 13. The images were recorded at 20× magnification with a monochrome CMOS camera using illumination from a 770 nm LED on the left side of the figure, and using illumination from a 595 nm LED on the right side of the figure. Hematoxylin strongly absorbs at 595 nm while Cy7 has minimal absorbance such that the image on the right reflects hematoxylin absorbance and the image on the right side reflects the anti-histone IHC staining with the Cy7 CDC, as in FIG. 13. Presenting both the hematoxylin and anti-histone IHC staining in monochrome provides a better comparison of staining intensities across the microscope field. The staining patterns for antibody and hematoxylin look similar, as in FIG. 13, but the antibody staining for the anti-histone IHC appears to provide a more uniform level of nuclear staining across the field. Since the counterstain purpose is often to identify all cell nuclei without respect to cell type, uniform staining is a desirable property, providing an unexpected advantage of the IHC-based counterstain over the conventional hematoxylin counterstain.

FIG. 16 shows a comparison of the uniformity of staining using conventional hematoxylin staining and counterstaining according to the disclosure.

FIG. 17 provides a comparison of the broad spectral absorbance of hematoxylin to several different detectable moieties having relatively narrower spectral absorbances.

FIG. 18 illustrates color images of Rhod614 (left panel) and Rhod634 (right panel) CDCs used in IHC with anti-ds DNA, on FFPE tonsil. Comparison with color images in FIG. 12 and FIG. 13 (right panels) shows the color similarity between hematoxylin and these two CDCs and demonstrates that either of these two CDCs might provide a hematoxylin counterstain replacement of similar coloration. Coloration similar to hematoxylin provides a familiar viewing experience for the microscopist, but is not necessary. Both CDCs, however, do provide narrower absorbance bands than hematoxylin, as demonstrated in FIG. 16, which reduces spectral overlaps, thereby improving visual color distinction and spectral unmixing of multiplex IHC images.

FIG. 19 illustrates color images of Rhod614 (left panel) and Rhod634 (right panel) CDCs used in IHC with anti-histone, on FFPE tonsil. Comparison with color images in FIG. 12 and FIG. 13 (right panels) shows the color similarity between hematoxylin and these two CDCs and demonstrates that either of these two CDCs might provide a hematoxylin counterstain replacement of similar coloration. Coloration similar to hematoxylin provides a familiar viewing experience for the microscopist, but is not necessary. Both CDCs, however, do provide narrower absorbance bands than hematoxylin, as demonstrated in FIG. 16, which reduces spectral overlaps, thereby improving visual color distinction and spectral unmixing of multiplex IHC images.

FIG. 20 provides a comparison of the spectral absorbances of several detectable moieties according to the disclosure.

FIG. 21 show a diagram comparing the spectral absorbances of several detectable moieties according to the disclosure.

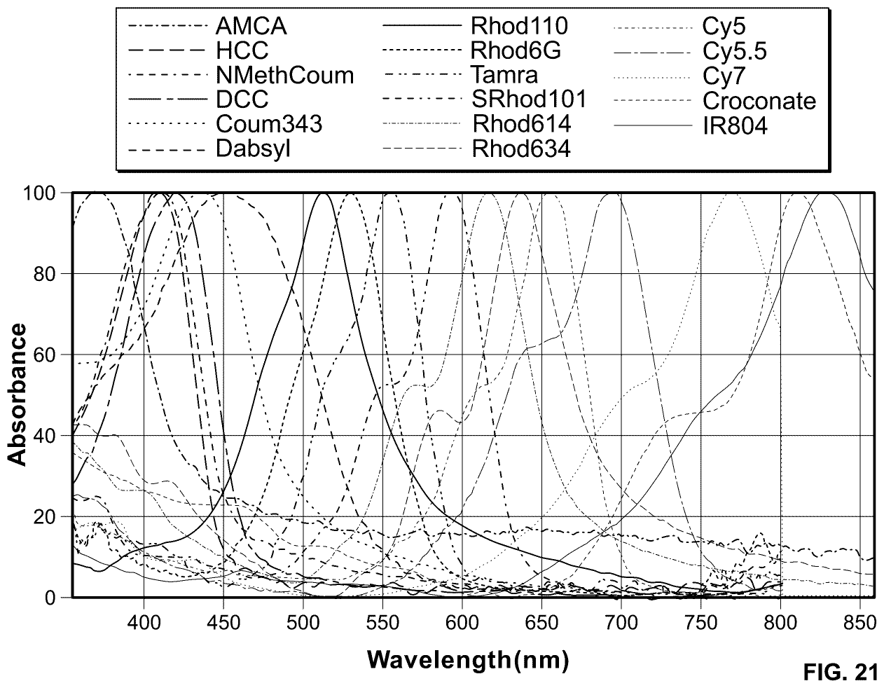

FIG. 22 shows 4 images recorded on a monochrome camera (dual-camera system), where the illumination channels were selected to align near the absorbance maxima of dabsyl, TAMRA, Rhod634, and Cy5.5, respectively. The fifth image is of the same microscope field using white light illumination recorded on a color camera (dual-camera system).

FIG. 23 shows a set of images of a tissue section was stained with the conventional hematoxylin counterstain in place of the anti-ds DNA counterstain, where the images of transmitted light using the 438, 549, 620, and 689 nm filtered LEDs are presented in the first four images from left to right, respectively. These illumination channels were selected to align near the absorbance maxima of dabsyl, TAMRA, hematoxylin, and Cy5.5, respectively. The fifth image is of the same microscope field using white light illumination recorded on a color camera (dual-camera system).

FIG. 24 shows results when hematoxylin staining time (5 s) was selected to provide similar counterstain absorbance of both hematoxylin and a Rhod634 counterstain according to the disclosure as shown by the absorbance spectra of these two sections.

FIG. 25 shows images of three different microscope fields from left to right, with the top images recorded under 525 nm LED illumination, where eosin absorbs light, and the corresponding lower images recorded under 770 nm LED illumination, where Cy7 absorbs light, reflecting the presence of actin.

FIG. 26 shows monochrome fluorescence images recorded on FFPE tonsil tissue stained with anti-ds DNA IHC using Cy7 CDC, TAMRA CDC, and AMCA CDC at 1/10 the typical chromophore concentrations.

FIG. 27 shows the excitation and emission spectra of DAPI and AMCA.

### DETAILED DESCRIPTION

Disclosed herein are detectable moieties and detectable conjugates comprising one or more detectable moieties. In some embodiments, the disclosed detectable moieties have a narrow wavelength and are suitable for multiplexing.

### Definitions

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein, alkaline phosphatase (AP) is an enzyme that removes (by hydrolysis) and transfers phosphate group organic esters by breaking the phosphate-oxygen bond, and temporarily forming an intermediate enzyme-substrate bond. For example, AP hydrolyzes naphthol phosphate esters (a substrate) to phenolic compounds and phosphates. The phenols couple to colorless diazonium salts (chromogen) to produce insoluble, colored azo dyes. In another embodiment, the AP hydrolyzes

As used herein, the term “antibody,” occasionally abbreviated “Ab,” refers to immunoglobulins or immunoglobulin-like molecules, including by way of example and without limitation, IgA, IgD, IgE, IgG and IgM, combinations thereof, and similar molecules produced during an immune response in any vertebrate, (e.g., in mammals such as humans, goats, rabbits and mice) and antibody fragments that specifically bind to a molecule of interest (or a group of highly similar molecules of interest) to the substantial exclusion of binding to other molecules. Antibody further refers to a polypeptide ligand comprising at least a light chain or heavy chain immunoglobulin variable region which specifically recognizes and binds an epitope of an antigen. Antibodies may be composed of a heavy and a light chain, each of which has a variable region, termed the variable heavy (VH) region and the variable light (VL) region. Together, the VH region and the VL region are responsible for binding the antigen recognized by the antibody. The term antibody also includes intact immunoglobulins and the variants and portions of them well known in the art.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, nucleic acids and proteins.

As used herein, the term a “biological sample” can be any solid or fluid sample obtained from, excreted by or secreted by any living organism, including without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as cancer). For example, a biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease). A biological sample can also be a sample obtained from any organ or tissue (including a biopsy or autopsy specimen, such as a tumor biopsy) or can include a cell (whether a primary cell or cultured cell) or medium conditioned by any cell, tissue or organ. In some examples, a biological sample is a nuclear extract. In certain examples, a sample is a quality control sample, such as one of the disclosed cell pellet section samples. In other examples, a sample is a test sample. Samples can be prepared using any method known in the art by of one of ordinary skill. The samples can be obtained from a subject for routine screening or from a subject that is suspected of having a disorder, such as a genetic abnormality, infection, or a neoplasia. The described embodiments of the disclosed method can also be applied to samples that do not have genetic abnormalities, diseases, disorders, etc., referred to as “normal” samples. Samples can include multiple targets that can be specifically bound by one or more detection probes.

As used herein, the term “conjugate” refers to two or more molecules or moieties (including macromolecules or supra-molecular molecules) that are covalently linked into a larger construct. In some embodiments, a conjugate includes one or more biomolecules (such as peptides, proteins, enzymes, sugars, polysaccharides, lipids, glycoproteins, and lipoproteins) covalently linked to one or more other molecules moieties.

As used herein, the terms “couple” or “coupling” refers to the joining, bonding (e.g. covalent bonding), or linking of one molecule or atom to another molecule or atom.

As used herein, the term “detectable moiety” refers to a molecule or material that can produce a detectable (such as visually, electronically or otherwise) signal that indicates the presence (i.e. qualitative analysis) and/or concentration (i.e. quantitative analysis) of the label in a sample.

As used herein, horseradish peroxidase (HRP) is an enzyme that can be conjugated to a labeled molecule. It produces a colored, fluorometric, or luminescent derivative of the labeled molecule when incubated with a proper substrate, allowing it to be detected and quantified. HRP acts in the presence of an electron donor to first form an enzyme substrate complex and then subsequently acts to oxidize an electronic donor. For example, HRP may act on 3,3′-diaminobenzidinetrahydrochloride (DAB) to produce a detectable color. HRP may also act upon a labeled tyramide conjugate, or tyramide like reactive conjugates (i.e. ferulate, coumaric, caffeic, cinnamate, dopamine, etc.), to deposit a colored or fluorescent or colorless reporter moiety for tyramide signal amplification (TSA).

As used herein, the terms “multiplex,” “multiplexed,” or “multiplexing” refer to detecting multiple targets in a sample concurrently, substantially simultaneously, or sequentially. Multiplexing can include identifying and/or quantifying multiple distinct nucleic acids (e.g., DNA, RNA, mRNA, miRNA) and polypeptides (e.g., proteins) both individually and in any and all combinations.

As used herein, a “quinone methide” is a quinone analog where one of the carbonyl oxygens on the corresponding quinone is replaced by a methylene group (—CH₂—) to form an alkene.

As used herein, the term “specific binding entity” refers to a member of a specific-binding pair. Specific binding pairs are pairs of molecules that are characterized in that they bind each other to the substantial exclusion of binding to other molecules (for example, specific binding pairs can have a binding constant that is at least 10-3 M greater, 10-4 M greater or 10-5 M greater than a binding constant for either of the two members of the binding pair with other molecules in a biological sample). Particular examples of specific binding moieties include specific binding proteins (for example, antibodies, lectins, avidins such as streptavidins, and protein A). Specific binding moieties can also include the molecules (or portions thereof) that are specifically bound by such specific binding proteins.

As used herein, the term “target” refers to any molecule for which the presence, location and/or concentration is or can be determined. Examples of target molecules include proteins, nucleic acid sequences, and haptens, such as haptens covalently bonded to proteins. Target molecules are typically detected using one or more conjugates of a specific binding molecule and a detectable label.

As used herein, the symbol “ [symbol] ” refers to a location a moiety is bonded to another moiety.

As used herein, the terms “band,” “absorption band” “peak,” “absorption peak,” “absorbance peak” and “first absorption band” can be used interchangeably and all refer to the lowest energy absorption band of the disclosed chromophores. All references to peak absorption wavelength and FWHM herein refer to the spectral width at half maximum absorbance of this first, or lowest energy, absorption band

Overview

The present disclosure is directed to labeling one or more targets within a biological sample with one or more detectable moieties, such as one or more different detectable moieties. In some embodiments, the one or more targets are one or more morphological markers and/or one or more biomarkers (each described herein). In some embodiments, the one or more targets includes two or more morphological markers, e.g., three or more morphological markers, five or more morphological markers, seven or more morphological markers, etc. In some embodiments, the one or more targets includes two or more morphological markers and/or one or more biomarkers, e.g., two or more biomarkers, three or more biomarkers, etc. In some embodiments, the one or more targets includes one or more morphological markers and/or two or more biomarkers, e.g., three or biomarkers, four or more biomarkers, etc.

In some embodiments, the labeling of one or more morphological markers in a biological sample is believed to provide context to the detection and visualization of one or more biomarkers in a biological sample. In some embodiments, the labeling of the one or more morphological markers provides positional context to one or more biomarkers. In some embodiments, the labeling of the one or more morphological markers allows for cell and/or tissue morphology to be detected and/or visualized concurrently with one or more biomarkers. In some embodiments, the labeling of one or more morphological markers serves as a surrogate for a special stain, e.g. a special stain that would stain a particular morphological structure or object in a cell. In other embodiments, the labeling of the one or more morphological markers services as a substitute for a special stain (e.g. mucicarmine) or a counterstain, e.g. hematoxylin (see below).

In some embodiments, the methods described herein facilitate the detection of one or more morphological markers and one or more biomarkers using bright-field microscopy. In some embodiments, the methods described herein facilitate the detection of one or more morphological markers and one or more biomarkers using one or more detectable conjugates. In some embodiments, the detectable conjugates include (i) a detectable moiety, and (ii) a tyramide moiety, a quinone methide precursor moiety, a derivative or analog of a tyramide moiety, or a derivative or analog of a quinone methide precursor moiety. In other embodiments, the detectable conjugates include (i) a detectable moiety, and (ii) a reactive functional group capable of participating in a click chemistry reaction. Suitable detectable conjugates and their methods of use are described herein.

In some embodiments, the detectable moieties coupled to each detectable conjugate have a predetermined full width at half maximum and a predetermined absorbance maximum (described herein). In some embodiments, the methods described herein utilize two or more detectable moieties whose absorbance maximum differ, such as by at least 10 nm, by at least 15 nm, by at least 20 nm, by at least 30 nm, by at least 40 nm, by at least 50 m, by at least 60 nm, by at least 70 nm, by at least 80 nm, by at least 90 nm, by at least 100 nm, by at least 120 nm, by at least 140 nm, by at least 160 nm, by at least 180 nm, by at least 200 nm, etc. In this manner, one or more labeled morphological markers and one or more labeled biomarkers may be distinguishable from one another, and free from substantial spectral overlap.

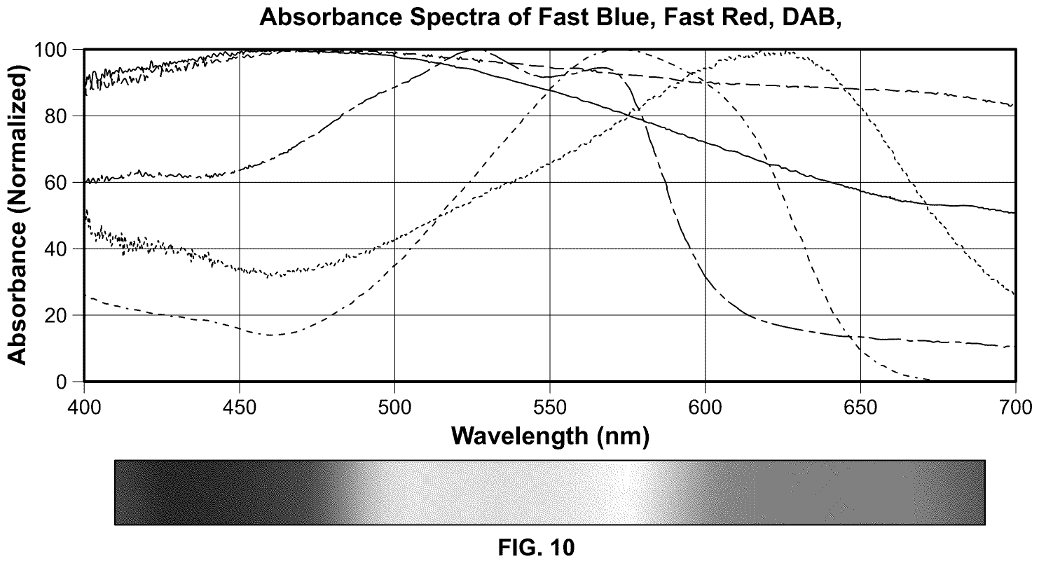

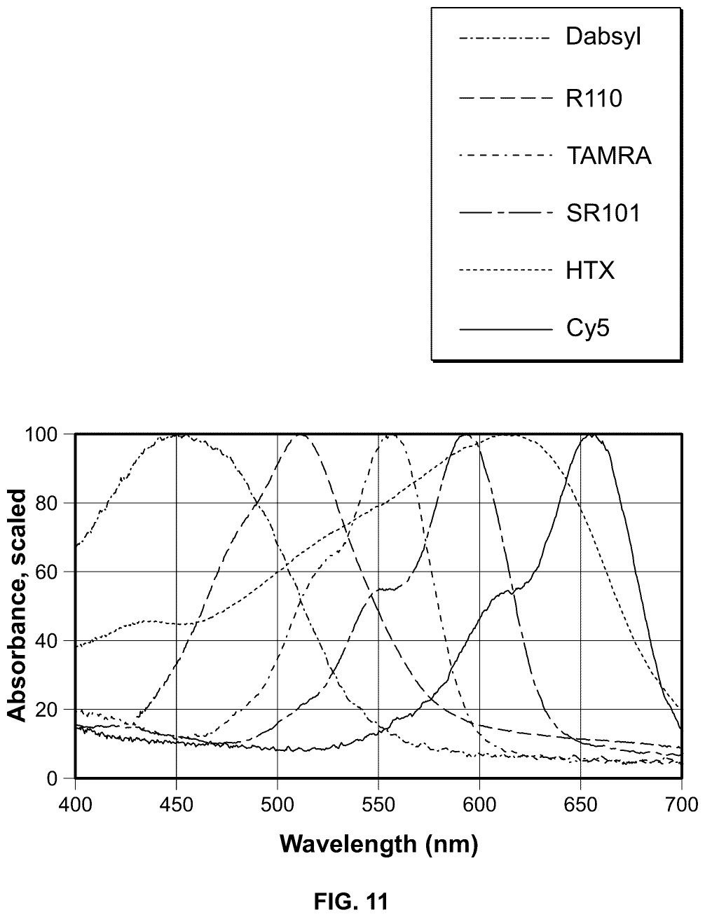

In some embodiments, the present disclosure enables labeling of one or more morphological markers and one or more biomarkers in a biological sample without the use of a counterstain, such as hematoxylin. As such, in some embodiments, the stained biological samples are substantially free of hematoxylin. The conventional bright-field nuclear counterstain, hematoxylin, which provides a measure of specimen cellular and tissue morphology, has a broad spectral absorbance that is believed to be problematic for multiplexing with either immunohistochemistry or in situ hybridization (see FIG. 10). The broad absorbance has considerable overlap with spectrally neighboring chromogens, making it difficult to clearly distinguish the individual stained biomarkers (see FIGS. 10 and 11). While hematoxylin serves as an effective counterstain, its broad spectra complicates visual evaluation of labeled biomarkers, especially when evaluating two or more labeled biomarkers.

Certain detectable moieties (such as those described herein) have relatively narrow absorbance bands and thus facilitate higher order bright-field multiplexing. For example, the absorbance spectra of five detectable moieties (Dabysl, R10, TAMRA, SR101, and Cy5) are plotted in FIG. 11. The hematoxylin absorbance spectrum is also included in FIG. 11, which serves to emphasize the broad nature of hematoxylin absorbance, and the consequent problem with spectral overlap between hematoxylin and the aforementioned five detectable moieties (compare hematoxylin to R110, TAMRA, SR101, and Cy5 in FIG. 11). The reduced spectral overlap between these detectable moieties (see discussion on full width half maxima and absorbance maxima herein) provides improved visual distinction of the biomarkers labeled with such detectable moieties. Counterstaining, however, is still required to provide context to the labeled biomarkers.

In IHC and ISH, hematoxylin staining is typically reduced to the point that it does not interfere with visualization or quantification of biomarker staining. For multiplex assays, hematoxylin is often reduced to the point that the nuclear staining is barely visible, thus “trading off” the ability to identity/quantity one or more labeled biomarkers with the ability to distinguish nuclear staining (and, hence, contextual information such as cell and/or tissue morphology). Despite the lowering of the hematoxylin staining level, there still exists some level of spectral crosstalk between hematoxylin and chromogens or detectable moieties.

In some embodiments of the present disclosure, morphological markers (described herein) are labeled using any of the detectable moieties described herein, thus obviating the need for counterstains, such as hematoxylin. Thus, one or more labeled morphological markers and one or more labeled biomarkers may be detected, visualized, and/or quantified with minimal spectral crosstalk.

Targets for Labeling

The presently disclosed methods are capable of labelling one or more targets within a biological sample, including “morphological markers” and “biomarkers,” as described herein.

Morphological Markers

In some embodiments, the one or more targets are protein markers, nucleic acid markers, or cellular components which allow for the identification of different morphological features on or within different types of cells (or cellular components) and/or on or within different types of tissues within a biological sample (herein after referred to as “morphological markers”). For example, a morphological feature may be a nucleus and different morphological markers, such as DNA, histone proteins, etc., may be used to facilitate or characterize identification, e.g. visualization, of the nucleus. In some embodiments, the two or more morphological markers characteristic of the same morphological feature, e.g., a nucleus, are stained or contacted with detectable moieties.

Non-limiting examples of morphological markers (which may be used to identify various morphological features) include DNA, histone proteins, markers for cytosol, markers for endoplasmic reticulum; nuclear membrane markers, markers of nucleoli or its substructures; markers for a nucleus and its substructures; markers of actin filaments, focal adhesions or their substructures; markers for centrosomes and centriolar satellites; markers for intermediate filaments or its substructures; markers for microtubule structures or substructures; markers for mitochondria; markers for localizing endoplasmic reticulum proteins across different cell lines; markers for the Golgi apparatus; markers used to localize Golgi apparatus-associated proteins across different cell lines; markers for the plasma membrane; markers for highly expressed single localizing plasma membrane proteins across different cell lines; and markers for vesicular organelles.

Specific non-limiting examples morphological markers are described below. In addition to the morphological markers enumerated herein, additional morphological markers and antibodies that bind particularly to those morphological markers may be selected by reference to the Human Protein Atlas (https://www.proteinatlas.org/). Methods for preparing the antibodies for use in a covalent detection scheme such as any one of tyramide, quinone methide or click detection are well known. Furthermore, antibodies available from Atlas Antibodies are generally available from Sigma-Aldrich.

DNA; [anti-ds DNA [DSD/958] (ab215896) antibody obtained from ABCAM (Cambridge, MA)]

Histone proteins; [anti-histone H3 (ab1791) antibody obtained from ABCAM (Cambridge, MA)]

Markers of cytosol (e.g. actin [anti-beta actin antibody (ab8226) obtained from ABCAM (Cambridge, MA)], Adenylosuccinate lyase, Ataxin 2, G3BP stress granule assembly factor 2, Aminoacyl tRNA synthetase complex interacting multifunctional protein 1, Tyrosyl-tRNA synthetase, Aspartyl-tRNA synthetase, SERPINEl mRNA binding protein 1, Coiled-coil domain containing 43, Glutamyl-prolyl-tRNA synthetase, Histidyl-tRNA synthetase, Ataxin 2 like, Adenosine monophosphate deaminase 2, and RAB GTPase activating protein 1);

TABLE 1
Cytosol Markers

| Cytosol | | | |
|---|---|---|---|
| Marker | Protein Target | Antibody | Source |
| ADSL | Adenylosuccinate | HPA000525 | Atlas |
| | lyase | | Antibodies |
| ATXN2 | Ataxin 2 | HPA018295 | Atlas |
| | | | Antibodies |
| G3BP2 | G3BP stress granule | HPA018304 | Atlas |
| | assembly factor 2 | | Antibodies |
| AIMP1 | Aminoacyl tRNA | HPA018476 | Atlas |
| | synthetase complex | | Antibodies |
| | interacting | | |
| | multifunctional | | |
| | protein 1 | | |
| YARS | Tyrosyl-tRNA | HPA018950 | Atlas |
| | synthetase | | Antibodies |
| DARS | Aspartyl-tRNA | HPA020451 | Atlas |
| | synthetase | | Antibodies |
| SERBP1 | SERPINE1 mRNA | HPA020559 | Atlas |
| | binding protein 1 | | Antibodies |
| CCDC43 | Coiled-coil domain | HPA023391 | Atlas |
| | containing 43 | | Antibodies |
| EPRS | Glutamyl-prolyl-tRNA | HPA030052 | Atlas |
| | synthetase | | Antibodies |
| HARS | Histidyl-tRNA | HPA036539 | Atlas |
| | synthetase | | Antibodies |
| ATXN2L | Ataxin 2 like | HPA041506 | Atlas |
| | | | Antibodies |
| AMPD2 | Adenosine | HPA045760 | Atlas |
| | monophosphate | | Antibodies |
| | deaminase 2 | | |
| RABGAP1 | RAB GTPase | HPA064860 | Atlas |
| | activating protein 1 | | Antibodies |

By way of example, two or more cytosol markers may be labeled (such as with any of the detectable moieties disclosed herein) to characterize the cytosol. In some embodiments, the labeling of the two or more cytosol markers may be combined with the labeling of one or more biomarkers, so as to characterize a cytosol morphological feature and/or one or more biomarkers.

Markers for endoplasmic reticulum (e.g. Heat shock protein 90 beta family member 1, Calnexin [anti-calnexin antibody—ER marker (ab22595) obtained from ABCAM, Kinectin 1, Protein disulfide isomerase family A member 3, Reticulocalbin 1, Ribosome binding protein 1, Sec61 translocon beta subunit, Cytochrome P450 family 51 subfamily A member 1);

TABLE 2
Endoplasmic Reticulum Markers

| Endoplasmic | | | |
|---|---|---|---|
| Reticulum | | | |
| Marker | Protein Target | Antibody | Source |
| HSP90B1 | Heat shock protein | HPA008424 | Atlas |
| | 90 beta family | | Antibodies |
| | member 1 | | |
| CANX | Calnexin | HPA009433 | Atlas |
| | | | Antibodies |
| KTN1 | Kinectin 1 | HPA003178 | Atlas |
| | | | Antibodies |
| PDIA3 | Protein disulfide | HPA00645 | Atlas |
| | isomerase family | | Antibodies |
| | A member 3 | | |
| RCN1 | Reticulocalbin 1 | HPA038474 | Atlas |
| | | | Antibodies |
| RRBP1 | Ribosome binding | HPA009206 | Atlas |
| | protein 1 | | Antibodies |
| SEC61B | Sec61 translocon | HPA049407 | Atlas |
| | beta subunit | | Antibodies |
| CYP51A1 | Cytochrome P450 | HPA041325 | Atlas |
| | family 51 subfamily | | Antibodies |
| | A member 1 | | |

By way of example, two or more endoplasmic reticulum markers may be labeled (such as with any of the detectable moieties disclosed herein) to characterize the endoplasmic reticulum. In some embodiments, the labeling of the two or more endoplasmic reticulum markers may be combined with the labeling of one or more biomarkers, so as to characterize an endoplasmic reticulum morphological feature and/or one or more biomarkers.

Nuclear membrane markers (e.g. Sad1 and UNC84 domain containing 2, Thymopoietin, Sad1 and UNC84 domain containing 1, LEM domain containing 2, Lamin B1 [anti-lamin B1 antibody (EPR8985(B) obtained from ABCAM)][anti-lamin antibody (abd1575) obtained from ABCAM], Torsin 1A interacting protein 1, Lamin B receptor, Lamin 1B2);

TABLE 3
Nuclear Membrane Markers

| Nuclear | | | |
|---|---|---|---|
| Membrane | | | |
| Marker | Protein Target | Antibody | Source |
| SUN2 | Sad1 and UNC84 | HPA001209 | Atlas |
| | domain containing 2 | | Antibodies |
| TMPO | Thymopoietin | HPA008150 | Atlas |
| | | | Antibodies |
| SUN1 | Sad1 and UNC84 | HPA008461 | Atlas |
| | domain containing 1 | | Antibodies |
| LEMD2 | LEM domain | HPA017340 | Atlas |
| | containing 2 | | Antibodies |
| LMNB1 | Lamin B1 | HPA050524 | Atlas |
| | | | Antibodies |
| TOR1AIP1 | Torsin 1A | HPA050546 | Atlas |
| | interacting protein 1 | | Antibodies |
| LBR | Lamin B receptor | HPA062236 | Atlas |
| | | | Antibodies |
| LMNB2 | Lamin B2 | HPA062477 | Atlas |
| | | | Antibodies |

By way of example, two or more nuclear membrane markers may be labeled (such as with any of the detectable moieties disclosed herein) to characterize the nuclear membrane. In some embodiments, the labeling of the two or more nuclear membrane markers may be combined with the labeling of one or more biomarkers, so as to characterize a nuclear membrane morphological feature and/or one or more biomarkers.

Markers of nucleoli or its substructures (e.g. DEAD-box helicase 47, Ribosome production factor 1 homolog, UTP6, small subunit processome component, Nucleolar protein 10, FtsJ RNA methyltransferase homolog 3, Upstream binding transcription factor RNA polymerase I);

TABLE 4
Nucleoli Markers

| Nucleoli | | | |
|---|---|---|---|
| Marker | Protein Target | Antibody | Source |
| DDX47 | DEAD-box helicase | HPA014855 | Atlas |
| | 47 | | Antibodies |
| RPF1 | Ribosome production | HPA024642 | Atlas |
| | factor 1 homolog | | Antibodies |
| UTP6 | UTP6, small subunit | HPA025936 | Atlas |
| | processome | | Antibodies |
| | component | | |
| NOL10 | Nucleolar protein 10 | HPA035286 | Atlas |
| | | | Antibodies |
| FTSJ3 | FtsJ RNA | HPA055544 | Atlas |
| | methyltransferase | | Antibodies |
| | homolog 3 | | |
| UBTF | Upstream binding | SC-13125 | Santa Cruz |
| | transcription factor, | | Biotechnologies |
| | RNA polymerase I | | |

By way of example, two or more nucleoli markers may be labeled (such as with any of the detectable moieties disclosed herein) to characterize the nucleoli or its substructures. In some embodiments, the labeling of the two or more nucleoli markers may be combined with the labeling of one or more biomarkers, so as to characterize a nucleoli morphological feature and/or one or more biomarkers.

Markers for a nucleus and its substructures (Poly(ADP-ribose) polymerase 1, Serine/arginine repetitive matrix 2, RNA binding motif protein 25, X-ray repair cross complementing 6, Heterogeneous nuclear ribonucleoprotein C (C1/C2), TATA-box binding protein associated factor 15, SWI/SNF-related, matrix-associated actin-dependent regulator of chromatin subfamily a containing DEAD/H box 1, C-terminal binding protein 1, SWI/SNF related matrix associated actin dependent regulator of chromatin subfamily c member 2, and PDS5 cohesion associated factor A);

TABLE 5
Nuclear Markers

| Nuclear | | | |
|---|---|---|---|
| Marker | Protein Target | Antibody | Source |
| PARP1 | Poly(ADP-ribose) | 1051-1 | Abcam |
| | polymerase 1 | | (Epitomics) |
| SRRM2 | Serine/arginine | HPA041411 | Atlas |
| | repetitive matrix 2 | | Antibodies |
| RBM25 | RNA binding motif | HPA003025 | Atlas |
| | protein 25 | | Antibodies |
| XRCC6 | X-ray repair cross | HPA047549 | Atlas |
| | complementing 6 | | Antibodies |
| HNRNPC | Heterogeneous | AMAb91010 | Atlas |
| | nuclear | | Antibodies |
| | ribonucleoprotein | | |
| | C (C1/C2) | | |
| TAF15 | TATA-box binding | HPA052059 | Atlas |
| | protein associated | | Antibodies |
| | factor 15 | | |
| SMARCAD1 | SWI/SNF-related, | HPA016737 | Atlas |
| | matrix-associated | | Antibodies |
| | actin-dependent | | |
| | regulator of | | |
| | chromatin, subfamily | | |
| | a, containing | | |
| | DEAD/H box 1 | | |
| CTBP1 | C-terminal binding | Sc-1785 | Santa Cruz |
| | protein 1 | | Biotechnologies |
| SMARCC2 | SWI/SNF related, | Sc-17838 | Santa Cruz |
| | matrix associated, | | Biotechnologies |
| | actin dependent | | |
| | regulator of | | |
| | chromatin subfamily | | |
| | c member 2 | | |
| PDS5A | PDS5 cohesin | HPA036661 | Atlas |
| | associated factor A | | Antibodies |

By way of example, two or more nuclear markers may be labeled (such as with any of the detectable moieties disclosed herein) to characterize the nucleus. In some embodiments, the labeling of the two or more nuclear markers may be combined with the labeling of one or more biomarkers, so as to characterize a nuclear morphological feature and/or one or more biomarkers.

Markers of actin filaments, focal adhesions or their substructures (Septin 9, Chondroitin sulfate N-acetylgalactosaminyltransferase 1, FYVE, RhoGEF and PH domain containing 4, Zyxin, N-acylsphingosine amidohydrolase 2, Vinculin);

TABLE 6
Actin Filament Markers

| Actin | | | |
|---|---|---|---|
| Filament | | | |
| Marker | Protein Target | Antibody | Source |
| SEPT9 | Septin 9 | HPA042564 | Atlas Antibodies |
| CSGALNACT1 | Chondroitin sulfate N- | HPA068462 | Atlas Antibodies |
| | acetylgalactosaminyltransferase 1 | | |
| FGD4 | FYVE, RhoGEF and PH | HPA039235 | Atlas Antibodies |
| | domain containing 4 | | |
| ZYX | Zyxin | HPA004835 | Atlas Antibodies |
| ASAH2 | N-acylsphingosine | HPA061171 | Atlas Antibodies |
| | amidohydrolase 2 | | |
| VCL | Vinculin | NCL-VINC | Leica |
| | | | Biosystems |
| | | | (Novocastra) |

By way of example, two or more markers of actin filaments, focal adhesions or their substructures may be labeled (such as with any of the detectable moieties disclosed herein) to characterize actin filaments, focal adhesions or their substructures or its substructures. In some embodiments, the labeling of the two or more markers actin filaments, focal adhesions or their substructures may be combined with the labeling of one or more biomarkers, so as to characterize actin filaments or focal adhesions as morphological features and/or one or more biomarkers.

Markers for centrosomes and centriolar satellites (McKusick-Kaufman syndrome, Outer dense fiber of sperm tails 2, Centrosomal protein 97, Kinesin family member 5B, Progesterone immunomodulatory binding factor 1);

TABLE 7
Centrosome Markers

| Centrosome | | | |
|---|---|---|---|
| Marker | Protein Target | Antibody | Source |
| MKKS | McKusick-Kaufman | HPA044233 | Atlas |
| | syndrome | | Antibodies |
| ODF2 | Outer dense fiber of | HPA048841 | Atlas |
| | sperm tails 2 | | Antibodies |
| CEP97 | Centrosomal protein | HPA002980 | Atlas |
| | 97 | | Antibodies |
| KIF5B | Kinesin family | HPA037589 | Atlas |
| | member 5B | | Antibodies |
| PIBF1 | Progesterone | HPA052269 | Atlas |
| | immunomodulatory | | Antibodies |
| | binding factor 1 | | |

Markers for intermediate filaments or its substructures (Keratin 19, Keratin 4, Desmin, Nestin, Keratin 17, Keratin 13), markers intermediate filament proteins across different cell lines (Vimentin, Keratin 8, Keratin 7, Keratin 19, Praja ring finger ubiquitin ligase 2, Keratin 17, Keratin 14, Nestin, Keratin 80, Keratin 13);

TABLE 8
Intermediate Filament Markers

| Intermediate | | | |
|---|---|---|---|
| Filament | | | |
| Marker | Protein Target | Antibody | Source |
| VIM | Vimentin | HPA001762 | Atlas |
| | | | Antibodies |
| KRT8 | Keratin 8 | HPA049866 | Atlas |
| | | | Antibodies |
| KRT7 | Keratin 7 | M7018 | Agilent |
| KRT19 | Keratin 19 | M0888 | Agilent |
| PJA2 | Praja ring finger | HPA040347 | Atlas |
| | ubiquitin ligase 2 | | Antibodies |
| KRT17 | Keratin 17 | HPA000453 | Atlas |
| | | | Antibodies |
| KRT14 | Keratin 14 | HPA000452 | Atlas |
| | | | Antibodies |
| NES | Nestin | HPA006286 | Atlas |
| | | | Antibodies |
| KRT80 | Keratin 80 | HPA077836 | Atlas |
| | | | Antibodies |
| KRT13 | Keratin 13 | HPA030877 | Atlas |
| | | | Antibodies |
| KRT4 | Keratin 4 | HPA034881 | Atlas |
| | | | Antibodies |

By way of example, two or more intermediate filament markers may be labeled (such as with any of the detectable moieties disclosed herein) to characterize intermediate filaments. In some embodiments, the labeling of the two or more intermediate filament markers may be combined with the labeling of one or more biomarkers, so as to characterize an intermediate filament morphological feature and/or one or more biomarkers.

Markers for microtubule structures or substructures (e.g. Tubulin alpha 1a, Dystrobrevin binding protein 1, Calmodulin regulated spectrin associated protein family member 2);

TABLE 9
Microtubule Markers

| Microtuble | | | |
|---|---|---|---|
| Marker | Protein Target | Antibody | Source |
| TUBA1A | Tubilin alpha 1a | HPA039247 | Atlas |
| | | | Antibodies |
| DTNBP1 | Dystrobrevin | HPA028053 | Atlas |
| | binding protein 1 | | Antibodies |
| CAMSAP2 | Calmodulin regulate | HPA026511 | Atlas |
| | spectrin associated | | Antibodies |
| | protein famil | | |
| | member 2 | | |

By way of example, two or more markers for microtubules may be labeled (such as with any of the detectable moieties disclosed herein) to characterize microtubule structures or substructures. In some embodiments, the labeling of the two or more markers for microtubules may be combined with the labeling of one or more biomarkers, so as to characterize a microtubule structure or substructure morphological feature and/or one or more biomarkers.

Markers for mitochondria (Citrate synthase, Leucine rich pentatricopeptide repeat containing, Solute carrier family 25 member 24, Translocase of inner mitochondrial membrane 44, Glutaryl-CoA dehydrogenase, TNF receptor associated protein 1);

TABLE 10
Mitochondrial Markers

| Mitochondria | | | |
|---|---|---|---|
| Marker | Protein Target | Antibody | Source |
| CS | Citrate Synthase | AMAb91005 | Atlas |
| | | | Antibodies |
| LRPPRC | Leucine rich | HPA036408 | Atlas |
| | pentatricopeptide | | Antibodies |
| | repeat containing | | |
| SLC25A24 | Solute carrier family | HPA028519 | Atlas |
| | 25 member 24 | | Antibodies |
| TIMM44 | Translocase of inner | HPA043052 | Atlas |
| | mitochondrial | | Antibodies |
| | membrane 44 | | |
| GCDH | Glutaryl-CoA | HPA043252 | Atlas |
| | dehydrogenase | | Antibodies |
| TRAP1 | TNF receptor | HPA041082 | Atlas |
| | associated protein 1 | | Antibodies |

By way of example, two or more mitochondrial markers may be labeled (such as with any of the detectable moieties disclosed herein) to characterize mitochondria. In some embodiments, the labeling of the two or more mitochondrial markers may be combined with the labeling of one or more biomarkers, so as to characterize mitochondria as a morphological feature and/or one or more biomarkers.

Markers for localizing endoplasmic reticulum proteins across different cell lines (Ribosomal protein L41, Calreticulin, Heat shock protein 90 beta family member 1, Prolyl 4-hydroxylase subunit beta, Protein kinase C substrate 80K-H, Ribophorin II, Ribophorin I, Sec61 translocon beta subunit, Dolichyl-diphosphooligosaccharide—protein glycosyltransferase non-catalytic subunit);

Markers for the Golgi apparatus (Golgin B1, Golgin A5, Polypeptide N-acetylgalactosaminyltransferase 2, Zinc finger protein like 1, Golgi reassembly stacking protein 2, Golgi membrane protein 1, Golgi integral membrane protein 4, B cell receptor associated protein 31);

Markers used to localize Golgi apparatus-associated proteins across different cell lines (e.g. Retention in endoplasmic reticulum sorting receptor 1, Stromal cell derived factor 4, Coatomer protein complex subunit epsilon, Caveolin 1, Transmembrane p24 trafficking protein 10, Serglycin, Transmembrane p24 trafficking protein 3, ATPase secretory pathway Ca2+ transporting 1, ADP ribosylation factor GTPase activating protein 2, Phosphatidylinositol 4-kinase beta);

Markers for the plasma membrane (Syntaxin 4, Solute carrier family 16 member 1, Ezrin, Erythrocyte membrane protein band 4.1 like 3, Catenin beta 1, Ankyrin 3, Solute carrier family 41 member 3);

Markers for highly expressed single localizing plasma membrane proteins across different cell lines (Adaptor related protein complex 2 mu 1 subunit, G protein subunit beta 2, Moesin, ATPase Na+/K+ transporting subunit beta 3, Phosphatidylethanolamine binding protein 1, Catenin beta 1, CD81 molecule, Solute carrier family 1 member 5, Ezrin, S100 calcium binding protein A4); and

Markers for vesicular organelles (e.g. Ankyrin repeat and FYVE domain containing 1, RAB5C member RAS oncogene family, Alkylglycerone phosphate synthase, Acyl-CoA binding domain containing 5, RAB7A, member RAS oncogene family, Perilipin 3).

In some embodiments, the one or more morphological markers is a histone protein (e.g. targeted with an anti-histone antibody). In some embodiments, the one or more morphological markers are DNA (e.g. targeted with an anti-DNA antibody). In some embodiments, the morphological markers are both a histone protein and DNA.

In some embodiments, the one or more morphological markers are cell membrane markers. Examples of cell membrane markers are sodium-potassium ATPase (which is responsible for the extracellular transport of sodium ions and the intracellular transport of potassium ions; and which may be targeted with anti-sodium potassium ATPase antibody); plasma membrane calcium ATPase (plasma membrane calcium ATPase (PMCA) regulates intracellular calcium concentrations by removing Ca2+ from the cell, and which may be targeted with an anti-calcium pump pan ATPase antibody); Cadherin (a transmembrane protein that mediates calcium-dependent cell-cell adhesion. The Ca2+ binding domains of cadherins are highly conserved, enabling the creation of antibodies that are effective across all members of the cadherin superfamily, and which may be targeted with an anti-pan Cadherin antibody); CD98 (transmembrane glycoprotein found in vertebrates; it forms part of the heterodimeric neutral amino acid transport systems; it may be targeted with an anti-CD98 antibody); caveolae (complex plasma membrane structures whose properties appear to place them between coated pits and lipid rafts, and which may be targeted by an anti-caveolin-1 antibody).

In some embodiments, the one or more morphological markers are cytoplasm markers. Examples of cytoplasm markers include microtubules (highly dynamic polymers composed of 13 protofilaments of α-tubulin and β-tubulin heterodimers that continuously grow and shrink during interphase and mitosis and which may be targeted by Anti-alpha Tubulin antibody); Vimentin (class-III intermediate filaments found in various non-epithelial cells, especially mesenchymal cells. Vimentin is attached to the nucleus, endoplasmic reticulum, and mitochondria, either laterally or terminally and which may be targeted by an anti-vimentin antibody); desmin (class-III intermediate filaments found in muscle cells. In adult striated muscle they form a fibrous network connecting myofibrils to each other and the plasma membrane from the periphery of the Z-line structures and which may be targeted by an anti-desmin antibody); cytokeratin (intermediate filaments present in all epithelial cells, and also in several non-epithelial cells. These may regulate the activity of kinases such as PKC and SRC via binding to integrin beta-1 (ITB1) and the receptor of activated protein kinase C and which may be targeted by an anti-cytokeratin 19 antibody).

In some embodiments, the one or more morphological markers are nuclear markers. Examples of nuclear markers include the nucleus (anti-KDM1/LSD1 antibody); nuclear pores (anti-NUP98 antibody); nuclear envelopes (anti-lamin A+C antibody); nuclear speckles (anti-SC35 antibody); nucleolus (anti-fibrillarian antibody); heterochromatin) anti-HP1 alpha antibody); and centromeres (anti-CENPA antibody).

In some embodiments, the one or more morphological markers are organelle markers. Examples of organelle markers include the endoplasmic reticulum (anti-calreticulin antibody); golgi apparatus (anti-GM130 antibody); mitochondria (anti-ATP5A antibody); ribosome (anti-RPS3 antibody); lysosome (anti-M6PR ANTIBODY); endosome (anti-EEA1 antibody); peroxisome (anti-catalase antibody); and autophagosome (anti-SQSTM1/p 62 antibody).

Biomarkers

In some embodiments, the one or more targets within the biological sample are biomarkers. The term “biomarker” as used herein refers to an indicator, e.g., predictive, diagnostic, and/or prognostic, which can be detected in a biological sample, for example, PD-L1. The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers. Included as illustrative embodiments are antigens, epitopes, cellular proteins, transmembrane proteins, and DNA or RNA sequences. The Her-2/neu gene and protein are both illustrative embodiments of biomarkers.

As noted above, the biomarker targets can be nucleic acid sequences or proteins. Throughout this disclosure when reference is made to a target biomarker protein it is understood that the nucleic acid sequences associated with that protein can also be used as a biomarker target. In some embodiments, the biomarker target is a protein or nucleic acid molecule from a pathogen, such as a virus, bacteria, or intracellular parasite, such as from a viral genome. For example, a biomarker target protein may be produced from a target nucleic acid sequence associated with (e.g., correlated with, causally implicated in, etc.) a disease.

A biomarker target nucleic acid sequence can vary substantially in size. Without limitation, the nucleic acid sequence can have a variable number of nucleic acid residues. For example, a biomarker target nucleic acid sequence can have at least about 10 nucleic acid residues, or at least about 20, 30, 50, 100, 150, 500, 1000 residues. Similarly, a biomarker target polypeptide can vary substantially in size. Without limitation, the biomarker target polypeptide will include at least one epitope that binds to a peptide specific antibody, or fragment thereof. In some embodiments that polypeptide can include at least two epitopes that bind to a peptide specific antibody, or fragment thereof.

In specific, non-limiting embodiments, a biomarker target protein is produced by a target nucleic acid sequence (e.g., genomic target nucleic acid sequence) associated with a neoplasm (for example, a cancer). Numerous chromosome abnormalities (including translocations and other rearrangements, amplification or deletion) have been identified in neoplastic cells, especially in cancer cells, such as B cell and T cell leukemias, lymphomas, breast cancer, colon cancer, neurological cancers and the like. Therefore, in some embodiments, at least a portion of the biomarker target molecule is produced by a nucleic acid sequence (e.g., genomic target nucleic acid sequence) amplified or deleted in at least a subset of cells in a sample.

Oncogenes are known to be responsible for several human malignancies. For example, chromosomal rearrangements involving the SYT gene located in the breakpoint region of chromosome 18q11.2 are common among synovial sarcoma soft tissue tumors. The t(18q11.2) translocation can be identified, for example, using probes with different labels: the first probe includes FPC nucleic acid molecules generated from a target nucleic acid sequence that extends distally from the SYT gene, and the second probe includes FPC nucleic acid generated from a target nucleic acid sequence that extends 3′ or proximal to the SYT gene. When probes corresponding to these target nucleic acid sequences (e.g., genomic target nucleic acid sequences) are used in an in situ hybridization procedure, normal cells, which lack a t(18q11.2) in the SYT gene region, exhibit two fusions (generated by the two labels in close proximity) signals, reflecting the two intact copies of SYT. Abnormal cells with a t(18q11.2) exhibit a single fusion signal.

In other embodiments, a biomarker target protein produced from a nucleic acid sequence (e.g., genomic target nucleic acid sequence) is selected that is a tumor suppressor gene that is deleted (lost) in malignant cells. For example, the p16 region (including D9S1749, D9S1747, p16(INK4A), p14(ARF), D9S1748, p15(INK4B), and D9S1752) located on chromosome 9p21 is deleted in certain bladder cancers. Chromosomal deletions involving the distal region of the short arm of chromosome 1 (that encompasses, for example, SHGC57243, TP73, EGFL3, ABL2, ANGPTL1, and SHGC-1322), and the pericentromeric region (e.g., 19p13-19q13) of chromosome 19 (that encompasses, for example, MAN2B1, ZNF443, ZNF44, CRX, GLTSCR2, and GLTSCR1) are characteristic molecular features of certain types of solid tumors of the central nervous system.

The aforementioned embodiments are provided solely for purpose of illustration and are not intended to be limiting. Numerous other cytogenetic abnormalities that correlate with neoplastic transformation and/or growth are known to those of ordinary skill in the art. Biomarker target proteins that are produced by nucleic acid sequences (e.g., genomic target nucleic acid sequences), which have been correlated with neoplastic transformation and which are useful in the disclosed methods, also include the EGFR gene (7p12; e.g., GENBANK™ Accession No. NC-000007, nucleotides 55054219-55242525), the C-MYC gene (8q24.21; e.g., GENBANK™ Accession No. NC-000008, nucleotides 128817498-128822856), D5S271 (5p15.2), lipoprotein lipase (LPL) gene (8p22; e.g., GENBANK™ Accession No. NC 000008, nucleotides 19841058-19869049), RB1 (13q14; e.g., GENBANK™ Accession No. NC 000013, nucleotides 47775912-47954023), p53 (17p13.1; e.g., GENBANK™ Accession No. NC-000017, complement, nucleotides 7512464-7531642)), N-MYC (2p24; e.g., GENBANK™ Accession No. NC-000002, complement, nucleotides 151835231-151854620), CHOP (12q13; e.g., GENBANK™ Accession No. NC 000012, complement, nucleotides 56196638-56200567), FUS (16p11.2; e.g., GENBANK™ Accession No. NC 000016, nucleotides 31098954-31110601), FKHR (13p14; e.g., GENBANK™ Accession No. NC-000013, complement, nucleotides 40027817-40138734), as well as, for example: ALK (2p23; e.g., GENBANK™ Accession No. NC-000002, complement, nucleotides 29269144-29997936), Ig heavy chain, CCND1 (11q13; e.g., GENBANK™ Accession No. NC-000011, nucleotides 69165054.69178423), BCL2 (18q21.3; e.g., GENBANK™ Accession No. NC 000018, complement, nucleotides 58941559-59137593), BCL6 (3q27; e.g., GENBANK™ Accession No. NC-000003, complement, nucleotides 188921859-188946169), MALF1, AP1 (1p32-p31; e.g., GENBANK™ Accession No. NC 000001, complement, nucleotides 59019051-59022373), TOP2A (17q21-q22; e.g., GENBANK™ Accession No. NC-000017, complement, nucleotides 35798321-35827695), TMPRSS (21q22.3; e.g., GENBANK™ Accession No. NC 000021, complement, nucleotides 41758351-41801948), ERG (21q22.3; e.g., GENBANK™ Accession No. NC-000021, complement, nucleotides 38675671-38955488); ETV1 (7p21.3; e.g., GENBANK™ Accession No. NC 000007, complement, nucleotides 13897379-13995289), EWS (22q12.2; e.g., GENBANK™ Accession No. NC 000022, nucleotides 27994271-28026505); FLI1 (11q24.1-q24.3; e.g., GENBANK™ Accession No. NC-000011, nucleotides 128069199-128187521), PAX3 (2q35-q37; e.g., GENBANK™ Accession No. NC-000002, complement, nucleotides 222772851-222871944), PAX7 (1p36.2-p36.12; e.g., GENBANK™ Accession No. NC 000001, nucleotides 18830087-18935219), PTEN (10q23.3; e.g., GENBANK™ Accession No. NC 000010, nucleotides 89613175-89716382), AKT2 (19q13.1-q13.2; e.g., GENBANK™ Accession No. NC-000019, complement, nucleotides 45431556-45483036), MYCL1 (1p34.2; e.g., GENBANK™ Accession No. NC 000001, complement, nucleotides 40133685-40140274), REL (2p13-p12; e.g., GENBANK™ Accession No. NC 000002, nucleotides 60962256-61003682) and CSF1R (5q33-q35; e.g., GENBANK™ Accession No. NC-000005, complement, nucleotides 149413051-149473128).

In other embodiments, a biomarker target protein is selected from a virus or other microorganism associated with a disease or condition. Detection of the virus- or microorganism-derived target nucleic acid sequence (e.g., genomic target nucleic acid sequence) in a cell or biological sample is indicative of the presence of the organism. For example, the biomarker target peptide, polypeptide or protein can be selected from the genome of an oncogenic or pathogenic virus, a bacterium or an intracellular parasite (such as Plasmodium falciparum and other Plasmodium species, Leishmania (sp.), Cryptosporidium parvum, Entamoeba histolytica, and Giardia lamblia, as well as Toxoplasma, Eimeria, Theileria, and Babesia species).

In some embodiments, the biomarker target protein is produced from a nucleic acid sequence (e.g., genomic target nucleic acid sequence) from a viral genome. Exemplary viruses and corresponding genomic sequences (GENBANK™ RefSeq Accession No. in parentheses) include human adenovirus A (NC-001460), human adenovirus B (NC 004001), human adenovirus C(NC-001405), human adenovirus D (NC-002067), human adenovirus E (NC-003266), human adenovirus F (NC-001454), human astrovirus (NC 001943), human BK polyomavirus (V01109; GI:60851) human bocavirus (NC-007455), human coronavirus 229E (NC-002645), human coronavirus HKU1 (NC-006577), human coronavirus NL63 (NC-005831), human coronavirus OC43 (NC-005147), human enterovirus A (NC-001612), human enterovirus B (NC-001472), human enterovirus C(NC-001428), human enterovirus D (NC-001430), human erythrovirus V9 (NC 004295), human foamy virus (NC-001736), human herpesvirus 1 (Herpes simplex virus type 1) (NC-001806), human herpesvirus 2 (Herpes simplex virus type 2) (NC-001798), human herpesvirus 3 (Varicella zoster virus) (NC-001348), human herpesvirus 4 type 1 (Epstein-Barr virus type 1) (NC-007605), human herpesvirus 4 type 2 (Epstein-Barr virus type 2) (NC-009334), human herpesvirus 5 strain AD 169 (NC-001347), human herpesvirus 5 strain Merlin Strain (NC-006273), human herpesvirus 6A (NC-001664), human herpesvirus 6B (NC-000898), human herpesvirus 7 (NC-001716), human herpesvirus 8 type M (NC 003409), human herpesvirus 8 type P (NC-009333), human immunodeficiency virus 1 (NC 001802), human immunodeficiency virus 2 (NC-001722), human metapneumovirus (NC 004148), human papillomavirus-1 (NC-001356), human papillomavirus-18 (NC-001357), human papillomavirus-2 (NC-001352), human papillomavirus-54 (NC-001676), human papillomavirus-61 (NC-001694), human papillomavirus-cand90 (NC-004104), human papillomavirus RTRX7 (NC-004761), human papillomavirus type 10 (NC-001576), human papillomavirus type 101 (NC-008189), human papillomavirus type 103 (NC-008188), human papillomavirus type 107 (NC-009239), human papillomavirus type 16 (NC-001526), human papillomavirus type 24 (NC-001683), human papillomavirus type 26 (NC 001583), human papillomavirus type 32 (NC-001586), human papillomavirus type 34 (NC 001587), human papillomavirus type 4 (NC-001457), human papillomavirus type 41 (NC 001354), human papillomavirus type 48 (NC-001690), human papillomavirus type 49 (NC-001591), human papillomavirus type 5 (NC-001531), human papillomavirus type 50 (NC-001691), human papillomavirus type 53 (NC-001593), human papillomavirus type 60 (NC-001693), human papillomavirus type 63 (NC-001458), human papillomavirus type 6b (NC 001355), human papillomavirus type 7 (NC-001595), human papillomavirus type 71 (NC 002644), human papillomavirus type 9 (NC-001596), human papillomavirus type 92 (NC 004500), human papillomavirus type 96 (NC-005134), human parainfluenza virus 1 (NC 003461), human parainfluenza virus 2 (NC 003443), human parainfluenza virus 3 (NC 001796), human parechovirus (NC-001897), human parvovirus 4 (NC-007018), human parvovirus B19 (NC-000883), human respiratory syncytial virus (NC-001781), human rhinovirus A (NC-001617), human rhinovirus B (NC-001490), human spumaretrovirus (NC-001795), human T-lymphotropic virus 1 (NC-001436), human T-lymphotropic virus 2 (NC-001488).

In certain embodiments, the biomarker target protein is produced from a nucleic acid sequence (e.g., genomic target nucleic acid sequence) from an oncogenic virus, such as Epstein-Barr Virus (EBV) or a Human Papilloma Virus (HPV, e.g., HPV16, HPV18). In other embodiments, the target protein produced from a nucleic acid sequence (e.g., genomic target nucleic acid sequence) is from a pathogenic virus, such as a Respiratory Syncytial Virus, a Hepatitis Virus (e.g., Hepatitis C Virus), a Coronavirus (e.g., SARS virus), an Adenovirus, a Polyomavirus, a Cytomegalovirus (CMV), or a Herpes Simplex Virus (HSV).

Detectable Moieties

The presently disclosed methods utilize one or more detectable moieties. In some embodiments, the detectable moieties are a component of a detectable conjugate. In some embodiments, the detectable conjugates which may be used in the presently disclosed methods include the detectable moiety and one of a tyramide moiety (or a derivative or analog thereof), a quinone methide precursor moiety (or a derivative or analog thereof), or a functional group capable of participating in a “click chemistry” reaction (see also U.S. Pat. No. 10,041,950, and in U.S. Publication Nos. 2019/0204330, 2017/0089911, and 2019/0187130, the disclosures of which are hereby incorporated by reference herein in their entireties). In other embodiments, the detectable conjugates which may be used in the presently disclosed methods include the detectable moiety and one of a hapten, an enzyme, or an antibody.

In some embodiments, suitable detectable moieties may be characterized according to a full width of their first absorbance peak at the half maximum absorbance, referred to herein as FWHM (“full-width half-max”). FWHM is an expression of the extent of function given by the difference between the two extreme values of the independent variable at which the dependent variable is equal to half of its maximum value. In other words, it is the width of a spectrum curve measured between those points on the y-axis which are half the maximum amplitude. It is given by the distance between points on the curve at which the function reaches half its maximum value. Essentially, FWHM is a parameter commonly used to describe the width of a “bump” on a curve or function. In some embodiments, while an absorbance maximum (λₘₐₓ) may describe the wavelength of maximum absorption of a detectable moiety, the FWHM describes the breadth of the spectral absorbance.

In some embodiments, the detectable moieties have a narrow FWHM. In some embodiments, the detectable moiety has a first absorbance peak having a full width at half maximum which is less than the FWHM of a traditional dye or chromogen (e.g. one typically deposited by precipitation). For example, a traditional chromogen (e.g. DAB, Fast Red, Fast Blue, or a nanoparticulate silver stain as used in SISH techniques) may have a FWHM of about 200 nm or more; while the detectable moieties of the present disclosure may have a FWHM of less than about 200 nm, for example, less than about 150 nm, less than about 130 nm, less than about 100 nm, less than about 80 nm, or less than about 60 nm.

In some embodiments, the FWHM of the detectable moieties have a FWHM which is 40% less than a FWHM of a conventional dye or chromogen (e.g. hematoxylin, eosin or a special stain); 50% less than a FWHM of a conventional dye or chromogen; 55% less than a FWHM of a conventional dye or chromogen; 65% less than a FWHM of a conventional dye or chromogen; 70% less than a FWHM of a conventional dye or chromogen; 75% less than a FWHM of a conventional dye or chromogen; 80% less than the FWHM of a conventional dye or chromogen; 85% less than a FWHM of a conventional dye or chromogen; 90% less than a FWHM of a conventional dye or chromogen; or 95% less than a FWHM of a conventional dye or chromogen.

In some embodiments, the detectable moieties have a first absorbance peak with FWHM of less than about 200 nm. In some embodiments, the detectable moieties have a first absorbance peak with FWHM of less than about 190 nm. In some embodiments, the detectable moieties have a first absorbance peak with FWHM of less than about 180 nm. In some embodiments, the detectable moieties have a first absorbance peak with FWHM of less than about 170 nm. In some embodiments, the detectable moieties have a first absorbance peak with FWHM of less than about 160 nm. In some embodiments, the detectable moieties have a first absorbance peak with FWHM of less than about 150 nm. In some embodiments, the detectable moieties have a first absorbance peak with FWHM of less than about 140 nm. In some embodiments, the detectable moieties have a first absorbance peak with FWHM of less than about 130 nm. In some embodiments, the detectable moieties have a first absorbance peak with FWHM of less than about 120 nm. In some embodiments, the detectable moieties have a first absorbance peak with FWHM of less than about 110 nm. In some embodiments, the detectable moieties have a first absorbance peak with FWHM of less than about 100 nm. In some embodiments, the detectable moieties have a first absorbance peak with FWHM of less than about 90 nm. In some embodiments, the detectable moieties have a first absorbance peak with FWHM of less than about 80 nm. In some embodiments, the detectable moieties have a first absorbance peak with FWHM of less than about 70 nm. In some embodiments, the detectable moieties have a first absorbance peak with FWHM of less than about 60 nm. In some embodiments, the detectable moieties have a first absorbance peak with FWHM of less than about 50 nm.

Detectable Moieties within the Ultraviolet Spectrum

In some embodiments, the detectable moieties have a peak absorbance wavelength within the ultraviolet spectrum. In some embodiments, the detectable moieties have a peak absorbance peak absorbance wavelength of less than about 420 nm. In some embodiments, the detectable moieties have a peak absorbance wavelength of less than about 415 nm. In some embodiments, the detectable moieties have a peak absorbance wavelength of less than about 410 nm. In some embodiments, the detectable moieties have a peak absorbance wavelength of less than about 400 nm. In some embodiments, the detectable moieties have a peak absorbance wavelength of less than about 405 nm. In some embodiments, the detectable moiety of the disclosed compounds has a peak absorbance wavelength of less than about 395 nm. In some embodiments, the detectable moieties have a peak absorbance wavelength of less than about 390 nm. In some embodiments, the detectable moieties have a peak absorbance wavelength of less than about 385 nm. In some embodiments, the detectable moieties have a peak absorbance wavelength of less than about 380 nm. In some embodiments, the detectable moieties have a peak absorbance wavelength of less than about 375 nm. In some embodiments, the detectable moiety of the disclosed compounds has a peak absorbance wavelength of less than about 370 nm. In some embodiments, the detectable moieties have a peak absorbance wavelength ranging from between about 100 nm to about 400 nm, from about 100 nm to about 390 nm, from about 100 nm to about 380 nm, or from about 100 nm to about 370 nm.

In some embodiments, the detectable moieties have a peak absorbance wavelength of less than about 420 nm and a first absorbance peak with FWHM of less than 160 nm. In some embodiments, the detectable moieties have a peak absorbance wavelength of less than about 415 nm and a first absorbance peak with FWHM of less than 160 nm. In some embodiments, the detectable moieties have a peak absorbance wavelength of less than about 410 nm and a first absorbance peak with FWHM of less than 160 nm. In some embodiments, the detectable moieties have a peak absorbance wavelength of less than about 400 nm and a first absorbance peak with FWHM of less than 160 nm. In some embodiments, the detectable moieties have a peak absorbance wavelength of less than about 405 nm and a first absorbance peak with FWHM of less than 160 nm. In some embodiments, the detectable moiety of the disclosed compounds has a peak absorbance wavelength of less than about 395 nm and a first absorbance peak with FWHM of less than 160 nm. In some embodiments, the detectable moieties have a peak absorbance wavelength of less than about 390 nm and a first absorbance peak with FWHM of less than 160 nm. In some embodiments, the detectable moieties have a peak absorbance wavelength of less than about 385 nm and a first absorbance peak with FWHM of less than 160 nm. In some embodiments, the detectable moieties have a peak absorbance wavelength of less than about 380 nm and a first absorbance peak with FWHM of less than 160 nm. In some embodiments, the detectable moieties have a peak absorbance wavelength of less than about 375 nm and a first absorbance peak with FWHM of less than 160 nm. In some embodiments, the detectable moiety of the disclosed compounds has a peak absorbance wavelength of less than about 370 nm and a first absorbance peak with FWHM of less than 160 nm.

In some embodiments, the detectable moieties have a peak absorbance wavelength of less than about 420 nm and a first absorbance peak with FWHM of less than 130 nm. In some embodiments, the detectable moieties have a peak absorbance wavelength of less than about 415 nm and a first absorbance peak with FWHM of less than 130 nm. In some embodiments, the detectable moieties have a peak absorbance wavelength of less than about 410 nm and a first absorbance peak with FWHM of less than 130 nm. In some embodiments, the detectable moieties have a peak absorbance wavelength of less than about 400 nm and a first absorbance peak with FWHM of less than 130 nm. In some embodiments, the detectable moieties have a peak absorbance wavelength of less than about 405 nm and a first absorbance peak with FWHM of less than 130 nm. In some embodiments, the detectable moiety of the disclosed compounds has a peak absorbance wavelength of less than about 395 nm and a first absorbance peak with FWHM of less than 130 nm. In some embodiments, the detectable moieties have a peak absorbance wavelength of less than about 390 nm and a first absorbance peak with FWHM of less than 130 nm. In some embodiments, the detectable moieties have a peak absorbance wavelength of less than about 385 nm and a first absorbance peak with FWHM of less than 130 nm. In some embodiments, the detectable moieties have a peak absorbance wavelength of less than about 380 nm and a first absorbance peak with FWHM of less than 130 nm. In some embodiments, the detectable moieties have a peak absorbance wavelength of less than about 375 nm and a first absorbance peak with FWHM of less than 130 nm. In some embodiments, the detectable moiety of the disclosed compounds has a peak absorbance wavelength of less than about 370 nm and a first absorbance peak with FWHM of less than 130 nm.

In some embodiments, the detectable moieties have a peak absorbance wavelength of less than about 420 nm and a first absorbance peak with FWHM of less than 100 nm. In some embodiments, the detectable moieties have a peak absorbance wavelength of less than about 415 nm and a first absorbance peak with FWHM of less than 100 nm. In some embodiments, the detectable moieties have a peak absorbance wavelength of less than about 410 nm and a first absorbance peak with FWHM of less than 100 nm. In some embodiments, the detectable moieties have a peak absorbance wavelength of less than about 400 nm and a first absorbance peak with FWHM of less than 100 nm. In some embodiments, the detectable moieties have a peak absorbance wavelength of less than about 405 nm and a first absorbance peak with FWHM of less than 100 nm. In some embodiments, the detectable moiety of the disclosed compounds has a peak absorbance wavelength of less than about 395 nm and a first absorbance peak with FWHM of less than 100 nm. In some embodiments, the detectable moieties have a peak absorbance wavelength of less than about 390 nm and a first absorbance peak with FWHM of less than 100 nm. In some embodiments, the detectable moieties have a peak absorbance wavelength of less than about 385 nm and a first absorbance peak with FWHM of less than 100 nm. In some embodiments, the detectable moieties have a peak absorbance wavelength of less than about 380 nm and a first absorbance peak with FWHM of less than 100 nm. In some embodiments, the detectable moieties have a peak absorbance wavelength of less than about 375 nm and a first absorbance peak with FWHM of less than 100 nm. In some embodiments, the detectable moiety of the disclosed compounds has a peak absorbance wavelength of less than about 370 nm and a first absorbance peak with FWHM of less than 100 nm.

In some embodiments, the detectable moieties have a peak absorbance wavelength of less than about 420 nm and a first absorbance peak with FWHM of less than 80 nm. In some embodiments, the detectable moieties have a peak absorbance wavelength of less than about 415 nm and a first absorbance peak with FWHM of less than 80 nm. In some embodiments, the detectable moieties have a peak absorbance wavelength of less than about 410 nm and a first absorbance peak with FWHM of less than 80 nm. In some embodiments, the detectable moieties have a peak absorbance wavelength of less than about 400 nm and a first absorbance peak with FWHM of less than 80 nm. In some embodiments, the detectable moieties have a peak absorbance wavelength of less than about 405 nm and a first absorbance peak with FWHM of less than 80 nm. In some embodiments, the detectable moiety of the disclosed compounds has a peak absorbance wavelength of less than about 395 nm and a first absorbance peak with FWHM of less than 80 nm. In some embodiments, the detectable moieties have a peak absorbance wavelength of less than about 390 nm and a first absorbance peak with FWHM of less than 80 nm. In some embodiments, the detectable moieties have a peak absorbance wavelength of less than about 385 nm and a first absorbance peak with FWHM of less than 80 nm. In some embodiments, the detectable moieties have a peak absorbance wavelength of less than about 380 nm and a first absorbance peak with FWHM of less than 80 nm. In some embodiments, the detectable moieties have a peak absorbance wavelength of less than about 375 nm and a first absorbance peak with FWHM of less than 80 nm. In some embodiments, the detectable moiety of the disclosed compounds has a peak absorbance wavelength of less than about 370 nm and a first absorbance peak with FWHM of less than 80 nm.

In some embodiments, the detectable moiety includes or is derived from a coumarin (i.e. the detectable moiety includes a coumarin core). Examples of suitable detectable moieties having a coumarin core are described in U.S. Pat. No. 10,041,950, the disclosure of which is hereby incorporated by reference herein in its entirety. In some embodiments, the coumarin core is a coumarinamine core. In some embodiments, the coumarin core is a 7-coumarinamine core. In some embodiments, the coumarin core is a coumarinol core. In some embodiments, the coumarin core is a 7-coumarinol core.

In some embodiments, the coumarin core includes (or is modified to include) one or more electron withdrawing groups (where each electron withdrawing group may be the same or different). In some embodiments, the coumarin core includes (or is modified to include) one electron withdrawing group. In some embodiments, the coumarin core includes (or is modified to include) two electron withdrawing groups. In some embodiments, the coumarin core includes (or is modifying to include) three electron withdrawing groups. In some embodiments, the coumarin core includes (or is modifying to include) three different electron withdrawing groups. In some embodiments, the coumarin core includes (or is modified to include) four electron withdrawing groups. In some embodiments, the one or more electron withdrawing groups have an electronegatively ranging from between about 1.5 to about 3.5 each.

In some embodiments, the coumarin core includes (or is modified to include) one or more electron donating groups (where each electron donating group may be the same or different). In some embodiments, the coumarin core includes (or is modified to include) one electron donating group. In some embodiments, the coumarin core includes (or is modified to include) two electron donating groups. In some embodiments, the coumarin core includes (or is modifying to include) three electron donating groups. In some embodiments, the coumarin core includes (or is modifying to include) three different electron donating groups. In some embodiments, the coumarin core includes (or is modified to include) four electron donating groups. In some embodiments, the one or more electron donating groups have an electronegatively ranging from between about 1.5 to about 3.5 each. In some embodiments, one or more electronic withdrawing and/or donating groups are incorporated to facilitate a shift towards the “red” spectrum or the “blue” spectrum.

In some embodiments, the detectable moieties having the coumarin core have a wavelength ranging from about 300 nm to about 460 nm. In some embodiments, the detectable moieties having the coumarin core have a wavelength ranging from about 320 nm to about 440 nm. In some embodiments, the detectable moieties having the coumarin core have a wavelength ranging from about 340 nm to about 430 nm. These ranges may be altered or shift as more or less electronegative is introduced to the coumarin core.

In some embodiments, the detectable moieties having the coumarin core have a peak absorbance wavelength of about 460 nm+/−10 nm. In some embodiments, the detectable moieties having the coumarin core have a peak absorbance wavelength of about 455+/−10 nm. In some embodiments, the detectable moieties having the coumarin core have a peak absorbance wavelength of about 450 nm+/−10 nm. In some embodiments, the detectable moieties having the coumarin core have a peak absorbance wavelength of about 445 nm+/−10 nm. In some embodiments, the detectable moieties having the coumarin core have a peak absorbance wavelength of about 440 nm+/−10 nm. In some embodiments, the detectable moieties having the coumarin core have a peak absorbance wavelength of about 435 nm+/−10 nm. In some embodiments, the detectable moieties having the coumarin core have a peak absorbance wavelength of about 430 nm+/−10 nm. In some embodiments, the detectable moieties having the coumarin core have a peak absorbance wavelength of about 425 nm+/−10 nm. In some embodiments, the detectable moieties having the coumarin core have a peak absorbance wavelength of about 420 nm+/−10 nm. In some embodiments, the detectable moieties having the coumarin core have a peak absorbance wavelength of about 415 nm+/−10 nm. In some embodiments, the detectable moieties having the coumarin core have a peak absorbance wavelength of about 410 nm+/−10 nm. In some embodiments, the detectable moieties having the coumarin core have a peak absorbance wavelength of about 405 nm+/−10 nm. In some embodiments, the detectable moieties having the coumarin core have a peak absorbance wavelength of about 400 nm+/−10 nm. In some embodiments, the detectable moieties having the coumarin core have a peak absorbance wavelength of about 395 nm+/−10 nm. In some embodiments, the detectable moieties having the coumarin core have a peak absorbance wavelength of about 390 nm+/−10 nm. In some embodiments, the detectable moieties having the coumarin core have a peak absorbance wavelength of about 385 nm+/−10 nm. In some embodiments, the detectable moieties having the coumarin core have a peak absorbance wavelength of about 380 nm+/−10 nm. In some embodiments, the detectable moieties having the coumarin core have a peak absorbance wavelength of about 375 nm+/−10 nm. In some embodiments, the detectable moieties having the coumarin core have a peak absorbance wavelength of about 370 nm+/−10 nm. In some embodiments, the detectable moieties having the coumarin core have a peak absorbance wavelength of about 365 nm+/−10 nm. In some embodiments, the detectable moieties having the coumarin core have a peak absorbance wavelength of about 360 nm+/−10 nm. In some embodiments, the detectable moieties having the coumarin core have a peak absorbance wavelength of about 355 nm+/−10 nm. In some embodiments, the detectable moieties having the coumarin core have a peak absorbance wavelength of about 350 nm+/−10 nm. In some embodiments, the detectable moieties having the coumarin core have a peak absorbance wavelength of about 345 nm+/−10 nm. In some embodiments, the detectable moieties having the coumarin core have a peak absorbance wavelength of about 340 nm+/−10 nm. In some embodiments, the detectable moieties having the coumarin core have a peak absorbance wavelength of about 335 nm+/−10 nm. In some embodiments, the detectable moieties having the coumarin core have a peak absorbance wavelength of about 330 nm+/−10 nm.

In some embodiments, the detectable moieties having the coumarin core have a peak absorbance wavelength of about 460 nm+/−10 nm and a first absorbance peak with FWHM of less than 160 nm. In some embodiments, the detectable moieties having the coumarin core have a peak absorbance wavelength of about 455+/−10 nm and a first absorbance peak with FWHM of less than 160 nm. In some embodiments, the detectable moieties having the coumarin core have a peak absorbance wavelength of about 450 nm+/−10 nm and a first absorbance peak with FWHM of less than 160 nm. In some embodiments, the detectable moieties having the coumarin core have a peak absorbance wavelength of about 445 nm+/−10 nm and a first absorbance peak with FWHM of less than 160 nm. In some embodiments, the detectable moieties having the coumarin core have a peak absorbance wavelength of about 440 nm+/−10 nm and a first absorbance peak with FWHM of less than 160 nm. In some embodiments, the detectable moieties having the coumarin core have a peak absorbance wavelength of about 435 nm+/−10 nm and a first absorbance peak with FWHM of less than 160 nm. In some embodiments, the detectable moieties having the coumarin core have a peak absorbance wavelength of about 430 nm+/−10 nm and a first absorbance peak with FWHM of less than 160 nm. In some embodiments, the detectable moieties having the coumarin core have a peak absorbance wavelength of about 425 nm+/−10 nm and a first absorbance peak with FWHM of less than 160 nm. In some embodiments, the detectable moieties having the coumarin core have a peak absorbance wavelength of about 420 nm+/−10 nm and a first absorbance peak with FWHM of less than 160 nm. In some embodiments, the detectable moieties having the coumarin core have a peak absorbance wavelength of about 415 nm+/−10 nm and a first absorbance peak with FWHM of less than 160 nm. In some embodiments, the detectable moieties having the coumarin core have a peak absorbance wavelength of about 410 nm and a first absorbance peak with FWHM of less than 160 nm. In some embodiments, the detectable moieties having the coumarin core have a peak absorbance wavelength of about 405 nm+/−10 nm and a first absorbance peak with FWHM of less than 160 nm. In some embodiments, the detectable moieties having the coumarin core have a peak absorbance wavelength of about 400 nm+/−10 nm and a first absorbance peak with FWHM of less than 160 nm. In some embodiments, the detectable moieties having the coumarin core have a peak absorbance wavelength of about 395 nm+/−10 nm and a first absorbance peak with FWHM of less than 160 nm. In some embodiments, the detectable moieties having the coumarin core have a peak absorbance wavelength of about 390 nm+/−10 nm and a first absorbance peak with FWHM of less than 160 nm. In some embodiments, the detectable moieties having the coumarin core have a peak absorbance wavelength of about 385 nm+/−10 nm and a first absorbance peak with FWHM of less than 160 nm. In some embodiments, the detectable moieties having the coumarin core have a peak absorbance wavelength of about 380 nm+/−10 nm and a first absorbance peak with FWHM of less than 160 nm. In some embodiments, the detectable moieties having the coumarin core have a peak absorbance wavelength of about 375 nm+/−10 nm and a first absorbance peak with FWHM of less than 160 nm. In some embodiments, the detectable moieties having the coumarin core have a peak absorbance wavelength of about 370 nm+/−10 nm and a first absorbance peak with FWHM of less than 160 nm. In some embodiments, the detectable moieties having the coumarin core have a peak absorbance wavelength of about 365 nm+/−10 nm and a first absorbance peak with FWHM of less than 160 nm. In some embodiments, the detectable moieties having the coumarin core have a peak absorbance wavelength of about 3160 nm+/−10 nm and a first absorbance peak with FWHM of less than 160 nm. In some embodiments, the detectable moieties having the coumarin core have a peak absorbance wavelength of about 355 nm+/−10 nm and a first absorbance peak with FWHM of less than 160 nm. In some embodiments, the detectable moieties having the coumarin core have a peak absorbance wavelength of about 350 nm+/−10 nm and a first absorbance peak with FWHM of less than 160 nm. In some embodiments, the detectable moieties having the coumarin core have a peak absorbance wavelength of about 345 nm+/−10 nm and a first absorbance peak with FWHM of less than 160 nm. In some embodiments, the detectable moieties having the coumarin core have a peak absorbance wavelength of about 340 nm+/−10 nm and a first absorbance peak with FWHM of less than 160 nm. In some embodiments, the detectable moieties having the coumarin core have a peak absorbance wavelength of about 335 nm+/−10 nm and a first absorbance peak with FWHM of less than 160 nm. In some embodiments, the detectable moieties having the coumarin core have a peak absorbance wavelength of about 330 nm+/−10 nm and a first absorbance peak with FWHM of less than 160 nm.

In some embodiments, the detectable moieties having the coumarin core have a peak absorbance wavelength of about 460 nm+/−10 nm and a first absorbance peak with FWHM of less than 130 nm. In some embodiments, the detectable moieties having the coumarin core have a peak absorbance wavelength of about 455+/−10 nm and a first absorbance peak with FWHM of less than 130 nm. In some embodiments, the detectable moieties having the coumarin core have a peak absorbance wavelength of about 450 nm+/−10 nm and a first absorbance peak with FWHM of less than 130 nm. In some embodiments, the detectable moieties having the coumarin core have a peak absorbance wavelength of about 445 nm+/−10 nm and a first absorbance peak with FWHM of less than 130 nm. In some embodiments, the detectable moieties having the coumarin core have a peak absorbance wavelength of about 440 nm+/−10 nm and a first absorbance peak with FWHM of less than 130 nm. In some embodiments, the detectable moieties having the coumarin core have a peak absorbance wavelength of about 435 nm+/−10 nm and a first absorbance peak with FWHM of less than 130 nm. In some embodiments, the detectable moieties having the coumarin core have a peak absorbance wavelength of about 430 nm+/−10 nm and a first absorbance peak with FWHM of less than 130 nm. In some embodiments, the detectable moieties having the coumarin core have a peak absorbance wavelength of about 425 nm+/−10 nm and a first absorbance peak with FWHM of less than 130 nm. In some embodiments, the detectable moieties having the coumarin core have a peak absorbance wavelength of about 420 nm+/−10 nm and a first absorbance peak with FWHM of less than 130 nm. In some embodiments, the detectable moieties having the coumarin core have a peak absorbance wavelength of about 415 nm+/−10 nm and a first absorbance peak with FWHM of less than 130 nm. In some embodiments, the detectable moieties having the coumarin core have a peak absorbance wavelength of about 410 nm and a first absorbance peak with FWHM of less than 130 nm. In some embodiments, the detectable moieties having the coumarin core have a peak absorbance wavelength of about 405 nm+/−10 nm and a first absorbance peak with FWHM of less than 130 nm. In some embodiments, the detectable moieties having the coumarin core have a peak absorbance wavelength of about 400 nm+/−10 nm and a first absorbance peak with FWHM of less than 130 nm. In some embodiments, the detectable moieties having the coumarin core have a peak absorbance wavelength of about 395 nm+/−10 nm and a first absorbance peak with FWHM of less than 130 nm. In some embodiments, the detectable moieties having the coumarin core have a peak absorbance wavelength of about 390 nm+/−10 nm and a first absorbance peak with FWHM of less than 130 nm. In some embodiments, the detectable moieties having the coumarin core have a peak absorbance wavelength of about 385 nm+/−10 nm and a first absorbance peak with FWHM of less than 130 nm. In some embodiments, the detectable moieties having the coumarin core have a peak absorbance wavelength of about 380 nm+/−10 nm and a first absorbance peak with FWHM of less than 130 nm. In some embodiments, the detectable moieties having the coumarin core have a peak absorbance wavelength of about 375 nm+/−10 nm and a first absorbance peak with FWHM of less than 130 nm. In some embodiments, the detectable moieties having the coumarin core have a peak absorbance wavelength of about 370 nm+/−10 nm and a first absorbance peak with FWHM of less than 130 nm. In some embodiments, the detectable moieties having the coumarin core have a peak absorbance wavelength of about 365 nm+/−10 nm and a first absorbance peak with FWHM of less than 130 nm. In some embodiments, the detectable moieties having the coumarin core have a peak absorbance wavelength of about 3130 nm+/−10 nm and a first absorbance peak with FWHM of less than 130 nm. In some embodiments, the detectable moieties having the coumarin core have a peak absorbance wavelength of about 355 nm+/−10 nm and a first absorbance peak with FWHM of less than 130 nm. In some embodiments, the detectable moieties having the coumarin core have a peak absorbance wavelength of about 350 nm+/−10 nm and a first absorbance peak with FWHM of less than 130 nm. In some embodiments, the detectable moieties having the coumarin core have a peak absorbance wavelength of about 345 nm+/−10 nm and a first absorbance peak with FWHM of less than 130 nm. In some embodiments, the detectable moieties having the coumarin core have a peak absorbance wavelength of about 340 nm+/−10 nm and a first absorbance peak with FWHM of less than 130 nm. In some embodiments, the detectable moieties having the coumarin core have a peak absorbance wavelength of about 335 nm+/−10 nm and a first absorbance peak with FWHM of less than 130 nm. In some embodiments, the detectable moieties having the coumarin core have a peak absorbance wavelength of about 330 nm+/−10 nm and a first absorbance peak with FWHM of less than 130 nm.

In some embodiments, the detectable moieties having the coumarin core have a peak absorbance wavelength of about 460 nm+/−10 nm and a first absorbance peak with FWHM of less than 100 nm. In some embodiments, the detectable moieties having the coumarin core have a peak absorbance wavelength of about 455+/−10 nm and a first absorbance peak with FWHM of less than 100 nm. In some embodiments, the detectable moieties having the coumarin core have a peak absorbance wavelength of about 450 nm+/−10 nm and a first absorbance peak with FWHM of less than 100 nm. In some embodiments, the detectable moieties having the coumarin core have a peak absorbance wavelength of about 445 nm+/−10 nm and a first absorbance peak with FWHM of less than 100 nm. In some embodiments, the detectable moieties having the coumarin core have a peak absorbance wavelength of about 440 nm+/−10 nm and a first absorbance peak with FWHM of less than 100 nm. In some embodiments, the detectable moieties having the coumarin core have a peak absorbance wavelength of about 435 nm+/−10 nm and a first absorbance peak with FWHM of less than 100 nm. In some embodiments, the detectable moieties having the coumarin core have a peak absorbance wavelength of about 430 nm+/−10 nm and a first absorbance peak with FWHM of less than 100 nm. In some embodiments, the detectable moieties having the coumarin core have a peak absorbance wavelength of about 425 nm+/−10 nm and a first absorbance peak with FWHM of less than 100 nm. In some embodiments, the detectable moieties having the coumarin core have a peak absorbance wavelength of about 420 nm+/−10 nm and a first absorbance peak with FWHM of less than 100 nm. In some embodiments, the detectable moieties having the coumarin core have a peak absorbance wavelength of about 415 nm+/−10 nm and a first absorbance peak with FWHM of less than 100 nm. In some embodiments, the detectable moieties having the coumarin core have a peak absorbance wavelength of about 410 nm and a first absorbance peak with FWHM of less than 100 nm. In some embodiments, the detectable moieties having the coumarin core have a peak absorbance wavelength of about 405 nm+/−10 nm and a first absorbance peak with FWHM of less than 100 nm. In some embodiments, the detectable moieties having the coumarin core have a peak absorbance wavelength of about 400 nm+/−10 nm and a first absorbance peak with FWHM of less than 100 nm. In some embodiments, the detectable moieties having the coumarin core have a peak absorbance wavelength of about 395 nm+/−10 nm and a first absorbance peak with FWHM of less than 100 nm. In some embodiments, the detectable moieties having the coumarin core have a peak absorbance wavelength of about 390 nm+/−10 nm and a first absorbance peak with FWHM of less than 100 nm. In some embodiments, the detectable moieties having the coumarin core have a peak absorbance wavelength of about 385 nm+/−10 nm and a first absorbance peak with FWHM of less than 100 nm. In some embodiments, the detectable moieties having the coumarin core have a peak absorbance wavelength of about 380 nm+/−10 nm and a first absorbance peak with FWHM of less than 100 nm. In some embodiments, the detectable moieties having the coumarin core have a peak absorbance wavelength of about 375 nm+/−10 nm and a first absorbance peak with FWHM of less than 100 nm. In some embodiments, the detectable moieties having the coumarin core have a peak absorbance wavelength of about 370 nm+/−10 nm and a first absorbance peak with FWHM of less than 100 nm. In some embodiments, the detectable moieties having the coumarin core have a peak absorbance wavelength of about 365 nm+/−10 nm and a first absorbance peak with FWHM of less than 100 nm. In some embodiments, the detectable moieties having the coumarin core have a peak absorbance wavelength of about 360 nm+/−10 nm and a first absorbance peak with FWHM of less than 100 nm. In some embodiments, the detectable moieties having the coumarin core have a peak absorbance wavelength of about 355 nm+/−10 nm and a first absorbance peak with FWHM of less than 100 nm. In some embodiments, the detectable moieties having the coumarin core have a peak absorbance wavelength of about 350 nm+/−10 nm and a first absorbance peak with FWHM of less than 100 nm. In some embodiments, the detectable moieties having the coumarin core have a peak absorbance wavelength of about 345 nm+/−10 nm and a first absorbance peak with FWHM of less than 100 nm. In some embodiments, the detectable moieties having the coumarin core have a peak absorbance wavelength of about 340 nm+/−10 nm and a first absorbance peak with FWHM of less than 100 nm. In some embodiments, the detectable moieties having the coumarin core have a peak absorbance wavelength of about 335 nm+/−10 nm and a first absorbance peak with FWHM of less than 100 nm. In some embodiments, the detectable moieties having the coumarin core have a peak absorbance wavelength of about 330 nm+/−10 nm and a first absorbance peak with FWHM of less than 100 nm.

Examples of detectable moieties having a coumarin core include:

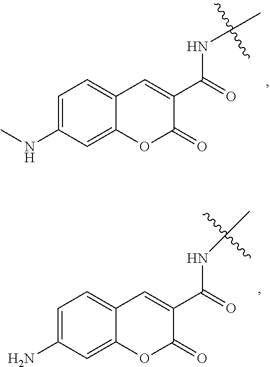 [SMILES: *NC(=O)c1cc2ccc(NC)cc2oc1=O ; *NC(=O)c1cc2ccc(N)cc2oc1=O]

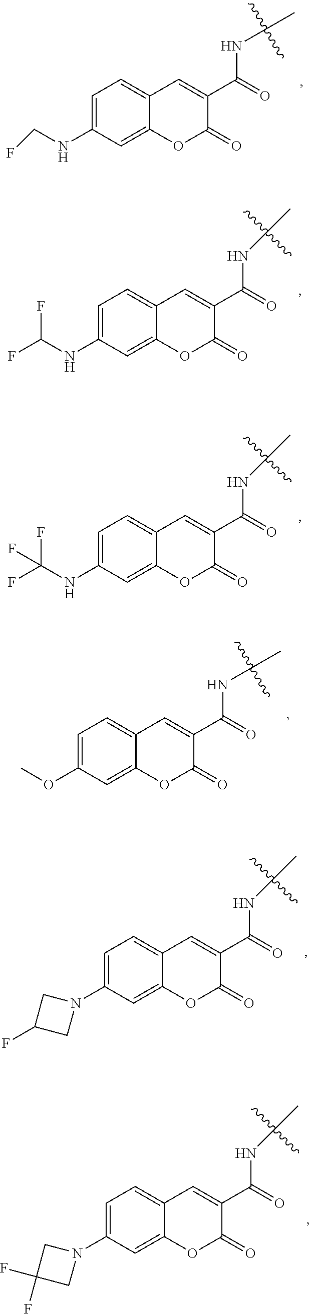 [SMILES: *NC(=O)c1cc2ccc(NCF)cc2oc1=O ; *NC(=O)c1cc2ccc(NC(F)F)cc2oc1=O ; *NC(=O)c1cc2ccc(NC(F)(F)F)cc2oc1=O ; *NC(=O)c1cc2ccc(OC)cc2oc1=O ; *NC(=O)c1cc2ccc(N3CC(F)C3)cc2oc1=O ; *NC(=O)c1cc2ccc(N3CC(F)(F)C3)cc2oc1=O]

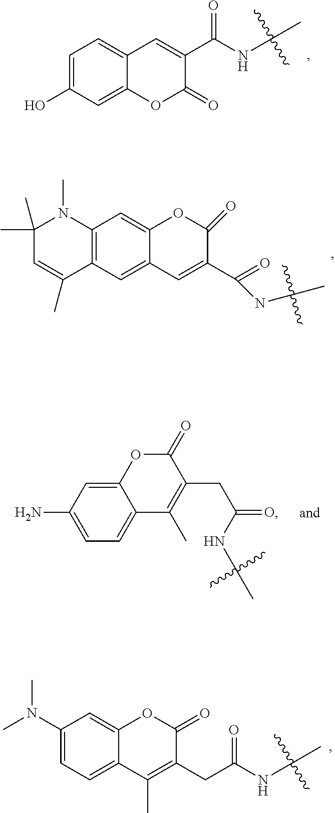 [SMILES: *NC(=O)c1cc2ccc(O)cc2oc1=O ; *NC(=O)c1cc2cc3c(cc2oc1=O)N(C)C(C)(C)C=C3C ; *NC(=O)Cc1c(C)c2ccc(N)cc2oc1=O ; *NC(=O)Cc1c(C)c2ccc(N(C)C)cc2oc1=O]

where the symbol “ [symbol] ” refers to the site in which the detectable moiety (here, the coumarin core) is coupled (directly or indirectly) to another moiety of the detectable conjugate (e.g. to a tyramide moiety, to a quinone methide moiety, to a functional group capable or participating in a “click chemistry” reaction, to an antibody, to an enzyme, to a hapten, etc.).

Other suitable detectable moieties having a coumarin core are described in U.S. Pat. No. 10,041,950, the disclosure of which is incorporated by reference herein in its entirety, provided those coumarin-based compounds have a first absorbance peak with FWHM of less than about 200 nm.

Yet other examples are disclosed herein.

Detectable Moieties within the Visible Spectrum

In some embodiments, the detectable moieties have a peak absorbance wavelength within the visible spectrum. In some embodiments, the detectable moieties have a peak absorbance peak absorbance wavelength of between about 400 nm to about 760 nm. In some embodiments, the detectable moieties have a peak absorbance wavelength of between about 440 nm to about 720 nm. In some embodiments, the detectable moieties have a peak absorbance wavelength of between about 460 nm to about 680 nm. In some embodiments, the detectable moieties have a peak absorbance wavelength of between about 500 nm to about 640 nm. In some embodiments, the detectable moieties have a peak absorbance wavelength of between about 540 nm to about 600 nm.

In some embodiments, the detectable moieties have a peak absorbance wavelength within the visible spectrum. In some embodiments, the detectable moieties have a peak absorbance peak absorbance wavelength of between about 400 nm to about 760 nm and a first absorbance peak with FWHM a first absorbance peak with FWHM of less than 160 nm. In some embodiments, the detectable moieties have a peak absorbance wavelength of between about 440 nm to about 720 nm and a first absorbance peak with FWHM of less than 160 nm. In some embodiments, the detectable moieties have a peak absorbance wavelength of between about 460 nm to about 680 nm and a first absorbance peak with FWHM of less than 160 nm. In some embodiments, the detectable moieties have a peak absorbance wavelength of between about 500 nm to about 640 nm and a first absorbance peak with FWHM of less than 160 nm. In some embodiments, the detectable moieties have a peak absorbance wavelength of between about 540 nm to about 600 nm and a first absorbance peak with FWHM of less than 160 nm.

In some embodiments, the detectable moieties have a peak absorbance peak absorbance wavelength of between about 400 nm to about 760 nm and a first absorbance peak with FWHM of less than 130 nm. In some embodiments, the detectable moieties have a peak absorbance wavelength of between about 440 nm to about 720 nm and a first absorbance peak with FWHM of less than 130 nm. In some embodiments, the detectable moieties have a peak absorbance wavelength of between about 460 nm to about 680 nm and a first absorbance peak with FWHM of less than 130 nm. In some embodiments, the detectable moieties have a peak absorbance wavelength of between about 500 nm to about 640 nm and a first absorbance peak with FWHM of less than 130 nm.

In some embodiments, the detectable moieties have a peak absorbance peak absorbance wavelength of between about 400 nm to about 760 nm and a first absorbance peak with FWHM of less than 100 nm. In some embodiments, the detectable moieties have a peak absorbance wavelength of between about 440 nm to about 720 nm and a first absorbance peak with FWHM of less than 100 nm. In some embodiments, the detectable moieties have a peak absorbance wavelength of between about 460 nm to about 680 nm and a first absorbance peak with FWHM of less than 100 nm. In some embodiments, the detectable moieties have a peak absorbance wavelength of between about 500 nm to about 640 nm and a first absorbance peak with FWHM of less than 100 nm.

In some embodiments, the detectable moieties have a peak absorbance peak absorbance wavelength of between about 400 nm to about 760 nm and a first absorbance peak with FWHM of less than 80 nm. In some embodiments, the detectable moieties have a peak absorbance wavelength of between about 440 nm to about 720 nm and a first absorbance peak with FWHM of less than 80 nm. In some embodiments, the detectable moieties have a peak absorbance wavelength of between about 460 nm to about 680 nm and a first absorbance peak with FWHM of less than 80 nm. In some embodiments, the detectable moieties have a peak absorbance wavelength of between about 500 nm to about 640 nm and a first absorbance peak with FWHM of less than 80 nm.

In some embodiments, the detectable moiety includes or is derived from a phenoxazine or a phenoxazinone (i.e. the detectable moiety includes a phenoxazine or a phenoxazinone core). In some embodiments, the detectable moiety derived from a phenoxazine or a phenoxazinone is a 4-Hydroxy-3-phenoxazinone or is a 7-amino-4-Hydroxy-3-phenoxazinone.

In some embodiments, the phenoxazine or a phenoxazinone core includes (or is modified to include) one or more electron withdrawing groups (where each electron withdrawing group may be the same or different). In some embodiments, the phenoxazine or a phenoxazinone core includes (or is modified to include) one electron withdrawing group. In some embodiments, the phenoxazine or a phenoxazinone core includes (or is modified to include) two electron withdrawing groups. In some embodiments, the phenoxazine or a phenoxazinone core includes (or is modifying to include) three electron withdrawing groups. In some embodiments, the phenoxazine or a phenoxazinone core includes (or is modifying to include) three different electron withdrawing groups. In some embodiments, the phenoxazine or a phenoxazinone core includes (or is modified to include) four electron withdrawing groups.

In some embodiments, the phenoxazine or a phenoxazinone core includes (or is modified to include) one or more electron donating groups (where each electron withdrawing group may be the same or different). In some embodiments, the phenoxazine or a phenoxazinone core includes (or is modified to include) one electron donating group. In some embodiments, the phenoxazine or a phenoxazinone core includes (or is modified to include) two electron donating groups. In some embodiments, the phenoxazine or a phenoxazinone core includes (or is modifying to include) three electron donating groups. In some embodiments, the phenoxazine or a phenoxazinone core includes (or is modifying to include) three different electron donating groups. In some embodiments, the phenoxazine or a phenoxazinone core includes (or is modified to include) four electron donating groups.

In some embodiments, the detectable moieties having the phenoxazine or a phenoxazinone core have a peak absorbance wavelength ranging from about 580 nm to about 700 nm. In some embodiments, the detectable moieties having the phenoxazine or a phenoxazinone core have a peak absorbance wavelength ranging from about 600 nm to about 680 nm. In some embodiments, the detectable moieties having the phenoxazine or a phenoxazinone core have a peak absorbance wavelength ranging from about 620 nm to about 660 nm.

In some embodiments, the detectable moieties having the phenoxazine or a phenoxazinone core have a peak absorbance wavelength of about 700+/−10 nm. In some embodiments, the detectable moieties having the phenoxazine or a phenoxazinone core have a peak absorbance wavelength of about 695+/−10 nm. In some embodiments, the detectable moieties having the phenoxazine or a phenoxazinone core have a peak absorbance wavelength of about 690+/−10 nm. In some embodiments, the detectable moieties having the phenoxazine or a phenoxazinone core have a peak absorbance wavelength of about 685+/−10 nm. In some embodiments, the detectable moieties having the phenoxazine or a phenoxazinone core have a peak absorbance wavelength of about 680+/−10 nm. In some embodiments, the detectable moieties having the phenoxazine or a phenoxazinone core have a peak absorbance wavelength of about 675+/−10 nm. In some embodiments, the detectable moieties having the phenoxazine or a phenoxazinone core have a peak absorbance wavelength of about 670+/−10 nm. In some embodiments, the detectable moieties having the phenoxazine or a phenoxazinone core have a peak absorbance wavelength of about 665+/−10 nm. In some embodiments, the detectable moieties having the phenoxazine or a phenoxazinone core have a peak absorbance wavelength of about 660+/−10 nm. In some embodiments, the detectable moieties having the phenoxazine or a phenoxazinone core have a peak absorbance wavelength of about 655+/−10 nm. In some embodiments, the detectable moieties having the phenoxazine or a phenoxazinone core have a peak absorbance wavelength of about 650+/−10 nm. In some embodiments, the detectable moieties having the phenoxazine or a phenoxazinone core have a peak absorbance wavelength of about 645+/−10 nm. In some embodiments, the detectable moieties having the phenoxazine or a phenoxazinone core have a peak absorbance wavelength of about 640+/−10 nm. In some embodiments, the detectable moieties having the phenoxazine or a phenoxazinone core have a peak absorbance wavelength of about 635+/−10 nm. In some embodiments, the detectable moieties having the phenoxazine or a phenoxazinone core have a peak absorbance wavelength of about 630+/−10 nm. In some embodiments, the detectable moieties having the phenoxazine or a phenoxazinone core have a peak absorbance wavelength of about 625+/−10 nm. In some embodiments, the detectable moieties having the phenoxazine or a phenoxazinone core have a peak absorbance wavelength of about 620+/−10 nm. In some embodiments, the detectable moieties having the phenoxazine or a phenoxazinone core have a peak absorbance wavelength of about 615+/−10 nm. In some embodiments, the detectable moieties having the phenoxazine or a phenoxazinone core have a peak absorbance wavelength of about 610+/−10 nm. In some embodiments, the detectable moieties having the phenoxazine or a phenoxazinone core have a peak absorbance wavelength of about 605+/−10 nm. In some embodiments, the detectable moieties having the phenoxazine or a phenoxazinone core have a peak absorbance wavelength of about 600+/−10 nm. In some embodiments, the detectable moieties having the phenoxazine or a phenoxazinone core have a peak absorbance wavelength of about 595+/−10 nm. In some embodiments, the detectable moieties having the phenoxazine or a phenoxazinone core have a peak absorbance wavelength of about 590+/−10 nm. In some embodiments, the detectable moieties having the phenoxazine or a phenoxazinone core have a peak absorbance wavelength of about 585+/−10 nm. In some embodiments, the detectable moieties having the phenoxazine or a phenoxazinone core have a peak absorbance wavelength of about 580+/−10 nm.

In some embodiments, the detectable moieties having the phenoxazine or a phenoxazinone core have a peak absorbance wavelength of about 700+/−10 nm and a first absorbance peak with FWHM of less than 160 nm. In some embodiments, the detectable moieties having the phenoxazine or a phenoxazinone core have a peak absorbance wavelength of about 695+/−10 nm and a first absorbance peak with FWHM of less than 160 nm. In some embodiments, the detectable moieties having the phenoxazine or a phenoxazinone core have a peak absorbance wavelength of about 690+/−10 nm and a first absorbance peak with FWHM of less than 160 nm. In some embodiments, the detectable moieties having the phenoxazine or a phenoxazinone core have a peak absorbance wavelength of about 685+/−10 nm and a first absorbance peak with FWHM of less than 160 nm. In some embodiments, the detectable moieties having the phenoxazine or a phenoxazinone core have a peak absorbance wavelength of about 680+/−10 nm and a first absorbance peak with FWHM of less than 160 nm. In some embodiments, the detectable moieties having the phenoxazine or a phenoxazinone core have a peak absorbance wavelength of about 675+/−10 nm and a first absorbance peak with FWHM of less than 160 nm. In some embodiments, the detectable moieties having the phenoxazine or a phenoxazinone core have a peak absorbance wavelength of about 670+/−10 nm and a first absorbance peak with FWHM of less than 160 nm. In some embodiments, the detectable moieties having the phenoxazine or a phenoxazinone core have a peak absorbance wavelength of about 665+/−10 nmm and a first absorbance peak with FWHM of less than 160 nm. In some embodiments, the detectable moieties having the phenoxazine or a phenoxazinone core have a peak absorbance wavelength of about 660+/−10 nm and a first absorbance peak with FWHM of less than 160 nm. In some embodiments, the detectable moieties having the phenoxazine or a phenoxazinone core have a peak absorbance wavelength of about 655+/−10 nm and a first absorbance peak with FWHM of less than 160 nm. In some embodiments, the detectable moieties having the phenoxazine or a phenoxazinone core have a peak absorbance wavelength of about 650+/−10 nm and a first absorbance peak with FWHM of less than 160 nm. In some embodiments, the detectable moieties having the phenoxazine or a phenoxazinone core have a peak absorbance wavelength of about 645+/−10 nm and a first absorbance peak with FWHM of less than 160 nm. In some embodiments, the detectable moieties having the phenoxazine or a phenoxazinone core have a peak absorbance wavelength of about 640+/−10 nm and a first absorbance peak with FWHM of less than 160 nm. In some embodiments, the detectable moieties having the phenoxazine or a phenoxazinone core have a peak absorbance wavelength of about 635+/−10 nm and a first absorbance peak with FWHM of less than 160 nm. In some embodiments, the detectable moieties having the phenoxazine or a phenoxazinone core have a peak absorbance wavelength of about 630+/−10 nm and a first absorbance peak with FWHM of less than 160 nm. In some embodiments, the detectable moieties having the phenoxazine or a phenoxazinone core have a peak absorbance wavelength of about 625+/−10 nm and a first absorbance peak with FWHM of less than 160 nm. In some embodiments, the detectable moieties having the phenoxazine or a phenoxazinone core have a peak absorbance wavelength of about 620+/−10 nm and a first absorbance peak with FWHM of less than 160 nm. In some embodiments, the detectable moieties having the phenoxazine or a phenoxazinone core have a peak absorbance wavelength of about 615+/−10 nm and a first absorbance peak with FWHM of less than 160 nm. In some embodiments, the detectable moieties having the phenoxazine or a phenoxazinone core have a peak absorbance wavelength of about 610+/−10 nm and a first absorbance peak with FWHM of less than 160 nm. In some embodiments, the detectable moieties having the phenoxazine or a phenoxazinone core have a peak absorbance wavelength of about 605+/−10 nm and a first absorbance peak with FWHM of less than 160 nm. In some embodiments, the detectable moieties having the phenoxazine or a phenoxazinone core have a peak absorbance wavelength of about 600+/−10 nm and a first absorbance peak with FWHM of less than 160 nm. In some embodiments, the detectable moieties having the phenoxazine or a phenoxazinone core have a peak absorbance wavelength of about 595+/−10 nm and a first absorbance peak with FWHM of less than 160 nm. In some embodiments, the detectable moieties having the phenoxazine or a phenoxazinone core have a peak absorbance wavelength of about 590+/−10 nm and a first absorbance peak with FWHM of less than 160 nm. In some embodiments, the detectable moieties having the phenoxazine or a phenoxazinone core have a peak absorbance wavelength of about 585+/−10 nm and a first absorbance peak with FWHM of less than 160 nm. In some embodiments, the detectable moieties having the phenoxazine or a phenoxazinone core have a peak absorbance wavelength of about 580+/−10 nm and a first absorbance peak with FWHM of less than 160 nm.

In some embodiments, the detectable moieties having the phenoxazine or a phenoxazinone core have a peak absorbance wavelength of about 700+/−10 nm and a first absorbance peak with FWHM of less than 130 nm. In some embodiments, the detectable moieties having the phenoxazine or a phenoxazinone core have a peak absorbance wavelength of about 695+/−10 nm and a first absorbance peak with FWHM of less than 130 nm. In some embodiments, the detectable moieties having the phenoxazine or a phenoxazinone core have a peak absorbance wavelength of about 690+/−10 nm and a first absorbance peak with FWHM of less than 130 nm. In some embodiments, the detectable moieties having the phenoxazine or a phenoxazinone core have a peak absorbance wavelength of about 685+/−10 nm and a first absorbance peak with FWHM of less than 130 nm. In some embodiments, the detectable moieties having the phenoxazine or a phenoxazinone core have a peak absorbance wavelength of about 680+/−10 nm and a first absorbance peak with FWHM of less than 130 nm. In some embodiments, the detectable moieties having the phenoxazine or a phenoxazinone core have a peak absorbance wavelength of about 675+/−10 nm and a first absorbance peak with FWHM of less than 130 nm. In some embodiments, the detectable moieties having the phenoxazine or a phenoxazinone core have a peak absorbance wavelength of about 670+/−10 nm and a first absorbance peak with FWHM of less than 130 nm. In some embodiments, the detectable moieties having the phenoxazine or a phenoxazinone core have a peak absorbance wavelength of about 665+/−10 nmm and a first absorbance peak with FWHM of less than 130 nm. In some embodiments, the detectable moieties having the phenoxazine or a phenoxazinone core have a peak absorbance wavelength of about 660+/−10 nm and a first absorbance peak with FWHM of less than 130 nm. In some embodiments, the detectable moieties having the phenoxazine or a phenoxazinone core have a peak absorbance wavelength of about 655+/−10 nm and a first absorbance peak with FWHM of less than 130 nm. In some embodiments, the detectable moieties having the phenoxazine or a phenoxazinone core have a peak absorbance wavelength of about 650+/−10 nm and a first absorbance peak with FWHM of less than 130 nm. In some embodiments, the detectable moieties having the phenoxazine or a phenoxazinone core have a peak absorbance wavelength of about 645+/−10 nm and a first absorbance peak with FWHM of less than 130 nm. In some embodiments, the detectable moieties having the phenoxazine or a phenoxazinone core have a peak absorbance wavelength of about 640+/−10 nm and a first absorbance peak with FWHM of less than 130 nm. In some embodiments, the detectable moieties having the phenoxazine or a phenoxazinone core have a peak absorbance wavelength of about 635+/−10 nm and a first absorbance peak with FWHM of less than 130 nm. In some embodiments, the detectable moieties having the phenoxazine or a phenoxazinone core have a peak absorbance wavelength of about 630+/−10 nm and a first absorbance peak with FWHM of less than 130 nm. In some embodiments, the detectable moieties having the phenoxazine or a phenoxazinone core have a peak absorbance wavelength of about 625+/−10 nm and a first absorbance peak with FWHM of less than 130 nm. In some embodiments, the detectable moieties having the phenoxazine or a phenoxazinone core have a peak absorbance wavelength of about 620+/−10 nm and a first absorbance peak with FWHM of less than 130 nm. In some embodiments, the detectable moieties having the phenoxazine or a phenoxazinone core have a peak absorbance wavelength of about 615+/−10 nm and a first absorbance peak with FWHM of less than 130 nm. In some embodiments, the detectable moieties having the phenoxazine or a phenoxazinone core have a peak absorbance wavelength of about 610+/−10 nm and a first absorbance peak with FWHM of less than 130 nm. In some embodiments, the detectable moieties having the phenoxazine or a phenoxazinone core have a peak absorbance wavelength of about 605+/−10 nm and a first absorbance peak with FWHM of less than 130 nm. In some embodiments, the detectable moieties having the phenoxazine or a phenoxazinone core have a peak absorbance wavelength of about 600+/−10 nm and a first absorbance peak with FWHM of less than 130 nm. In some embodiments, the detectable moieties having the phenoxazine or a phenoxazinone core have a peak absorbance wavelength of about 595+/−10 nm and a first absorbance peak with FWHM of less than 130 nm. In some embodiments, the detectable moieties having the phenoxazine or a phenoxazinone core have a peak absorbance wavelength of about 590+/−10 nm and a first absorbance peak with FWHM of less than 130 nm. In some embodiments, the detectable moieties having the phenoxazine or a phenoxazinone core have a peak absorbance wavelength of about 585+/−10 nm and a first absorbance peak with FWHM of less than 130 nm. In some embodiments, the detectable moieties having the phenoxazine or a phenoxazinone core have a peak absorbance wavelength of about 580+/−10 nm and a first absorbance peak with FWHM of less than 130 nm.

In some embodiments, the detectable moieties having the phenoxazine or a phenoxazinone core have a peak absorbance wavelength of about 700+/−10 nm and a first absorbance peak with FWHM of less than 100 nm. In some embodiments, the detectable moieties having the phenoxazine or a phenoxazinone core have a peak absorbance wavelength of about 695+/−10 nm and a first absorbance peak with FWHM of less than 100 nm. In some embodiments, the detectable moieties having the phenoxazine or a phenoxazinone core have a peak absorbance wavelength of about 690+/−10 nm and a first absorbance peak with FWHM of less than 100 nm. In some embodiments, the detectable moieties having the phenoxazine or a phenoxazinone core have a peak absorbance wavelength of about 685+/−10 nm and a first absorbance peak with FWHM of less than 100 nm. In some embodiments, the detectable moieties having the phenoxazine or a phenoxazinone core have a peak absorbance wavelength of about 680+/−10 nm and a first absorbance peak with FWHM of less than 100 nm. In some embodiments, the detectable moieties having the phenoxazine or a phenoxazinone core have a peak absorbance wavelength of about 675+/−10 nm and a first absorbance peak with FWHM of less than 100 nm. In some embodiments, the detectable moieties having the phenoxazine or a phenoxazinone core have a peak absorbance wavelength of about 670+/−10 nm and a first absorbance peak with FWHM of less than 100 nm. In some embodiments, the detectable moieties having the phenoxazine or a phenoxazinone core have a peak absorbance wavelength of about 665+/−10 nmm and a first absorbance peak with FWHM of less than 100 nm. In some embodiments, the detectable moieties having the phenoxazine or a phenoxazinone core have a peak absorbance wavelength of about 660+/−10 nm and a first absorbance peak with FWHM of less than 100 nm. In some embodiments, the detectable moieties having the phenoxazine or a phenoxazinone core have a peak absorbance wavelength of about 655+/−10 nm and a first absorbance peak with FWHM of less than 100 nm. In some embodiments, the detectable moieties having the phenoxazine or a phenoxazinone core have a peak absorbance wavelength of about 650+/−10 nm and a first absorbance peak with FWHM of less than 100 nm. In some embodiments, the detectable moieties having the phenoxazine or a phenoxazinone core have a peak absorbance wavelength of about 645+/−10 nm and a first absorbance peak with FWHM of less than 100 nm. In some embodiments, the detectable moieties having the phenoxazine or a phenoxazinone core have a peak absorbance wavelength of about 640+/−10 nm and a first absorbance peak with FWHM of less than 100 nm. In some embodiments, the detectable moieties having the phenoxazine or a phenoxazinone core have a peak absorbance wavelength of about 635+/−10 nm and a first absorbance peak with FWHM of less than 100 nm. In some embodiments, the detectable moieties having the phenoxazine or a phenoxazinone core have a peak absorbance wavelength of about 630+/−10 nm and a first absorbance peak with FWHM of less than 100 nm. In some embodiments, the detectable moieties having the phenoxazine or a phenoxazinone core have a peak absorbance wavelength of about 625+/−10 nm and a first absorbance peak with FWHM of less than 100 nm. In some embodiments, the detectable moieties having the phenoxazine or a phenoxazinone core have a peak absorbance wavelength of about 620+/−10 nm and a first absorbance peak with FWHM of less than 100 nm. In some embodiments, the detectable moieties having the phenoxazine or a phenoxazinone core have a peak absorbance wavelength of about 615+/−10 nm and a first absorbance peak with FWHM of less than 100 nm. In some embodiments, the detectable moieties having the phenoxazine or a phenoxazinone core have a peak absorbance wavelength of about 610+/−10 nm and a first absorbance peak with FWHM of less than 100 nm. In some embodiments, the detectable moieties having the phenoxazine or a phenoxazinone core have a peak absorbance wavelength of about 605+/−10 nm and a first absorbance peak with FWHM of less than 100 nm. In some embodiments, the detectable moieties having the phenoxazine or a phenoxazinone core have a peak absorbance wavelength of about 600+/−10 nm and a first absorbance peak with FWHM of less than 100 nm. In some embodiments, the detectable moieties having the phenoxazine or a phenoxazinone core have a peak absorbance wavelength of about 595+/−10 nm and a first absorbance peak with FWHM of less than 100 nm. In some embodiments, the detectable moieties having the phenoxazine or a phenoxazinone core have a peak absorbance wavelength of about 590+/−10 nm and a first absorbance peak with FWHM of less than 100 nm. In some embodiments, the detectable moieties having the phenoxazine or a phenoxazinone core have a peak absorbance wavelength of about 585+/−10 nm and a first absorbance peak with FWHM of less than 100 nm. In some embodiments, the detectable moieties having the phenoxazine or a phenoxazinone core have a peak absorbance wavelength of about 580+/−10 nm and a first absorbance peak with FWHM of less than 100 nm.

In some embodiments, the detectable moiety includes or is derived from a thioninium, phenoxazine, or phenoxathiin-3-one core (i.e. the detectable moiety includes a thioninium or phenoxathiin-3-one core).

In some embodiments, the thioninium, phenoxazine, or phenoxathiin-3-one core includes (or is modified to include) one or more electron withdrawing groups (where each electron withdrawing group may be the same or different). In some embodiments, the thioninium, phenoxazine, or phenoxathiin-3-one core includes (or is modified to include) one electron withdrawing group. In some embodiments, the thioninium, phenoxazine, or phenoxathiin-3-one core includes (or is modified to include) two electron withdrawing groups. In some embodiments, the thioninium, phenoxazine, or phenoxathiin-3-one core includes (or is modifying to include) three electron withdrawing groups. In some embodiments, the thioninium, phenoxazine, or phenoxathiin-3-one core includes (or is modifying to include) three different electron withdrawing groups. In some embodiments, the thioninium, phenoxazine, or phenoxathiin-3-one core includes (or is modified to include) four electron withdrawing groups.

In some embodiments, the thioninium, phenoxazine, or phenoxathiin-3-one core includes (or is modified to include) one or more electron donating groups (where each electron withdrawing group may be the same or different). In some embodiments, the thioninium, phenoxazine, or phenoxathiin-3-one core includes (or is modified to include) one electron donating group. In some embodiments, the thioninium, phenoxazine, or phenoxathiin-3-one core includes (or is modified to include) two electron donating groups. In some embodiments, the thioninium, phenoxazine, or phenoxathiin-3-one core includes (or is modifying to include) three electron donating groups. In some embodiments, the thioninium, phenoxazine, or phenoxathiin-3-one core includes (or is modifying to include) three different electron donating groups. In some embodiments, the thioninium, phenoxazine, or phenoxathiin-3-one core includes (or is modified to include) four electron donating groups.

In some embodiments, the detectable moieties having the thioninium, phenoxazine, or phenoxathiin-3-one core have a peak absorbance wavelength ranging from about 580 nm to about 720 nm. In some embodiments, the detectable moieties having the thioninium, phenoxazine, or phenoxathiin-3-one core have a peak absorbance wavelength ranging from about 600 nm to about 720 nm. In some embodiments, the detectable moieties having the thioninium, phenoxazine, or phenoxathiin-3-one core have a peak absorbance wavelength ranging from about 630 nm to about 720 nm. In some embodiments, the detectable moieties having the thioninium, phenoxazine, or phenoxathiin-3-one core have a peak absorbance wavelength ranging from about 645 nm to about 700 nm. In some embodiments, the detectable moieties having the thioninium, phenoxazine, or phenoxathiin-3-one core have a peak absorbance wavelength ranging from about 665 nm to about 690 nm.

In some embodiments, the detectable moieties having the thioninium, phenoxazine, or phenoxathiin-3-one core have a wavelength ranging from about 580 nm to about 720 nm and a first absorbance peak with FWHM of less than 160 nm. In some embodiments, the detectable moieties having the thioninium, phenoxazine, or phenoxathiin-3-one core have a wavelength ranging from about 600 nm to about 720 nm and a first absorbance peak with FWHM of less than 160 nm. In some embodiments, the detectable moieties having the thioninium, phenoxazine, or phenoxathiin-3-one core have a wavelength ranging from about 630 nm to about 720 nm and a first absorbance peak with FWHM of less than 160 nm. In some embodiments, the detectable moieties having the thioninium, phenoxazine, or phenoxathiin-3-one core have a wavelength ranging from about 645 nm to about 700 nm and a first absorbance peak with FWHM of less than 160 nm. In some embodiments, the detectable moieties having the thioninium, phenoxazine, or phenoxathiin-3-one core have a wavelength ranging from about 665 nm to about 690 nm and a first absorbance peak with FWHM of less than 160 nm.

In some embodiments, the detectable moieties having the thioninium, phenoxazine, or phenoxathiin-3-one core have a wavelength ranging from about 580 nm to about 720 nm and a first absorbance peak with FWHM of less than 130 nm. In some embodiments, the detectable moieties having the thioninium, phenoxazine, or phenoxathiin-3-one core have a wavelength ranging from about 600 nm to about 720 nm and a first absorbance peak with FWHM of less than 130 nm. In some embodiments, the detectable moieties having the thioninium, phenoxazine, or phenoxathiin-3-one core have a wavelength ranging from about 630 nm to about 720 nm and a first absorbance peak with FWHM of less than 130 nm. In some embodiments, the detectable moieties having the thioninium, phenoxazine, or phenoxathiin-3-one core have a wavelength ranging from about 645 nm to about 700 nm and a first absorbance peak with FWHM of less than 130 nm. In some embodiments, the detectable moieties having the thioninium, phenoxazine, or phenoxathiin-3-one core have a wavelength ranging from about 665 nm to about 690 nm and a first absorbance peak with FWHM of less than 130 nm.

In some embodiments, the detectable moieties having the thioninium, phenoxazine, or phenoxathiin-3-one core have a wavelength ranging from about 580 nm to about 720 nm and a first absorbance peak with FWHM of less than 100 nm. In some embodiments, the detectable moieties having the thioninium, phenoxazine, or phenoxathiin-3-one core have a wavelength ranging from about 600 nm to about 720 nm and a first absorbance peak with FWHM of less than 100 nm. In some embodiments, the detectable moieties having the thioninium, phenoxazine, or phenoxathiin-3-one core have a wavelength ranging from about 630 nm to about 720 nm and a first absorbance peak with FWHM of less than 100 nm. In some embodiments, the detectable moieties having the thioninium, phenoxazine, or phenoxathiin-3-one core have a wavelength ranging from about 645 nm to about 700 nm and a first absorbance peak with FWHM of less than 100 nm. In some embodiments, the detectable moieties having the thioninium, phenoxazine, or phenoxathiin-3-one core have a wavelength ranging from about 665 nm to about 690 nm and a first absorbance peak with FWHM of less than 100 nm.

In some embodiments, the detectable moieties having the thioninium, phenoxazine, or phenoxathiin-3-one core have a peak absorbance wavelength of about 720+/−10 nm. In some embodiments, the detectable moieties having thioninium, phenoxazine, or phenoxathiin-3-one core have a peak absorbance wavelength of about 715+/−10 nm. In some embodiments, the detectable moieties having the thioninium, phenoxazine, or phenoxathiin-3-one core have a peak absorbance wavelength of about 710+/−10 nm. In some embodiments, the detectable moieties having the thioninium, phenoxazine, or phenoxathiin-3-one core have a peak absorbance wavelength of about 705+/−10 nm. In some embodiments, the detectable moieties having the thioninium, phenoxazine, or phenoxathiin-3-one core have a peak absorbance wavelength of about 700+/−10 nm. In some embodiments, the detectable moieties having the thioninium, phenoxazine, or phenoxathiin-3-one core have a peak absorbance wavelength of about 695+/−10 nm. In some embodiments, the detectable moieties having the thioninium, phenoxazine, or phenoxathiin-3-one core have a peak absorbance wavelength of about 690+/−10 nm. In some embodiments, the detectable moieties having the thioninium, phenoxazine, or phenoxathiin-3-one core have a peak absorbance wavelength of about 685+/−10 nm. In some embodiments, the detectable moieties having the thioninium, phenoxazine, or phenoxathiin-3-one core have a peak absorbance wavelength of about 680+/−10 nm. In some embodiments, the detectable moieties having the thioninium, phenoxazine, or phenoxathiin-3-one core have a peak absorbance wavelength of about 675+/−10 nm. In some embodiments, the detectable moieties having the thioninium, phenoxazine, or phenoxathiin-3-one core have a peak absorbance wavelength of about 670+/−10 nm. In some embodiments, the detectable moieties having the thioninium, phenoxazine, or phenoxathiin-3-one core have a peak absorbance wavelength of about 665+/−10 nm. In some embodiments, the detectable moieties having the thioninium, phenoxazine, or phenoxathiin-3-one core have a peak absorbance wavelength of about 660+/−10 nm. In some embodiments, the detectable moieties having the thioninium, phenoxazine, or phenoxathiin-3-one core have a peak absorbance wavelength of about 655+/−10 nm. In some embodiments, the detectable moieties having the thioninium, phenoxazine, or phenoxathiin-3-one core have a peak absorbance wavelength of about 650+/−10 nm. In some embodiments, the detectable moieties having the thioninium, phenoxazine, or phenoxathiin-3-one core have a peak absorbance wavelength of about 645+/−10 nm. In some embodiments, the detectable moieties having the thioninium, phenoxazine, or phenoxathiin-3-one core have a peak absorbance wavelength of about 640+/−10 nm. In some embodiments, the detectable moieties having the thioninium, phenoxazine, or phenoxathiin-3-one core have a peak absorbance wavelength of about 635+/−10 nm. In some embodiments, the detectable moieties having the thioninium, phenoxazine, or phenoxathiin-3-one core have a peak absorbance wavelength of about 630+/−10 nm. In some embodiments, the detectable moieties having the thioninium, phenoxazine, or phenoxathiin-3-one core have a peak absorbance wavelength of about 625+/−10 nm. In some embodiments, the detectable moieties having the thioninium, phenoxazine, or phenoxathiin-3-one core have a peak absorbance wavelength of about 620+/−10 nm. In some embodiments, the detectable moieties having the thioninium, phenoxazine, or phenoxathiin-3-one core have a peak absorbance wavelength of about 615+/−10 nm. In some embodiments, the detectable moieties having the thioninium, phenoxazine, or phenoxathiin-3-one core have a peak absorbance wavelength of about 610+/−10 nm. In some embodiments, the detectable moieties having the thioninium, phenoxazine, or phenoxathiin-3-one core have a peak absorbance wavelength of about 605+/−10 nm. In some embodiments, the detectable moieties having the thioninium, phenoxazine, or phenoxathiin-3-one core have a peak absorbance wavelength of about 600+/−10 nm. In some embodiments, the detectable moieties having the thioninium, phenoxazine, or phenoxathiin-3-one core have a peak absorbance wavelength of about 595+/−10 nm. In some embodiments, the detectable moieties having the thioninium, phenoxazine, or phenoxathiin-3-one core have a peak absorbance wavelength of about 590+/−10 nm. In some embodiments, the detectable moieties having the thioninium, phenoxazine, or phenoxathiin-3-one core have a peak absorbance wavelength of about 585+/−10 nm. In some embodiments, the detectable moieties having the thioninium, phenoxazine, or phenoxathiin-3-one core have a peak absorbance wavelength of about 580+/−10 nm.

In some embodiments, the detectable moieties having the thioninium, phenoxazine, or phenoxathiin-3-one core have a peak absorbance wavelength of about 720+/−10 nm and a first absorbance peak with FWHM of less than 160 nm. In some embodiments, the detectable moieties having thioninium, phenoxazine, or phenoxathiin-3-one core have a peak absorbance wavelength of about 715+/−10 nm and a first absorbance peak with FWHM of less than 160 nm. In some embodiments, the detectable moieties having the thioninium, phenoxazine, or phenoxathiin-3-one core have a peak absorbance wavelength of about 710+/−10 nm and a first absorbance peak with FWHM of less than 160 nm. In some embodiments, the detectable moieties having the thioninium, phenoxazine, or phenoxathiin-3-one core have a peak absorbance wavelength of about 705+/−10 nm and a first absorbance peak with FWHM of less than 160 nm. In some embodiments, the detectable moieties having the thioninium, phenoxazine, or phenoxathiin-3-one core have a peak absorbance wavelength of about 700+/−10 nm and a first absorbance peak with FWHM of less than 160 nm. In some embodiments, the detectable moieties having the thioninium, phenoxazine, or phenoxathiin-3-one core have a peak absorbance wavelength of about 695+/−10 nm and a first absorbance peak with FWHM of less than 160 nm. In some embodiments, the detectable moieties having the thioninium, phenoxazine, or phenoxathiin-3-one core have a peak absorbance wavelength of about 690+/−10 nm and a first absorbance peak with FWHM of less than 160 nm. In some embodiments, the detectable moieties having the thioninium, phenoxazine, or phenoxathiin-3-one core have a peak absorbance wavelength of about 685+/−10 nm and a first absorbance peak with FWHM of less than 160 nm. In some embodiments, the detectable moieties having the thioninium, phenoxazine, or phenoxathiin-3-one core have a peak absorbance wavelength of about 680+/−10 nm and a first absorbance peak with FWHM of less than 160 nm. In some embodiments, the detectable moieties having the thioninium, phenoxazine, or phenoxathiin-3-one core have a peak absorbance wavelength of about 675+/−10 nm and a first absorbance peak with FWHM of less than 160 nm. In some embodiments, the detectable moieties having the thioninium, phenoxazine, or phenoxathiin-3-one core have a peak absorbance wavelength of about 670+/−10 nm and a first absorbance peak with FWHM of less than 160 nm. In some embodiments, the detectable moieties having the thioninium, phenoxazine, or phenoxathiin-3-one core have a peak absorbance wavelength of about 665+/−10 nm and a first absorbance peak with FWHM of less than 160 nm. In some embodiments, the detectable moieties having the thioninium, phenoxazine, or phenoxathiin-3-one core have a peak absorbance wavelength of about 660+/−10 nm and a first absorbance peak with FWHM of less than 160 nm. In some embodiments, the detectable moieties having the thioninium, phenoxazine, or phenoxathiin-3-one core have a peak absorbance wavelength of about 655+/−10 nm and a first absorbance peak with FWHM of less than 160 nm. In some embodiments, the detectable moieties having the thioninium, phenoxazine, or phenoxathiin-3-one core have a peak absorbance wavelength of about 650+/−10 nm and a first absorbance peak with FWHM of less than 160 nm. In some embodiments, the detectable moieties having the thioninium, phenoxazine, or phenoxathiin-3-one core have a peak absorbance wavelength of about 645+/−10 nm and a first absorbance peak with FWHM of less than 160 nm. In some embodiments, the detectable moieties having the thioninium, phenoxazine, or phenoxathiin-3-one core have a peak absorbance wavelength of about 640+/−10 nm and a first absorbance peak with FWHM of less than 160 nm. In some embodiments, the detectable moieties having the thioninium, phenoxazine, or phenoxathiin-3-one core have a peak absorbance wavelength of about 635+/−10 nm and a first absorbance peak with FWHM of less than 160 nm. In some embodiments, the detectable moieties having the thioninium, phenoxazine, or phenoxathiin-3-one core have a peak absorbance wavelength of about 630+/−10 nm and a first absorbance peak with FWHM of less than 160 nm. In some embodiments, the detectable moieties having the thioninium, phenoxazine, or phenoxathiin-3-one core have a peak absorbance wavelength of about 625+/−10 nm and a first absorbance peak with FWHM of less than 160 nm. In some embodiments, the detectable moieties having the thioninium, phenoxazine, or phenoxathiin-3-one core have a peak absorbance wavelength of about 620+/−10 nm and a first absorbance peak with FWHM of less than 160 nm. In some embodiments, the detectable moieties having the thioninium, phenoxazine, or phenoxathiin-3-one core have a peak absorbance wavelength of about 615+/−10 nm and a first absorbance peak with FWHM of less than 160 nm. In some embodiments, the detectable moieties having the thioninium, phenoxazine, or phenoxathiin-3-one core have a peak absorbance wavelength of about 610+/−10 nm and a first absorbance peak with FWHM of less than 160 nm. In some embodiments, the detectable moieties having the thioninium, phenoxazine, or phenoxathiin-3-one core have a peak absorbance wavelength of about 605+/−10 nm and a first absorbance peak with FWHM of less than 160 nm. In some embodiments, the detectable moieties having the thioninium, phenoxazine, or phenoxathiin-3-one core have a peak absorbance wavelength of about 600+/−10 nm and a first absorbance peak with FWHM of less than 160 nm. In some embodiments, the detectable moieties having the thioninium, phenoxazine, or phenoxathiin-3-one core have a peak absorbance wavelength of about 595+/−10 nm and a first absorbance peak with FWHM of less than 160 nm. In some embodiments, the detectable moieties having the thioninium, phenoxazine, or phenoxathiin-3-one core have a peak absorbance wavelength of about 590+/−10 nm and a first absorbance peak with FWHM of less than 160 nm. In some embodiments, the detectable moieties having the thioninium, phenoxazine, or phenoxathiin-3-one core have a peak absorbance wavelength of about 585+/−10 nm and a first absorbance peak with FWHM of less than 160 nm. In some embodiments, the detectable moieties having the thioninium, phenoxazine, or phenoxathiin-3-one core have a peak absorbance wavelength of about 580+/−10 nm and a first absorbance peak with FWHM of less than 160 nm.

In some embodiments, the detectable moieties having the thioninium, phenoxazine, or phenoxathiin-3-one core have a peak absorbance wavelength of about 720+/−10 nm and a first absorbance peak with FWHM of less than 130 nm. In some embodiments, the detectable moieties having thioninium, phenoxazine, or phenoxathiin-3-one core have a peak absorbance wavelength of about 715+/−10 nm and a first absorbance peak with FWHM of less than 130 nm. In some embodiments, the detectable moieties having the thioninium, phenoxazine, or phenoxathiin-3-one core have a peak absorbance wavelength of about 710+/−10 nm and a first absorbance peak with FWHM of less than 130 nm. In some embodiments, the detectable moieties having the thioninium, phenoxazine, or phenoxathiin-3-one core have a peak absorbance wavelength of about 705+/−10 nm and a first absorbance peak with FWHM of less than 130 nm. In some embodiments, the detectable moieties having the thioninium, phenoxazine, or phenoxathiin-3-one core have a peak absorbance wavelength of about 700+/−10 nm and a first absorbance peak with FWHM of less than 130 nm. In some embodiments, the detectable moieties having the thioninium, phenoxazine, or phenoxathiin-3-one core have a peak absorbance wavelength of about 695+/−10 nm and a first absorbance peak with FWHM of less than 130 nm. In some embodiments, the detectable moieties having the thioninium, phenoxazine, or phenoxathiin-3-one core have a peak absorbance wavelength of about 690+/−10 nm and a first absorbance peak with FWHM of less than 130 nm. In some embodiments, the detectable moieties having the thioninium, phenoxazine, or phenoxathiin-3-one core have a peak absorbance wavelength of about 685+/−10 nm and a first absorbance peak with FWHM of less than 130 nm. In some embodiments, the detectable moieties having the thioninium, phenoxazine, or phenoxathiin-3-one core have a peak absorbance wavelength of about 680+/−10 nm and a first absorbance peak with FWHM of less than 130 nm. In some embodiments, the detectable moieties having the thioninium, phenoxazine, or phenoxathiin-3-one core have a peak absorbance wavelength of about 675+/−10 nm and a first absorbance peak with FWHM of less than 130 nm. In some embodiments, the detectable moieties having the thioninium, phenoxazine, or phenoxathiin-3-one core have a peak absorbance wavelength of about 670+/−10 nm and a first absorbance peak with FWHM of less than 130 nm. In some embodiments, the detectable moieties having the thioninium, phenoxazine, or phenoxathiin-3-one core have a peak absorbance wavelength of about 665+/−10 nm and a first absorbance peak with FWHM of less than 130 nm. In some embodiments, the detectable moieties having the thioninium, phenoxazine, or phenoxathiin-3-one core have a peak absorbance wavelength of about 660+/−10 nm and a first absorbance peak with FWHM of less than 130 nm. In some embodiments, the detectable moieties having the thioninium, phenoxazine, or phenoxathiin-3-one core have a peak absorbance wavelength of about 655+/−10 nm and a first absorbance peak with FWHM of less than 130 nm. In some embodiments, the detectable moieties having the thioninium, phenoxazine, or phenoxathiin-3-one core have a peak absorbance wavelength of about 650+/−10 nm and a first absorbance peak with FWHM of less than 130 nm. In some embodiments, the detectable moieties having the thioninium, phenoxazine, or phenoxathiin-3-one core have a peak absorbance wavelength of about 645+/−10 nm and a first absorbance peak with FWHM of less than 130 nm. In some embodiments, the detectable moieties having the thioninium, phenoxazine, or phenoxathiin-3-one core have a peak absorbance wavelength of about 640+/−10 nm and a first absorbance peak with FWHM of less than 130 nm. In some embodiments, the detectable moieties having the thioninium, phenoxazine, or phenoxathiin-3-one core have a peak absorbance wavelength of about 635+/−10 nm and a first absorbance peak with FWHM of less than 130 nm. In some embodiments, the detectable moieties having the thioninium, phenoxazine, or phenoxathiin-3-one core have a peak absorbance wavelength of about 630+/−10 nm and a first absorbance peak with FWHM of less than 130 nm. In some embodiments, the detectable moieties having the thioninium, phenoxazine, or phenoxathiin-3-one core have a peak absorbance wavelength of about 625+/−10 nm and a first absorbance peak with FWHM of less than 130 nm. In some embodiments, the detectable moieties having the thioninium, phenoxazine, or phenoxathiin-3-one core have a peak absorbance wavelength of about 620+/−10 nm and a first absorbance peak with FWHM of less than 130 nm. In some embodiments, the detectable moieties having the thioninium, phenoxazine, or phenoxathiin-3-one core have a peak absorbance wavelength of about 615+/−10 nm and a first absorbance peak with FWHM of less than 130 nm. In some embodiments, the detectable moieties having the thioninium, phenoxazine, or phenoxathiin-3-one core have a peak absorbance wavelength of about 610+/−10 nm and a first absorbance peak with FWHM of less than 130 nm. In some embodiments, the detectable moieties having the thioninium, phenoxazine, or phenoxathiin-3-one core have a peak absorbance wavelength of about 605+/−10 nm and a first absorbance peak with FWHM of less than 130 nm. In some embodiments, the detectable moieties having the thioninium, phenoxazine, or phenoxathiin-3-one core have a peak absorbance wavelength of about 600+/−10 nm and a first absorbance peak with FWHM of less than 130 nm. In some embodiments, the detectable moieties having the thioninium, phenoxazine, or phenoxathiin-3-one core have a peak absorbance wavelength of about 595+/−10 nm and a first absorbance peak with FWHM of less than 130 nm. In some embodiments, the detectable moieties having the thioninium, phenoxazine, or phenoxathiin-3-one core have a peak absorbance wavelength of about 590+/−10 nm and a first absorbance peak with FWHM of less than 130 nm. In some embodiments, the detectable moieties having the thioninium, phenoxazine, or phenoxathiin-3-one core have a peak absorbance wavelength of about 585+/−10 nm and a first absorbance peak with FWHM of less than 130 nm. In some embodiments, the detectable moieties having the thioninium, phenoxazine, or phenoxathiin-3-one core have a peak absorbance wavelength of about 580+/−10 nm and a first absorbance peak with FWHM of less than 130 nm.

In some embodiments, the detectable moieties having the thioninium, phenoxazine, or phenoxathiin-3-one core have a peak absorbance wavelength of about 720+/−10 nm and a first absorbance peak with FWHM of less than 100 nm. In some embodiments, the detectable moieties having thioninium, phenoxazine, or phenoxathiin-3-one core have a peak absorbance wavelength of about 715+/−10 nm and a first absorbance peak with FWHM of less than 100 nm. In some embodiments, the detectable moieties having the thioninium, phenoxazine, or phenoxathiin-3-one core have a peak absorbance wavelength of about 710+/−10 nm and a first absorbance peak with FWHM of less than 100 nm. In some embodiments, the detectable moieties having the thioninium, phenoxazine, or phenoxathiin-3-one core have a peak absorbance wavelength of about 705+/−10 nm and a first absorbance peak with FWHM of less than 100 nm. In some embodiments, the detectable moieties having the thioninium, phenoxazine, or phenoxathiin-3-one core have a peak absorbance wavelength of about 700+/−10 nm and a first absorbance peak with FWHM of less than 100 nm. In some embodiments, the detectable moieties having the thioninium, phenoxazine, or phenoxathiin-3-one core have a peak absorbance wavelength of about 695+/−10 nm and a first absorbance peak with FWHM of less than 100 nm. In some embodiments, the detectable moieties having the thioninium, phenoxazine, or phenoxathiin-3-one core have a peak absorbance wavelength of about 690+/−10 nm and a first absorbance peak with FWHM of less than 100 nm. In some embodiments, the detectable moieties having the thioninium, phenoxazine, or phenoxathiin-3-one core have a peak absorbance wavelength of about 685+/−10 nm and a first absorbance peak with FWHM of less than 100 nm. In some embodiments, the detectable moieties having the thioninium, phenoxazine, or phenoxathiin-3-one core have a peak absorbance wavelength of about 680+/−10 nm and a first absorbance peak with FWHM of less than 100 nm. In some embodiments, the detectable moieties having the thioninium, phenoxazine, or phenoxathiin-3-one core have a peak absorbance wavelength of about 675+/−10 nm and a first absorbance peak with FWHM of less than 100 nm. In some embodiments, the detectable moieties having the thioninium, phenoxazine, or phenoxathiin-3-one core have a peak absorbance wavelength of about 670+/−10 nm and a first absorbance peak with FWHM of less than 100 nm. In some embodiments, the detectable moieties having the thioninium, phenoxazine, or phenoxathiin-3-one core have a peak absorbance wavelength of about 665+/−10 nm and a first absorbance peak with FWHM of less than 100 nm. In some embodiments, the detectable moieties having the thioninium, phenoxazine, or phenoxathiin-3-one core have a peak absorbance wavelength of about 660+/−10 nm and a first absorbance peak with FWHM of less than 100 nm. In some embodiments, the detectable moieties having the thioninium, phenoxazine, or phenoxathiin-3-one core have a peak absorbance wavelength of about 655+/−10 nm and a first absorbance peak with FWHM of less than 100 nm. In some embodiments, the detectable moieties having the thioninium, phenoxazine, or phenoxathiin-3-one core have a peak absorbance wavelength of about 650+/−10 nm and a first absorbance peak with FWHM of less than 100 nm. In some embodiments, the detectable moieties having the thioninium, phenoxazine, or phenoxathiin-3-one core have a peak absorbance wavelength of about 645+/−10 nm and a first absorbance peak with FWHM of less than 100 nm. In some embodiments, the detectable moieties having the thioninium, phenoxazine, or phenoxathiin-3-one core have a peak absorbance wavelength of about 640+/−10 nm and a first absorbance peak with FWHM of less than 100 nm. In some embodiments, the detectable moieties having the thioninium, phenoxazine, or phenoxathiin-3-one core have a peak absorbance wavelength of about 635+/−10 nm and a first absorbance peak with FWHM of less than 100 nm. In some embodiments, the detectable moieties having the thioninium, phenoxazine, or phenoxathiin-3-one core have a peak absorbance wavelength of about 630+/−10 nm and a first absorbance peak with FWHM of less than 100 nm. In some embodiments, the detectable moieties having the thioninium, phenoxazine, or phenoxathiin-3-one core have a peak absorbance wavelength of about 625+/−10 nm and a first absorbance peak with FWHM of less than 100 nm. In some embodiments, the detectable moieties having the thioninium, phenoxazine, or phenoxathiin-3-one core have a peak absorbance wavelength of about 620+/−10 nm and a first absorbance peak with FWHM of less than 100 nm. In some embodiments, the detectable moieties having the thioninium, phenoxazine, or phenoxathiin-3-one core have a peak absorbance wavelength of about 615+/−10 nm and a first absorbance peak with FWHM of less than 100 nm. In some embodiments, the detectable moieties having the thioninium, phenoxazine, or phenoxathiin-3-one core have a peak absorbance wavelength of about 610+/−10 nm and a first absorbance peak with FWHM of less than 100 nm. In some embodiments, the detectable moieties having the thioninium, phenoxazine, or phenoxathiin-3-one core have a peak absorbance wavelength of about 605+/−10 nm and a first absorbance peak with FWHM of less than 100 nm. In some embodiments, the detectable moieties having the thioninium, phenoxazine, or phenoxathiin-3-one core have a peak absorbance wavelength of about 600+/−10 nm and a first absorbance peak with FWHM of less than 100 nm. In some embodiments, the detectable moieties having the thioninium, phenoxazine, or phenoxathiin-3-one core have a peak absorbance wavelength of about 595+/−10 nm and a first absorbance peak with FWHM of less than 100 nm. In some embodiments, the detectable moieties having the thioninium, phenoxazine, or phenoxathiin-3-one core have a peak absorbance wavelength of about 590+/−10 nm and a first absorbance peak with FWHM of less than 100 nm. In some embodiments, the detectable moieties having the thioninium, phenoxazine, or phenoxathiin-3-one core have a peak absorbance wavelength of about 585+/−10 nm and a first absorbance peak with FWHM of less than 100 nm. In some embodiments, the detectable moieties having the thioninium, phenoxazine, or phenoxathiin-3-one core have a peak absorbance wavelength of about 580+/−10 nm and a first absorbance peak with FWHM of less than 100 nm.

In some embodiments, the detectable moiety includes or is derived from a xanthene core (i.e. the detectable moiety includes a xanthene core).

In some embodiments, the xanthene core includes (or is modified to include) one or more electron withdrawing groups (where each electron withdrawing group may be the same or different). In some embodiments, the xanthene core includes (or is modified to include) one electron withdrawing group. In some embodiments, the xanthene core includes (or is modified to include) two electron withdrawing groups. In some embodiments, the xanthene core includes (or is modifying to include) three electron withdrawing groups. In some embodiments, the xanthene core includes (or is modifying to include) three different electron withdrawing groups. In some embodiments, the xanthene core includes (or is modified to include) four electron withdrawing groups.

In some embodiments, the xanthene core includes (or is modified to include) one or more electron donating groups (where each electron donating group may be the same or different). In some embodiments, the xanthene core includes (or is modified to include) one electron donating group. In some embodiments, the xanthene core includes (or is modified to include) two electron donating groups. In some embodiments, the xanthene core includes (or is modifying to include) three electron donating groups. In some embodiments, the xanthene core includes (or is modifying to include) three different electron donating groups. In some embodiments, the xanthene core includes (or is modified to include) four electron donating groups.

In some embodiments, the detectable moieties having the xanthene core have a peak absorbance wavelength ranging from about 580 nm to about 650 nm. In some embodiments, the detectable moieties having the xanthene core have a wavelength ranging from about 590 nm to about 640 nm. In some embodiments, the detectable moieties having the xanthene core have a wavelength ranging from about 600 nm to about 630 nm. In some embodiments, the aforementioned absorbances may be shifted by between about 5 to about 10 nm to the red spectrum when a conjugate including a detectable moiety including a xanthene core is applied to issue.

In some embodiments, the detectable moieties having the xanthene core have a peak absorbance wavelength ranging from about 580 nm to about 650 nm and a first absorbance peak with FWHM of less than 160 nm. In some embodiments, the detectable moieties having the xanthene core have a wavelength ranging from about 590 nm to about 640 nm and a first absorbance peak with FWHM of less than 160 nm. In some embodiments, the detectable moieties having the xanthene core have a wavelength ranging from about 600 nm to about 630 nm and a first absorbance peak with FWHM of less than 160 nm. In some embodiments, the aforementioned absorbances may be shifted by between about 5 to about 10 nm to the red spectrum when a conjugate including a detectable moiety including a xanthene core is applied to issue.

In some embodiments, the detectable moieties having the xanthene core have a peak absorbance wavelength ranging from about 580 nm to about 650 nm and a first absorbance peak with FWHM of less than 130 nm. In some embodiments, the detectable moieties having the xanthene core have a wavelength ranging from about 590 nm to about 640 nm and a first absorbance peak with FWHM of less than 130 nm. In some embodiments, the detectable moieties having the xanthene core have a wavelength ranging from about 600 nm to about 630 nm and a first absorbance peak with FWHM of less than 130 nm. In some embodiments, the aforementioned absorbances may be shifted by between about 5 to about 10 nm to the red spectrum when a conjugate including a detectable moiety including a xanthene core is applied to issue.

In some embodiments, the detectable moieties having the xanthene core have a peak absorbance wavelength ranging from about 580 nm to about 650 nm and a first absorbance peak with FWHM of less than 100 nm. In some embodiments, the detectable moieties having the xanthene core have a wavelength ranging from about 590 nm to about 640 nm and a first absorbance peak with FWHM of less than 100 nm. In some embodiments, the detectable moieties having the xanthene core have a wavelength ranging from about 600 nm to about 630 nm and a first absorbance peak with FWHM of less than 100 nm. In some embodiments, the aforementioned absorbances may be shifted by between about 5 to about 10 nm to the red spectrum when a conjugate including a detectable moiety including a xanthene core is applied to issue.

In some embodiments, the detectable moieties having the xanthene core have a peak absorbance wavelength of about 650+/−10 nm. In some embodiments, the detectable moieties having the xanthene core have a peak absorbance wavelength of about 645+/−10 nm. In some embodiments, the detectable moieties having the xanthene core have a peak absorbance wavelength of about 640+/−10 nm. In some embodiments, the detectable moieties having the xanthene core have a peak absorbance wavelength of about 635+/−10 nm. In some embodiments, the detectable moieties having the xanthene core have a peak absorbance wavelength of about 630+/−10 nm. In some embodiments, the detectable moieties having the xanthene core have a peak absorbance wavelength of about 625+/−10 nm. In some embodiments, the detectable moieties having the xanthene core have a peak absorbance wavelength of about 620+/−10 nm. In some embodiments, the detectable moieties having the xanthene core have a peak absorbance wavelength of about 615+/−10 nm. In some embodiments, the detectable moieties having the xanthene core have a peak absorbance wavelength of about 610+/−10 nm. In some embodiments, the detectable moieties having the xanthene core have a peak absorbance wavelength of about 605+/−10 nm. In some embodiments, the detectable moieties having the xanthene core have a peak absorbance wavelength of about 600+/−10 nm. In some embodiments, the detectable moieties having the xanthene core have a peak absorbance wavelength of about 595+/−10 nm. In some embodiments, the detectable moieties having the xanthene core have a peak absorbance wavelength of about 590+/−10 nm. In some embodiments, the detectable moieties having the xanthene core have a peak absorbance wavelength of about 585+/−10 nm. In some embodiments, the detectable moieties having the xanthene core have a peak absorbance wavelength of about 580+/−10 nm.

In some embodiments, the detectable moieties having the xanthene core have a peak absorbance wavelength of about 650+/−10 nm and a first absorbance peak with FWHM of less than 160 nm. In some embodiments, the detectable moieties having the xanthene core have a peak absorbance wavelength of about 645+/−10 nm and a first absorbance peak with FWHM of less than 160 nm. In some embodiments, the detectable moieties having the xanthene core have a peak absorbance wavelength of about 640+/−10 nm and a first absorbance peak with FWHM of less than 160 nm. In some embodiments, the detectable moieties having the xanthene core have a peak absorbance wavelength of about 635+/−10 nm and a first absorbance peak with FWHM of less than 160 nm. In some embodiments, the detectable moieties having the xanthene core have a peak absorbance wavelength of about 630+/−10 nm and a first absorbance peak with FWHM of less than 160 nm. In some embodiments, the detectable moieties having the xanthene core have a peak absorbance wavelength of about 625+/−10 nm and a first absorbance peak with FWHM of less than 160 nm. In some embodiments, the detectable moieties having the xanthene core have a peak absorbance wavelength of about 620+/−10 nm and a first absorbance peak with FWHM of less than 160 nm. In some embodiments, the detectable moieties having the xanthene core have a peak absorbance wavelength of about 615+/−10 nm and a first absorbance peak with FWHM of less than 160 nm. In some embodiments, the detectable moieties having the xanthene core have a peak absorbance wavelength of about 610+/−10 nm and a first absorbance peak with FWHM of less than 160 nm. In some embodiments, the detectable moieties having the xanthene core have a peak absorbance wavelength of about 605+/−10 nm and a first absorbance peak with FWHM of less than 160 nm. In some embodiments, the detectable moieties having the xanthene core have a peak absorbance wavelength of about 600+/−10 nm and a first absorbance peak with FWHM of less than 160 nm. In some embodiments, the detectable moieties having the xanthene core have a peak absorbance wavelength of about 595+/−10 nm and a first absorbance peak with FWHM of less than 160 nm. In some embodiments, the detectable moieties having the xanthene core have a peak absorbance wavelength of about 590+/−10 nm and a first absorbance peak with FWHM of less than 160 nm. In some embodiments, the detectable moieties having the xanthene core have a peak absorbance wavelength of about 585+/−10 nm and a first absorbance peak with FWHM of less than 160 nm. In some embodiments, the detectable moieties having the xanthene core have a peak absorbance wavelength of about 580+/−10 nm and a first absorbance peak with FWHM of less than 160 nm.

In some embodiments, the detectable moieties having the xanthene core have a peak absorbance wavelength of about 650+/−10 nm and a first absorbance peak with FWHM of less than 130 nm. In some embodiments, the detectable moieties having the xanthene core have a peak absorbance wavelength of about 645+/−10 nm and a first absorbance peak with FWHM of less than 130 nm. In some embodiments, the detectable moieties having the xanthene core have a peak absorbance wavelength of about 640+/−10 nm and a first absorbance peak with FWHM of less than 130 nm. In some embodiments, the detectable moieties having the xanthene core have a peak absorbance wavelength of about 635+/−10 nm and a first absorbance peak with FWHM of less than 130 nm. In some embodiments, the detectable moieties having the xanthene core have a peak absorbance wavelength of about 630+/−10 nm and a first absorbance peak with FWHM of less than 130 nm. In some embodiments, the detectable moieties having the xanthene core have a peak absorbance wavelength of about 625+/−10 nm and a first absorbance peak with FWHM of less than 130 nm. In some embodiments, the detectable moieties having the xanthene core have a peak absorbance wavelength of about 620+/−10 nm and a first absorbance peak with FWHM of less than 130 nm. In some embodiments, the detectable moieties having the xanthene core have a peak absorbance wavelength of about 615+/−10 nm and a first absorbance peak with FWHM of less than 130 nm. In some embodiments, the detectable moieties having the xanthene core have a peak absorbance wavelength of about 610+/−10 nm and a first absorbance peak with FWHM of less than 130 nm. In some embodiments, the detectable moieties having the xanthene core have a peak absorbance wavelength of about 605+/−10 nm and a first absorbance peak with FWHM of less than 130 nm. In some embodiments, the detectable moieties having the xanthene core have a peak absorbance wavelength of about 600+/−10 nm and a first absorbance peak with FWHM of less than 130 nm. In some embodiments, the detectable moieties having the xanthene core have a peak absorbance wavelength of about 595+/−10 nm and a first absorbance peak with FWHM of less than 130 nm. In some embodiments, the detectable moieties having the xanthene core have a peak absorbance wavelength of about 590+/−10 nm and a first absorbance peak with FWHM of less than 130 nm. In some embodiments, the detectable moieties having the xanthene core have a peak absorbance wavelength of about 585+/−10 nm and a first absorbance peak with FWHM of less than 130 nm. In some embodiments, the detectable moieties having the xanthene core have a peak absorbance wavelength of about 580+/−10 nm and a first absorbance peak with FWHM of less than 130 nm.

In some embodiments, the detectable moieties having the xanthene core have a peak absorbance wavelength of about 650+/−10 nm and a first absorbance peak with FWHM of less than 100 nm. In some embodiments, the detectable moieties having the xanthene core have a peak absorbance wavelength of about 645+/−10 nm and a first absorbance peak with FWHM of less than 100 nm. In some embodiments, the detectable moieties having the xanthene core have a peak absorbance wavelength of about 640+/−10 nm and a first absorbance peak with FWHM of less than 100 nm. In some embodiments, the detectable moieties having the xanthene core have a peak absorbance wavelength of about 635+/−10 nm and a first absorbance peak with FWHM of less than 100 nm. In some embodiments, the detectable moieties having the xanthene core have a peak absorbance wavelength of about 630+/−10 nm and a first absorbance peak with FWHM of less than 100 nm. In some embodiments, the detectable moieties having the xanthene core have a peak absorbance wavelength of about 625+/−10 nm and a first absorbance peak with FWHM of less than 100 nm. In some embodiments, the detectable moieties having the xanthene core have a peak absorbance wavelength of about 620+/−10 nm and a first absorbance peak with FWHM of less than 100 nm. In some embodiments, the detectable moieties having the xanthene core have a peak absorbance wavelength of about 615+/−10 nm and a first absorbance peak with FWHM of less than 100 nm. In some embodiments, the detectable moieties having the xanthene core have a peak absorbance wavelength of about 610+/−10 nm and a first absorbance peak with FWHM of less than 100 nm. In some embodiments, the detectable moieties having the xanthene core have a peak absorbance wavelength of about 605+/−10 nm and a first absorbance peak with FWHM of less than 100 nm. In some embodiments, the detectable moieties having the xanthene core have a peak absorbance wavelength of about 600+/−10 nm and a first absorbance peak with FWHM of less than 100 nm. In some embodiments, the detectable moieties having the xanthene core have a peak absorbance wavelength of about 595+/−10 nm and a first absorbance peak with FWHM of less than 100 nm. In some embodiments, the detectable moieties having the xanthene core have a peak absorbance wavelength of about 590+/−10 nm and a first absorbance peak with FWHM of less than 100 nm. In some embodiments, the detectable moieties having the xanthene core have a peak absorbance wavelength of about 585+/−10 nm and a first absorbance peak with FWHM of less than 100 nm. In some embodiments, the detectable moieties having the xanthene core have a peak absorbance wavelength of about 580+/−10 nm and a first absorbance peak with FWHM of less than 100 nm.

Non-limiting examples of compounds having a phenoxazinone, a 4-Hydroxy-3-phenoxazinone, a 7-amino-4-Hydroxy-3-phenoxazinone, a thioninium, a phenoxazine, a phenoxathiin-3-one core, or a xanthene are set forth below:

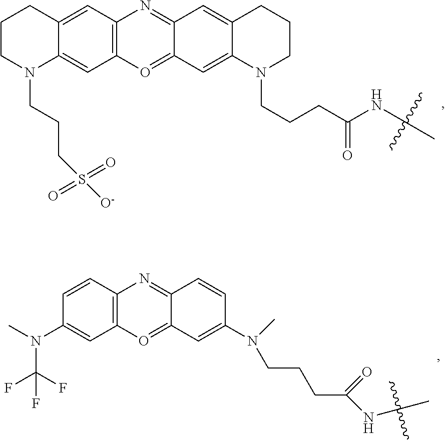

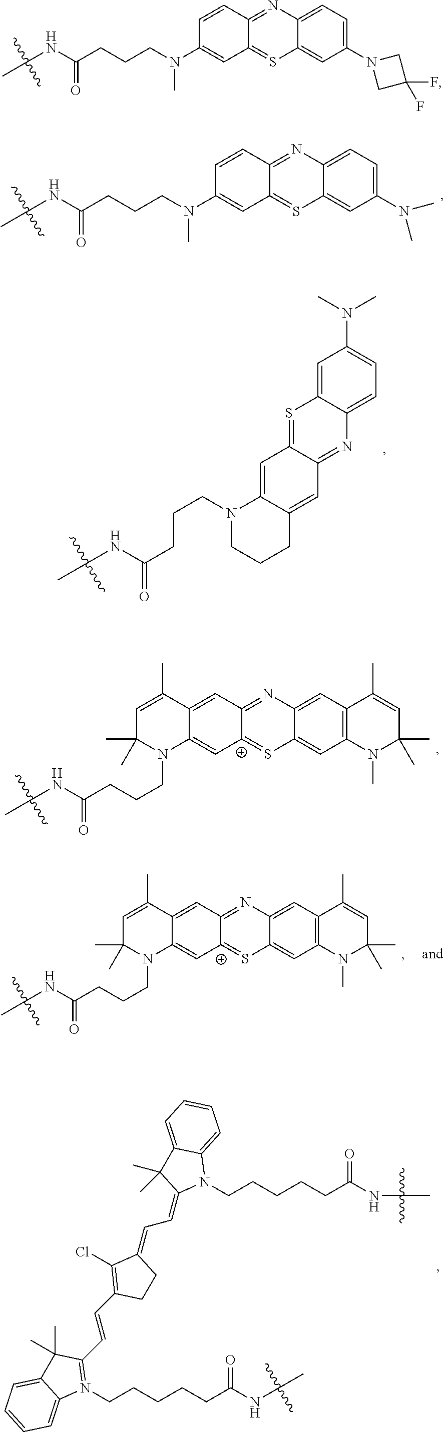

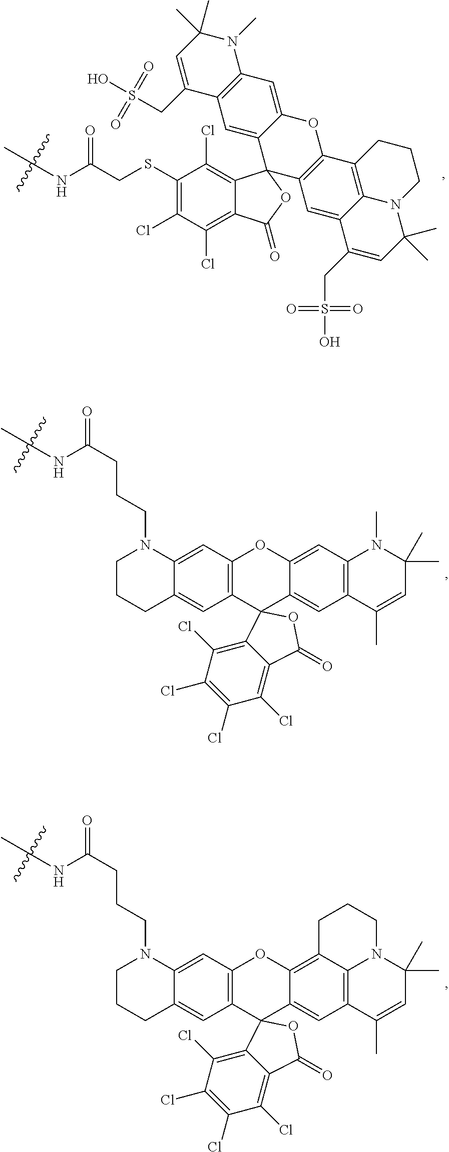 [SMILES: *NC(=O)CSc1c(Cl)c(Cl)c2c(c1Cl)C1(OC2=O)c2cc3c(cc2Oc2c1cc1c4c2CCCN4C(C)(C)C=C1CS(=O)(=O)O)N(C)C(C)(C)C=C3CS(=O)(=O)O ; *NC(=O)CCCN1CCCc2cc3c(cc21)Oc1cc2c(cc1C31OC(=O)c3c(Cl)c(Cl)c(Cl)c(Cl)c31)C(C)=CC(C)(C)N2C ; *NC(=O)CCCN1CCCc2cc3c(cc21)Oc1c(cc2c4c1CCCN4C(C)(C)C=C2C)C31OC(=O)c2c(Cl)c(Cl)c(Cl)c(Cl)c21]

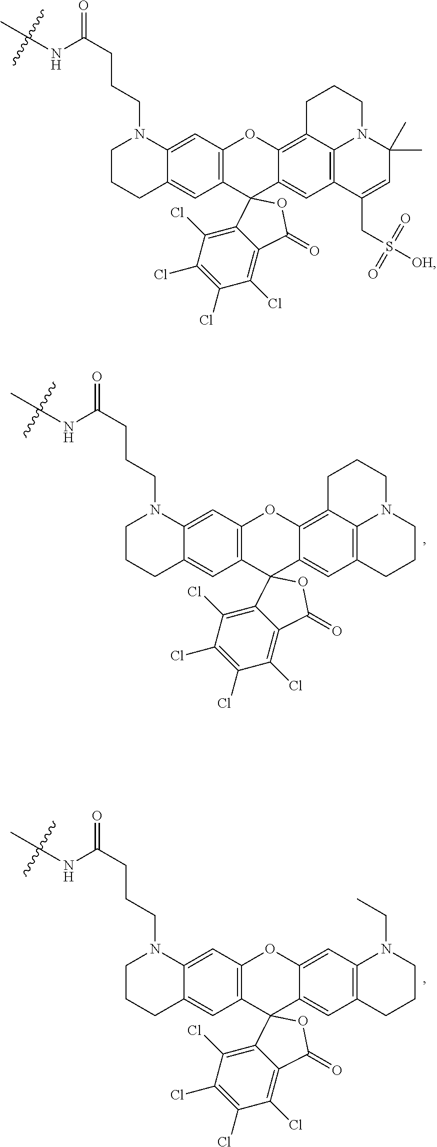 [SMILES: *NC(=O)CCCN1CCCc2cc3c(cc21)Oc1c(cc2c4c1CCCN4C(C)(C)C=C2CS(=O)(=O)O)C31OC(=O)c2c(Cl)c(Cl)c(Cl)c(Cl)c21 ; *NC(=O)CCCN1CCCc2cc3c(cc21)Oc1c(cc2c4c1CCCN4CCC2)C31OC(=O)c2c(Cl)c(Cl)c(Cl)c(Cl)c21 ; *NC(=O)CCCN1CCCc2cc3c(cc21)Oc1cc2c(cc1C31OC(=O)c3c(Cl)c(Cl)c(Cl)c(Cl)c31)CCCN2CC]

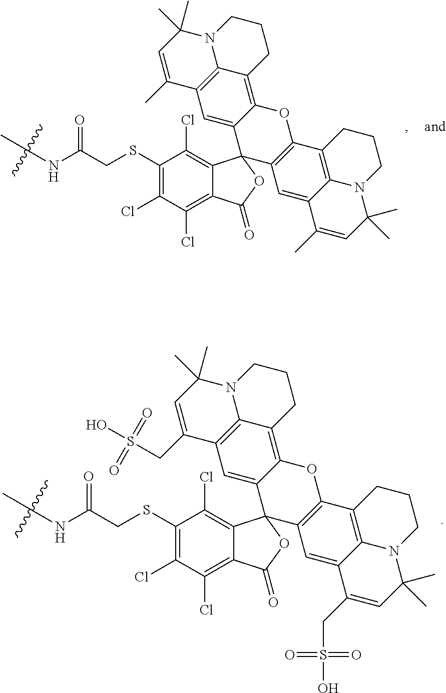 [SMILES: *NC(=O)CSc1c(Cl)c(Cl)c2c(c1Cl)C1(OC2=O)c2cc3c4c(c2Oc2c1cc1c5c2CCCN5C(C)(C)C=C1C)CCCN4C(C)(C)C=C3C ; *NC(=O)CSc1c(Cl)c(Cl)c2c(c1Cl)C1(OC2=O)c2cc3c4c(c2Oc2c1cc1c5c2CCCN5C(C)(C)C=C1CS(=O)(=O)O)CCCN4C(C)(C)C=C3CS(=O)(=O)O]

where the symbol “ [symbol] ” refers to the site in which the detectable moiety (here, the phenoxazinone, the 4-Hydroxy-3-phenoxazinone, the 7-amino-4-Hydroxy-3-phenoxazinone, the thioninium, the phenoxazine, the phenoxathiin-3-one core, or the xanthene core) is coupled (directly or indirectly) to another moiety of the detectable conjugate (e.g. to a tyramide moiety, to a quinone methide moiety, to a functional group capable or participating in a “click chemistry” reaction, to an antibody, to an enzyme, to a hapten, etc.). Yet other examples are disclosed herein.

Detectable Moieties within the Infrared Spectrum

In some embodiments, the detectable moieties have a wavelength within the infrared spectrum. In some embodiments, the detectable moieties have a wavelength of greater than about 740 nm. In some embodiments, the detectable moieties have a wavelength of greater than about 750 nm. In some embodiments, the detectable moieties have a wavelength of greater than about 760 nm. In some embodiments, the detectable moieties have a wavelength of greater than about 765 nm. In some embodiments, the detectable moieties have a wavelength of greater than about 770 nm. In some embodiments, the detectable moieties have a wavelength of greater than about 775 nm. In some embodiments, the detectable moieties have a wavelength of greater than about 780 nm. In some embodiments, the detectable moieties have a wavelength of greater than about 785 nm. In some embodiments, the detectable moieties have a wavelength of greater than about 790 nm. In some embodiments the detectable moieties have a wavelength ranging from between about 760 nm to about 1 mm, from about 770 nm to about 1 mm, or from about 780 nm to about 1 mm.

In some embodiments, the detectable moieties have a wavelength of greater than about 740 nm and a first absorbance peak with FWHM of less than 160 nm. In some embodiments, the detectable moieties have a wavelength of greater than about 750 nm and a first absorbance peak with FWHM of less than 160 nm. In some embodiments, the detectable moieties have a wavelength of greater than about 760 nm and a first absorbance peak with FWHM of less than 160 nm. In some embodiments, the detectable moieties have a wavelength of greater than about 765 nm and a first absorbance peak with FWHM of less than 160 nm. In some embodiments, the detectable moieties have a wavelength of greater than about 770 nm and a first absorbance peak with FWHM of less than 160 nm. In some embodiments, the detectable moieties have a wavelength of greater than about 775 nm and a first absorbance peak with FWHM of less than 160 nm. In some embodiments, the detectable moieties have a wavelength of greater than about 780 nm and a first absorbance peak with FWHM of less than 160 nm. In some embodiments, the detectable moieties have a wavelength of greater than about 785 nm and a first absorbance peak with FWHM of less than 160 nm. In some embodiments, the detectable moieties have a wavelength of greater than about 790 nm and a first absorbance peak with FWHM of less than 160 nm.

In some embodiments, the detectable moieties have a wavelength of greater than about 740 nm and a first absorbance peak with FWHM of less than 130 nm. In some embodiments, the detectable moieties have a wavelength of greater than about 750 nm and a first absorbance peak with FWHM of less than 130 nm. In some embodiments, the detectable moieties have a wavelength of greater than about 760 nm and a first absorbance peak with FWHM of less than 130 nm. In some embodiments, the detectable moieties have a wavelength of greater than about 765 nm and a first absorbance peak with FWHM of less than 130 nm. In some embodiments, the detectable moieties have a wavelength of greater than about 770 nm and a first absorbance peak with FWHM of less than 130 nm. In some embodiments, the detectable moieties have a wavelength of greater than about 775 nm and a first absorbance peak with FWHM of less than 130 nm. In some embodiments, the detectable moieties have a wavelength of greater than about 780 nm and a first absorbance peak with FWHM of less than 130 nm. In some embodiments, the detectable moieties have a wavelength of greater than about 785 nm and a first absorbance peak with FWHM of less than 130 nm. In some embodiments, the detectable moieties have a wavelength of greater than about 790 nm and a first absorbance peak with FWHM of less than 130 nm.

In some embodiments, the detectable moieties have a wavelength of greater than about 740 nm and a first absorbance peak with FWHM of less than 100 nm. In some embodiments, the detectable moieties have a wavelength of greater than about 750 nm and a first absorbance peak with FWHM of less than 100 nm. In some embodiments, the detectable moieties have a wavelength of greater than about 760 nm and a first absorbance peak with FWHM of less than 100 nm. In some embodiments, the detectable moieties have a wavelength of greater than about 765 nm and a first absorbance peak with FWHM of less than 100 nm. In some embodiments, the detectable moieties have a wavelength of greater than about 770 nm and a first absorbance peak with FWHM of less than 100 nm. In some embodiments, the detectable moieties have a wavelength of greater than about 775 nm and a first absorbance peak with FWHM of less than 100 nm. In some embodiments, the detectable moieties have a wavelength of greater than about 780 nm and a first absorbance peak with FWHM of less than 100 nm. In some embodiments, the detectable moieties have a wavelength of greater than about 785 nm and a first absorbance peak with FWHM of less than 100 nm. In some embodiments, the detectable moieties have a wavelength of greater than about 790 nm and a first absorbance peak with FWHM of less than 100 nm.

In some embodiments, the detectable moiety includes or is derived from a heptamethine cyanine core (i.e. the detectable moiety includes a heptamethine cyanine core).

In some embodiments, the heptamethine cyanine core (includes (or is modified to include) one or more electron withdrawing groups (where each electron withdrawing group may be the same or different). In some embodiments, the heptamethine cyanine core includes (or is modified to include) one electron withdrawing group. In some embodiments, the heptamethine cyanine core includes (or is modified to include) two electron withdrawing groups. In some embodiments, the heptamethine cyanine core includes (or is modifying to include) three electron withdrawing groups. In some embodiments, the heptamethine cyanine core includes (or is modifying to include) three different electron withdrawing groups. In some embodiments, the heptamethine cyanine core includes (or is modified to include) four electron withdrawing groups.

In some embodiments, the heptamethine cyanine core (includes (or is modified to include) one or more electron donating groups (where each electron withdrawing group may be the same or different). In some embodiments, the heptamethine cyanine core includes (or is modified to include) one electron donating group. In some embodiments, the heptamethine cyanine core includes (or is modified to include) two electron donating groups. In some embodiments, the heptamethine cyanine core includes (or is modifying to include) three electron donating groups. In some embodiments, the heptamethine cyanine core includes (or is modifying to include) three different electron donating groups. In some embodiments, the heptamethine cyanine core includes (or is modified to include) four electron donating groups.

In some embodiments, the detectable moieties having the heptamethine cyanine core have a wavelength ranging from about 780 nm to about 950 nm. In some embodiments, the detectable moieties having the heptamethine cyanine core have a wavelength ranging from about 810 nm to about 920 nm. In some embodiments, the detectable moieties having the heptamethine cyanine have a wavelength ranging from about 840 nm to about 880 nm.

In some embodiments, the detectable moieties having the heptamethine cyanine core have a wavelength ranging from about 780 nm to about 950 nm and a first absorbance peak with FWHM of less than 160 nm. In some embodiments, the detectable moieties having the heptamethine cyanine core have a wavelength ranging from about 810 nm to about 920 nm and a first absorbance peak with FWHM of less than 160 nm In some embodiments, the detectable moieties having the heptamethine cyanine have a wavelength ranging from about 840 nm to about 880 nm and a first absorbance peak with FWHM of less than 160 nm.

In some embodiments, the detectable moieties having the heptamethine cyanine core have a wavelength ranging from about 780 nm to about 950 nm and a first absorbance peak with FWHM of less than 130 nm. In some embodiments, the detectable moieties having the heptamethine cyanine core have a wavelength ranging from about 810 nm to about 920 nm and a first absorbance peak with FWHM of less than 130 nm In some embodiments, the detectable moieties having the heptamethine cyanine have a wavelength ranging from about 840 nm to about 880 nm and a first absorbance peak with FWHM of less than 130 nm.

In some embodiments, the detectable moieties having the heptamethine cyanine core have a wavelength ranging from about 780 nm to about 950 nm and a first absorbance peak with FWHM of less than 100 nm. In some embodiments, the detectable moieties having the heptamethine cyanine core have a wavelength ranging from about 810 nm to about 920 nm and a first absorbance peak with FWHM of less than 100 nm In some embodiments, the detectable moieties having the heptamethine cyanine have a wavelength ranging from about 840 nm to about 880 nm and a first absorbance peak with FWHM of less than 100 nm.

In some embodiments, the detectable moieties having the heptamethine cyanine core have a peak absorbance wavelength of about 950+/−10 nm. In some embodiments, the detectable moieties having the heptamethine cyanine core have a peak absorbance wavelength of about 945+/−10 nm. In some embodiments, the detectable moieties having the heptamethine cyanine core have a peak absorbance wavelength of about 940+/−10 nm. In some embodiments, the detectable moieties having the heptamethine cyanine core have a peak absorbance wavelength of about 935+/−10 nm. In some embodiments, the detectable moieties having the heptamethine cyanine core have a peak absorbance wavelength of about 930+/−10 nm. In some embodiments, the detectable moieties having the heptamethine cyanine core have a peak absorbance wavelength of about 925+/−10 nm. In some embodiments, the detectable moieties having the heptamethine cyanine core have a peak absorbance wavelength of about 920+/−10 nm. In some embodiments, the detectable moieties having the heptamethine cyanine core have a peak absorbance wavelength of about 915+/−10 nm. In some embodiments, the detectable moieties having the heptamethine cyanine core have a peak absorbance wavelength of about 910+/−10 nm. In some embodiments, the detectable moieties having the heptamethine cyanine core have a peak absorbance wavelength of about 905+/−10 nm. In some embodiments, the detectable moieties having the heptamethine cyanine core have a peak absorbance wavelength of about 900+/−10 nm. In some embodiments, the detectable moieties having the heptamethine cyanine core have a peak absorbance wavelength of about 895+/−10 nm. In some embodiments, the detectable moieties having the heptamethine cyanine core have a peak absorbance wavelength of about 890+/−10 nm. In some embodiments, the detectable moieties having the heptamethine cyanine core have a peak absorbance wavelength of about 885+/−10 nm. In some embodiments, the detectable moieties having the heptamethine cyanine core have a peak absorbance wavelength of about 880+/−10 nm. In some embodiments, the detectable moieties having the heptamethine cyanine core have a peak absorbance wavelength of about 870+/−10 nm. In some embodiments, the detectable moieties having the heptamethine cyanine core have a peak absorbance wavelength of about 865+/−10 nm. In some embodiments, the detectable moieties having the heptamethine cyanine core have a peak absorbance wavelength of about 860+/−10 nm. In some embodiments, the detectable moieties having the heptamethine cyanine core have a peak absorbance wavelength of about 855+/−10 nm. In some embodiments, the detectable moieties having the heptamethine cyanine core have a peak absorbance wavelength of about 850+/−10 nm. In some embodiments, the detectable moieties having the heptamethine cyanine core have a peak absorbance wavelength of about 845+/−10 nm. In some embodiments, the detectable moieties having the heptamethine cyanine core have a peak absorbance wavelength of about 840+/−10 nm. In some embodiments, the detectable moieties having the heptamethine cyanine core have a peak absorbance wavelength of about 835+/−10 nm. In some embodiments, the detectable moieties having the heptamethine cyanine core have a peak absorbance wavelength of about 830+/−10 nm. In some embodiments, the detectable moieties having the heptamethine cyanine core have a peak absorbance wavelength of about 825+/−10 nm. In some embodiments, the detectable moieties having the heptamethine cyanine core have a peak absorbance wavelength of about 820+/−10 nm. In some embodiments, the detectable moieties having the heptamethine cyanine core have a peak absorbance wavelength of about 815+/−10 nm. In some embodiments, the detectable moieties having the heptamethine cyanine core have a peak absorbance wavelength of about 800+/−10 nm. In some embodiments, the detectable moieties having the heptamethine cyanine core have a peak absorbance wavelength of about 795+/−10 nm. In some embodiments, the detectable moieties having the heptamethine cyanine core have a peak absorbance wavelength of about 790+/−10 nm. In some embodiments, the detectable moieties having the heptamethine cyanine core have a peak absorbance wavelength of about 785+/−10 nm. In some embodiments, the detectable moieties having the heptamethine cyanine core have a peak absorbance wavelength of about 780+/−10 nm.

In some embodiments, the detectable moieties having the heptamethine cyanine core have a peak absorbance wavelength of about 950+/−10 nm and a first absorbance peak with FWHM of less than 160 nm. In some embodiments, the detectable moieties having the heptamethine cyanine core have a peak absorbance wavelength of about 945+/−10 nm and a first absorbance peak with FWHM of less than 160 nm. In some embodiments, the detectable moieties having the heptamethine cyanine core have a peak absorbance wavelength of about 940+/−10 nm and a first absorbance peak with FWHM of less than 160 nm. In some embodiments, the detectable moieties having the heptamethine cyanine core have a peak absorbance wavelength of about 935+/−10 nm and a first absorbance peak with FWHM of less than 160 nm. In some embodiments, the detectable moieties having the heptamethine cyanine core have a peak absorbance wavelength of about 930+/−10 nm and a first absorbance peak with FWHM of less than 160 nm. In some embodiments, the detectable moieties having the heptamethine cyanine core have a peak absorbance wavelength of about 925+/−10 nm and a first absorbance peak with FWHM of less than 160 nm. In some embodiments, the detectable moieties having the heptamethine cyanine core have a peak absorbance wavelength of about 920+/−10 nm and a first absorbance peak with FWHM of less than 160 nm. In some embodiments, the detectable moieties having the heptamethine cyanine core have a peak absorbance wavelength of about 915+/−10 nm and a first absorbance peak with FWHM of less than 160 nm. In some embodiments, the detectable moieties having the heptamethine cyanine core have a peak absorbance wavelength of about 910+/−10 nm and a first absorbance peak with FWHM of less than 160 nm. In some embodiments, the detectable moieties having the heptamethine cyanine core have a peak absorbance wavelength of about 905+/−10 nm and a first absorbance peak with FWHM of less than 160 nm. In some embodiments, the detectable moieties having the heptamethine cyanine core have a peak absorbance wavelength of about 900+/−10 nm and a first absorbance peak with FWHM of less than 160 nm. In some embodiments, the detectable moieties having the heptamethine cyanine core have a peak absorbance wavelength of about 895+/−10 nm and a first absorbance peak with FWHM of less than 160 nm. In some embodiments, the detectable moieties having the heptamethine cyanine core have a peak absorbance wavelength of about 890+/−10 nm and a first absorbance peak with FWHM of less than 160 nm. In some embodiments, the detectable moieties having the heptamethine cyanine core have a peak absorbance wavelength of about 885+/−10 nm and a first absorbance peak with FWHM of less than 160 nm. In some embodiments, the detectable moieties having the heptamethine cyanine core have a peak absorbance wavelength of about 880+/−10 nm and a first absorbance peak with FWHM of less than 160 nm. In some embodiments, the detectable moieties having the heptamethine cyanine core have a peak absorbance wavelength of about 870+/−10 nm and a first absorbance peak with FWHM of less than 160 nm. In some embodiments, the detectable moieties having the heptamethine cyanine core have a peak absorbance wavelength of about 865+/−10 nm and a first absorbance peak with FWHM of less than 160 nm. In some embodiments, the detectable moieties having the heptamethine cyanine core have a peak absorbance wavelength of about 860+/−10 nm and a first absorbance peak with FWHM of less than 160 nm. In some embodiments, the detectable moieties having the heptamethine cyanine core have a peak absorbance wavelength of about 855+/−10 nm and a first absorbance peak with FWHM of less than 160 nm. In some embodiments, the detectable moieties having the heptamethine cyanine core have a peak absorbance wavelength of about 850+/−10 nm and a first absorbance peak with FWHM of less than 160 nm. In some embodiments, the detectable moieties having the heptamethine cyanine core have a peak absorbance wavelength of about 845+/−10 nm and a first absorbance peak with FWHM of less than 160 nm. In some embodiments, the detectable moieties having the heptamethine cyanine core have a peak absorbance wavelength of about 840+/−10 nm and a first absorbance peak with FWHM of less than 160 nm. In some embodiments, the detectable moieties having the heptamethine cyanine core have a peak absorbance wavelength of about 835+/−10 nm and a first absorbance peak with FWHM of less than 160 nm. In some embodiments, the detectable moieties having the heptamethine cyanine core have a peak absorbance wavelength of about 830+/−10 nm and a first absorbance peak with FWHM of less than 160 nm. In some embodiments, the detectable moieties having the heptamethine cyanine core have a peak absorbance wavelength of about 825+/−10 nm and a first absorbance peak with FWHM of less than 160 nm. In some embodiments, the detectable moieties having the heptamethine cyanine core have a peak absorbance wavelength of about 820+/−10 nm and a first absorbance peak with FWHM of less than 160 nm. In some embodiments, the detectable moieties having the heptamethine cyanine core have a peak absorbance wavelength of about 815+/−10 nm and a first absorbance peak with FWHM of less than 160 nm. In some embodiments, the detectable moieties having the heptamethine cyanine core have a peak absorbance wavelength of about 800+/−10 nm and a first absorbance peak with FWHM of less than 160 nm. In some embodiments, the detectable moieties having the heptamethine cyanine core have a peak absorbance wavelength of about 795+/−10 nm and a first absorbance peak with FWHM of less than 160 nm. In some embodiments, the detectable moieties having the heptamethine cyanine core have a peak absorbance wavelength of about 790+/−10 nm and a first absorbance peak with FWHM of less than 160 nm. In some embodiments, the detectable moieties having the heptamethine cyanine core have a peak absorbance wavelength of about 785+/−10 nm and a first absorbance peak with FWHM of less than 160 nm. In some embodiments, the detectable moieties having the heptamethine cyanine core have a peak absorbance wavelength of about 780+/−10 nm and a first absorbance peak with FWHM of less than 160 nm.

In some embodiments, the detectable moieties having the heptamethine cyanine core have a peak absorbance wavelength of about 950+/−10 nm and a first absorbance peak with FWHM of less than 130 nm. In some embodiments, the detectable moieties having the heptamethine cyanine core have a peak absorbance wavelength of about 945+/−10 nm and a first absorbance peak with FWHM of less than 130 nm. In some embodiments, the detectable moieties having the heptamethine cyanine core have a peak absorbance wavelength of about 940+/−10 nm and a first absorbance peak with FWHM of less than 130 nm. In some embodiments, the detectable moieties having the heptamethine cyanine core have a peak absorbance wavelength of about 935+/−10 nm and a first absorbance peak with FWHM of less than 130 nm. In some embodiments, the detectable moieties having the heptamethine cyanine core have a peak absorbance wavelength of about 930+/−10 nm and a first absorbance peak with FWHM of less than 130 nm. In some embodiments, the detectable moieties having the heptamethine cyanine core have a peak absorbance wavelength of about 925+/−10 nm and a first absorbance peak with FWHM of less than 130 nm. In some embodiments, the detectable moieties having the heptamethine cyanine core have a peak absorbance wavelength of about 920+/−10 nm and a first absorbance peak with FWHM of less than 130 nm. In some embodiments, the detectable moieties having the heptamethine cyanine core have a peak absorbance wavelength of about 915+/−10 nm and a first absorbance peak with FWHM of less than 130 nm. In some embodiments, the detectable moieties having the heptamethine cyanine core have a peak absorbance wavelength of about 910+/−10 nm and a first absorbance peak with FWHM of less than 130 nm. In some embodiments, the detectable moieties having the heptamethine cyanine core have a peak absorbance wavelength of about 905+/−10 nm and a first absorbance peak with FWHM of less than 130 nm. In some embodiments, the detectable moieties having the heptamethine cyanine core have a peak absorbance wavelength of about 900+/−10 nm and a first absorbance peak with FWHM of less than 130 nm. In some embodiments, the detectable moieties having the heptamethine cyanine core have a peak absorbance wavelength of about 895+/−10 nm and a first absorbance peak with FWHM of less than 130 nm. In some embodiments, the detectable moieties having the heptamethine cyanine core have a peak absorbance wavelength of about 890+/−10 nm and a first absorbance peak with FWHM of less than 130 nm. In some embodiments, the detectable moieties having the heptamethine cyanine core have a peak absorbance wavelength of about 885+/−10 nm and a first absorbance peak with FWHM of less than 130 nm. In some embodiments, the detectable moieties having the heptamethine cyanine core have a peak absorbance wavelength of about 880+/−10 nm and a first absorbance peak with FWHM of less than 130 nm. In some embodiments, the detectable moieties having the heptamethine cyanine core have a peak absorbance wavelength of about 870+/−10 nm and a first absorbance peak with FWHM of less than 130 nm. In some embodiments, the detectable moieties having the heptamethine cyanine core have a peak absorbance wavelength of about 865+/−10 nm and a first absorbance peak with FWHM of less than 130 nm. In some embodiments, the detectable moieties having the heptamethine cyanine core have a peak absorbance wavelength of about 860+/−10 nm and a first absorbance peak with FWHM of less than 130 nm. In some embodiments, the detectable moieties having the heptamethine cyanine core have a peak absorbance wavelength of about 855+/−10 nm and a first absorbance peak with FWHM of less than 130 nm. In some embodiments, the detectable moieties having the heptamethine cyanine core have a peak absorbance wavelength of about 850+/−10 nm and a first absorbance peak with FWHM of less than 130 nm. In some embodiments, the detectable moieties having the heptamethine cyanine core have a peak absorbance wavelength of about 845+/−10 nm and a first absorbance peak with FWHM of less than 130 nm. In some embodiments, the detectable moieties having the heptamethine cyanine core have a peak absorbance wavelength of about 840+/−10 nm and a first absorbance peak with FWHM of less than 130 nm. In some embodiments, the detectable moieties having the heptamethine cyanine core have a peak absorbance wavelength of about 835+/−10 nm and a first absorbance peak with FWHM of less than 130 nm. In some embodiments, the detectable moieties having the heptamethine cyanine core have a peak absorbance wavelength of about 830+/−10 nm and a first absorbance peak with FWHM of less than 130 nm. In some embodiments, the detectable moieties having the heptamethine cyanine core have a peak absorbance wavelength of about 825+/−10 nm and a first absorbance peak with FWHM of less than 130 nm. In some embodiments, the detectable moieties having the heptamethine cyanine core have a peak absorbance wavelength of about 820+/−10 nm and a first absorbance peak with FWHM of less than 130 nm. In some embodiments, the detectable moieties having the heptamethine cyanine core have a peak absorbance wavelength of about 815+/−10 nm and a first absorbance peak with FWHM of less than 130 nm. In some embodiments, the detectable moieties having the heptamethine cyanine core have a peak absorbance wavelength of about 800+/−10 nm and a first absorbance peak with FWHM of less than 130 nm. In some embodiments, the detectable moieties having the heptamethine cyanine core have a peak absorbance wavelength of about 795+/−10 nm and a first absorbance peak with FWHM of less than 130 nm. In some embodiments, the detectable moieties having the heptamethine cyanine core have a peak absorbance wavelength of about 790+/−10 nm and a first absorbance peak with FWHM of less than 130 nm. In some embodiments, the detectable moieties having the heptamethine cyanine core have a peak absorbance wavelength of about 785+/−10 nm and a first absorbance peak with FWHM of less than 130 nm. In some embodiments, the detectable moieties having the heptamethine cyanine core have a peak absorbance wavelength of about 780+/−10 nm and a first absorbance peak with FWHM of less than 130 nm.

In some embodiments, the detectable moieties having the heptamethine cyanine core have a peak absorbance wavelength of about 950+/−10 nm and a first absorbance peak with FWHM of less than 100 nm. In some embodiments, the detectable moieties having the heptamethine cyanine core have a peak absorbance wavelength of about 945+/−10 nm and a first absorbance peak with FWHM of less than 100 nm. In some embodiments, the detectable moieties having the heptamethine cyanine core have a peak absorbance wavelength of about 940+/−10 nm and a first absorbance peak with FWHM of less than 100 nm. In some embodiments, the detectable moieties having the heptamethine cyanine core have a peak absorbance wavelength of about 935+/−10 nm and a first absorbance peak with FWHM of less than 100 nm. In some embodiments, the detectable moieties having the heptamethine cyanine core have a peak absorbance wavelength of about 930+/−10 nm and a first absorbance peak with FWHM of less than 100 nm. In some embodiments, the detectable moieties having the heptamethine cyanine core have a peak absorbance wavelength of about 925+/−10 nm and a first absorbance peak with FWHM of less than 100 nm. In some embodiments, the detectable moieties having the heptamethine cyanine core have a peak absorbance wavelength of about 920+/−10 nm and a first absorbance peak with FWHM of less than 100 nm. In some embodiments, the detectable moieties having the heptamethine cyanine core have a peak absorbance wavelength of about 915+/−10 nm and a first absorbance peak with FWHM of less than 100 nm. In some embodiments, the detectable moieties having the heptamethine cyanine core have a peak absorbance wavelength of about 910+/−10 nm and a first absorbance peak with FWHM of less than 100 nm. In some embodiments, the detectable moieties having the heptamethine cyanine core have a peak absorbance wavelength of about 905+/−10 nm and a first absorbance peak with FWHM of less than 100 nm. In some embodiments, the detectable moieties having the heptamethine cyanine core have a peak absorbance wavelength of about 900+/−10 nm and a first absorbance peak with FWHM of less than 100 nm. In some embodiments, the detectable moieties having the heptamethine cyanine core have a peak absorbance wavelength of about 895+/−10 nm and a first absorbance peak with FWHM of less than 100 nm. In some embodiments, the detectable moieties having the heptamethine cyanine core have a peak absorbance wavelength of about 890+/−10 nm and a first absorbance peak with FWHM of less than 100 nm. In some embodiments, the detectable moieties having the heptamethine cyanine core have a peak absorbance wavelength of about 885+/−10 nm and a first absorbance peak with FWHM of less than 100 nm. In some embodiments, the detectable moieties having the heptamethine cyanine core have a peak absorbance wavelength of about 880+/−10 nm and a first absorbance peak with FWHM of less than 100 nm. In some embodiments, the detectable moieties having the heptamethine cyanine core have a peak absorbance wavelength of about 870+/−10 nm and a first absorbance peak with FWHM of less than 100 nm. In some embodiments, the detectable moieties having the heptamethine cyanine core have a peak absorbance wavelength of about 865+/−10 nm and a first absorbance peak with FWHM of less than 100 nm. In some embodiments, the detectable moieties having the heptamethine cyanine core have a peak absorbance wavelength of about 860+/−10 nm and a first absorbance peak with FWHM of less than 100 nm. In some embodiments, the detectable moieties having the heptamethine cyanine core have a peak absorbance wavelength of about 855+/−10 nm and a first absorbance peak with FWHM of less than 100 nm. In some embodiments, the detectable moieties having the heptamethine cyanine core have a peak absorbance wavelength of about 850+/−10 nm and a first absorbance peak with FWHM of less than 100 nm. In some embodiments, the detectable moieties having the heptamethine cyanine core have a peak absorbance wavelength of about 845+/−10 nm and a first absorbance peak with FWHM of less than 100 nm. In some embodiments, the detectable moieties having the heptamethine cyanine core have a peak absorbance wavelength of about 840+/−10 nm and a first absorbance peak with FWHM of less than 100 nm. In some embodiments, the detectable moieties having the heptamethine cyanine core have a peak absorbance wavelength of about 835+/−10 nm and a first absorbance peak with FWHM of less than 100 nm. In some embodiments, the detectable moieties having the heptamethine cyanine core have a peak absorbance wavelength of about 830+/−10 nm and a first absorbance peak with FWHM of less than 100 nm. In some embodiments, the detectable moieties having the heptamethine cyanine core have a peak absorbance wavelength of about 825+/−10 nm and a first absorbance peak with FWHM of less than 100 nm. In some embodiments, the detectable moieties having the heptamethine cyanine core have a peak absorbance wavelength of about 820+/−10 nm and a first absorbance peak with FWHM of less than 100 nm. In some embodiments, the detectable moieties having the heptamethine cyanine core have a peak absorbance wavelength of about 815+/−10 nm and a first absorbance peak with FWHM of less than 100 nm. In some embodiments, the detectable moieties having the heptamethine cyanine core have a peak absorbance wavelength of about 800+/−10 nm and a first absorbance peak with FWHM of less than 100 nm. In some embodiments, the detectable moieties having the heptamethine cyanine core have a peak absorbance wavelength of about 795+/−10 nm and a first absorbance peak with FWHM of less than 100 nm. In some embodiments, the detectable moieties having the heptamethine cyanine core have a peak absorbance wavelength of about 790+/−10 nm and a first absorbance peak with FWHM of less than 100 nm. In some embodiments, the detectable moieties having the heptamethine cyanine core have a peak absorbance wavelength of about 785+/−10 nm and a first absorbance peak with FWHM of less than 100 nm. In some embodiments, the detectable moieties having the heptamethine cyanine core have a peak absorbance wavelength of about 780+/−10 nm and a first absorbance peak with FWHM of less than 100 nm.

In some embodiments, the detectable moiety includes or is derived from a croconate core (i.e. the detectable moiety includes a croconate core).

In some embodiments, the croconate core (includes (or is modified to include) one or more electron withdrawing groups (where each electron withdrawing group may be the same or different). In some embodiments, the croconate core includes (or is modified to include) one electron withdrawing group. In some embodiments, the croconate core includes (or is modified to include) two electron withdrawing groups. In some embodiments, the croconate core includes (or is modifying to include) three electron withdrawing groups. In some embodiments, the croconate core includes (or is modifying to include) three different electron withdrawing groups. In some embodiments, the croconate core includes (or is modified to include) four electron withdrawing groups.

In some embodiments, the croconate core (includes (or is modified to include) one or more electron donating groups (where each electron withdrawing group may be the same or different). In some embodiments, the croconate core includes (or is modified to include) one electron donating group. In some embodiments, the croconate core includes (or is modified to include) two electron donating groups. In some embodiments, the croconate core includes (or is modifying to include) three electron donating groups. In some embodiments, the croconate core includes (or is modifying to include) three different electron donating groups. In some embodiments, the croconate core includes (or is modified to include) four electron donating groups.

In some embodiments, the detectable moieties having the croconate core have a wavelength ranging from about 780 nm to about 900 nm. In some embodiments, the detectable moieties having the croconate core have a wavelength ranging from about 800 nm to about 880 nm. In some embodiments, the detectable moieties having the croconate core have a wavelength ranging from about 820 nm to about 860 nm.

In some embodiments, the detectable moieties having the croconate core have a wavelength ranging from about 780 nm to about 900 nm and a first absorbance peak with FWHM of less than 160 nm. In some embodiments, the detectable moieties having the croconate core have a wavelength ranging from about 800 nm to about 880 nm and a first absorbance peak with FWHM of less than 160 nm. In some embodiments, the detectable moieties having the croconate core have a wavelength ranging from about 820 nm to about 860 nm and a first absorbance peak with FWHM of less than 160 nm. In some embodiments, the detectable moieties having the croconate core have a wavelength ranging from about 780 nm to about 900 nm and a first absorbance peak with FWHM of less than 130 nm. In some embodiments, the detectable moieties having the croconate core have a wavelength ranging from about 800 nm to about 880 nm and a first absorbance peak with FWHM of less than 130 nm. In some embodiments, the detectable moieties having the croconate core have a wavelength ranging from about 820 nm to about 860 nm and a first absorbance peak with FWHM of less than 130 nm.

In some embodiments, the detectable moieties having the croconate core have a peak absorbance wavelength of about 900+/−10 nm. In some embodiments, the detectable moieties having the croconate core have a peak absorbance wavelength of about 895+/−10 nm. In some embodiments, the detectable moieties having the croconate core have a peak absorbance wavelength of about 890+/−10 nm. In some embodiments, the detectable moieties having the croconate core have a peak absorbance wavelength of about 885+/−10 nm. In some embodiments, the detectable moieties having the croconate core have a peak absorbance wavelength of about 880+/−10 nm. In some embodiments, the detectable moieties having the croconate core have a peak absorbance wavelength of about 870+/−10 nm. In some embodiments, the detectable moieties having the croconate core have a peak absorbance wavelength of about 865+/−10 nm. In some embodiments, the detectable moieties having the croconate core have a peak absorbance wavelength of about 860+/−10 nm. In some embodiments, the detectable moieties having the croconate core have a peak absorbance wavelength of about 855+/−10 nm. In some embodiments, the detectable moieties having the croconate core have a peak absorbance wavelength of about 850+/−10 nm. In some embodiments, the detectable moieties having the croconate core have a peak absorbance wavelength of about 845+/−10 nm. In some embodiments, the detectable moieties having the croconate core have a peak absorbance wavelength of about 840+/−10 nm. In some embodiments, the detectable moieties having the croconate core have a peak absorbance wavelength of about 835+/−10 nm. In some embodiments, the detectable moieties having the croconate core have a peak absorbance wavelength of about 830+/−10 nm. In some embodiments, the detectable moieties having the croconate core have a peak absorbance wavelength of about 825+/−10 nm. In some embodiments, the detectable moieties having the croconate core have a peak absorbance wavelength of about 820+/−10 nm. In some embodiments, the detectable moieties having the croconate core have a peak absorbance wavelength of about 815+/−10 nm. In some embodiments, the detectable moieties having the croconate core have a peak absorbance wavelength of about 800+/−10 nm. In some embodiments, the detectable moieties having the croconate core have a peak absorbance wavelength of about 795+/−10 nm. In some embodiments, the detectable moieties having the croconate core have a peak absorbance wavelength of about 790+/−10 nm. In some embodiments, the detectable moieties having the croconate core have a peak absorbance wavelength of about 785+/−10 nm. In some embodiments, the detectable moieties having the croconate core have a peak absorbance wavelength of about 780+/−10 nm.

In some embodiments, the detectable moieties having the croconate core have a peak absorbance wavelength of about 900+/−10 nm and a first absorbance peak with FWHM of less than 160 nm. In some embodiments, the detectable moieties having the croconate core have a peak absorbance wavelength of about 895+/−10 nm and a first absorbance peak with FWHM of less than 160 nm. In some embodiments, the detectable moieties having the croconate core have a peak absorbance wavelength of about 890+/−10 nm and a first absorbance peak with FWHM of less than 160 nm. In some embodiments, the detectable moieties having the croconate core have a peak absorbance wavelength of about 885+/−10 nm and a first absorbance peak with FWHM of less than 160 nm. In some embodiments, the detectable moieties having the croconate core have a peak absorbance wavelength of about 880+/−10 nm and a first absorbance peak with FWHM of less than 160 nm. In some embodiments, the detectable moieties having the croconate core have a peak absorbance wavelength of about 870+/−10 nm and a first absorbance peak with FWHM of less than 160 nm. In some embodiments, the detectable moieties having the croconate core have a peak absorbance wavelength of about 865+/−10 nm and a first absorbance peak with FWHM of less than 160 nm. In some embodiments, the detectable moieties having the croconate core have a peak absorbance wavelength of about 860+/−10 nm and a first absorbance peak with FWHM of less than 160 nm. In some embodiments, the detectable moieties having the croconate core have a peak absorbance wavelength of about 855+/−10 nm and a first absorbance peak with FWHM of less than 160 nm. In some embodiments, the detectable moieties having the croconate core have a peak absorbance wavelength of about 850+/−10 nm and a first absorbance peak with FWHM of less than 160 nm. In some embodiments, the detectable moieties having the croconate core have a peak absorbance wavelength of about 845+/−10 nm and a first absorbance peak with FWHM of less than 160 nm. In some embodiments, the detectable moieties having the croconate core have a peak absorbance wavelength of about 840+/−10 nm and a first absorbance peak with FWHM of less than 160 nm. In some embodiments, the detectable moieties having the croconate core have a peak absorbance wavelength of about 835+/−10 nm and a first absorbance peak with FWHM of less than 160 nm. In some embodiments, the detectable moieties having the croconate core have a peak absorbance wavelength of about 830+/−10 nm and a first absorbance peak with FWHM of less than 160 nm. In some embodiments, the detectable moieties having the croconate core have a peak absorbance wavelength of about 825+/−10 nm and a first absorbance peak with FWHM of less than 160 nm. In some embodiments, the detectable moieties having the croconate core have a peak absorbance wavelength of about 820+/−10 nm and a first absorbance peak with FWHM of less than 160 nm. In some embodiments, the detectable moieties having the croconate core have a peak absorbance wavelength of about 815+/−10 nm and a first absorbance peak with FWHM of less than 160 nm. In some embodiments, the detectable moieties having the croconate core have a peak absorbance wavelength of about 800+/−10 nm and a first absorbance peak with FWHM of less than 160 nm. In some embodiments, the detectable moieties having the croconate core have a peak absorbance wavelength of about 795+/−10 nm and a first absorbance peak with FWHM of less than 160 nm. In some embodiments, the detectable moieties having the croconate core have a peak absorbance wavelength of about 790+/−10 nm and a first absorbance peak with FWHM of less than 160 nm. In some embodiments, the detectable moieties having the croconate core have a peak absorbance wavelength of about 785+/−10 nm and a first absorbance peak with FWHM of less than 160 nm. In some embodiments, the detectable moieties having the croconate core have a peak absorbance wavelength of about 780+/−10 nm and a first absorbance peak with FWHM of less than 160 nm.

In some embodiments, the detectable moieties having the croconate core have a peak absorbance wavelength of about 900+/−10 nm and a first absorbance peak with FWHM of less than 130 nm. In some embodiments, the detectable moieties having the croconate core have a peak absorbance wavelength of about 895+/−10 nm and a first absorbance peak with FWHM of less than 130 nm. In some embodiments, the detectable moieties having the croconate core have a peak absorbance wavelength of about 890+/−10 nm and a first absorbance peak with FWHM of less than 130 nm. In some embodiments, the detectable moieties having the croconate core have a peak absorbance wavelength of about 885+/−10 nm and a first absorbance peak with FWHM of less than 130 nm. In some embodiments, the detectable moieties having the croconate core have a peak absorbance wavelength of about 880+/−10 nm and a first absorbance peak with FWHM of less than 130 nm. In some embodiments, the detectable moieties having the croconate core have a peak absorbance wavelength of about 870+/−10 nm and a first absorbance peak with FWHM of less than 130 nm. In some embodiments, the detectable moieties having the croconate core have a peak absorbance wavelength of about 865+/−10 nm and a first absorbance peak with FWHM of less than 130 nm. In some embodiments, the detectable moieties having the croconate core have a peak absorbance wavelength of about 860+/−10 nm and a first absorbance peak with FWHM of less than 130 nm. In some embodiments, the detectable moieties having the croconate core have a peak absorbance wavelength of about 855+/−10 nm and a first absorbance peak with FWHM of less than 130 nm. In some embodiments, the detectable moieties having the croconate core have a peak absorbance wavelength of about 850+/−10 nm and a first absorbance peak with FWHM of less than 130 nm. In some embodiments, the detectable moieties having the croconate core have a peak absorbance wavelength of about 845+/−10 nm and a first absorbance peak with FWHM of less than 130 nm. In some embodiments, the detectable moieties having the croconate core have a peak absorbance wavelength of about 840+/−10 nm and a first absorbance peak with FWHM of less than 130 nm. In some embodiments, the detectable moieties having the croconate core have a peak absorbance wavelength of about 835+/−10 nm and a first absorbance peak with FWHM of less than 130 nm. In some embodiments, the detectable moieties having the croconate core have a peak absorbance wavelength of about 830+/−10 nm and a first absorbance peak with FWHM of less than 130 nm. In some embodiments, the detectable moieties having the croconate core have a peak absorbance wavelength of about 825+/−10 nm and a first absorbance peak with FWHM of less than 130 nm. In some embodiments, the detectable moieties having the croconate core have a peak absorbance wavelength of about 820+/−10 nm and a first absorbance peak with FWHM of less than 130 nm. In some embodiments, the detectable moieties having the croconate core have a peak absorbance wavelength of about 815+/−10 nm and a first absorbance peak with FWHM of less than 130 nm. In some embodiments, the detectable moieties having the croconate core have a peak absorbance wavelength of about 800+/−10 nm and a first absorbance peak with FWHM of less than 130 nm. In some embodiments, the detectable moieties having the croconate core have a peak absorbance wavelength of about 795+/−10 nm and a first absorbance peak with FWHM of less than 130 nm. In some embodiments, the detectable moieties having the croconate core have a peak absorbance wavelength of about 790+/−10 nm and a first absorbance peak with FWHM of less than 130 nm. In some embodiments, the detectable moieties having the croconate core have a peak absorbance wavelength of about 785+/−10 nm and a first absorbance peak with FWHM of less than 130 nm. In some embodiments, the detectable moieties having the croconate core have a peak absorbance wavelength of about 780+/−10 nm and a first absorbance peak with FWHM of less than 130 nm.

In some embodiments, the detectable moieties having the croconate core have a peak absorbance wavelength of about 900+/−10 nm and a first absorbance peak with FWHM of less than 100 nm. In some embodiments, the detectable moieties having the croconate core have a peak absorbance wavelength of about 895+/−10 nm and a first absorbance peak with FWHM of less than 100 nm. In some embodiments, the detectable moieties having the croconate core have a peak absorbance wavelength of about 890+/−10 nm and a first absorbance peak with FWHM of less than 100 nm. In some embodiments, the detectable moieties having the croconate core have a peak absorbance wavelength of about 885+/−10 nm and a first absorbance peak with FWHM of less than 100 nm. In some embodiments, the detectable moieties having the croconate core have a peak absorbance wavelength of about 880+/−10 nm and a first absorbance peak with FWHM of less than 100 nm. In some embodiments, the detectable moieties having the croconate core have a peak absorbance wavelength of about 870+/−10 nm and a first absorbance peak with FWHM of less than 100 nm. In some embodiments, the detectable moieties having the croconate core have a peak absorbance wavelength of about 865+/−10 nm and a first absorbance peak with FWHM of less than 100 nm. In some embodiments, the detectable moieties having the croconate core have a peak absorbance wavelength of about 860+/−10 nm and a first absorbance peak with FWHM of less than 100 nm. In some embodiments, the detectable moieties having the croconate core have a peak absorbance wavelength of about 855+/−10 nm and a first absorbance peak with FWHM of less than 100 nm. In some embodiments, the detectable moieties having the croconate core have a peak absorbance wavelength of about 850+/−10 nm and a first absorbance peak with FWHM of less than 100 nm. In some embodiments, the detectable moieties having the croconate core have a peak absorbance wavelength of about 845+/−10 nm and a first absorbance peak with FWHM of less than 100 nm. In some embodiments, the detectable moieties having the croconate core have a peak absorbance wavelength of about 840+/−10 nm and a first absorbance peak with FWHM of less than 100 nm. In some embodiments, the detectable moieties having the croconate core have a peak absorbance wavelength of about 835+/−10 nm and a first absorbance peak with FWHM of less than 100 nm. In some embodiments, the detectable moieties having the croconate core have a peak absorbance wavelength of about 830+/−10 nm and a first absorbance peak with FWHM of less than 100 nm. In some embodiments, the detectable moieties having the croconate core have a peak absorbance wavelength of about 825+/−10 nm and a first absorbance peak with FWHM of less than 100 nm. In some embodiments, the detectable moieties having the croconate core have a peak absorbance wavelength of about 820+/−10 nm and a first absorbance peak with FWHM of less than 100 nm. In some embodiments, the detectable moieties having the croconate core have a peak absorbance wavelength of about 815+/−10 nm and a first absorbance peak with FWHM of less than 100 nm. In some embodiments, the detectable moieties having the croconate core have a peak absorbance wavelength of about 800+/−10 nm and a first absorbance peak with FWHM of less than 100 nm. In some embodiments, the detectable moieties having the croconate core have a peak absorbance wavelength of about 795+/−10 nm and a first absorbance peak with FWHM of less than 100 nm. In some embodiments, the detectable moieties having the croconate core have a peak absorbance wavelength of about 790+/−10 nm and a first absorbance peak with FWHM of less than 100 nm. In some embodiments, the detectable moieties having the croconate core have a peak absorbance wavelength of about 785+/−10 nm and a first absorbance peak with FWHM of less than 100 nm. In some embodiments, the detectable moieties having the croconate core have a peak absorbance wavelength of about 780+/−10 nm and a first absorbance peak with FWHM of less than 100 nm.

Non-limiting examples of detectable moieties include a heptamethine cyanine core or a croconate core include:

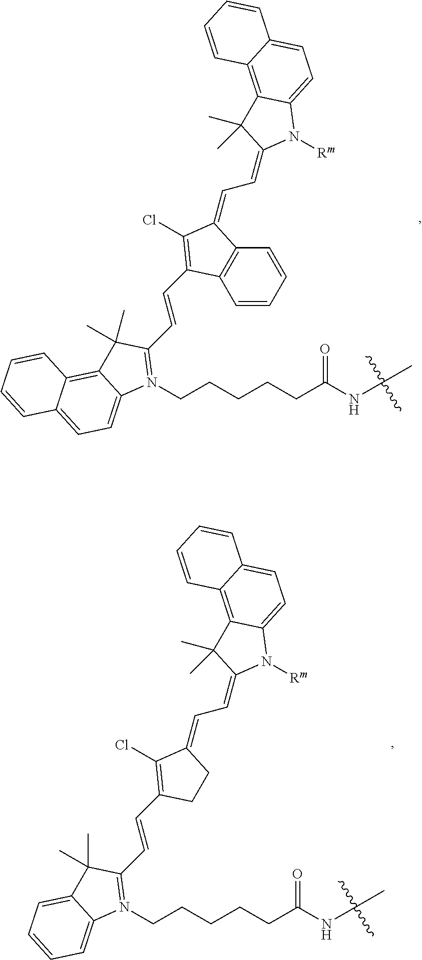

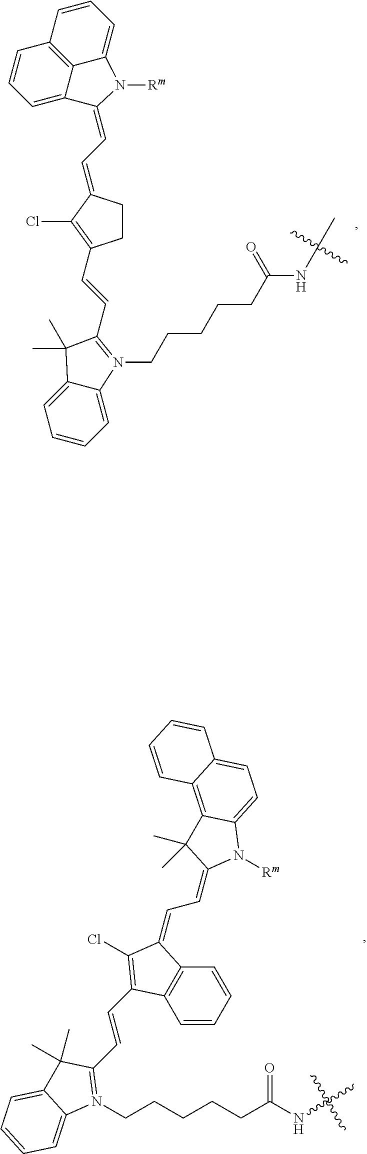

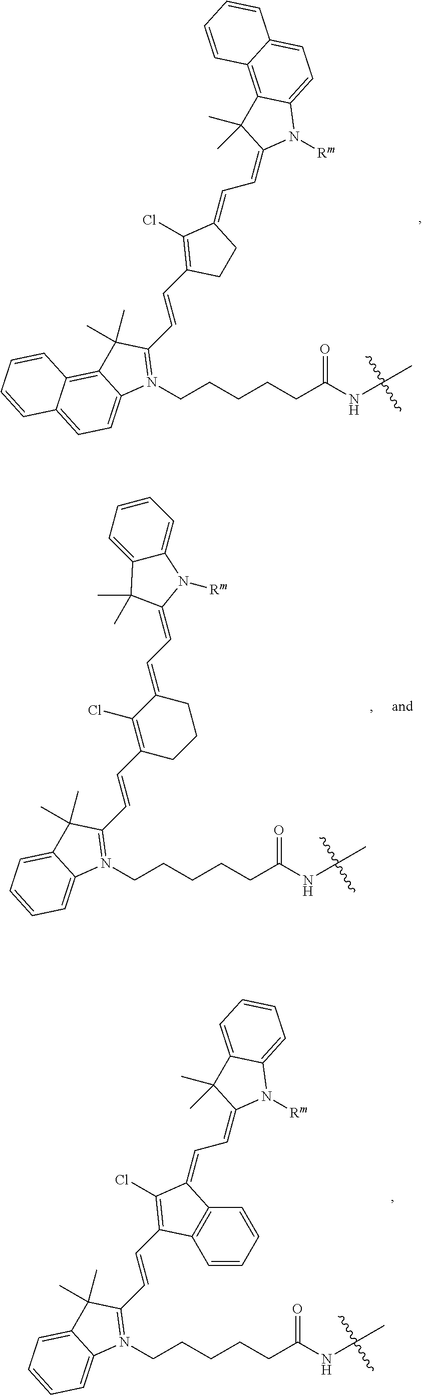

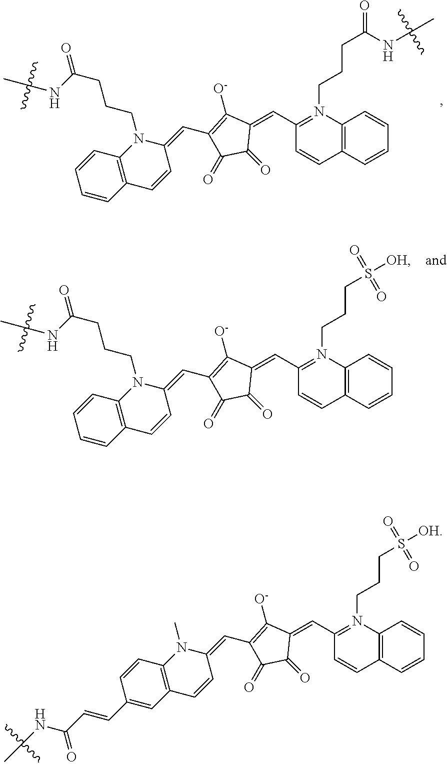

where the symbol “ [symbol] ” refers to the site in which the detectable moiety (here, the heptamethine cyanine core or the croconate core include) is coupled (directly or indirectly) to another moiety of the detectable conjugate (e.g. to a tyramide moiety, to a quinone methide moiety, to a functional group capable or participating in a “click chemistry” reaction, to an antibody, to an enzyme, to a hapten, etc.). Yet other examples are disclosed herein.

Other detectable moieties suitable for use with the presently disclosed methods include any of those having a diazo-core, such as those disclosed in U.S. Pat. No. 10,041,950, the disclosure of which is incorporated by reference herein in its entirety. An example of such a compound is tartrazine:

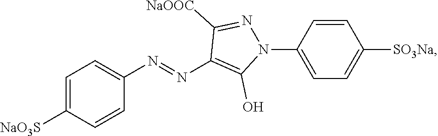

which has a peak absorbance wavelength of about 472 nm and a first absorbance peak with FWHM of less than about 70 nm.

Other detectable moieties suitable for use with the presently disclosed methods include any of those having a triarylmethane-core, such as those disclosed in U.S. Pat. No. 10,041,950, the disclosure of which is incorporated by reference herein in its entirety. Other detectable moieties suitable for use with the presently disclosed methods include tetramethylrhodamines and diarylrhodamine, such as those disclosed in U.S. Pat. No. 10,041,950, the disclosure of which is incorporated by reference herein in its entirety.

Non-limiting examples of detectable conjugates including (i) a tyramide or a quinone methide precursor moiety, coupled to (ii) a detectable moiety include the following:

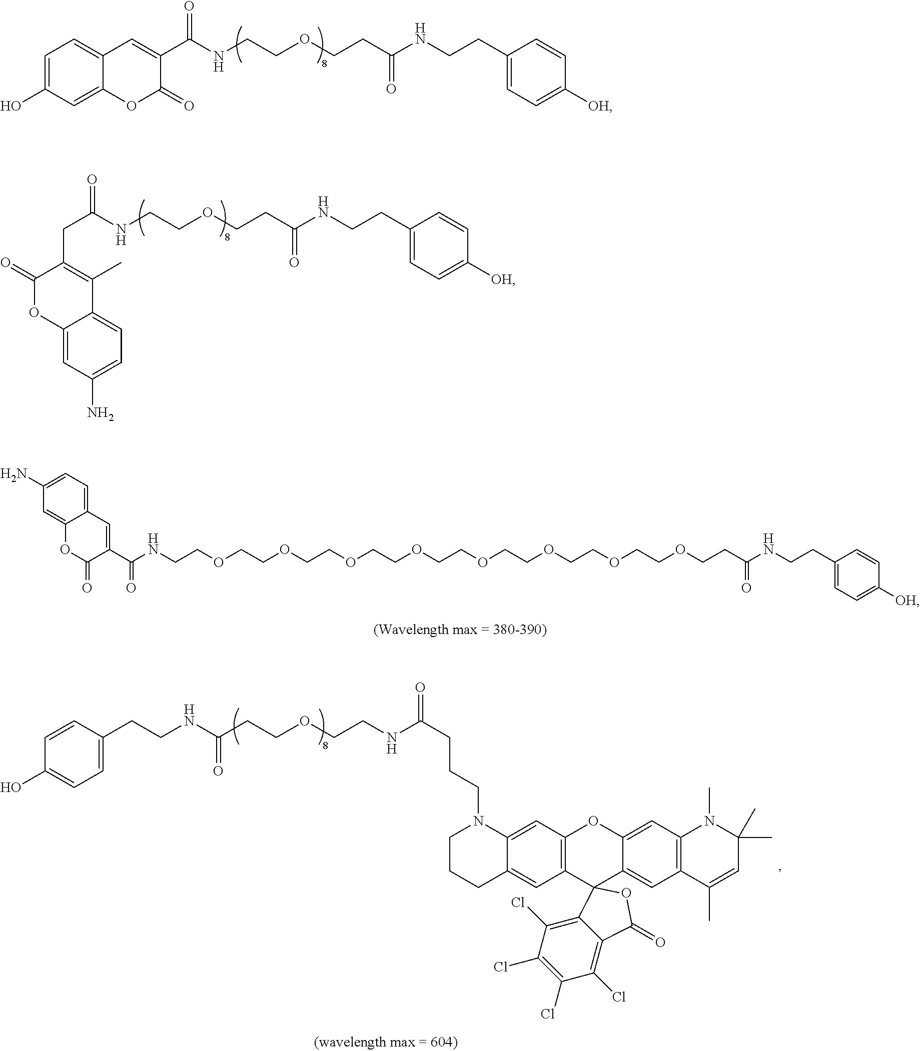

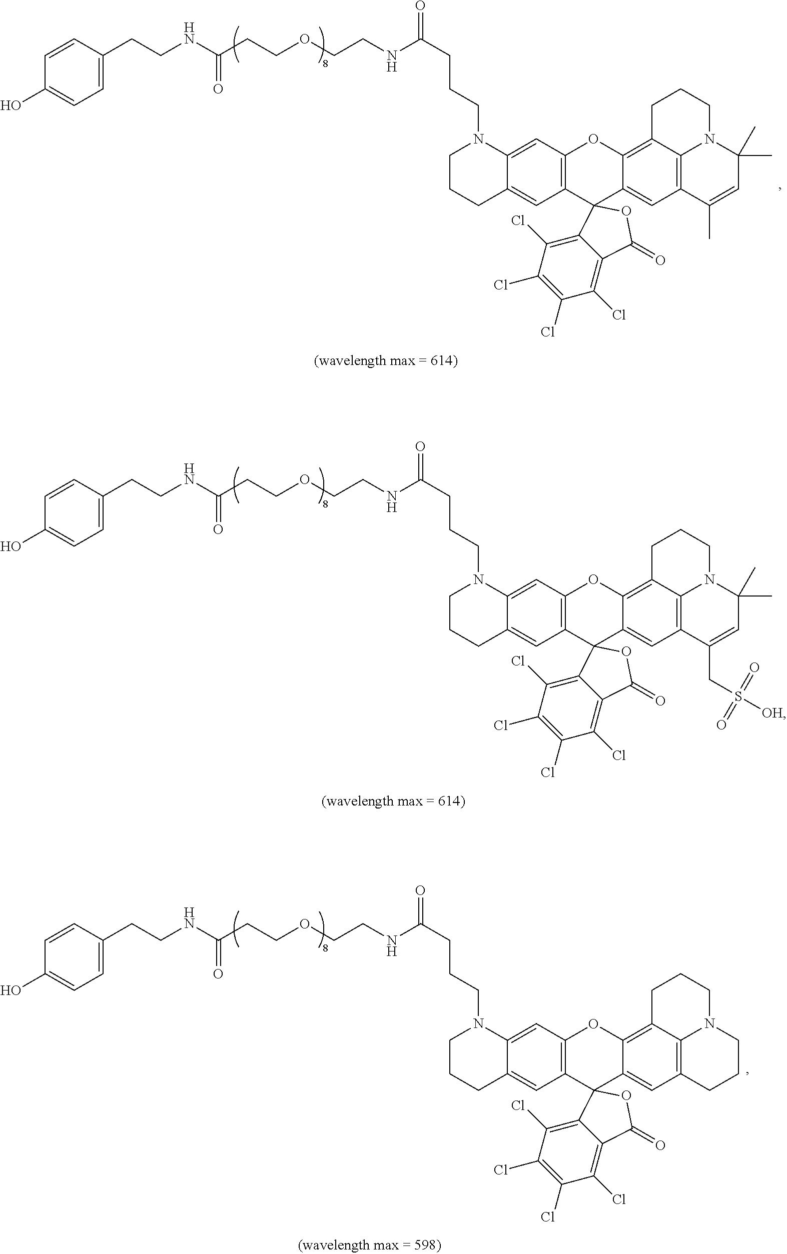

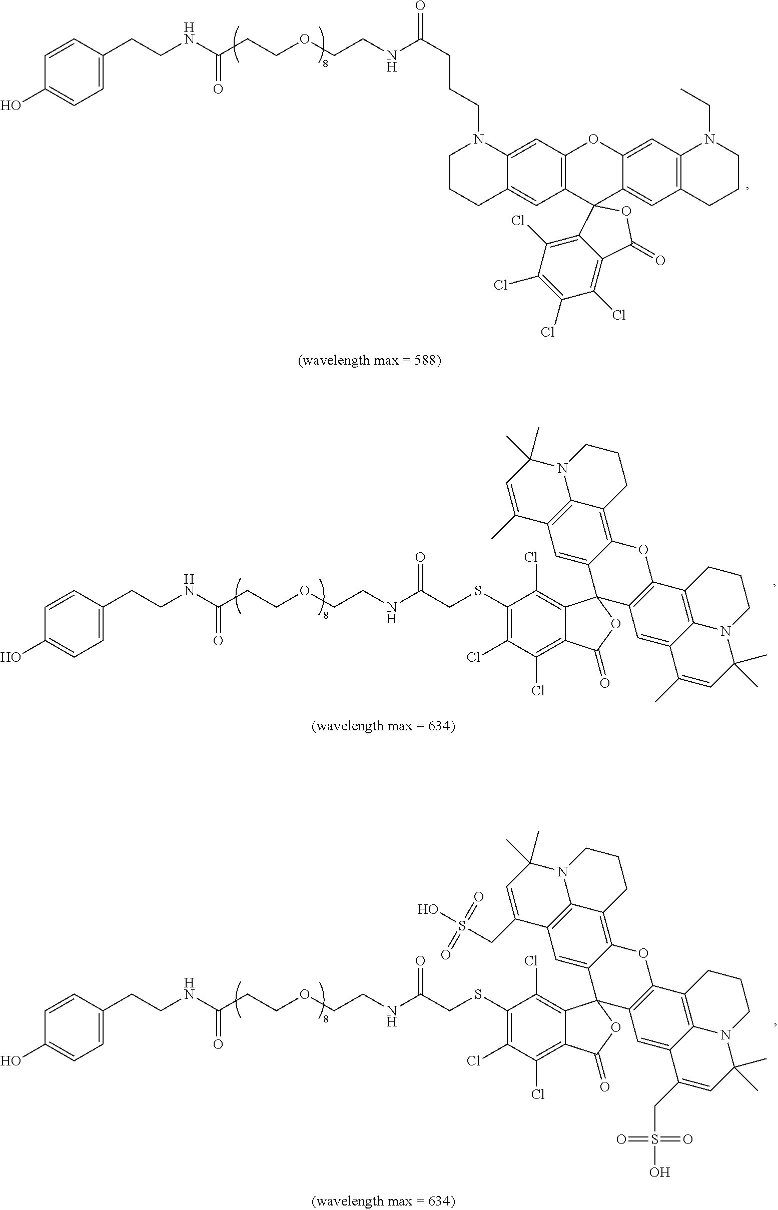

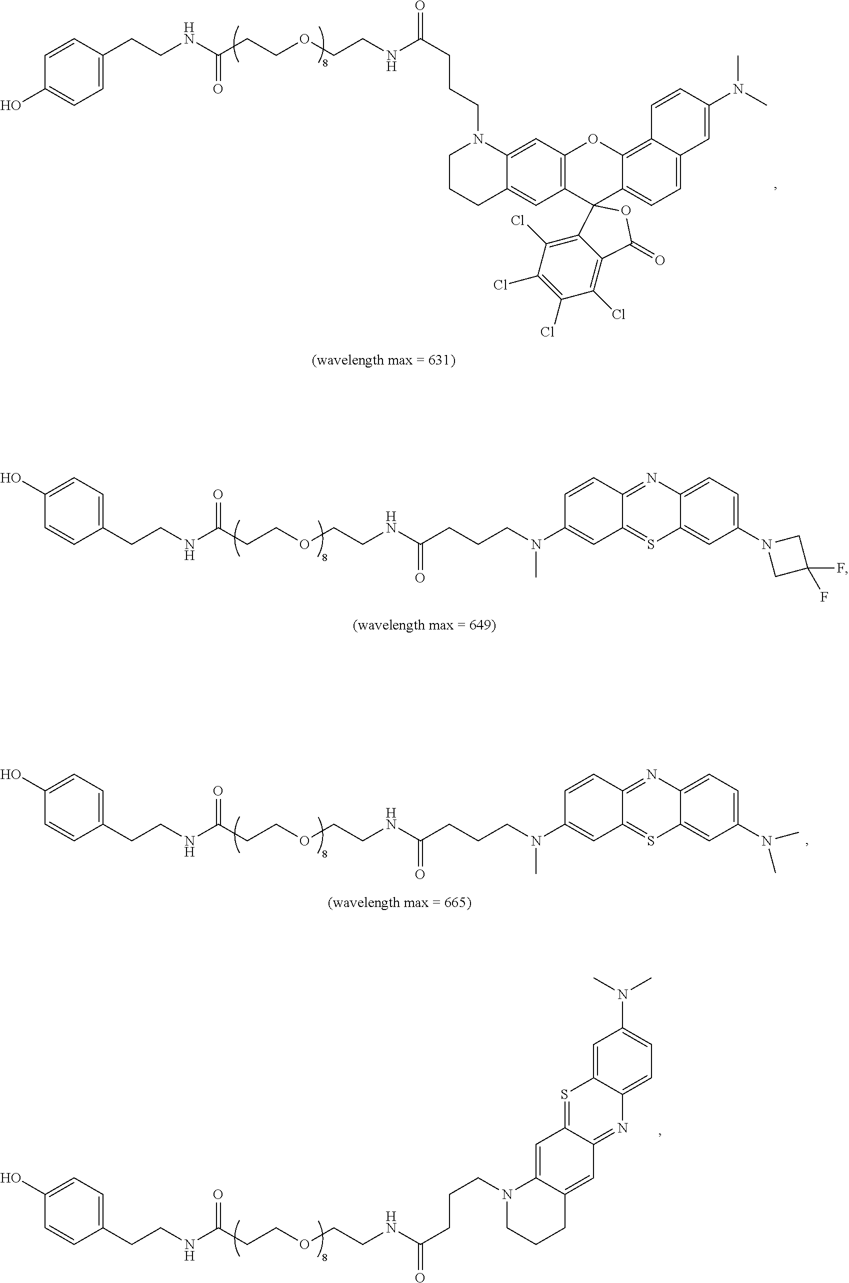

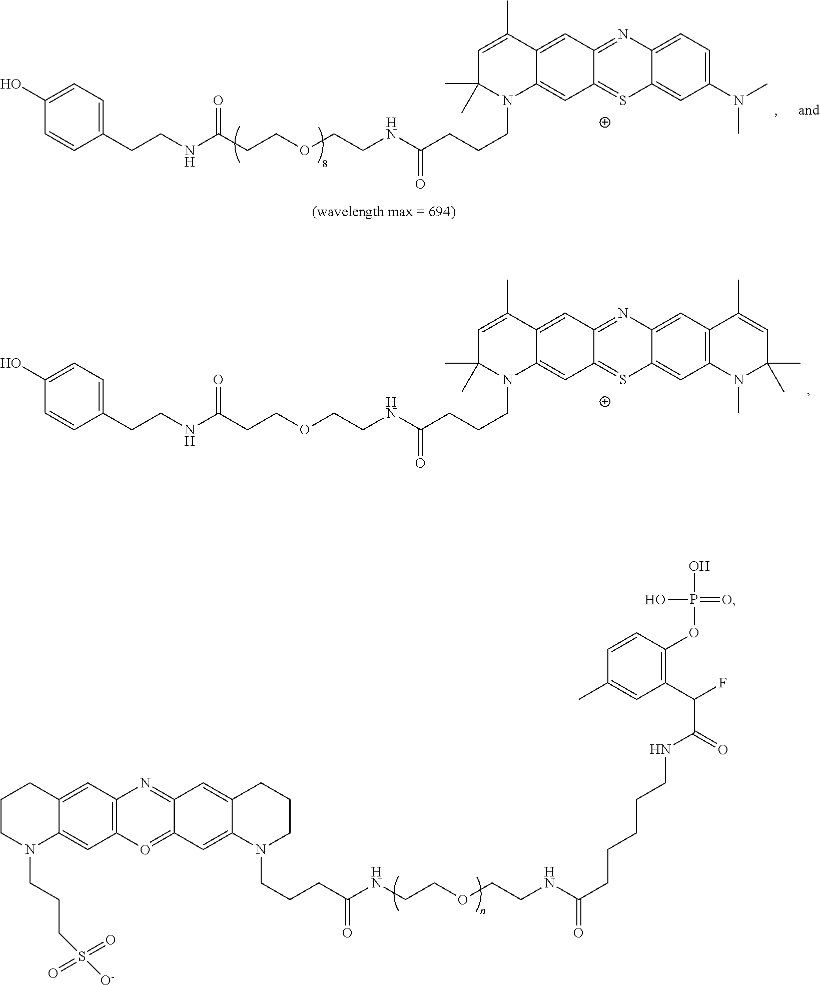

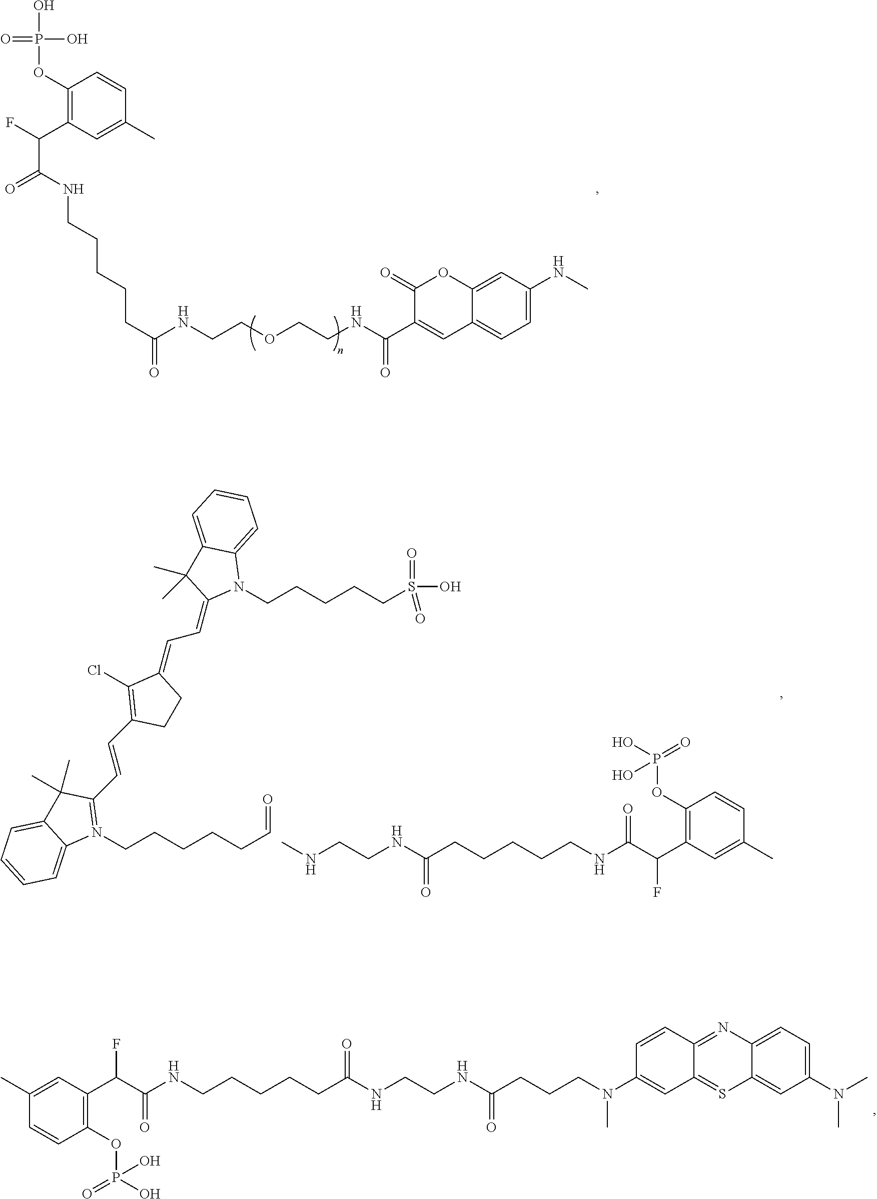

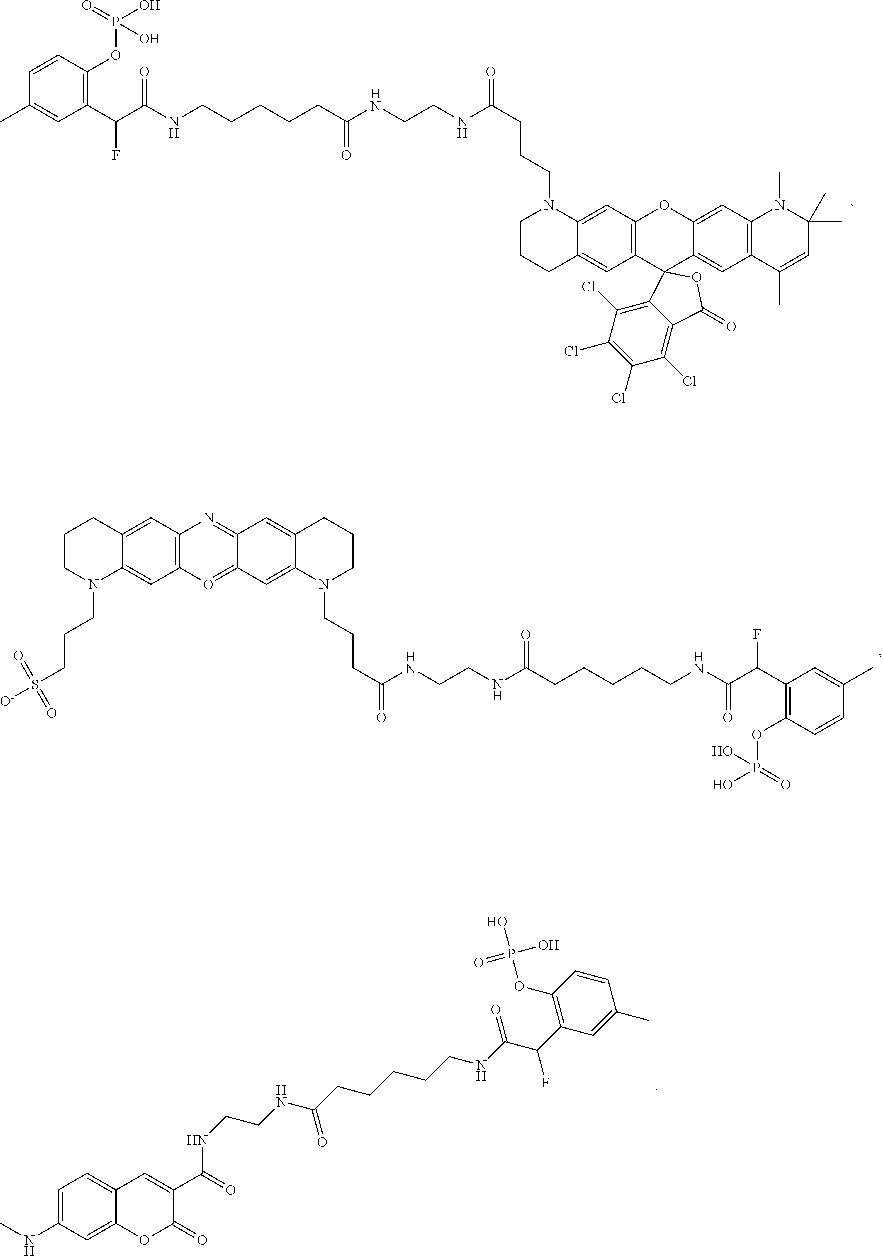

Non-limiting examples of detectable conjugates including (i) a functional group capable of participating in a click chemistry reaction, coupled to (ii) a detectable moiety include the following:

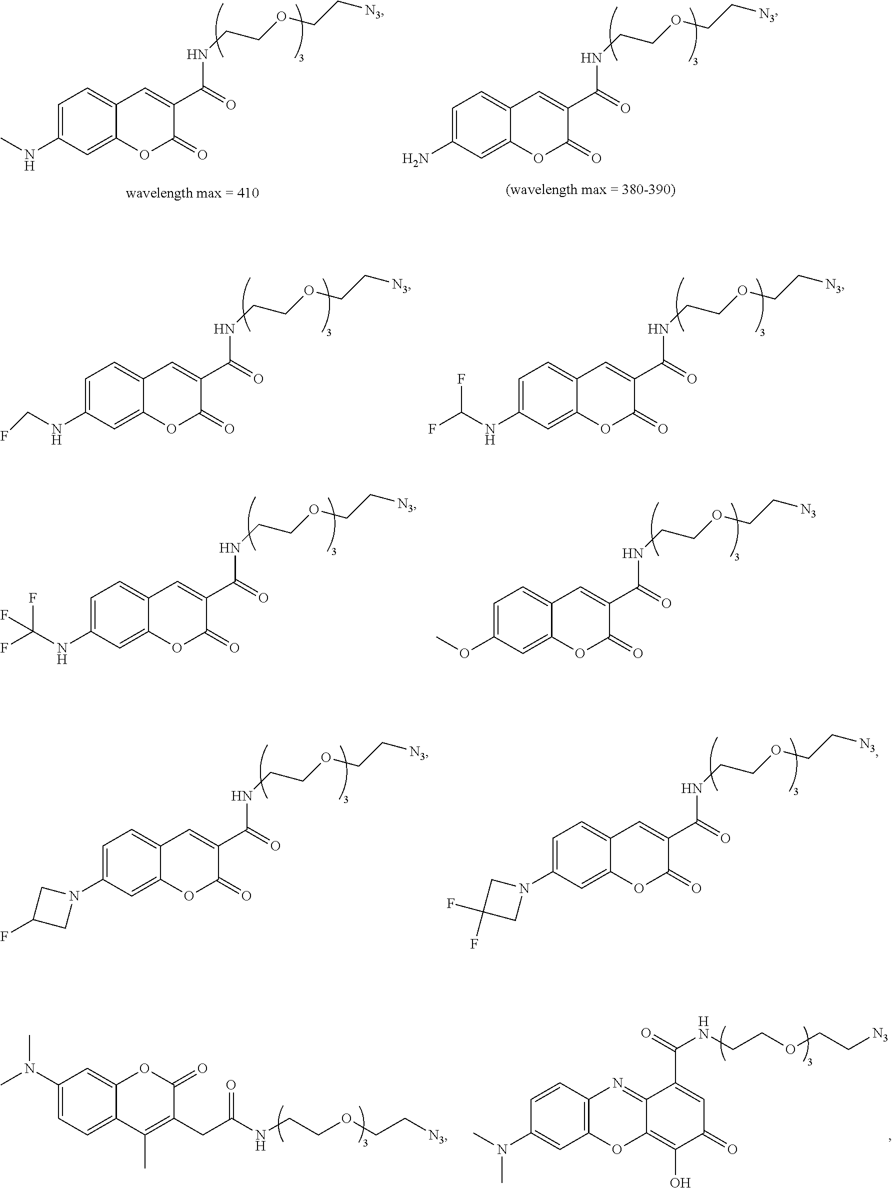

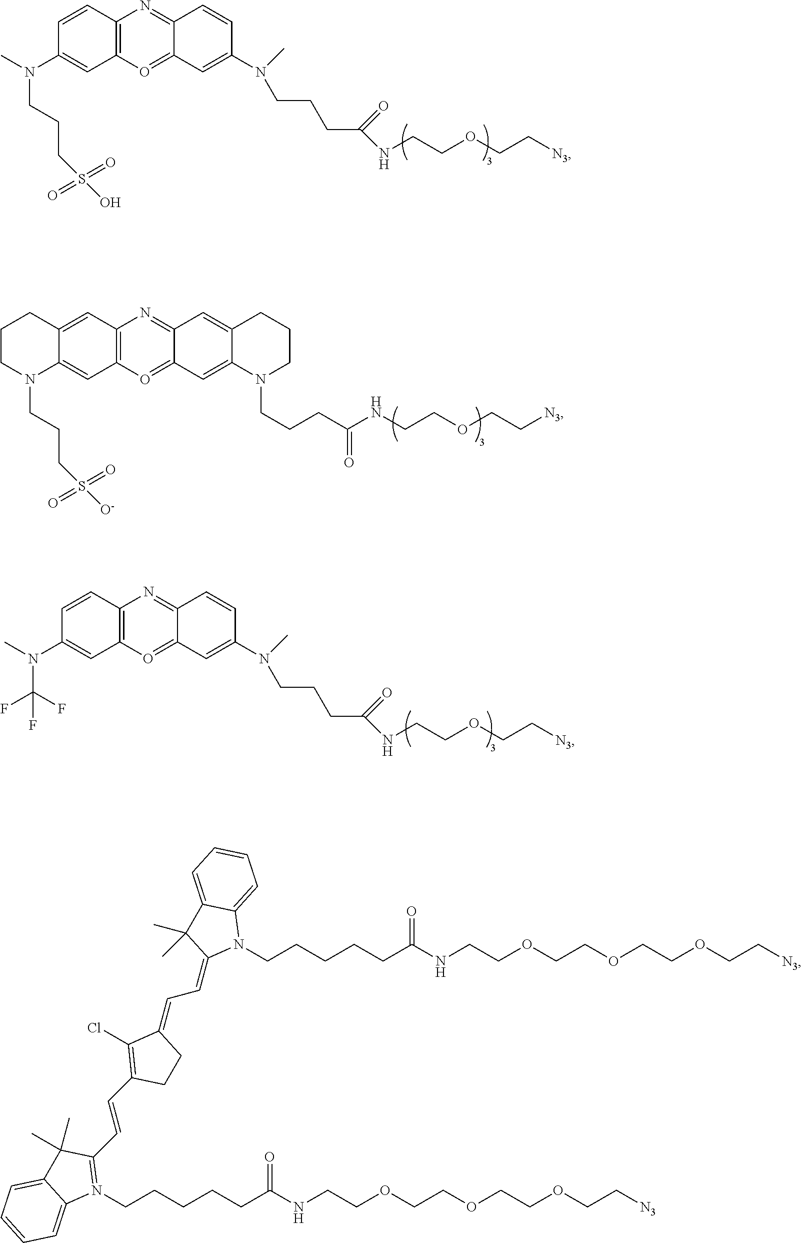

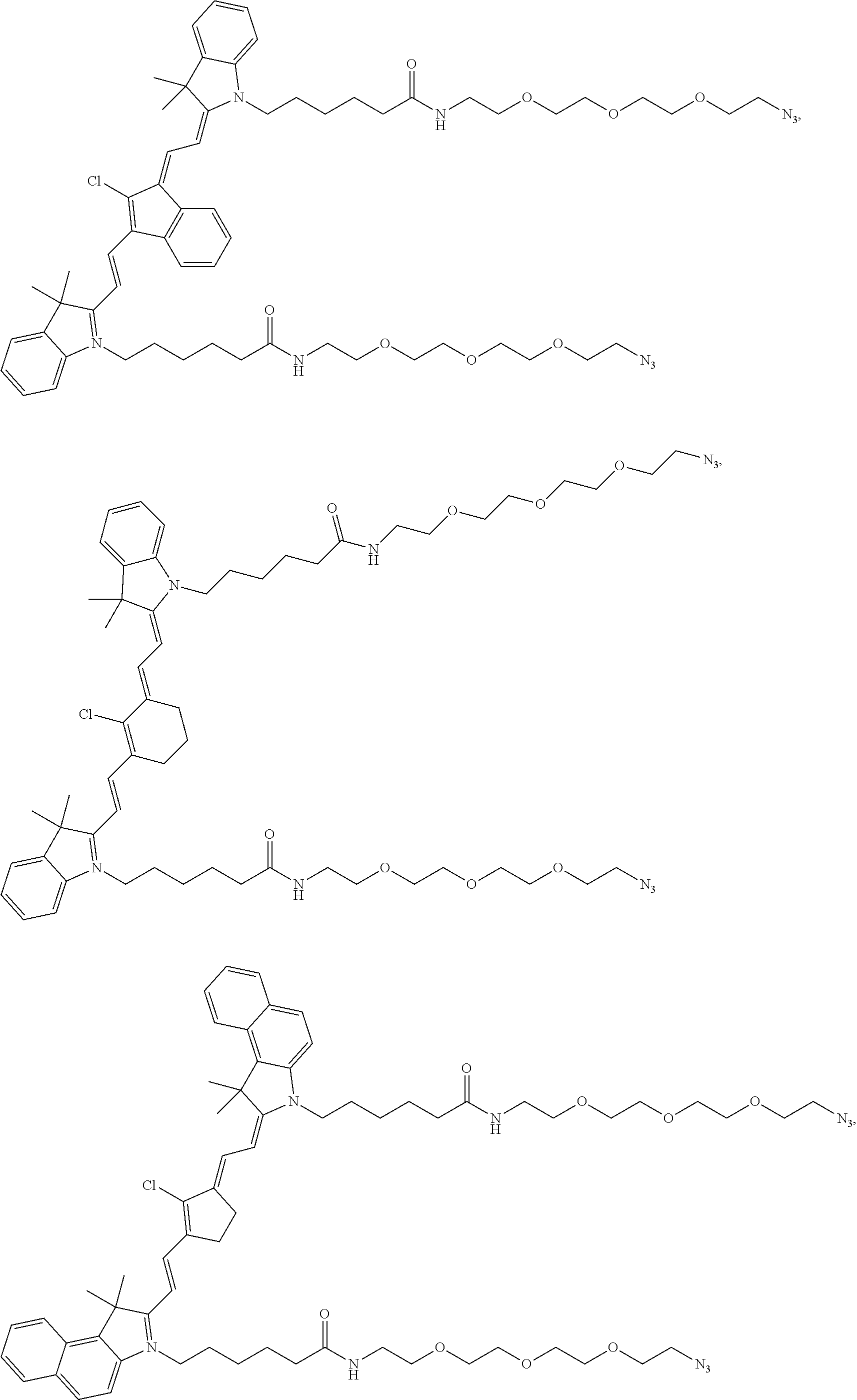

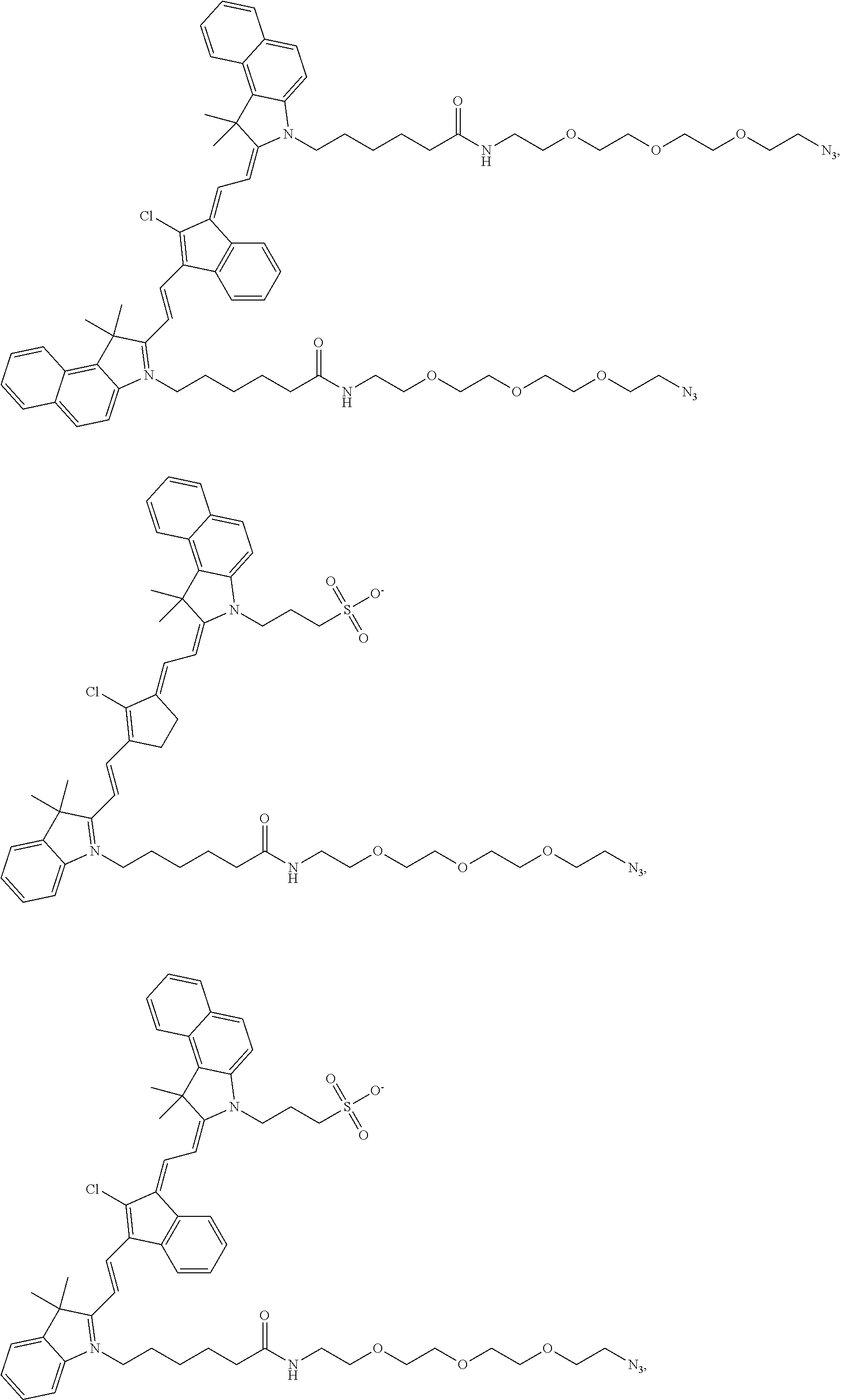

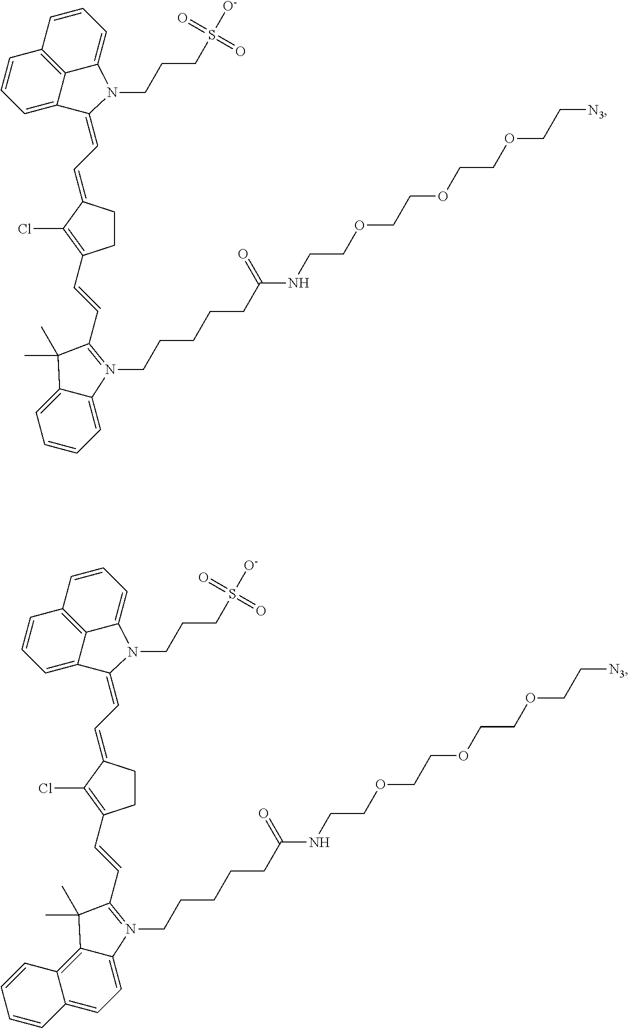

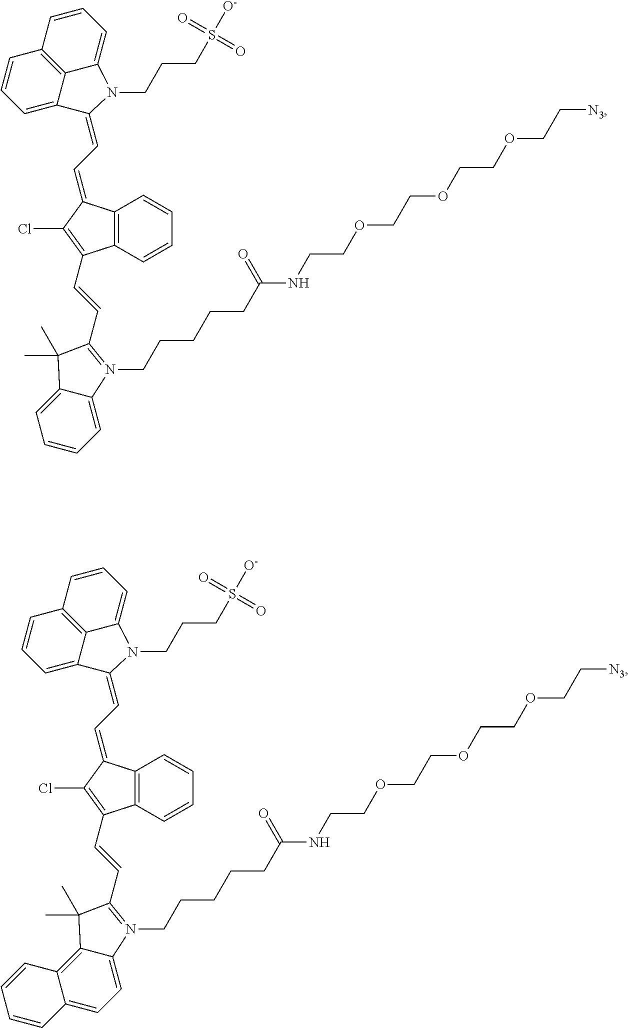

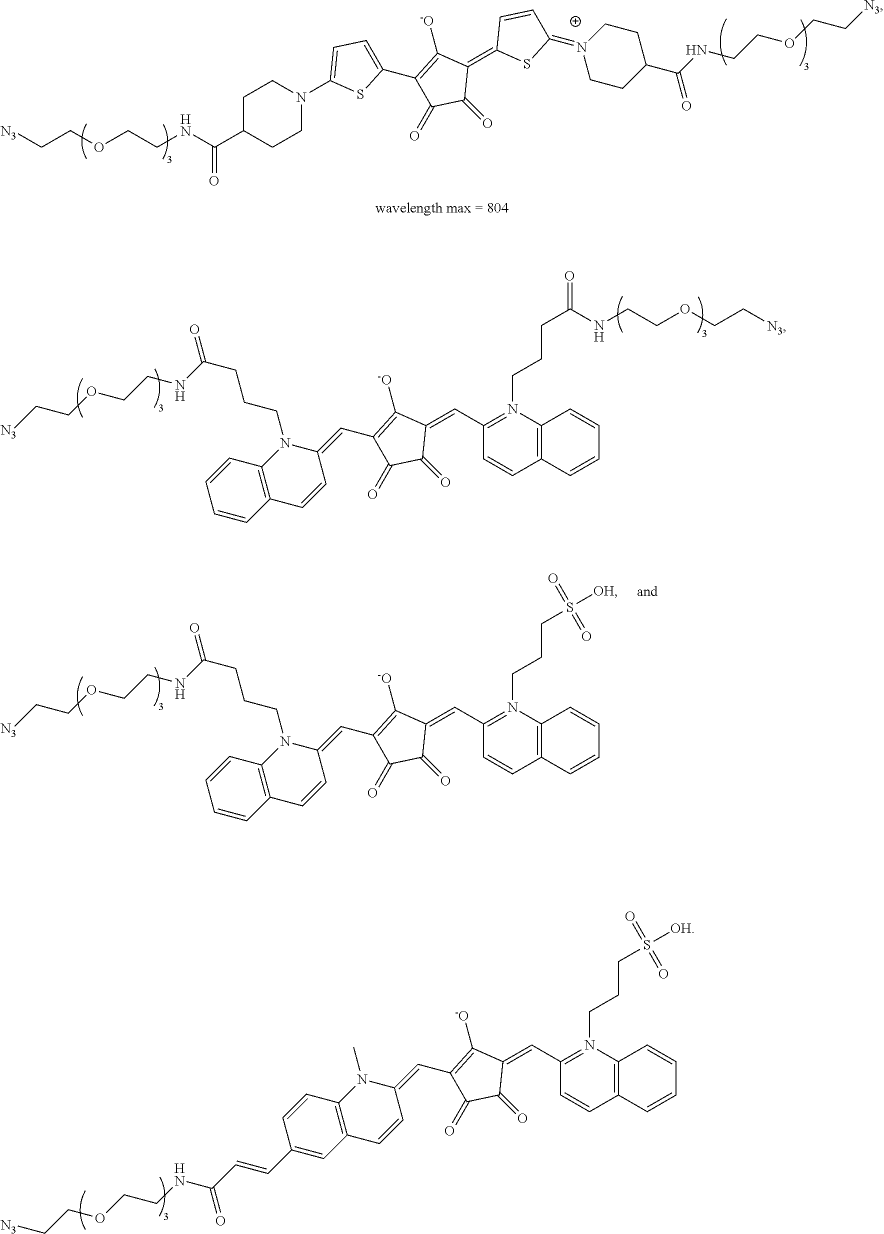

The skilled artisan will appreciate that while each of the exemplified compounds includes an azide group (i.e. N₃), that another functional group capable of participating in a “click chemistry” reaction may be substituted for the azide group, including any of the click functional groups listed in Table 11 below:

TABLE 11
Reactive Functional Groups Capable of
Particpating in a Click Chemistry Reaction
Alkyne
Azide
diarylcyclooctyne (″DBCO″)
Alkene
Trans-cyclooctene (″TCO″)
Maleimide
DBCO
Aldehyde or ketone
Tetrazine
Thiol
1,3-Nitrone
Hydrazine
Hydroxylamine
Tetrazine

In some embodiments, the detectable conjugates are selected from the following:

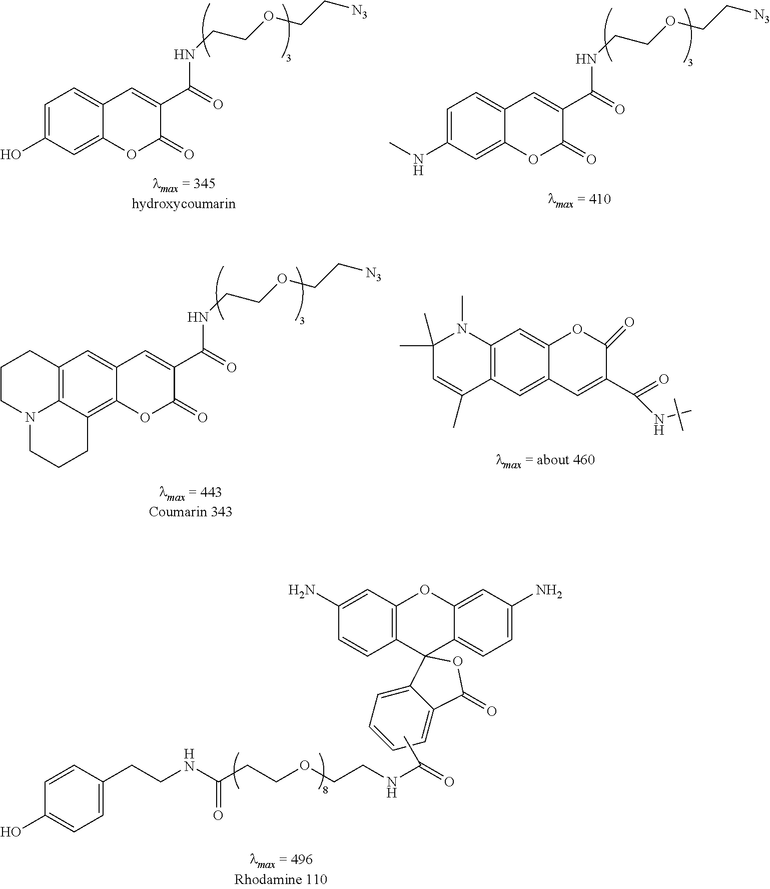

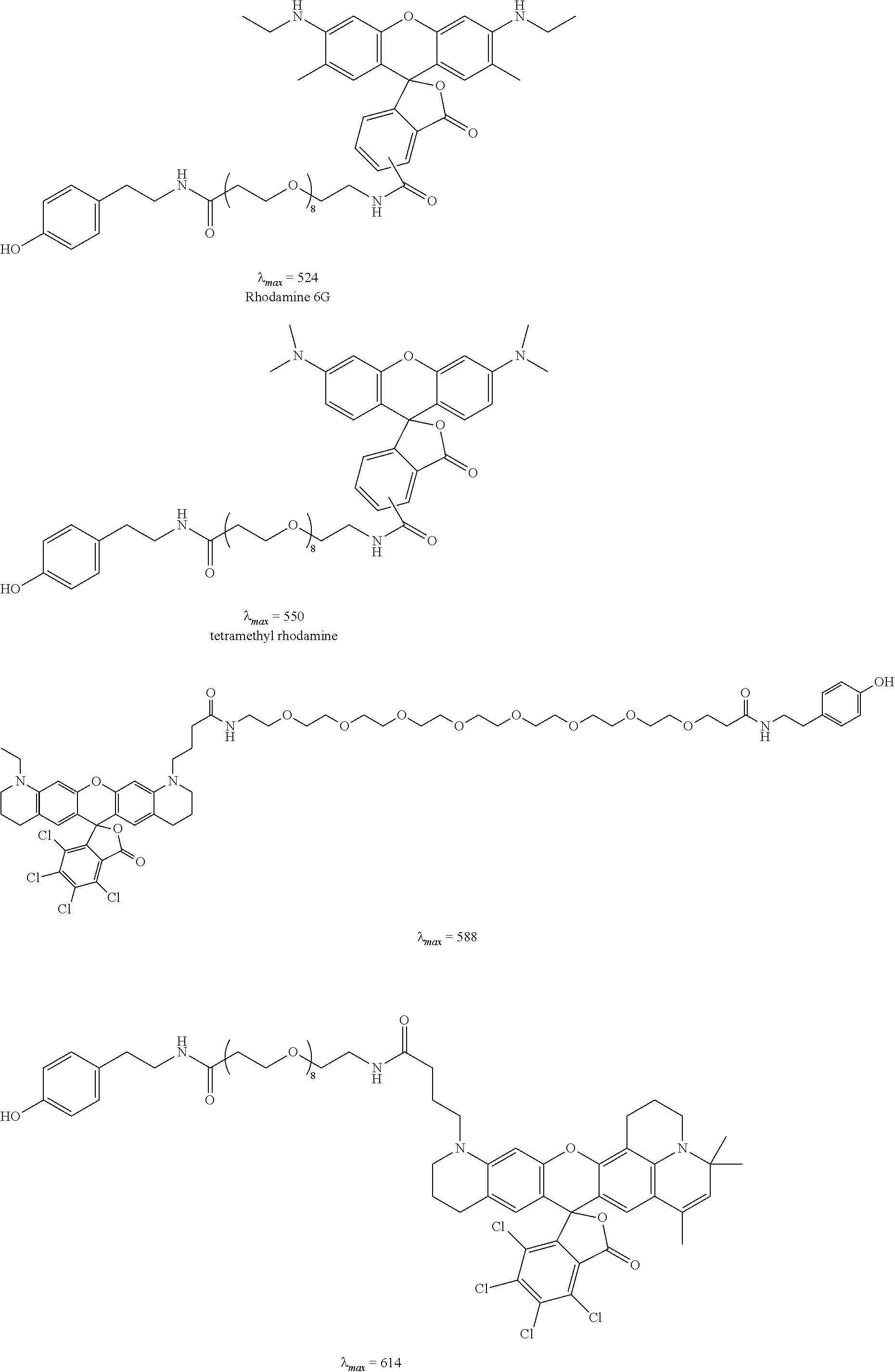

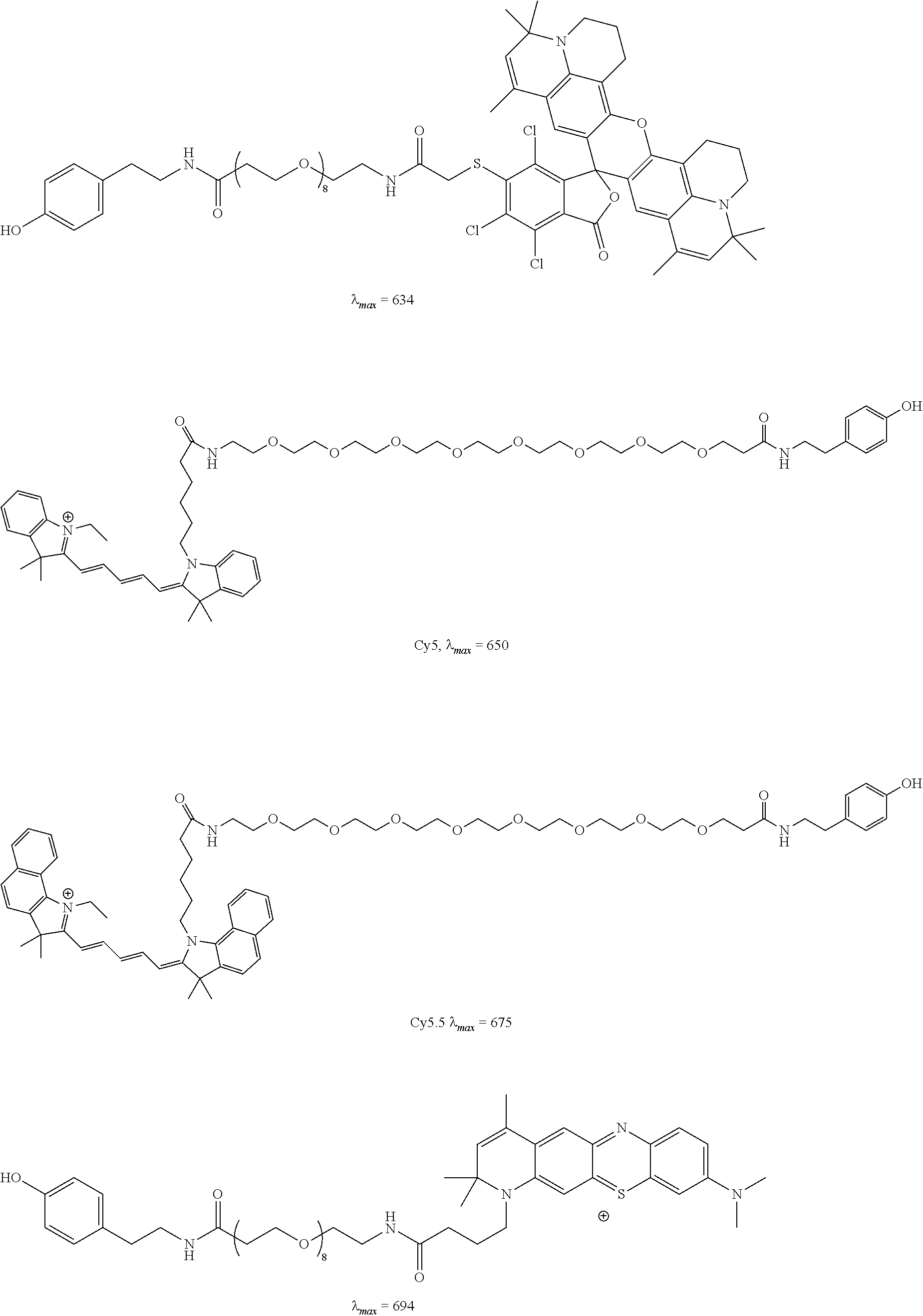

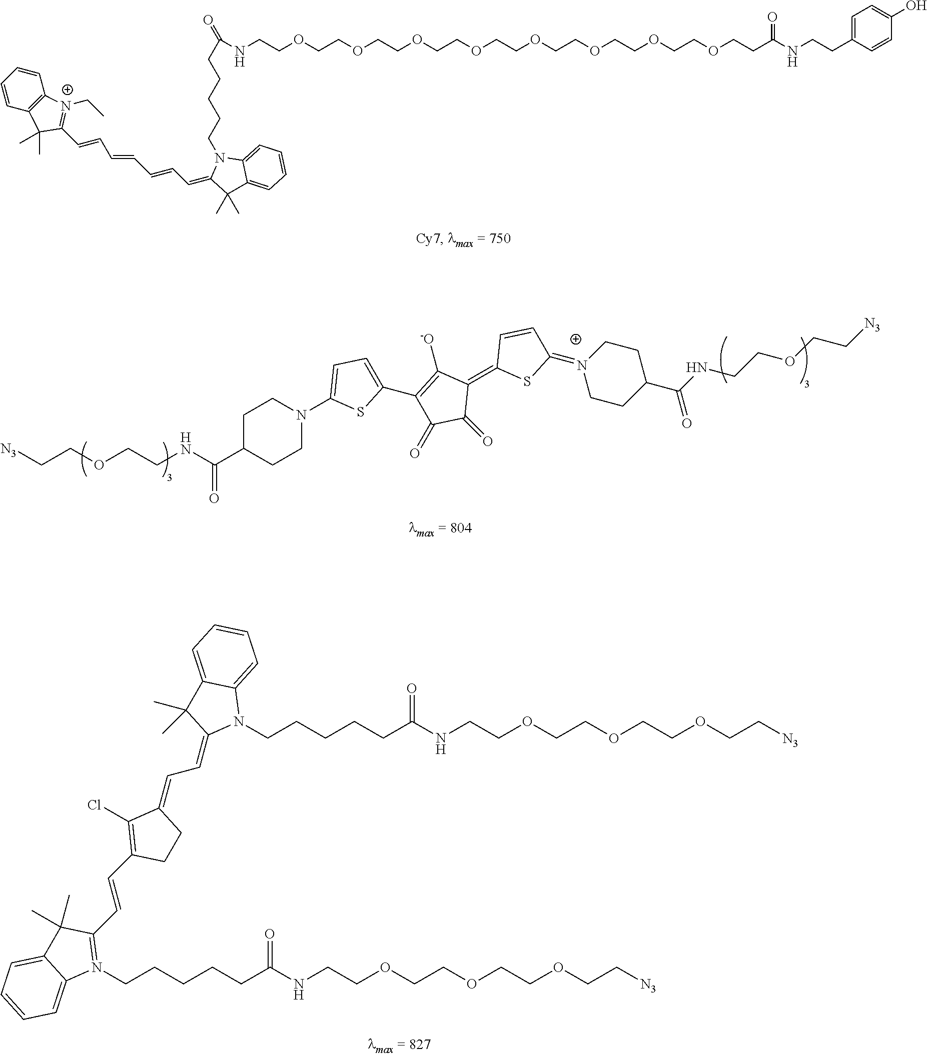

Methods

The present disclosure also provides methods of detecting one or more morphological markers and/or one or more biomarkers in a biological sample. In some embodiments, the present disclosure provides methods of labeling one morphological marker and two or more biomarkers with different detectable moieties. In some embodiments, the present disclosure provides methods of labeling one morphological marker and three or more biomarkers with different detectable moieties. In some embodiments, the present disclosure provides methods of labeling one morphological marker and four or more biomarkers with different detectable moieties. In some embodiments, the present disclosure provides methods of labeling one morphological marker and five or more biomarkers with different detectable moieties.

In some embodiments, the present disclosure provides methods of labeling two or more morphological markers (such as two or more morphological markers characteristic of the same or different morphological features) and two or more biomarkers with different detectable moieties. In some embodiments, the present disclosure provides methods of labeling two or more morphological markers (such as two or more morphological markers characteristic of the same or different morphological features) and three or more biomarkers with different detectable moieties. In some embodiments, the present disclosure provides methods of labeling two or more morphological markers (such as two or more morphological markers characteristic of the same or different morphological features) and four or more biomarkers with different detectable moieties. In some embodiments, the present disclosure provides methods of labeling two or more morphological markers (such as two or more morphological markers characteristic of the same or different morphological features) and five or more biomarkers with different detectable moieties. In some embodiments, the present disclosure provides methods of labeling two or more morphological markers and seven or more biomarkers with different detectable moieties. In some embodiments, the present disclosure provides methods of labeling two or more morphological markers (such as two or more morphological markers characteristic of the same or different morphological features) and nine or more biomarkers with different detectable moieties. In some embodiments, the present disclosure provides methods of labeling two or more morphological markers and ten or more biomarkers with different detectable moieties.

In some embodiments, the present disclosure provides methods of labeling two or more morphological markers (such as those characteristic of the same morphological feature) and one or more biomarkers with different detectable moieties. In some embodiments, the present disclosure provides methods of labeling three or more morphological markers (such as those characteristic of the same morphological feature) and one or more biomarkers with different detectable moieties. In some embodiments, the present disclosure provides methods of labeling four or more morphological markers (such as those characteristic of the same morphological feature) and one or more biomarkers with different detectable moieties. In some embodiments, the present disclosure provides methods of labeling five or more morphological markers (such as those characteristic of the same morphological feature) and one or more biomarkers with different detectable moieties. In some embodiments, the present disclosure provides methods of labeling six or more morphological markers (such as those characteristic of the same morphological feature) and one or more biomarkers with different detectable moieties. In some embodiments, the present disclosure provides methods of labeling seven or more morphological markers (such as those characteristic of the same morphological feature) and one or more biomarkers with different detectable moieties. In some embodiments, the present disclosure provides methods of labeling eight or more morphological markers (such as those characteristic of the same morphological feature) and one or more biomarkers with different detectable moieties. In some embodiments, the present disclosure provides methods of labeling nine or more morphological markers (such as those characteristic of the same morphological feature) and one or more biomarkers with different detectable moieties. In some embodiments, the present disclosure provides methods of labeling ten or more morphological markers (such as those characteristic of the same morphological feature) and one or more biomarkers with different detectable moieties. In some embodiments, the present disclosure provides methods of labeling eleven or more morphological markers (such as those characteristic of the same morphological feature) and one or more biomarkers with different detectable moieties.

In some embodiments, the present disclosure provides methods of labeling two or more morphological markers (such as those characteristic of the same morphological feature). In some embodiments, the present disclosure provides methods of labeling three or more morphological markers (such as those characteristic of the same morphological feature). In some embodiments, the present disclosure provides methods of labeling four or more morphological markers (such as those characteristic of the same morphological feature). In some embodiments, the present disclosure provides methods of labeling five or more morphological markers (such as those characteristic of the same morphological feature). In some embodiments, the present disclosure provides methods of labeling six or more morphological markers (such as those characteristic of the same morphological feature). In some embodiments, the present disclosure provides methods of labeling seven or more morphological markers (such as those characteristic of the same morphological feature). In some embodiments, the present disclosure provides methods of labeling eight or more morphological markers (such as those characteristic of the same morphological feature). In some embodiments, the present disclosure provides methods of labeling nine or more morphological markers (such as those characteristic of the same morphological feature). In some embodiments, the present disclosure provides methods of labeling ten or more morphological markers (such as those characteristic of the same morphological feature). In some embodiments, the present disclosure provides methods of labeling eleven or more morphological markers (such as those characteristic of the same morphological feature).

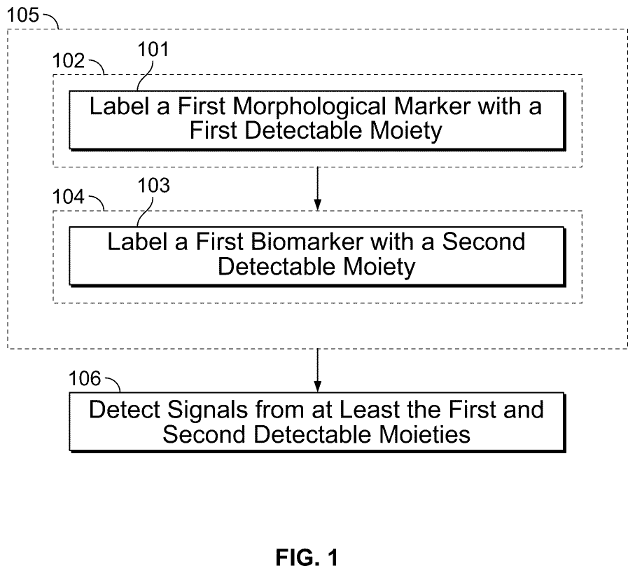

With reference to FIG. 1, in some embodiments, a first morphological marker is labeled with a first detectable moiety (step 101). In some embodiments, step 101 is repeated a plurality of times (step 102) to label one or more morphological markers with one or more detectable moieties (where the one or more detectable moieties may each be the same or different).

Next, a first biomarker is labeled with a second detectable moiety (step 103), where at least the first and second detectable moieties are different. In some embodiments, step 103 is repeated a plurality of times (step 104) to label one or more biomarkers with one or more detectable moieties, where each of the one or more detectable moieties are different from each other and from those detectable moieties used to label the one or more morphological markers. In some embodiments, steps 101, 102, 103, and 104 may also repeated as needed (step 105). Subsequently, the signals of at least the first and second detectable moieties are detected (step 106).

In some embodiments, steps 103 or 104 are performed first; and steps 101 and 102 are performed subsequently. In other embodiments, steps 101 and 103 are performed sequentially, and then both steps 101 and 103 are repeated one or more additional times. In yet other embodiments, steps 101 and 103 are performed simultaneously.

In some embodiments, the first and second detectable moieties are selected such that the first and second detectable moieties have different peak absorbance wavelengths and which do not substantially overlap (e.g. the different peak absorbance wavelengths differ by at least 20 nm, by at least 30 nm, by at least 40 nm, by at least 50 nm, by at least 60 nm, by at least 70 nm, by at least 80 nm, by at least 90 nm, by at least 100 nm, by at least 110 nm, by at least 120 nm, by at least 130 nm, by at least 140 nm, by at least 150 nm, by at least 170 nm, by at least 190 nm, by at least 210 nm, by at least 230 nm, by at least 250 nm, by at least 270 nm, by at least 290 nm, by at least 310 nm, etc.

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 200 nm, e.g. less than 160 nm, less than 130 nm, less than 100 nm, etc. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 200 nm, e.g. less than 160 nm, less than 130 nm, less than 100 nm, etc. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 200 nm, e.g. less than 160 nm, less than 130 nm, less than 100 nm, etc. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 200 nm, e.g. less than 160 nm, less than 130 nm, less than 100 nm, etc. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 200 nm, e.g. less than 160 nm, less than 130 nm, less than 100 nm, etc.

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 160 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 160 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 160 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 160 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 160 nm.

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 130 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 130 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 130 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 130 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 130 nm.

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 100 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 100 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 100 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 100 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 100 nm.

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 80 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 80 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 80 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 80 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 80 nm.

Two methods of detecting one or more morphological markers and one or more biomarkers in a biological sample are described herein. The first method utilizes detectable moieties (including any of those described herein) conjugated to a tyramide or quinone methide precursor moiety (either directly or indirectly through one or more linkers). The second method utilizes detectable moieties (including any of those described herein) conjugated (either directly or indirectly through one or more linkers) to a reactive functional group capable of participating in a click chemistry reaction. Methods and reagents for detecting targets in biological samples using tyramide chemistry, quinone methide chemistry, and click chemistry are described in U.S. Pat. No. 10,041,950, and in U.S. Publication Nos. 2019/0204330, 2017/0089911, and 2019/0187130, the disclosures of which are hereby incorporated by reference herein in their entireties.

In both methods, the one or more morphological markers and the one or more biomarkers in the biological sample are first labeled with an enzyme. Said another way, a first step in either method is forming one or more morphological marker-enzyme complexes and one or more biomarker-enzyme complexes. In some embodiments, the one or more morphological marker-enzyme complexes and the one or more biomarker-enzyme complexes serve as intermediates for further reaction in either of the two methods described herein. Suitable enzymes for labeling the one or more morphological markers and the one or more biomarkers include, but are not limited to, horseradish peroxidase (HRP), alkaline phosphatase (AP), acid phosphatase, glucose oxidase, β-galactosidase, β-glucuronidase or β-lactamase. In some embodiments, the one or more morphological markers and the one or more biomarkers are labeled with horseradish peroxidase or alkaline phosphatase. In some embodiments, the one or more morphological markers and the one or more biomarkers are each labeled with the same enzyme. In other embodiments, the one or more morphological markers and the one or more biomarkers are labeled with different enzymes.

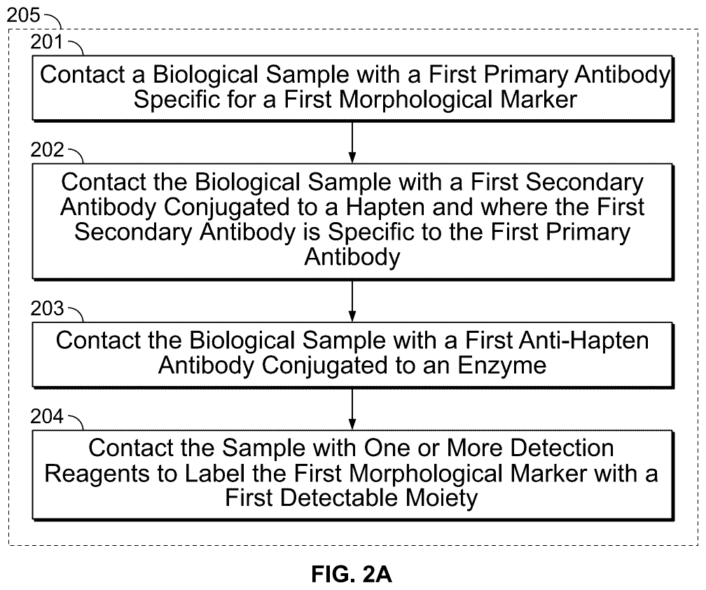

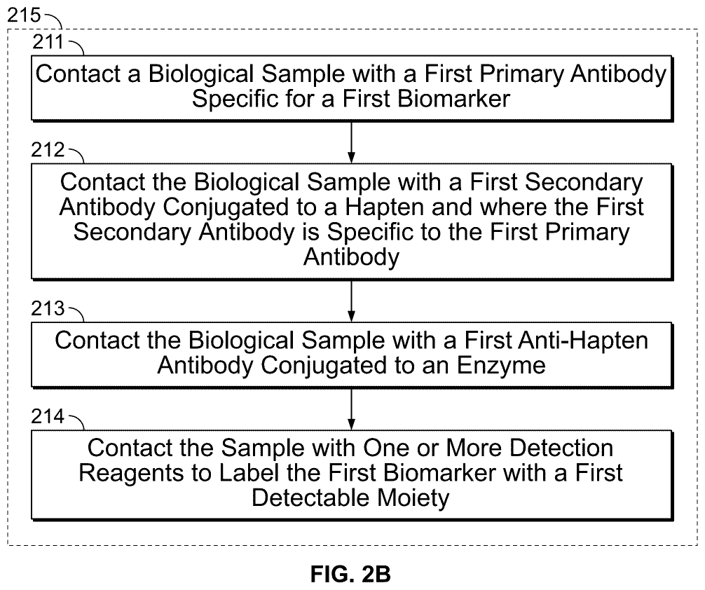

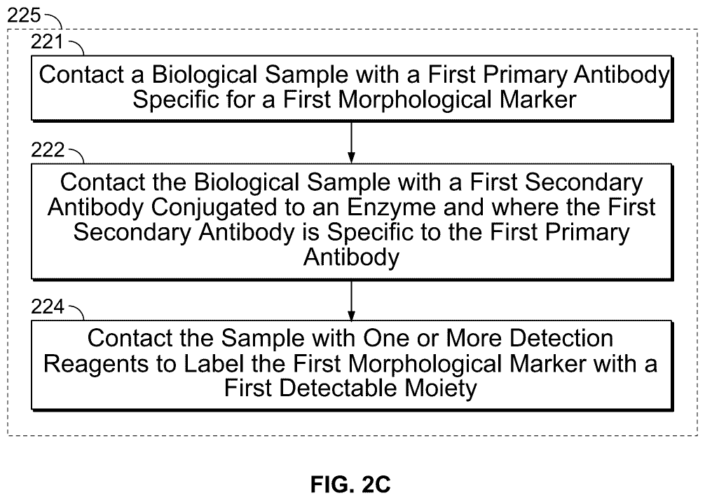

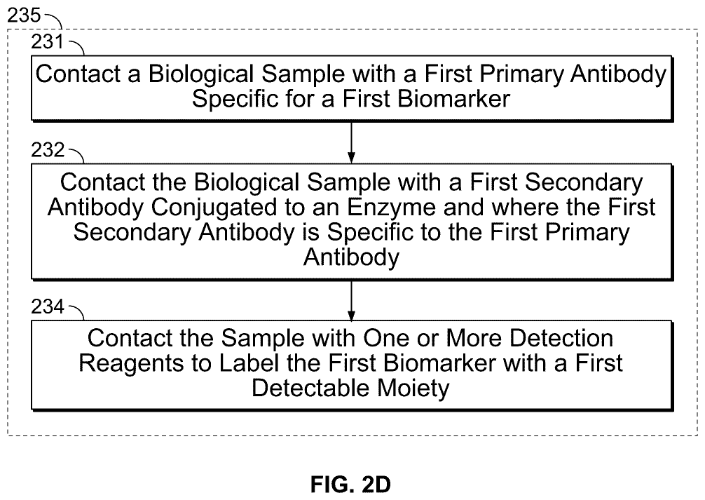

To facilitate the labeling of the one or more morphological markers and the one or more biomarkers in the biological sample with one or more enzymes, in some embodiments, one or more specific binding entities specific to the one or more morphological markers and the one or more biomarkers are introduced to the biological sample (either sequentially or simultaneously). With reference to FIGS. 2A, 2B, 2C, and 2D, in some embodiments the one or more specific binding entities specific to the one or more morphological markers and one or more biomarkers are one or more primary antibodies (step 201, 211, 221, and 231). Upon introduction of the one or more primary antibodies, one or more secondary antibodies conjugated to a label (directly or indirectly through a linker) may be introduced, where each secondary antibody is specific to the one or more primary antibodies (e.g. the secondary antibodies are anti-primary antibody antibodies) (steps 202, 212, 222, and 232). In some embodiments, the label of the secondary antibody is an enzyme, including any of those described above (see steps 222 and 232 of FIGS. 2C and 2D).

In other embodiments, the label of the secondary antibody is a hapten (steps 202 or 212 of FIGS. 2A and 2B). Non-limiting examples of haptens include an oxazole, a pyrazole, a thiazole, a benzofurazan, a triterpene, a urea, a thiourea other than a rhodamine thiourea, a nitroaryl other than dinitrophenyl or trinitrophenyl, a rotenoid, a cyclolignan, a heterobiaryl, an azoaryl, a benzodiazepine, 2,3,6,7-tetrahydro-11-oxo-1H,5H,11H-[l]benzopyrano[6,7,8-ij]quinolizine-10-carboxylic acid, or 7-diethylamino-3-carboxycoumarin. Other suitable haptens are disclosed in U.S. Pat. No. 8,846,320, the disclosure of which is hereby incorporated by reference herein in its entirety. In those embodiments where the secondary antibody is conjugated to a hapten, anti-hapten antibodies conjugated to an enzyme (including any of those described above) may be introduced to the biological sample to label the one or more morphological markers and the one or more biomarkers with the one or more enzymes (steps 203 or 213). Subsequently, suitable detection reagents may be introduced to the biological sample to facilitate the labeling of each of the one or more morphological markers (now coupled indirectly to an enzyme) and one or more biomarkers (also now coupled indirectly to an enzyme) with a detectable moiety (including any of the detectable moieties described herein) (steps 204, 214, 224, and 234). Each of the steps in FIGS. 2A, 2B, 2C, and 2D may be repeated one or more times as needed (see steps 205, 215, 225, and 235).

In some embodiments, the one or more specific binding entities are primary antibody conjugates and/or nucleic acid probe conjugates. In some embodiments, the one or more specific binding entities are primary antibody conjugates coupled to an enzyme. In some embodiments, the primary antibody conjugates are conjugated to horseradish peroxidase or alkaline phosphatase. In other embodiments, the one or more specific binding entities are nucleic acid probes conjugated to an enzyme, e.g. horseradish peroxidase or alkaline phosphatase. Introduction of the one or more specific binding entities conjugated to an enzyme facilitates the formation of one or more morphological marker-enzyme complexes and one or more biomarker-enzyme complexes.

In some embodiments, the one or more specific binding entities are primary antibody conjugates coupled to a hapten and/or one or more nucleic acid probes conjugated to a hapten (including any of those haptens described in U.S. Pat. No. 8,846,320, the disclosure of which is hereby incorporated by reference herein in its entirety). In these embodiments, the introduction of the one or more specific binding entities conjugated to haptens facilitates for the formation of one or more hapten-labeled morphological markers and one or more hapten-labeled biomarkers. In these embodiments, one or more anti-hapten antibody-enzyme conjugates specific to the haptens of the one or more hapten-labeled morphological markers and the one or more hapten-labeled biomarkers are introduced to the biological sample so as to label the one or more hapten-labeled morphological markers and the one or more hapten-labeled biomarkers with an enzyme to provide one or more morphological marker-enzyme complexes and one or more biomarker-enzyme complexes. The primary antibody conjugates, secondary antibodies, and/or nucleic acid probes may be introduced to a sample according to procedures known to those of ordinary skill in the art to effect labeling of one or more targets in a biological sample with an enzyme and as illustrated herein.

Each of the aforementioned methods are described in more detail herein. In some embodiments, both methods may be used to label the one or more morphological markers and the one or more biomarkers with detectable labels, i.e. mixed chemistries may be utilized to facilitate labeling of the one or more morphological markers and the one or more biomarkers in the biological sample.

Methods of Detecting One or More Morphological Markers and One or More Biomarkers in a Biological Sample Using Tyramide and/or Quinone Methide Conjugates

In some embodiments, the present disclosure provides methods of detecting one or more morphological markers and one or more biomarkers using detectable conjugates comprising (i) a tyramide and/or quinone methide precursor moiety, and (ii) a detectable moiety, including any of the detectable moieties described herein.

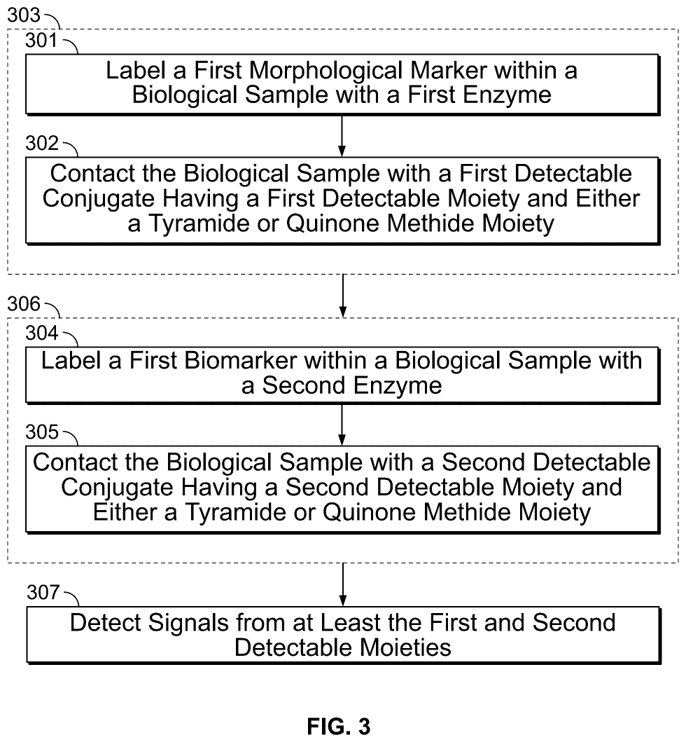

In some embodiments, and with reference to FIG. 3, a biological sample having a first morphological marker is labeled with a first enzyme (step 301) to form a first morphological marker-enzyme complex. Methods of labeling a first morphological marker with a first enzyme are described above and also illustrated in FIGS. 2A and 2C. In some embodiments, the first morphological marker is selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers. In some embodiments, the first morphological marker is selected from the group consisting of DNA and histone proteins.

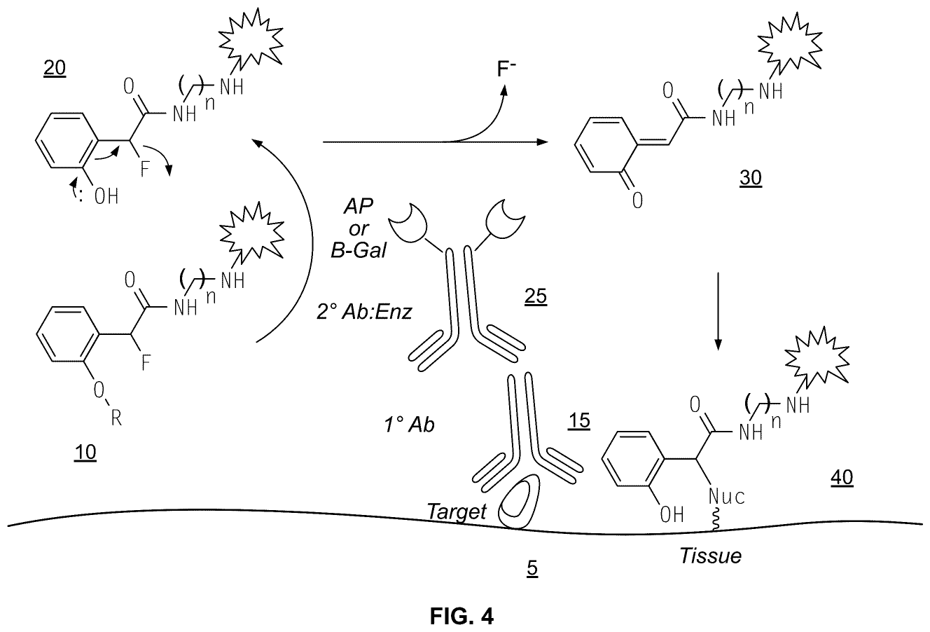

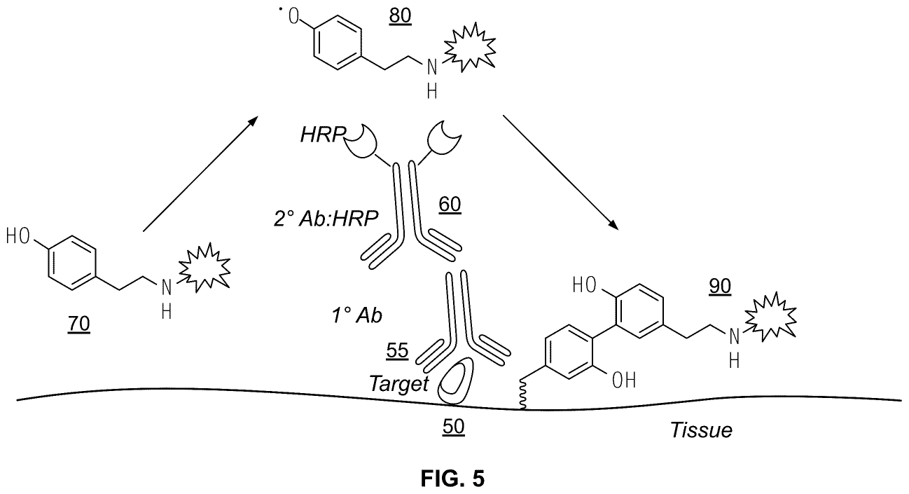

The biological sample is then contacted with a first detectable conjugate (step 302), the first detectable conjugate comprising a first detectable moiety (including any of those described herein) and either a tyramide, a quinone methide, or a derivative or analog thereof. Examples of detectable conjugates including a tyramide moiety, a quinone methide precursor moiety, or a derivative or analog thereof are described herein. Upon interaction of the first enzyme of the first morphological marker-enzyme complex with the tyramide or the quinone methide portion of the first detectable conjugate, at least the first detectable moiety of the detectable conjugate is deposited proximal to or onto the first morphological marker target (see also FIGS. 4 and 5 which illustrate the deposition of a detectable moiety proximal to or onto a target molecule within a biological sample, where the target molecule 5 or 50 may be a morphological marker).

The aforementioned process (steps 301 and 302) may be repeated (step 303) for any number of morphological markers within the biological sample. In some embodiments, each morphological marker is labeled with a different detectable moiety. In other embodiments, each morphological marker is labeled with the same detectable moiety. For instance, if the morphological markers are DNA and histone proteins, in some embodiments, it may be desirable to label both the DNA and histone protein markers with the same detectable moiety such that nuclear components of cells are stained with a single detectable moiety.

Next, the biological sample having a first biomarker is labeled with a second enzyme (step 304) to form a first biomarker-enzyme complex. Methods of labeling a first biomarker with a second enzyme are described above and also illustrated in FIGS. 2B and 2D. The biological sample including the first biomarker-enzyme complex is then contacted with a second detectable conjugate (step 305), the second detectable conjugate comprising a second detectable moiety (including any of those described herein) and either a tyramide, a quinone methide, or a derivative or analog thereof. Upon interaction of the second enzyme of the first biomarker-enzyme complex with the tyramide or the quinone methide portion of the second detectable conjugate, at least the second detectable moiety of the second detectable conjugate is deposited proximal to or onto the first biomarker target (see also FIGS. 4 and 5 which illustrate the deposition of a detectable moiety proximal to or onto a target molecule within a biological sample, where the target molecule 5 or 50 may be a biomarker).

The steps of labeling a biomarker with an enzyme (step 304) and subsequently a detectable moiety (step 305) may be repeated (step 306) any number of times and for any different types of biomarkers (e.g. protein, nucleic acid) within the biological sample. In some embodiments, each biomarker is labeled with a different detectable moiety (and where each label for each biomarker is also different than each label for each morphological marker). For example, a Ki-67 biomarker may be labeled with a detectable moiety having a first absorbance peak with FWHM of less than 200 nm (e.g. less than 160 nm, less than 130 nm, less than 100 nm, etc.) and a peak absorbance wavelength between 440 nm and 470 nm; and a PD-L1 biomarker may be labeled with a detectable moiety having a first absorbance peak with FWHM of less than 130 nm (e.g. less than 160 nm, less than 130 nm, less than 100 nm, etc.) and a peak absorbance wavelength between 590 nm and 620 nm. Following this example further, a Ki-67 biomarker may be labeled with a detectable moiety having a first absorbance peak with FWHM of less than 130 nm (e.g. less than 160 nm, less than 130 nm, less than 100 nm, etc.). and a peak absorbance wavelength between 440 nm and 470 nm; a PD-L1 biomarker may be labeled with a detectable moiety having a first absorbance peak with FWHM of less than 130 nm (e.g. less than 160 nm, less than 130 nm, less than 100 nm, etc.) and a peak absorbance wavelength between 590 nm and 620 nm; and the morphological marker (e.g. DNA or histone proteins) may be labeled with a detectable moiety having a first absorbance peak with FWHM of less than 130 nm (e.g. less than 160 nm, less than 130 nm, less than 100 nm, etc.) and a peak absorbance wavelength of between 510 nm and 540 nm.

Finally, signals from the first and second detectable moieties are detected (e.g. such as using bright-field microscopy) (step 307). Methods of detecting one or more signals from one or more detectable moieties are described in U.S. Pat. No. 10,778,913, the disclosure of which is hereby incorporated by reference herein in its entirety and described further herein.

In some embodiments, the first and second detectable moieties of the first and second detectable conjugates are selected such that the first and second detectable moieties have different peak absorbance wavelengths and which do not substantially overlap (e.g. the different peak absorbance wavelengths different by at least about 20 nm, by at least about 25 nm, by at least about 30 nm, by at least about 40 nm, by at least about 50 nm, by at least about 60 nm, by at least about 70 nm, by at least about 80 nm, by at least about 90 nm, by at least about 100 nm, by at least about 110 nm, by at least about 120 nm, by at least about 130 nm, by at least about 140 nm, by at least about 150 nm, by at least about 170 nm, by at least about 190 nm, by at least about 210 nm, by at least about 230 nm, by at least about 250 nm, by at least about 270 nm, by at least about 290 nm, by at least about 310 nm, etc.).

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 200 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 200 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 200 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 200 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 200 nm.

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 130 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 130 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 60 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 130 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 130 nm.

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 100 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 100 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 100 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 100 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 100 nm.

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 80 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 80 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 80 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 80 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 80 nm.

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 60 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 60 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 30 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 60 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 60 nm.

In some embodiments, the first detectable moiety comprises a coumarin core. In some embodiments, the second detectable moiety is within the visible spectrum or within the infrared spectrum. In some embodiments, the second detectable moiety is within the ultraviolet spectrum. In some embodiments, the first and second detectable moieties have absorbance maximums (λₘₐₓ) that are separated by at least 20 nm.

In some embodiments, the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core. In some embodiments, the second detectable moiety is within the ultraviolet spectrum or within the infrared spectrum. In some embodiments, the second detectable moiety is within the visible spectrum. In some embodiments, wherein the first and second detectable moieties have absorbance maximums (λₘₐₓ) that are separated by at least 20 nm.

In some embodiments, the first detectable moiety comprises a heptamethine cyanine core or a croconate core. In some embodiments, the second detectable moiety is within the visible spectrum or within the ultraviolet spectrum. In some embodiments, the second detectable moiety is within the infrared spectrum. In some embodiments, the first and second detectable moieties have absorbance maximums (λₘₐₓ) that are separated by at least 20 nm.

As an alternative to the workflow depicted in FIG. 3, in some embodiments the labeling of the first morphological marker with first enzyme may occur simultaneously with or sequentially with the labeling of the first biomarker with the second enzyme. Then, first and second detectable conjugates may be added to the biological sample simultaneously or sequentially so as to label with the first morphological marker and the first biomarker with the first and second detectable moieties, respectively, again provided that the first and second detectable moieties have different peak absorbance wavelengths as noted herein.

FIGS. 4 and 5 further illustrate the reactions that take place between the various components introduced to the biological sample. With reference to FIG. 4, a specific binding entity 15 is first introduced to a biological sample having a target 5 to form a target-detection probe complex. In some embodiments, the target 5 is a morphological marker and the formed target-detection probe complex is a morphological marker-detection probe complex. In other embodiments, the target 5 is a biomarker and the formed target-detection probe complex is a biomarker-detection probe complex. In some embodiments, the specific binding entity 15 is a primary antibody. Subsequently, a labeling conjugate 25 is introduced to the biological sample, the labeling conjugate 25 comprising at least one enzyme conjugated thereto. In the embodiment depicted, the labeling conjugate 25 is a secondary antibody, where the secondary antibody is an anti-species antibody conjugated to an enzyme. Next, a detectable conjugate 10 is introduced, such as a detectable conjugate including any of the detectable moieties described herein coupled directly or indirectly to a quinone methide precursor moiety or a derivative or analog thereof. Upon interaction of the enzyme (e.g. AP or B-Gal) with the detectable conjugate 10, the detectable conjugate 10 undergoes a structural, conformational, or electronic change 20 to form a tissue reactive intermediate 30. In this particular embodiment, the detectable conjugate comprises a quinone methide precursor moiety that, upon interaction with the alkaline phosphatase enzyme (of the labeling conjugate 25), causes a fluorine leaving group to be ejected, resulting in the respective quinone methide intermediate 30. The quinone methide intermediate 30 then forms a covalent bond with the tissue proximal or directly on the tissue to form a detectable moiety complex 40. Signals from the detectable moiety complex 40 may then be detected according to methods known to those of ordinary skill in the art, such as those described in U.S. Pat. Nos. 10,041,950, and 10,778,913; and in U.S. Publication Nos. 2019/0204330, 2017/0089911, the disclosures of which are hereby incorporated by reference herein in its entirety. The steps of FIG. 4 may be repeated for one or more morphological markers and/or one or more biomarkers within a target.

With reference to FIG. 5, a specific binding entity 55 is first introduced to a biological sample having a target 50 to form a target-detection probe complex and the formed target-detection probe complex is a morphological marker-detection probe complex. In some embodiments, the target 50 is a morphological marker. In other embodiments, the target 50 is a biomarker and the formed target-detection probe complex is a biomarker-detection probe complex. In some embodiments, the specific binding entity 55 is a primary antibody. Subsequently, a labeling conjugate 60 is introduced to the biological sample, the labeling conjugate 60 comprising at least one enzyme conjugated thereto. In the embodiment depicted, the labeling conjugate is a secondary antibody, where the secondary antibody is an anti-species antibody conjugated to an enzyme. Next, a detectable conjugate 70 is introduced, such as a detectable conjugate including any of the detectable moieties described herein coupled directly or indirectly to a tyramide moiety or a derivative or analog thereof. Upon interaction of the enzyme with the detectable conjugate 70, a tissue reactive intermediate 80 is formed. In this particular embodiment, the detectable conjugate 70 comprises a tyramide moiety that, upon interaction with horseradish peroxidase enzyme, causes formation of the radical species 80. The radical intermediate 80 then forms a covalent bond with the tissue proximal or directly on the tissue to form a detectable moiety complex 90. Signals from the detectable moiety complex 90 may then be detected according to methods known to those of ordinary skill in the art, such as those described in U.S. Pat. Nos. 10,041,950 and 10,778,913, and in U.S. Publication Nos. 2019/0204330, 2017/0089911, and 2019/0187130, the disclosures of which are hereby incorporated by reference herein in its entirety. The steps of FIG. 5 may be repeated for one or more morphological markers and/or one or more biomarkers within a target. In some embodiments, the steps of FIG. 4 are used to label a morphological marker while the steps of FIG. 5 are used to label a biomarker. In other embodiments, the steps of FIG. 5 are used to label a morphological marker while the steps of FIG. 4 are used to label a biomarker.

In some embodiments, the biological samples are pre-treated with an enzyme inactivation composition to substantially or completely inactivate endogenous peroxidase activity. For example, some cells or tissues contain endogenous peroxidase. Using an HRP conjugated antibody may result in high, non-specific background staining. This non-specific background can be reduced by pre-treatment of the sample with an enzyme inactivation composition as disclosed herein. In some embodiments, the samples are pre-treated with hydrogen peroxide only (about 1% to about 3% by weight of an appropriate pre-treatment solution) to reduce endogenous peroxidase activity. Once the endogenous peroxidase activity has been reduced or inactivated, detection kits may be added, followed by inactivation of the enzymes present in the detection kits, as provided above. The disclosed enzyme inactivation composition and methods can also be used as a method to inactivate endogenous enzyme peroxidase activity. Additional inactivation compositions are described in U.S. Publication No. 2018/0120202, the disclosure of which is hereby incorporated by reference herein in its entirety.

In some embodiments if the specimen is a sample embedded in paraffin, the sample can be deparaffinized using appropriate deparaffinizing fluid(s). After a waste remover removes the deparaffinizing fluid(s), any number of substances can be successively applied to the specimen. The substances can be for pretreatment (e.g., protein-crosslinking, expose nucleic acids, etc.), denaturation, hybridization, washing (e.g., stringency wash), detection (e.g., link a visual or marker molecule to a probe), amplifying (e.g., amplifying proteins, genes, etc.), counterstaining, coverslipping, or the like.

Methods of Detecting Targets in a Sample Using a Pair of Click Conjugates

The present disclosure provides methods of detecting one or more morphological markers and one or more biomarkers within a biological sample using pairs of click conjugates. In these assays, one member of a pair of click conjugates comprises a detectable conjugate comprising: (i) a first functional group capable of participating in a click chemistry reaction, and (ii) a detectable moiety, including any of the detectable moieties described herein. Non-limiting examples of suitable detectable conjugates are described herein. Another member of the pair of click conjugates (hereinafter referred to as “tissue reactive conjugates”) comprises a conjugate comprising: (i) a tyramide moiety, a quinone methide precursor moiety, or a derivative or analog of a tyramide moiety or a quinone methide precursor moiety; and (ii) a second functional group capable of reacting the first functional group of the detectable conjugate. Suitable first and second functional groups coupled to the detectable conjugate and the tissue reactive conjugate and capable of reacting with each other are set forth in Table 12:

TABLE 12
First and second functional groups capable of reacting with
each other in a ″click chemistry″ reaction.

| Reactive Functional Group | Reactive Functional Group |
|---|---|
| on a First Member of a | on a Second Member of a |
| Pair of Click Conjugates | Pair of Click Conjugates |
| Alkyne | Azide |
| Azide | Alkyne |
| diarylcyclooctyne (″DBCO″) | Azide |
| Alkene | Tetrazine |
| Trans-cyclooctene (″TCO″) | Tetrazine |
| Maleimide | Thiol |
| DBCO | 1,3-Nitrone |
| Aldehyde or ketone | Hydrazine |
| Aldehyde or ketone | Hydroxylamine |
| Azide | DBCO |
| Tetrazine | TCO |
| Thiol | Maleimide |
| 1,3-Nitrone | DBCO |
| Hydrazine | Aldehyde or ketone |
| Hydroxylamine | Aldehyde or ketone |
| Tetrazine | Alkene |

Non-limiting examples of suitable tissue reactive conjugates are illustrated below:

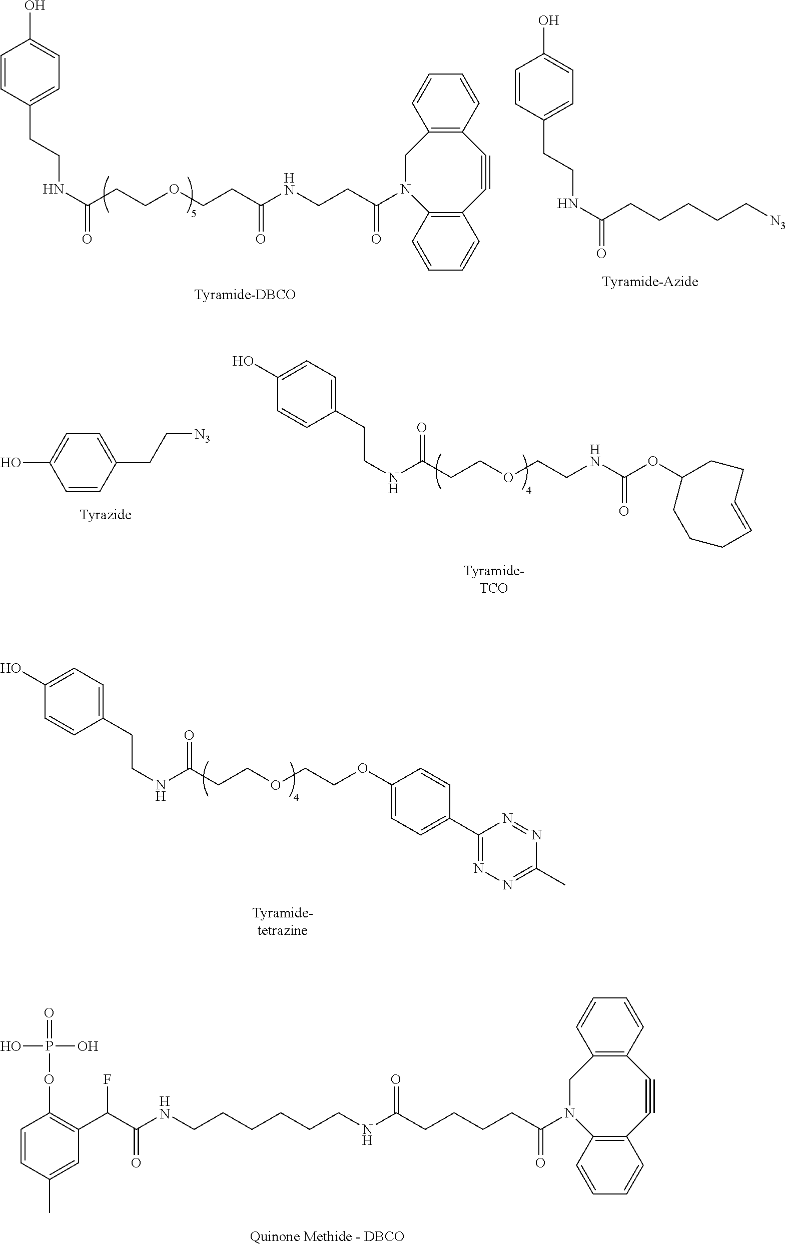

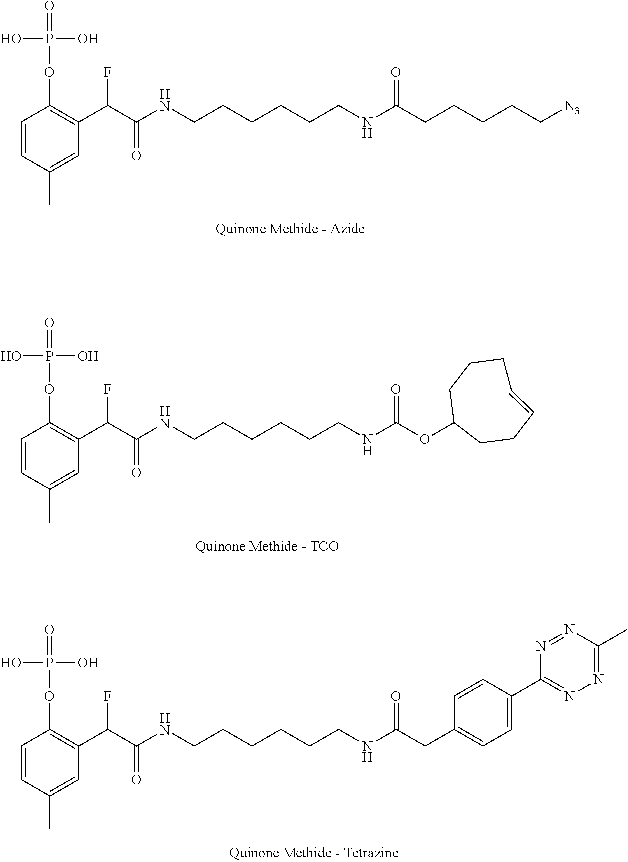 [SMILES: Cc1ccc(OP(=O)(O)O)c(C(F)C(=O)NCCCCCCNC(=O)CCCCCN=[N+]=[N-])c1 ; Cc1ccc(OP(=O)(O)O)c(C(F)C(=O)NCCCCCCNC(=O)OC2CC/C=C/CCC2)c1 ; Cc1ccc(OP(=O)(O)O)c(C(F)C(=O)NCCCCCCNC(=O)Cc2ccc(-c3nnc(C)nn3)cc2)c1]

Other suitable “tissue reactive conjugates” are described in U.S. Publication Nos. 2019/0204330, 2017/0089911, and 2019/0187130, the disclosures of which are hereby incorporated by reference herein their entireties.

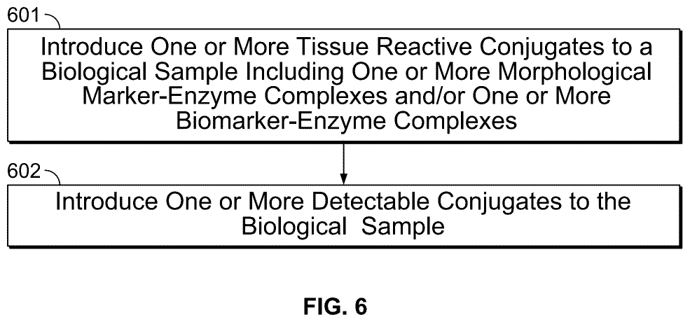

In general, as a first step of labeling one or more morphological markers and one or more biomarkers with detectable moieties comprises covalently depositing one or more tissue reactive conjugates onto tissue using quinone methide signal amplification (“QMSA”) and/or tyramide signal amplification (“TSA”) (see FIG. 6 at step 601). The introduction of the one or more tissue reactive conjugates introduces a first member of a pair of reactive functional groups to one or more morphological markers and one or more biomarkers. These amplification procedures are described in U.S. Publication Nos. 2019/0204330, 2017/0089911, and 2019/0187130, the disclosures of which are each hereby incorporated by reference in their entireties. Then, one or more detectable conjugates are introduced to the tissue (see FIG. 6 ay step 602). The “click” reaction between the two “click” conjugates (i.e. the tissue reactive conjugate and the detectable conjugate including the functional groups capable of reacting with each other) occurs rapidly, covalently binding the detectable moieties to tissue in the locations dictated by the QMSA or TSA chemistries. Each of these steps are described in greater detail herein.

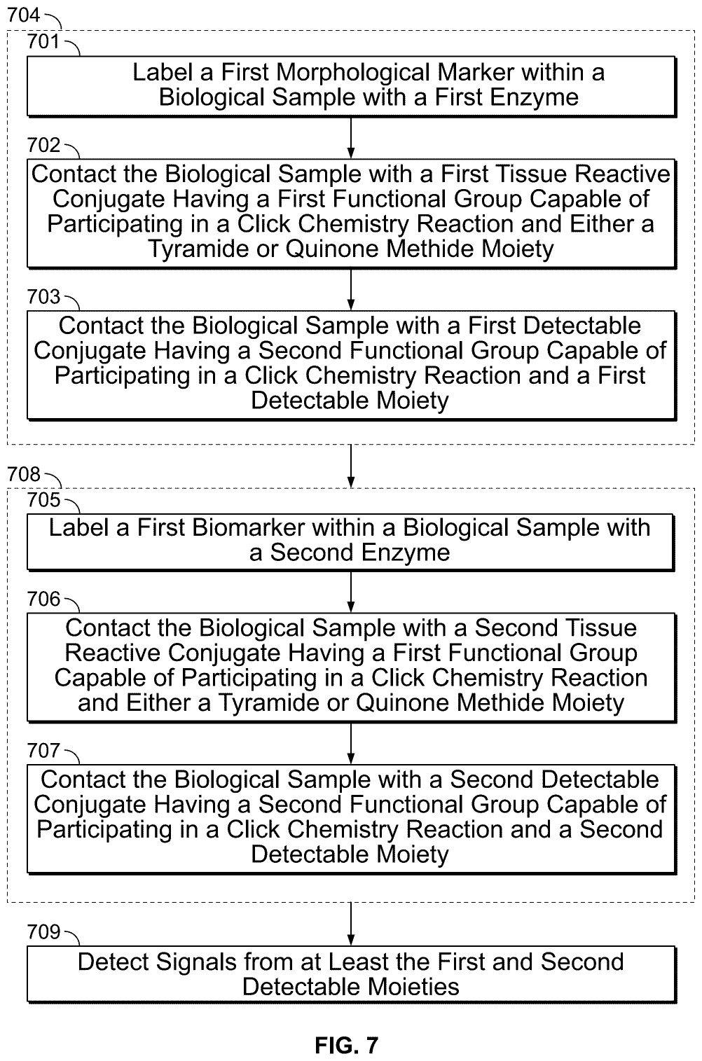

In some embodiments, and with reference to FIG. 7, a biological sample having a first morphological marker is labeled with a first enzyme (step 701) to form a first morphological marker-enzyme complex. Methods of labeling a first morphological marker with a first enzyme are described above and also illustrated in FIGS. 2A and 2C. In some embodiments, the first morphological marker is selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers. In some embodiments, the first morphological marker is selected from the group consisting of DNA and histone proteins.

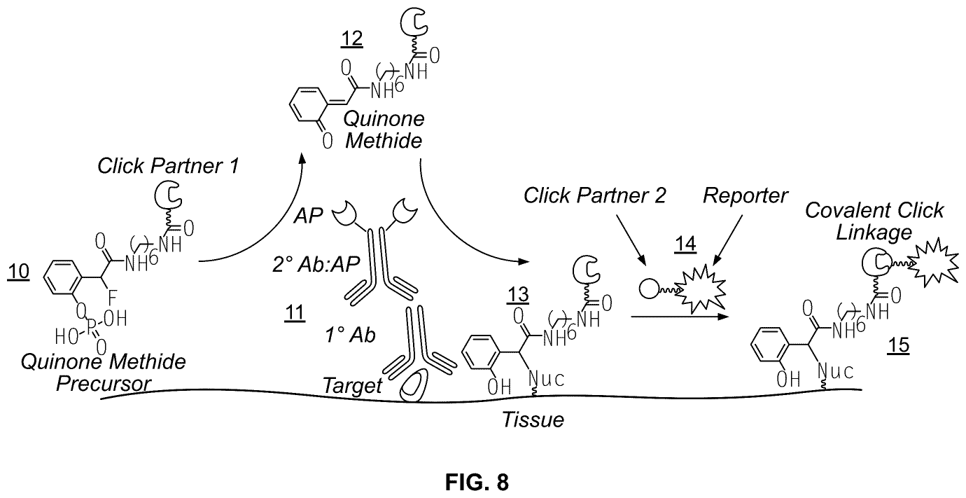

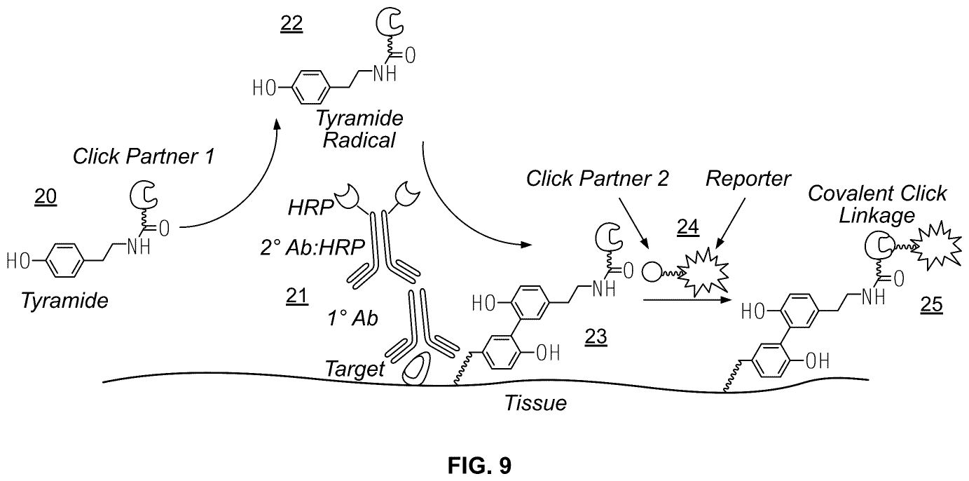

The biological sample is then contacted with a first tissue reactive conjugate (step 702), the first tissue reactive conjugate comprising a first functional group capable of participating in a click chemistry reaction (including any of those described in Tables 1 and 2 herein) and either a tyramide, a quinone methide, or a derivative or analog thereof. Non-limiting examples of tissue reactive conjugates are provided herein. Upon interaction of the first enzyme of the first morphological marker-enzyme complex with the tyramide or the quinone methide portion of the first tissue reactive conjugate, at least a first immobilized tissue-click conjugate complex is deposited proximal to or onto the first morphological marker target (see also FIGS. 8 and 9 which further illustrate the “click chemistry” reactions that may take place and the formation of the resulting “first immobilized tissue-click conjugate complex” and “first immobilized tissue-click adduct complex”). Following the formation of the first immobilized tissue-click conjugate complex, the biological sample is then contacted with a first detectable conjugate comprising: (i) a second functional group capable of reacting with the first reactive functional group of the first immobilized tissue-click conjugate complex, and (ii) a first detectable moiety (step 703). The reaction product of first immobilized tissue-click conjugate complex and first detectable conjugate produces a first immobilized tissue-click adduct complex which may be detected.

The aforementioned process (steps 701, 702, and 703) may be repeated (step 704) for any number of morphological markers within the biological sample. In some embodiments, each morphological marker is labeled with a different detectable moiety. In other embodiments, each morphological marker is labeled with the same detectable moiety. For instance, if the morphological markers are DNA and histone proteins, in some embodiments, it may be desirable to label both the DNA and histone protein markers with the same detectable moiety such that nuclear components of cells are stained with a single detectable moiety.

Next, the biological sample having a first biomarker is labeled with a second enzyme (step 705) to form a first biomarker-enzyme complex. Methods of labeling a first biomarker with a second enzyme are described above and also illustrated in FIGS. 2B and 2D. The biological sample is then contacted with a second tissue reactive conjugate (step 706), the second tissue reactive conjugate comprising a first functional group capable of participating in a click chemistry reaction (including any of those described in Tables 1 and 2 herein) and either a tyramide, a quinone methide, or a derivative or analog thereof. Non-limiting examples of tissue reactive conjugates are provided herein. Upon interaction of the second enzyme of the first biomarker-enzyme complex with the tyramide or the quinone methide portion of the second tissue reactive conjugate, at least a second immobilized tissue-click conjugate complex is deposited proximal to or onto the first biomarker target (see also FIGS. 8 and 9 which further illustrate the “click chemistry” reactions that may take place and the formation of the resulting “second immobilized tissue-click conjugate complex” and “second immobilized tissue-click adduct complex”). Following the formation of the second immobilized tissue-click conjugate complex, the biological sample is then contacted with a second detectable conjugate comprising: (i) a second functional group capable of reacting with the first reactive functional group of the second immobilized tissue-click conjugate complex, and (ii) a second detectable moiety (step 707). The reaction product of second immobilized tissue-click conjugate complex and second detectable conjugate produces a second immobilized tissue-click adduct complex which may be detected.

The aforementioned process (steps 705, 706, and 707) may be repeated (step 708) for any number of biomarkers within the biological sample. In some embodiments, each biomarker is labeled with a different detectable moiety (and where each label for each biomarker is also different than each label for each morphological marker). For example, a Ki-67 biomarker may be labeled with a detectable moiety having a first absorbance peak with FWHM of less than 50 nm and a peak absorbance wavelength between 440 nm and 470 nm; and a PD-L1 biomarker may be labeled with a detectable moiety having a first absorbance peak with FWHM of less than 50 nm and a peak absorbance wavelength between 590 nm and 620 nm. Following this example further, a Ki-67 biomarker may be labeled with a detectable moiety having a first absorbance peak with FWHM of less than 50 nm and a peak absorbance wavelength between 440 nm and 470 nm; a PD-L1 biomarker may be labeled with a detectable moiety having a first absorbance peak with FWHM of less than 50 nm and a peak absorbance wavelength between 590 nm and 620 nm; and the morphological marker (e.g. DNA or histone proteins) may be labeled with a detectable moiety having a first absorbance peak with FWHM of less than 50 nm and a peak absorbance wavelength of between 510 nm and 540 nm.

Finally, signals from the first and second detectable moieties are detected (e.g. such as using brightfield microscopy) (step 709). Methods of detecting one or more signals from one or more detectable moieties are described in U.S. Pat. No. 10,778,913, the disclosure of which is hereby incorporated by reference herein in its entirety.

In some embodiments, the first and second detectable moieties of the first and second detectable conjugates are selected such that the first and second detectable moieties have different peak absorbance wavelengths and which do not substantially overlap (e.g. the different peak absorbance wavelengths different by at least about 20 nm, by at least about 25 nm, by at least about 30 nm, by at least about 40 nm, by at least about 50 nm, by at least about 60 nm, by at least about 70 nm, by at least about 80 nm, by at least about 90 nm, by at least about 100 nm, by at least about 110 nm, by at least about 120 nm, by at least about 130 nm, by at least about 140 nm, by at least about 150 nm, by at least about 170 nm, by at least about 190 nm, by at least about 210 nm, by at least about 230 nm, by at least about 250 nm, by at least about 270 nm, by at least about 290 nm, by at least about 310 nm, etc.).

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 200 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 200 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 200 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 200 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 200 nm.

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 130 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 130 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 130 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 130 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 130 nm.

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 100 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 100 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 100 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 100 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 100 nm.

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 80 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 80 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 80 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 80 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 80 nm.

In some embodiments, the first and second detectable moieties have different peak absorbance wavelengths, wherein the different peak absorbance wavelengths of the first and second detectable moieties are separated by at least 20 nm, and wherein each of the first and second detectable moieties have FWHM of less than 60 nm. In some embodiments, the first and second detectable moieties have different peak absorbance wavelengths, wherein the different peak absorbance wavelengths of the first and second detectable moieties are separated by at least 30 nm, and wherein each of the first and second detectable moieties have FWHM of less than 60 nm. In some embodiments, the first and second detectable moieties have different peak absorbance wavelengths, wherein the different peak absorbance wavelengths of the first and second detectable moieties are separated by at least 40 nm, and wherein each of the first and second detectable moieties have FWHM of less than 60 nm. In some embodiments, the first and second detectable moieties have different peak absorbance wavelengths, wherein the different peak absorbance wavelengths of the first and second detectable moieties are separated by at least 50 nm, and wherein each of the first and second detectable moieties have FWHM of less than 60 nm. In some embodiments, the first and second detectable moieties have different peak absorbance wavelengths, wherein the different peak absorbance wavelengths of the first and second detectable moieties are separated by at least 70 nm, and wherein each of the first and second detectable moieties have FWHM of less than 60 nm.

In some embodiments, the first detectable moiety comprises a coumarin core. In some embodiments, the second detectable moiety is within the visible spectrum or within the infrared spectrum. In some embodiments, the second detectable moiety is within the ultraviolet spectrum. In some embodiments, the first and second detectable moieties have absorbance maximums (λₘₐₓ) that are separated by at least 20 nm.

In some embodiments, the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core. In some embodiments, the second detectable moiety is within the ultraviolet spectrum or within the infrared spectrum. In some embodiments, the second detectable moiety is within the visible spectrum. In some embodiments, wherein the first and second detectable moieties have absorbance maximums (λₘₐₓ) that are separated by at least 20 nm.

In some embodiments, the first detectable moiety comprises a heptamethine cyanine core or a croconate core. In some embodiments, the second detectable moiety is within the visible spectrum or within the ultraviolet spectrum. In some embodiments, the second detectable moiety is within the infrared spectrum. In some embodiments, the first and second detectable moieties have absorbance maximums (λₘₐₓ) that are separated by at least 20 nm.

FIGS. 8 and 9 further illustrate the reaction between a first member of a pair of click conjugates having a tissue reactive moiety (10, 20) and a target-bound enzyme (11, 21) to form an immobilized tissue-click conjugate complex (13, 23). This first part of the amplification process is similar to that used in QMSA and TSA amplification processes. FIGS. 8 and 9 illustrate the subsequent reaction between the immobilized tissue-click conjugate (13, 23) complex and a second member of the pair of click conjugates (14, 24), to provide an immobilized tissue-click adduct complex (15, 25) comprising a detectable reporter moiety.

With reference to FIG. 8 a tissue reactive conjugate comprising a reactive functional group (10) is brought into contact with a target-bound enzyme (11) to produce a reactive intermediate (12). In some embodiments, the target-bound enzyme (11) is a morphological marker-bound enzyme. In other embodiments, the target-bound enzyme (11) is a biomarker-bound enzyme. In this example, the reactive intermediate, a quinone methide, forms a covalent bond to a nucleophile on or within a biological sample, thus providing an immobilized tissue-click conjugate complex (13). The immobilized tissue-click conjugate complex may then react with a detectable conjugate having any of the detectable moieties described herein (14), provided that the tissue reactive conjugate 10 and the detectable conjugate 14 possess reactive functional groups that may react with each other to form a covalent bond. The reaction product of immobilized tissue-click conjugate complex 13 and click conjugate 14 produces the immobilized tissue-click adduct complex 15. The tissue-click adduct complex 15 may be detected by virtue of signals transmitted from the linked detectable moiety. In some embodiments, the steps of FIG. 8 may be repeated for any number of morphological markers and or biomarkers.

Similarly, and with reference to FIG. 9, a tissue reactive conjugate comprising a reactive functional group (20) is brought into contact with a target-bound enzyme (21), to produce a reactive intermediate (22), namely a tyramide radical species (or derivative thereof). In some embodiments, the target-bound enzyme (21) is a morphological marker-bound enzyme. In other embodiments, the target-bound enzyme (21) is a biomarker-bound enzyme. The tyramide radical intermediate may then form a covalent bond to a biological sample, thus providing an immobilized tissue-click conjugate complex (23). The immobilized tissue-click conjugate complex may then react with a detectable conjugate including any of the detectable moieties described herein (24), provided that tissue reactive conjugate and the detectable conjugate 20 and 24, respectively, possess reactive functional groups that may react with each other to form a covalent bond. The reaction product of immobilized tissue-click conjugate complex 23 and click conjugate 24 produces the tissue-click adduct complex 25. In some embodiments, the steps of FIG. 9 may be repeated for any number of morphological markers and or biomarkers. In some embodiments, the steps of FIG. 8 are used to label a morphological marker while the steps of FIG. 9 are used to label a biomarker. In other embodiments, the steps of FIG. 9 are used to label a morphological marker while the steps of FIG. 8 are used to label a biomarker.

Automation

The assays and methods of the present disclosure may be automated and may be combined with a specimen processing apparatus. The specimen processing apparatus can be an automated apparatus, such as the BENCHMARK XT instrument and DISCOVERY XT instrument sold by Ventana Medical Systems, Inc. Ventana Medical Systems, Inc. is the assignee of a number of United States patents disclosing systems and methods for performing automated analyses, including U.S. Pat. Nos. 5,650,327, 5,654,200, 6,296,809, 6,352,861, 6,827,901 and 6,943,029, and U.S. Published Patent Application Nos. 20030211630 and 20040052685, each of which is incorporated herein by reference in its entirety. Alternatively, specimens can be manually processed.

The specimen processing apparatus can apply fixatives to the specimen. Fixatives can include cross-linking agents (such as aldehydes, e.g., formaldehyde, paraformaldehyde, and glutaraldehyde, as well as non-aldehyde cross-linking agents), oxidizing agents (e.g., metallic ions and complexes, such as osmium tetroxide and chromic acid), protein-denaturing agents (e.g., acetic acid, methanol, and ethanol), fixatives of unknown mechanism (e.g., mercuric chloride, acetone, and picric acid), combination reagents (e.g., Carnoy's fixative, methacarn, Bouin's fluid, B5 fixative, Rossman's fluid, and Gendre's fluid), microwaves, and miscellaneous fixatives (e.g., excluded volume fixation and vapor fixation).

If the specimen is a sample embedded in paraffin, the sample can be deparaffinized with the specimen processing apparatus using appropriate deparaffinizing fluid(s). After the waste remover removes the deparaffinizing fluid(s), any number of substances can be successively applied to the specimen. The substances can be for pretreatment (e.g., protein-crosslinking, expose nucleic acids, etc.), denaturation, hybridization, washing (e.g., stringency wash), detection (e.g., link a visual or marker molecule to a probe), amplifying (e.g., amplifying proteins, genes, etc.), counterstaining, coverslipping, or the like.

The specimen processing apparatus can apply a wide range of substances to the specimen. The substances include, without limitation, stains, probes, reagents, rinses, and/or conditioners. The substances can be fluids (e.g., gases, liquids, or gas/liquid mixtures), or the like. The fluids can be solvents (e.g., polar solvents, non-polar solvents, etc.), solutions (e.g., aqueous solutions or other types of solutions), or the like. Reagents can include, without limitation, stains, wetting agents, antibodies (e.g., monoclonal antibodies, polyclonal antibodies, etc.), antigen recovering fluids (e.g., aqueous- or non-aqueous-based antigen retrieval solutions, antigen recovering buffers, etc.), or the like. Probes can be an isolated nucleic acid or an isolated synthetic oligonucleotide, attached to a detectable label. Labels can include radioactive isotopes, enzyme substrates, co-factors, ligands, chemiluminescent or fluorescent agents, haptens, and enzymes.

After the specimens are processed, a user can transport specimen-bearing slides to the imaging apparatus. The imaging apparatus used here is a brightfield imager slide scanner. One brightfield imager is the iScan Coreo™ brightfield scanner sold by Ventana Medical Systems, Inc. In automated embodiments, the imaging apparatus is a digital pathology device as disclosed in U.S. Pat. No. 9,575,301; U.S. Patent Application Publication No. 2014/0178169, filed on Feb. 3, 2014, entitled IMAGING SYSTEMS, CASSETTES, AND METHODS OF USING THE SAME; U.S. Pat. No. 9,575,301; and U.S. Patent Application Publication No. 2014/0178169 are incorporated by reference in their entities. In other embodiments, the imaging apparatus includes a digital camera coupled to a microscope.

Detection and/or Imaging

Certain aspects, or all, of the disclosed embodiments can be automated, and facilitated by computer analysis and/or image analysis system. In some applications, precise color or fluorescence ratios are measured. In some embodiments, light microscopy is utilized for image analysis. Certain disclosed embodiments involve acquiring digital images. This can be done by coupling a digital camera to a microscope. Digital images obtained of stained samples are analyzed using image analysis software. Color or fluorescence can be measured in several different ways. For example, color can be measured as red, blue, and green values; hue, saturation, and intensity values; and/or by measuring a specific wavelength or range of wavelengths using a spectral imaging camera. The samples also can be evaluated qualitatively and semi-quantitatively. Qualitative assessment includes assessing the staining intensity, identifying the positively-staining cells and the intracellular compartments involved in staining, and evaluating the overall sample or slide quality. Separate evaluations are performed on the test samples and this analysis can include a comparison to known average values to determine if the samples represent an abnormal state.

Suitable detection methods are described in U.S. Pat. No. 10,778,913, the disclosure of which is hereby incorporated by reference herein in its entirety. In some embodiments, a suitable detection system comprises an imaging apparatus, one or more lenses, and a display in communication with the imaging apparatus. The imaging apparatus includes means for sequentially emitting energy and means for capturing an image/video. In some embodiments, the means for capturing is positioned to capture specimen images, each corresponding to the specimen being exposed to energy. In some embodiments, the means for capturing can include one or more cameras positioned on a front side and/or a backside of the microscope slide carrying the biological sample. The display means, in some embodiments, includes a monitor or a screen. In some embodiments, the means for sequentially emitting energy includes multiple energy emitters. Each energy emitter can include one or more IR energy emitters, UV energy emitters, LED light emitters, combinations thereof, or other types of energy emitting devices. The imaging system can further include means for producing contrast enhanced color image data based on the specimen images captured by the means for capturing. The displaying means displays the specimen based on the contrast enhanced color image data.

Methods and Techniques

Immunohistochemistry—Single and Multiplex

Primary antibodies anti-ds DNA [DSD/958] (ab215896) and anti-histone H3 (ab1791) were obtained from ABCAM (Cambridge MA). Other primary antibody IHC reagents were obtained from Ventana Medical Systems, Inc. (VSMI; Tucson, AZ), including anti-CD20 (cat no. 760-2531), anti-CD3 (cat no. 790-4341), and anti-CD8 (cat no. 790-4460). Enzyme-antibody conjugates used with the detectable moieties were OmniMap anti-Ms HRP (RUO), DISCOVERY (VMSI Cat #760-4310), OmniMap anti-Rb HRP (RUO), DISCOVERY (VMSI Cat #760-4311), UltraMap anti-Ms Alk Phos, DISCOVERY (VMSI Cat #760-4312), and UltraMap anti-Rb Alk Phos, DISCOVERY (VMSI Cat #760-4314). Fully automated multiplexed detection was performed on a DISCOVERY Ultra system using the above primary antibodies and detection reagents. The DISCOVERY Universal Procedure was used to create a protocol for the single biomarker IHC and multiplex IHC. In general, IHC was performed at 37° C., unless otherwise noted, and reaction buffer wash solutions were diluted from 10× concentrate (cat. no. 950-300). A slide-mounted paraffin section was de-paraffinized by warming the slide to 70° C. for 3 cycles, each 8 min long. Antigen retrieval was performed by applying Cell Conditioning 1 (VMSI Cat. no. 950-124) and warming the slide to 94° C. for 64 min. Staining of each biomarker was performed in sequential steps that included incubation with primary antibody targeting that biomarker for 16-32 min, washing in reaction buffer to remove unbound antibody, incubation for 8 min with anti-species antibody targeting the primary antibody (either anti-mouse or anti-rabbit) conjugated to either peroxidase or alkaline phosphatase, depending on whether the chromophore is a tyramide or quinone methide derivative, respectively, washing with reaction buffer, incubation with tyramide-modified DBCO or tyramide-modified chromogenic reagent or quinone-methide-precursor-modified chromogenic reagent, and washing with reaction buffer. For tyramides, dilute H₂O₂ was added following tyramide addition to initiate the deposition, and incubated for 32 min. All deposition steps were followed by washing in reaction buffer. If tyramide-modified DBCO was used, the slide was further incubated with azide-modified chromogen for 32 min, and washed. If multiplex IHC, before staining the next biomarker in sequence, the slide was incubated with Cell Conditioning 2 (VMSI Cat #950-123) at 100° C. for 8 min, followed by washing in reaction buffer. Finally, slides could be manually dehydrated through an ethanol series (2×80% ethanol, 1 min each, 2×90% ethanol, 1 min each, 3×100% ethanol, 1 min each, 3× xylene, 1 min each), at ambient temperature. Primary antibodies and enzyme-antibody conjugates were used at the concentrations, volumes, and incubation times recommended by the manufacturer. Tyramide-modified detectable conjugates (such as those described herein), tyramide-chromogen or tyramide-DBCO, were added to slides in 100 μL volumes at concentrations ranging between 25 and 1,200 μM in VMSI Discovery TSA diluent (cat no. 000060900). Azide-modified detectable conjugates were added as 100 μL volumes in TSA diluent typically at the same concentration as used for the tyramide-DBCO. The concentrations of solutions of the detectable conjugates (including the detectable moieties described herein) reflected their peak absorbance extinction coefficients, and biomarker expression levels, and were typically 1,200 μM for 7-amino-4-methylcoumarin-3-acetyl (AMCA), 400 μM for 7-hydroxycoumrin-3-carboxyl (HCCA), 600-800 μM for 7-diethylaminocoumarin-3-carboxyl (DCC), and 50-300 μM for Cy7 detectable moieties. Quinone-methide-precursor-modified Cy5 was added to slides in 100 μL of 400 μM Cy5 detectable moiety in TSA diluent. When using the modified dyes as fluorophores, the concentrations were reduced approximately 10-fold. Fluorescence IHC (immunofluorescence) slides were also mounted in ProLong Glass antifade mountant (Thermo Fisher Scientific, Waltham, MA).

Conventional Histological Staining

Staining with hematoxylin or eosin (or both) was performed after IHC based on the following H&E staining procedure. If the IHC specimen went through a final dehydration in xylene, then the specimen slide was re-hydrated by soaking in 100% ethanol for 1 min, 90% ethanol for 1 min, 80% ethanol for 1 min, and water for 1 min. Slides were then soaked in hematoxylin solution (Ventana HE 600 Hematoxylin; order code 07024282001) for 2 min, water for 2 min, define solution (Leica Surgipath SelectTech Define MX-aq, cat. no. 3803595) for 1 min, water for 1 min, bluing solution (VWR Bluing reagent; cat. no. 95057-852) for 1 min, water for 1 min, 95% ethanol for 30 s, eosin solution (Ventana HE 600 Eosin; order code 06544304001) for 1 min, 70% ethanol for 1 min, twice in 100% ethanol for 1 min each, and 3 times in xylene for 1 min each. Slides were then allowed to air dry and mounted with Richard Allan Scientific Cytoseal XYL (ThermoFisher Scientifc, Kalamazoo, MI) covering with a type 1.5 coverslip, or mounted on a Sakura FineteK USA (Torrance, CA) Tissue-Tek Film Automated Coverslipper. If only hematoxylin staining was desired, staining of a hydrated slide was performed through the bluing and water wash step, incubating in hematoxylin solution several seconds to 2 min to achieve the desired depth of staining, and then skipping to dehydration through an ethanol/xylene series (2×80% ethanol, 1 min each, 2×90% ethanol, 1 min each, 3×100% ethanol, 1 min each, 3× xylene, 1 min each), at ambient temperature, and coverslipping. If only eosin staining was desired, staining of a hydrated slide was started at the 95% ethanol step and carried through completion, incubating in eosin solution several seconds to 1 min to achieve the desired depth of staining.

Microscopy and Single-Camera Monochrome Imaging of Conventional Histological Staining and Covalently Deposited Chromophore (CDC) Staining

Multispectral imaging of stained specimens was performed on an Olympus BX-51 microscope (Olympus, Waltham, MA) fitted with a CoolSNAP ES2 CCD camera with a 1392×1040 pixel sensor at 12-bit resolution (Teledyne Photometrics, Tucson, AZ) and LED illumination, as previously described [Morrison L E, Lefever M R, Behman U, Leibold T, Roberts E A, Horchner U B, Bauer D R. Brightfield Multiplex Immunohistochemistry with Multispectral Imaging. Lab Invest (2020) https://doi.org/10.1038/s41374-020-0429-0]. Microscope objectives were initially Olympus UPlanSApo 20× (NA 0.75) and 10× (NA 0.40) air objectives but were later updated with UPLXAPO 20× (NA 0.80) and UPLXAPO 10× (NA 0.4) objectives with improved chromatic aberration correction. Illumination was provided by a combination of optically filtered continuous light sources and LED illuminators. For the former, a Sutter Lambda 10-3 10-position filter wheel (Sutter Instruments, Novato, CA) was used with an Olympus 100 W tungsten halogen lamp to define up to nine wavelength channels. LED illumination was provided with a CoolLED (Andover, UK) pE-4000 16-channel illuminator and 2 Lumencor Spectra X light engines (Lumencor, Inc., Beaverton, OR), each containing 6 custom-selected LEDs. Illuminator outputs were focused onto 3 mm liquid light guides and the light guides combined into a single 3 mm diameter liquid light guide with one or two Lumencor combiners. The final light guide was connected to the illumination port of the microscope through a CoolLED pE collimator/microscope adapter. To reduce the illumination bandwidth further, each Lumencor LED was filtered with a single bandpass optical filter. Filter selection on the filter wheel and LED selection was achieved using manual controls with the option of computer control. Imaging of individual microscope fields on the CCD camera of light transmitted through the microscope was controlled by Micromanager software [Edelstein A D, Tsuchida M A, Amodaj N, Pinkard H, Wale R D, Stuuman N. Advanced methods of microscope control using pManager software. J Biol Methods 2014; 1:e10]. Image processing, including conversion between transmission and absorbance images and formation of color composite images, was performed with ImageJ software [Schneider C A, Rasband W S, Eliceiri K W. NIH Image to ImageJ: 25 years of image analysis. Nat Methods 2012; 9: 671-675].

Typically, a multi-color specimen was imaged with multiple filters on the filter wheel and/or LED, where each filter and/or LED provided a band of light at wavelengths near the maximum absorbance of one of the dyes used to the stain the specimen (e.g. eosin and HTX or other conventional stains applied to the specimen, plus each CDC. The number of different light channels utilized for imaging a multiplex IHC equaled at least the number of dyes (chromogens plus conventional stain components). For calculation of transmission and absorbance images, images were recorded using each light channel on an unstained region of the slide (e.g. to the side of the tissue/cellular specimen) before and/or after recording images with the same series of light channels at the desired region of interest within the stained specimen. Dividing transmitted light images of stained regions by images of unstained regions (100% transmission) provides transmission (T) images. Logarithmic conversion provides absorbance (A) images (A=−log 10T) for which A is proportion to dye concentration according to Beer's Law. Color composite images are produced by addition of the monochrome A-images, with appropriate weighting for the desired pseudo-coloring, to form red, green, and blue color planes of the composite image. These composite images provide a “fluorescence-like” representation, and can be converted to brightfield representations by anti-logarithmic conversion of A-images to T-images.

Microscopy and Dual-Camera Color/Monochrome Imaging of Conventional Histological Staining and Covalently Deposited Chromophore (CDC) Staining

Simultaneous 2-camera video imaging was achieved with a Kiralux 5 Megapixel color CMOS camera and a Kiralux 5 Megapixel monochrome CMOS camera (Thorlabs Inc., Newton, NJ) attached to a dual-camera mount for upright microscopes (Thorlabs; cat. no. 2SCM1-DC), attached to the camera port of an Olympus BX-63 microscope. Brightfield illumination was provided with an Olympus 100 W tungsten halogen lamp, with output limited to the visible spectrum using a hot mirror transmitting light between 420 nm and 690 nm (Newport Corp., Irvine, CA; cat. no. 20HMS-0), combined with the output of a CoolLED pE-4000 16 LED illuminator. Light from the tungsten lamp and LEDs matching the absorbance of the CDCs were combined using a CoolLED pE-Combiner and 50-50 neutral density beamsplitter (ChromaTechnology, Bellows Falls, VT). Alternatively, while light can be generated from several of the visible light LEDs in the CoolLED pE-4000 illuminator, without the need for the combiner. Light from the microscope objective was divided into two paths with a 50-50 beamsplitter, within the dual-camera mount, and directed to the color and monochrome cameras. When simultaneous viewing of visible white light and invisible CDC light was desired, light directed to the monochrome camera was filtered through a Newport FSR-UG5 colored glass filter, transmitting light below 400 nm and above 690 nm, and light directed to the color camera was filtered with a 420 nm long pass CGA-420 colored glass filter (Newport Corp., Irvine, CA) in addition to the camera's integrated visible light transmission filter. Complementary filtering was designed to exclude light near the borders of and outside the visible spectral range from the color camera and exclude most of the visible spectrum from the monochrome camera, ensuring that the tungsten illumination, where conventional stains or visible CDCs absorb, was only detected by the color camera, and illumination bands where invisible CDCs absorb were only detected by the monochrome camera. Video rate image acquisition and video rate overlay of the two camera images were achieved using ThorCam software (Thorlabs).

The dual-camera color/monochrome imaging system could also be illuminated as described for the single-camera monochrome imaging system with any combination of Lumencor illuminators, CoolLED illuminators, and/or continuous light sources plus bandpass filtering. Complementary filtering can be removed to permit either camera to receive any wavelength illumination. In such configurations the monochrome camera of the dual-camera system was employed as the monochrome camera in the single-camera monochrome imaging system for multi-spectral imaging.

Fluorescence Microscopy

Fluorescence images were recorded using the monochrome camera of the dual-camera system and employing the fluorescence optical paths of the BX-63 microscope equipped with an Olympus 75 W xenon lamp for fluorescence excitation. The Chroma Technology single bandpass filter sets ET-Cy7 (cat no. 49007), ET-Spectrum Orange (cat no. 49305), and ET-DAPI (cat no. 49000) were used to image Cy7, TAMRA, and DAPI fluorescence, respectively.

On-Slide Absorbance Measurements

Absorbance spectra of covalently deposited chromophores (CDCs) and conventional stains were recorded on slide-mounted specimens placed on the stage of an Olympus BX-63 microscope under illumination with Olympus 100 W tungsten halogen or 75 W xenon microscope lamps. Transmitted light was measured between 350 and 800 nm in approximately 0.5 nm increments using a Pryor Scientific Inc. (Rockland, MA) Lumaspec 800 power meter. The power meter was upgraded with an Ocean HDX UV to NIR spectrometer that permitted spectral measurements between 200 and 1100 nm. The spectrum of light transmitted through a stained region of the slide was divided by the spectrum transmitted through an unstained (no tissue) region to provide the transmission (T) spectrum, which was converted to the chromogen absorbance (A) spectrum using the relationship A=log 10(1/T).

### EXAMPLES

### Example 1

Introduction

The conventional bright-field nuclear counterstain, hematoxylin (HTX), provides a valuable measure of cellular and tissue context that aids in the interpretation of immunohistochemical (IHC) and in situ hybridization (ISH) staining of biomarkers and nucleic acid sequences. However, as for other conventional stains used in bright-field microscopy, the absorbance spectrum of HTX is quite broad, as show in FIG. 10 where the absorbance spectra of HTX and common bright-field chromogens are plotted. While HTX serves as an effective counterstain when used in combination with one or two chromogens, the broad spectra of the conventional chromogens complicate visual evaluation of higher order multiplexing due to broad regions of spectral overlap between any two dyes plotted in FIG. 10. The detectable moieties described herein have permitted the rapid development of chromogens with narrower absorbance bands, facilitating higher order bright-field multiplexing. Absorbance spectra of five detectable moieties are illustrated in FIG. 11. The reduced spectral overlap between the narrow band detectable moieties provides improved visual distinction of the stained biomarkers, particularly after imaging with a monochrome camera and spectrally unmixing the different chromogen absorbances. However, counterstaining is still required and, unfortunately, the popular conventional HTX counterstain is still utilized. The HTX absorbance spectrum is also included in FIG. 11, which serves to emphasize the broad nature of HTX absorbance, and the consequent problem with spectral overlap between HTX and all of the multiplexed narrow band chromogens. Even though the Detectable moieties have narrower absorbance bands, the HTX overlap still provides challenges to visual discrimination and spectral unmixing. To reduce this problem, the HTX staining level is typically lowered, which often makes detection of the counterstain difficult while still contributing some level of spectral crosstalk between HTX and the multiplexed chromogens.

The problem of broad counterstain absorbance has been addressed by developing counterstains based on IHC using primary antibodies targeting morphological nuclear components. Examples of morphological nuclear components are double-stranded DNA (ds DNA) and histones. DNA is the primary target of hematoxylin binding, and histones are proteins that associate with DNA to ultimately form nucleosomes and chromatin within the nucleus, so nuclear staining similar to HTX should be achieved with IHC targeting ds-DNA or histones. Detectable moieties with narrow absorbance bands (including any of those described herein), directed by the anti-ds DNA or anti-histone antibodies, have been used to achieve a counterstain that greatly reduces the amount of spectral crosstalk with other chromogens, thereby improving visual discrimination through the microscope and/or visualization and quantification via imaging and spectral unmixing.

Materials and Methods

Primary antibodies anti-ds DNA [DSD/958] (ab215896) and anti-histone H3 (ab1791) were obtained from ABCAM (Cambridge MA).

Results and Discussion

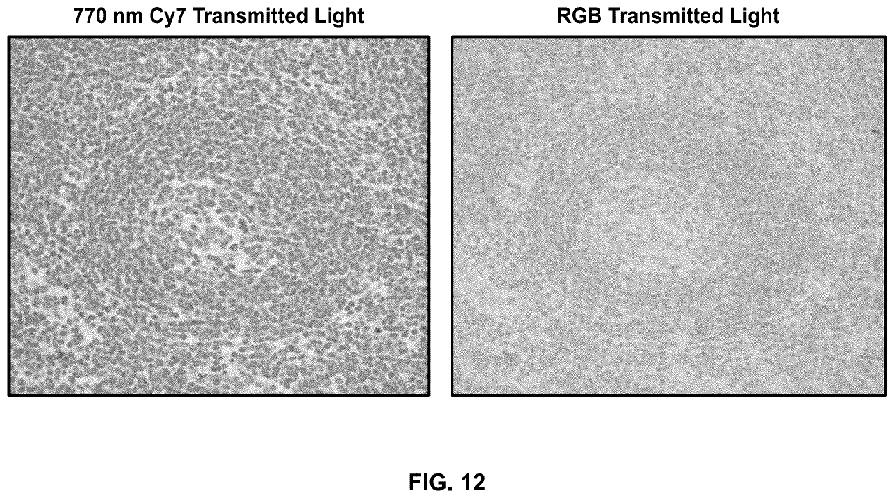

Anti-ds DNA and anti-histone H3 antibodies were tested in IHC with HTX counterstaining to determine if IHC targeting nuclear components could provide staining similar to HTX. The IHC employed a Cy7 CDC with primary absorbance in the invisible far red/near IR portion of the spectrum. The Cy7 absorbance maximum at 770 nm was well separated from the HTX absorbance in the red portion of the spectrum (absorbance maximum 618 nm) so that staining of each antibody and HTX could be separately evaluated. FIG. 12 shows brightfield microscope images of a formalin-fixed paraffin-embedded (FFPE) tonsil tissue specimen, stained using anti-ds DNA IHC and HTX. The images were recorded at 20× magnification using a monochrome CMOS camera with illumination from a 770 nm light emitting diode (LED) on the left side of FIG. 12 and a color (RGB) CMOS camera with white light illumination from a tungsten halogen lamp on the right side.

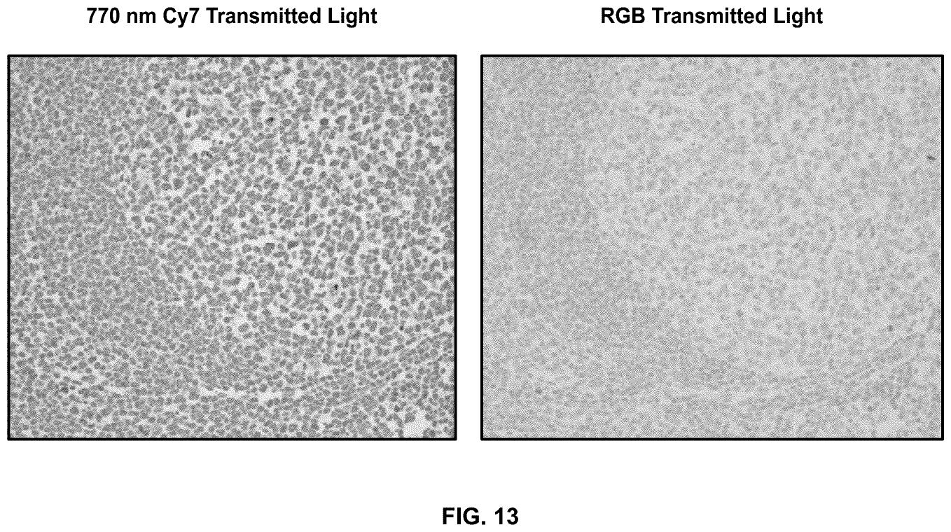

A two-camera system was used for imaging and allowed simultaneous imaging with monochrome and color cameras. Since each camera used the same CMOS sensor, except for Bayer filtering on the color camera, the cameras could be aligned, thereby permitting overlays of the two images at video display rates. The color camera faithfully reproduced what was observed visually through the microscope ocular, and only reflected the HTX stain, while the monochrome camera was filtered to record the invisible 770 nm light where only the Cy7 CDC stain absorbed. Visual evaluation of the two images, as displayed in FIG. 12, demonstrated that both the HTX counterstain and the anti-ds DNA IHC stained the nuclei of all cells similarly. Results of the dual staining of FFPE tonsil tissue using the anti-histone antibody are displayed in FIG. 13. As observed for the anti-ds DNA antibody, the anti-histone antibody stained all nuclei, reproducing the general staining pattern of the conventional HTX counterstain.

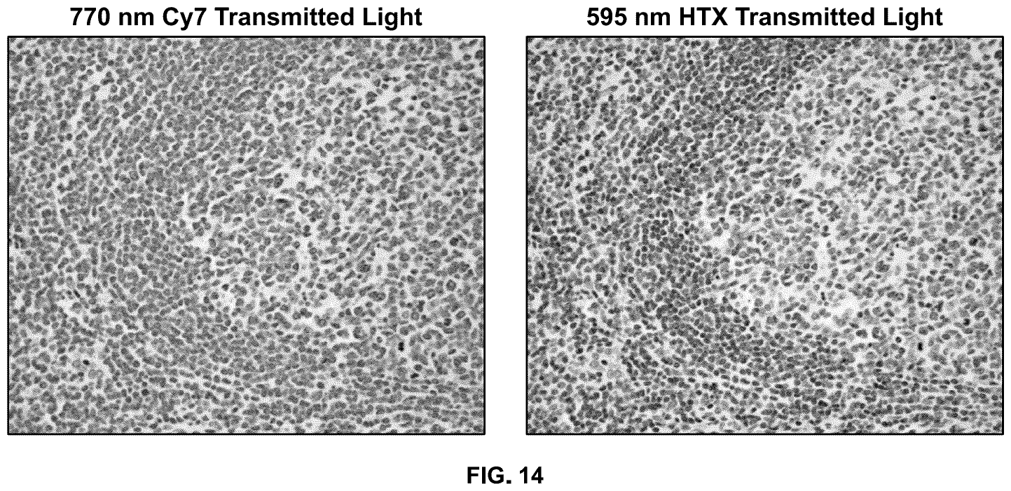

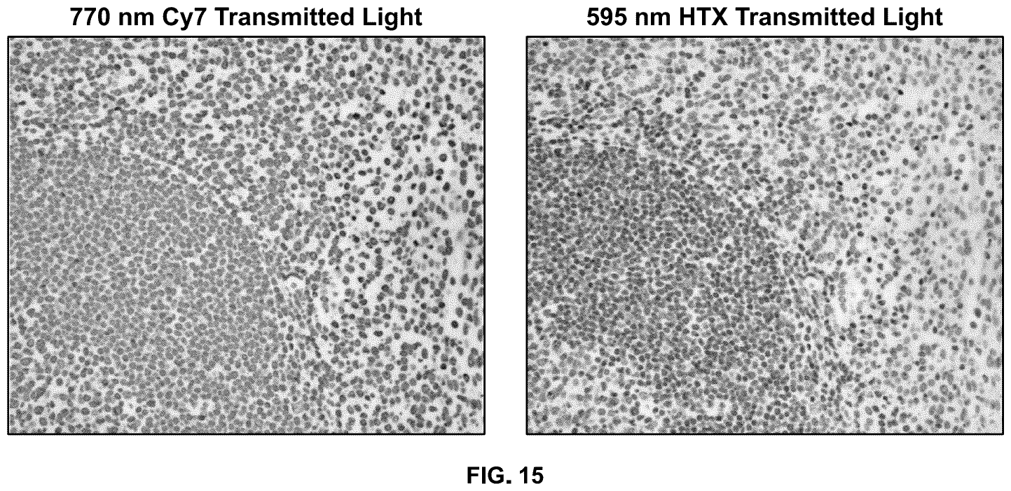

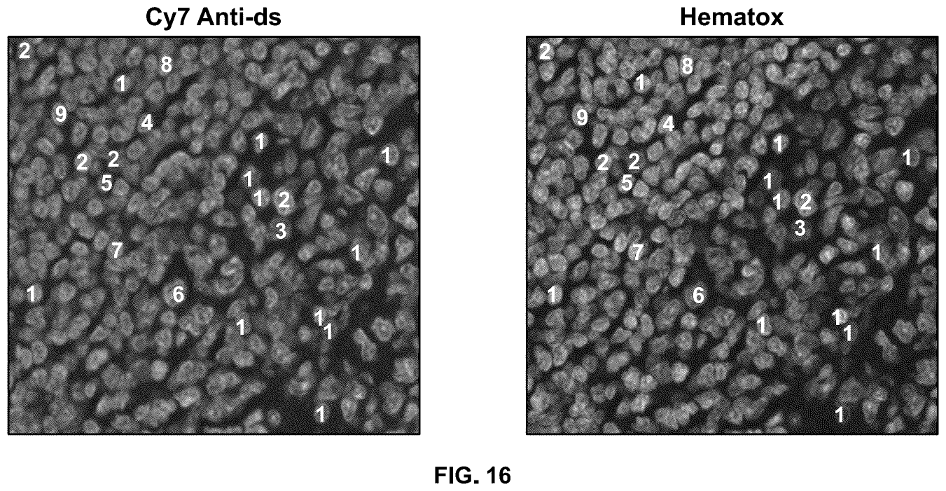

For a closer comparison of the antibody/Cy7 staining with the HTX staining, monochrome images of each were recorded using 770 nm LED illumination for Cy7 and 595 nm LED illumination for HTX. FIG. 14 shows two monochrome images of a 20× magnification field for the anti-ds DNA IHC/HTX stained FFPE tonsil slide; and FIG. 15 shows two monochrome images of a 20× magnification field for the anti-histone IHC/HTX stained FFPE tonsil slide. As shown in FIGS. 15 and 16, staining patterns for antibody and HTX appeared similar, but the antibody staining for the two antibodies appeared to provide a more uniform level of nuclear staining across the fields. Since the counterstain purpose was to identify all cell nuclei without respect to cell type, uniform staining was a desirable property providing an unexpected advantage of the IHC-based counterstain.

### Example 2

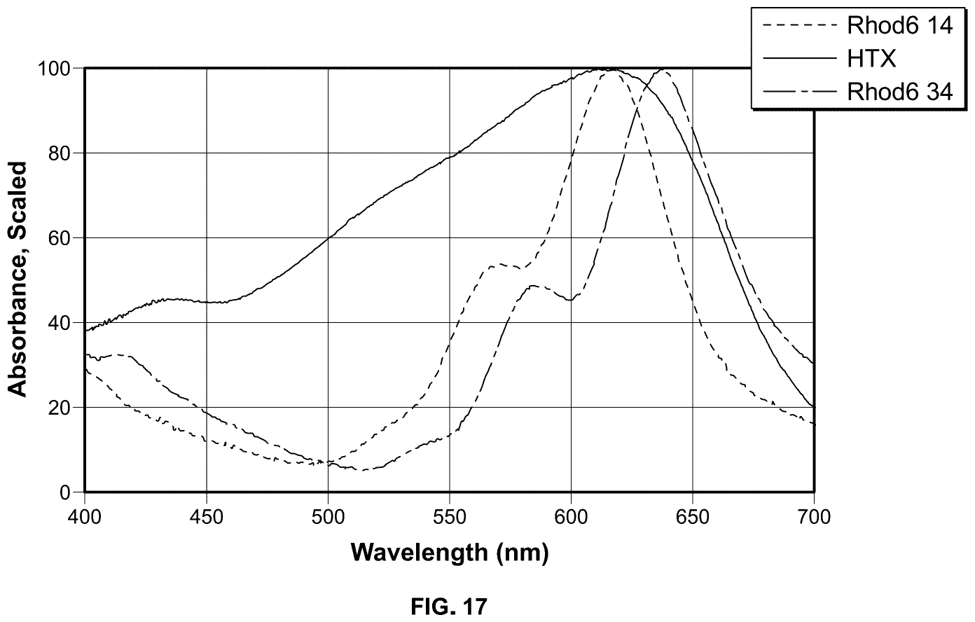

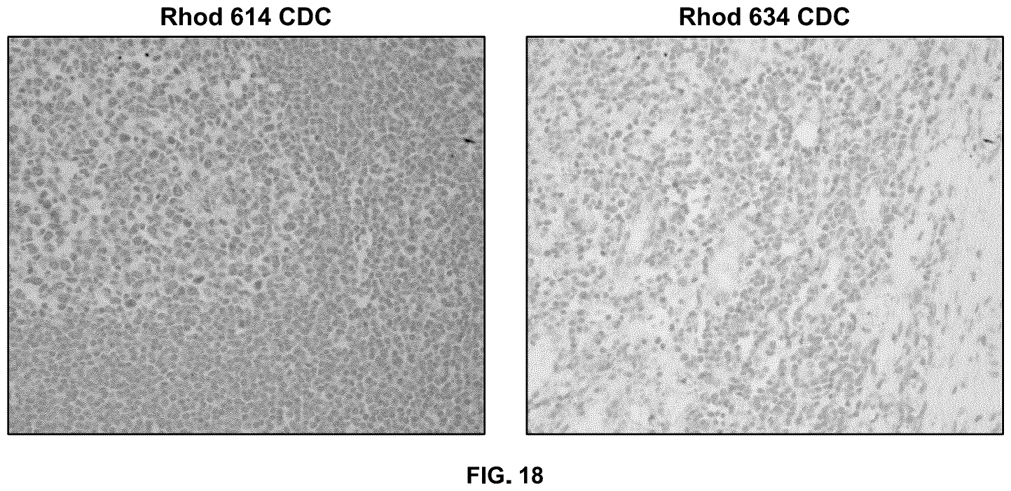

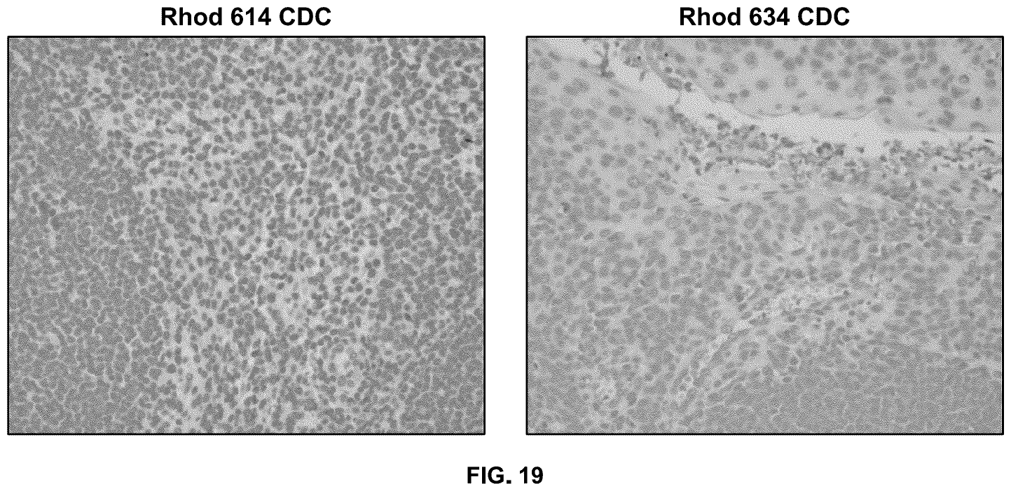

The examples in FIGS. 12 to 15 used an invisible near-IR absorbing chromogen C7, that permitted the comparison of HTX and IHC-based counterstaining. Detectable moieties could also have been designed for color similar to HTX to serve as a direct replacement in existing assays or simply provide the expected HTX coloration. Absorbance spectra of two HTX replacement detectable moieties are plotted in FIG. 17 with the HTX absorbance spectrum. The Rhod614 detectable moiety had an absorbance maximum which closely matched HTX and the Rhod634 detectable moiety had a red-shifted absorbance maximum. Also noted were the much narrower absorbance bands of both replacement detectable moieties compared to HTX. FIGS. 18 and 19 show color images of Rhod614 (left panels) and Rhod634 (right panels) of the detectable moieties used in IHC with anti-ds DNA and anti-histone, respectively, on FFPE tonsil. Comparison with color images in FIGS. 12 and 13 (right panels) show the color similarity between HTX and these detectable moieties.

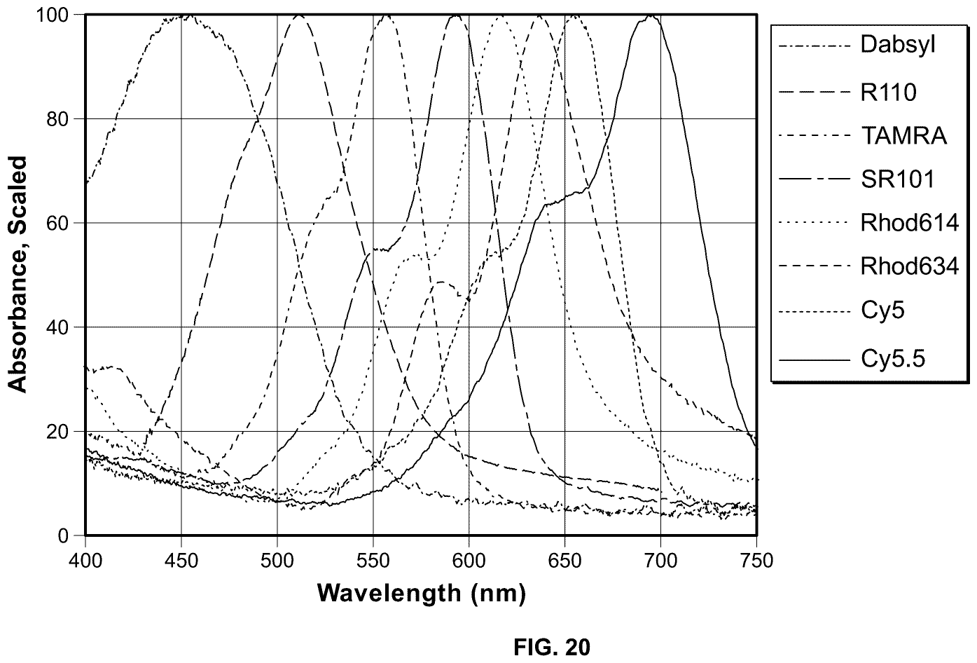

FIG. 20 illustrates absorbance spectra of the same five detectable moieties, previously used in multiplex IHC and shown in FIG. 11, with the spectra of the Rhod614 and Rhod634 IHC counterstains, showing considerably reduced spectral crosstalk between detectable moieties and counterstains. Replacing HTX with Rhod614 would have been expected to improve visualization and spectral unmixing of a multiplex using Dabsyl, Rh110, TAMRA, SRhod101, and Cy5 Detectable moieties. Alternatively, the red-shifted Rhod634 counterstain could have been employed and Cy5 CDC replaced with Cy5.5 to separate the chromogen absorbance bands more and further reduce spectral crosstalk. The absorbance spectrum of Cy5.5 CDC is also included in FIG. 20 to further illustrate this option.

As shown in Tables 13A and 13B below, which lists parameters for comparing HTX staining consistency with counterstaining according to the disclosed methods within a given cell (of FIG. 16), counterstaining according to the disclosed methods is actually more consistent in most instances than with hematoxylin.

TABLE 13A
Cy7 anti-DNA image data

| | | | | | | | within cell | | |
|---|---|---|---|---|---|---|---|---|---|
| | Area | | | | | | relative | within cell | (max- |
| cell label | (pixels) | Mean | StdDev | Min | Max | Median | Std Dev | max-min | min)/mean |
| 1 | 611 | 0.2022 | 0.0520 | 0.0600 | 0.3047 | 0.2157 | 0.2573 | 0.2447 | 1.2099 |
| 2 | 1424 | 0.2423 | 0.0461 | 0.0932 | 0.3769 | 0.2417 | 0.1901 | 0.2837 | 1.1708 |
| 3 | 1084 | 0.1503 | 0.0337 | 0.0745 | 0.2539 | 0.1454 | 0.2243 | 0.1793 | 1.1934 |
| 4 | 558 | 0.2232 | 0.0274 | 0.1430 | 0.2864 | 0.2230 | 0.1230 | 0.1435 | 0.6428 |
| 5 | 472 | 0.2514 | 0.0452 | 0.0910 | 0.3429 | 0.2571 | 0.1798 | 0.2519 | 1.0019 |
| 6 | 1348 | 0.2013 | 0.0461 | 0.0614 | 0.3660 | 0.1999 | 0.2289 | 0.3046 | 1.5131 |
| 7 | 512 | 0.2373 | 0.0270 | 0.1771 | 0.3247 | 0.2330 | 0.1138 | 0.1476 | 0.6219 |
| 8 | 708 | 0.2014 | 0.0347 | 0.1086 | 0.3021 | 0.2037 | 0.1720 | 0.1934 | 0.9603 |
| 9 | 760 | 0.2308 | 0.0348 | 0.1271 | 0.3264 | 0.2313 | 0.1508 | 0.1993 | 0.8635 |
| 10 | 638 | 0.2336 | 0.0385 | 0.0806 | 0.3167 | 0.2347 | 0.1649 | 0.2361 | 1.0108 |
| 11 | 1098 | 0.2044 | 0.0418 | 0.1117 | 0.3139 | 0.2047 | 0.2045 | 0.2022 | 0.9890 |
| 12 | 434 | 0.2492 | 0.0378 | 0.1518 | 0.3373 | 0.2518 | 0.1518 | 0.1855 | 0.7444 |
| 13 | 611 | 0.1649 | 0.0229 | 0.1090 | 0.2360 | 0.1622 | 0.1390 | 0.1270 | 0.7706 |
| 14 | 1085 | 0.2031 | 0.0436 | 0.0869 | 0.3248 | 0.1987 | 0.2149 | 0.2379 | 1.1715 |
| 15 | 832 | 0.1665 | 0.0360 | 0.0489 | 0.2707 | 0.1655 | 0.2164 | 0.2218 | 1.3318 |
| 16 | 774 | 0.1869 | 0.0334 | 0.0964 | 0.2798 | 0.1882 | 0.1789 | 0.1834 | 0.9813 |
| 17 | 1008 | 0.1601 | 0.0434 | 0.0747 | 0.2916 | 0.1596 | 0.2711 | 0.2168 | 1.3541 |
| 18 | 860 | 0.2323 | 0.0446 | 0.1102 | 0.3808 | 0.2259 | 0.1919 | 0.2706 | 1.1651 |
| 19 | 611 | 0.2221 | 0.0282 | 0.1259 | 0.2853 | 0.2232 | 0.1269 | 0.1594 | 0.7176 |
| 20 | 708 | 0.1886 | 0.0246 | 0.1222 | 0.2687 | 0.1892 | 0.1305 | 0.1465 | 0.7766 |
| 21 | 1162 | 0.1255 | 0.0343 | 0.0530 | 0.2125 | 0.1204 | 0.2736 | 0.1595 | 1.2708 |
| 22 | 861 | 0.2479 | 0.0366 | 0.1013 | 0.3380 | 0.2497 | 0.1478 | 0.2367 | 0.9551 |
| mean | | 0.2057 | | 0.1004 | 0.3064 | 0.2057 | 0.1842 | 0.2060 | 1.0189 |
| std | | 0.0354 | | 0.0328 | 0.0434 | 0.0369 | 0.0478 | 0.0488 | 0.2462 |

| | | | | | |
|---|---|---|---|---|---|
| relative StdDev | 0.1719 | 0.3272 | 0.1417 | 0.1793 | 0.2370 |

TABLE 13B

| | Area | | | | | | within cell | within cell | (max- |
|---|---|---|---|---|---|---|---|---|---|
| cell label | (pixels) | Mean | StdDev | Min | Max | Median | relative StdDev | max-min | min)/mean |
| 1 | 611 | 0.2781 | 0.0720 | 0.0414 | 0.4462 | 0.2835 | 0.2590 | 0.4048 | 1.4556 |
| 2 | 1424 | 0.2488 | 0.0513 | 0.0372 | 0.3698 | 0.2525 | 0.2062 | 0.3326 | 1.3368 |
| 3 | 1084 | 0.1149 | 0.0307 | 0.0498 | 0.2102 | 0.1101 | 0.2672 | 0.1603 | 1.3955 |
| 4 | 558 | 0.2986 | 0.0478 | 0.1545 | 0.4026 | 0.2981 | 0.1602 | 0.2482 | 0.8310 |
| 5 | 472 | 0.3228 | 0.0597 | 0.1231 | 0.4600 | 0.3334 | 0.1849 | 0.3369 | 1.0437 |
| 6 | 1348 | 0.1823 | 0.0458 | 0.0505 | 0.3007 | 0.1810 | 0.2511 | 0.2502 | 1.3727 |
| 7 | 512 | 0.3282 | 0.0424 | 0.1307 | 0.4163 | 0.3315 | 0.1292 | 0.2856 | 0.8704 |
| 8 | 708 | 0.2762 | 0.0494 | 0.1202 | 0.4269 | 0.2752 | 0.1789 | 0.3067 | 1.1105 |
| 9 | 760 | 0.2606 | 0.0467 | 0.1377 | 0.3998 | 0.2613 | 0.1793 | 0.2621 | 1.0058 |
| 10 | 638 | 0.2874 | 0.0678 | 0.0537 | 0.4096 | 0.2963 | 0.2358 | 0.3559 | 1.2382 |
| 11 | 1098 | 0.1795 | 0.0457 | 0.0581 | 0.2856 | 0.1843 | 0.2548 | 0.2274 | 1.2671 |
| 12 | 434 | 0.3358 | 0.0662 | 0.1969 | 0.4936 | 0.3368 | 0.1973 | 0.2966 | 0.8833 |
| 13 | 611 | 0.1453 | 0.0270 | 0.0857 | 0.2240 | 0.1426 | 0.1857 | 0.1383 | 0.9520 |
| 14 | 1085 | 0.1528 | 0.0408 | 0.0487 | 0.2881 | 0.1493 | 0.2670 | 0.2395 | 1.5671 |
| 15 | 832 | 0.1012 | 0.0323 | 0.0219 | 0.2071 | 0.0990 | 0.3191 | 0.1853 | 1.8307 |
| 16 | 774 | 0.2749 | 0.0576 | 0.1133 | 0.4181 | 0.2776 | 0.2096 | 0.3048 | 1.1087 |
| 17 | 1008 | 0.1233 | 0.0265 | 0.0566 | 0.2059 | 0.1222 | 0.2152 | 0.1493 | 1.2110 |
| 18 | 860 | 0.2046 | 0.0405 | 0.0950 | 0.3162 | 0.1995 | 0.1978 | 0.2211 | 1.0807 |
| 19 | 611 | 0.2950 | 0.0520 | 0.1560 | 0.4148 | 0.2889 | 0.1762 | 0.2589 | 0.8775 |
| 20 | 708 | 0.2841 | 0.0544 | 0.1164 | 0.4298 | 0.2818 | 0.1916 | 0.3135 | 1.1033 |
| 21 | 1162 | 0.1611 | 0.0433 | 0.0387 | 0.2848 | 0.1593 | 0.2690 | 0.2461 | 1.5277 |
| 22 | 861 | 0.2694 | 0.0605 | 0.0529 | 0.4117 | 0.2729 | 0.2246 | 0.3588 | 1.3322 |
| mean | | 0.2329 | | 0.0881 | 0.3555 | 0.2335 | 0.2163 | 0.2674 | 1.2001 |
| std | | 0.0751 | | 0.0484 | 0.0906 | 0.0773 | 0.0447 | 0.0707 | 0.2608 |

| | | | | | |
|---|---|---|---|---|---|
| relative Std Dev | 0.3222 | 0.5492 | 0.2547 | 0.3312 | 0.2644 |

### Example 3—Multiplex IHC with Counterstain

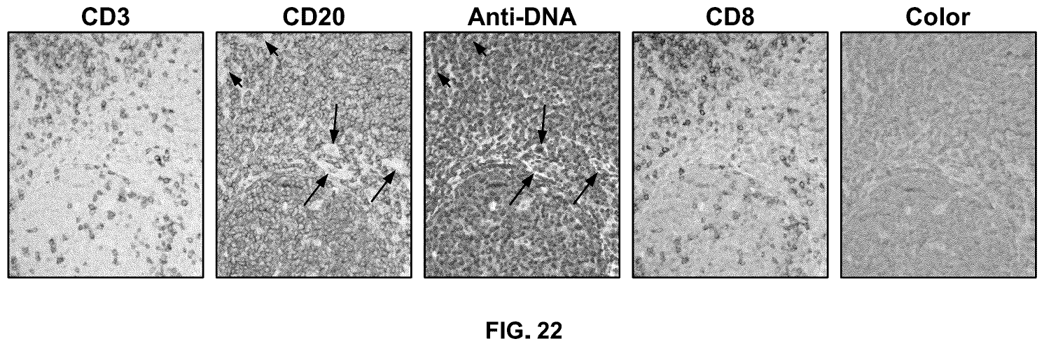

Multiplex IHC with either the anti-ds DNA counterstain or the hematoxylin counterstain were performed to compare counterstain performance, and especially to examine the effect of counterstain absorbance on interpretation of the multiplexed biomarker staining. Multiplex IHC to stain CD3, using the dabsyl CDC, CD20, using the TAMRA CDC, CD8, using the Cy5.5 CDC, and ds DNA, using the Rhod634, was performed on FFPE tonsil tissue. Images of transmitted light using the 438, 549, 620, and 689 nm filtered LEDs are presented in the first four images of FIG. 22, from left to right, respectively, recorded on a monochrome camera (dual-camera system). These illumination channels were selected to align near the absorbance maxima of dabsyl, TAMRA, Rhod634, and Cy5.5, respectively. The fifth image is of the same microscope field using white light illumination recorded on a color camera (dual-camera system). Notice in the CD3, CD20, and CD8 images the clear staining of the membranes where these three biomarkers reside—primarily t-cells for CD3 and CD8, and 1B-cells for CD20. The center nuclear regions of these membrane-stained cells are also light in color indicating little-to-no detectable absorbance due to the nuclear counterstain, Rhod634. Also, note the clear regions in the CD20 TAMRA image marked by arrows in this image and the Rhod634 counterstain image, which shows that although there are nuclei of CD20 negative cells in these regions, they do not show in the CD20 image, therefore not leading to misinterpretation of which cells are CD20 positive. The good separation of biomarker staining from counterstain is a result of the minimal overlap between Rhod634 counterstain absorbance and biomarker absorbance at the illumination wavelengths, as can be seen from the spectra plotted in FIG. 20.

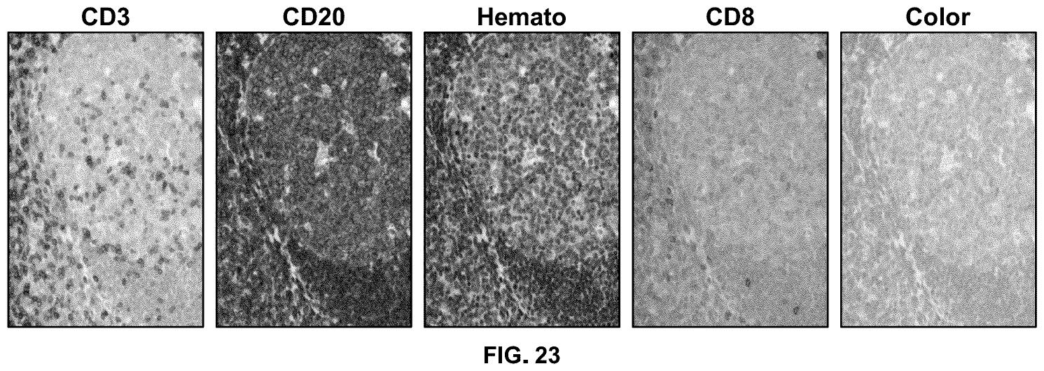

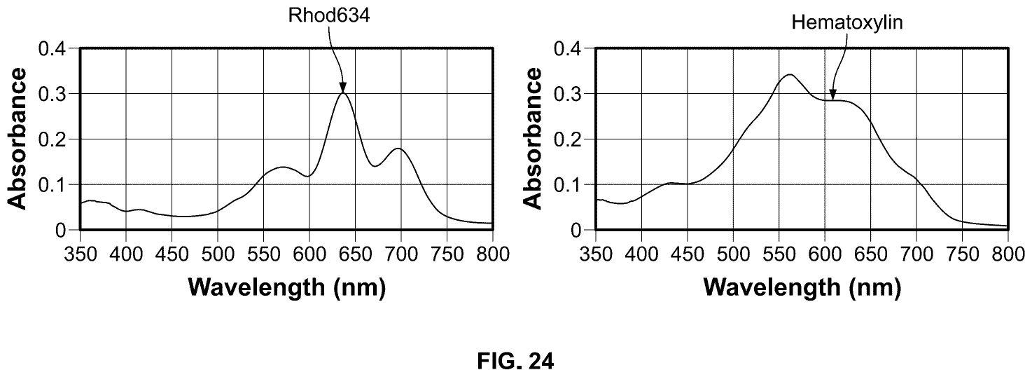

Multiplex IHC was also performed to stain the same three biomarkers with the same chromogens on another section of the same FFPE tonsil specimen. This section was stained with the conventional hematoxylin counterstain in place of the anti-ds DNA counterstain. Images of transmitted light using the 438, 549, 620, and 689 nm filtered LEDs are presented in the first four images of FIG. 23, from left to right, respectively. These illumination channels were selected to align near the absorbance maxima of dabsyl, TAMRA, hematoxylin, and Cy5.5, respectively. The fifth image is of the same microscope field using white light illumination recorded on a color camera (dual-camera system). Hematoxylin staining time (5 s) was selected to provide similar counterstain absorbance of both hematoxylin and Rhod634 as shown by the absorbance spectra of these two sections in FIG. 24. Notice in the CD3 and CD8 images there is distinct staining of the membranes but the nuclear regions are slightly darker than when the Rhod634 anti-ds DNA counterstain was used, indicating a small but noticeable level of hematoxylin absorbance at the dabsyl and Cy5.5 illumination wavelengths. Considerably more hematoxylin absorbance is evident at the TAMRA illumination wavelengths, to the extent that the characteristic CD20 membrane staining could be misinterpreted as staining of the entire cell, or that CD20 negative cells could be interpreted as CD20 positive. This high level of hematoxylin absorbance in the TAMRA illumination channel results from the broad spectral absorbance of hematoxylin as evidenced in the absorbance spectra plotted in FIG. 11. To reduce this problem, considerably lower levels of hematoxylin staining are used typically to counterstain IHC specimens. However, this makes identification of all cells in a specimen difficult to distinguish due to faint staining, thereby reducing the ability to interpret the general tissue morphology, as well as identify biomarker negative cells. Employing the IHC-based nuclear counterstain allows a choice of counterstain spectral characteristics, and in the case of Rhod634, provides narrow-band absorbance that negligibly interferes with visualization and interpretation of the biomarker chromogen staining.

### Example 4—Cytoplasmic Counterstain

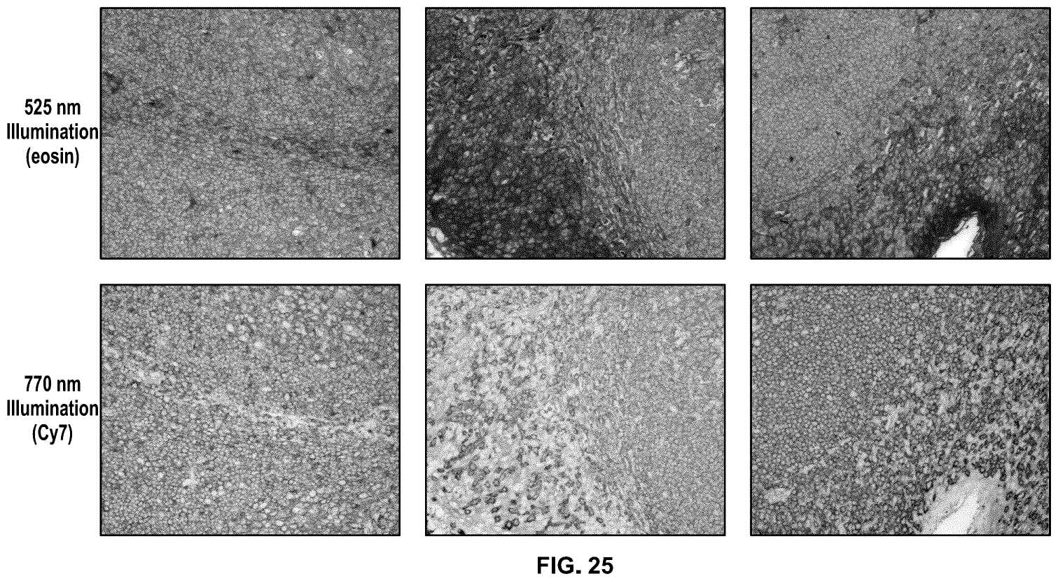

A ubiquitous cytoplasmic counterstain may be preferable to a nuclear counterstain when looking at biomarkers expressed at low levels within the nucleus. Actin is a highly conserved protein present in essentially all eukaryotic cells, with beta-actin (ACTB) being one of two cytoplasmic forms (Gunning, P W, Ghoshdastider, U, Whitaker, S, Popp, D and Robinson, R C. The evolution of compositionally and functionally distinct actin filaments. J. Cell. Sci. (2015) 128:2009-2019). As such, IHC staining based on anti-ACTB should provide a good cytoplasmic counterstain. To demonstrate this, anti-actin IHC using the Cy7 CDC was performed on FFPE tonsil tissue followed by eosin staining. Eosin is well separated spectrally from Cy7 and stains cytoplasm in addition to connective tissue. FIG. 25 shows images of three different microscope fields from left to right, with the top images recorded under 525 nm LED illumination, where eosin absorbs light, and the corresponding lower images recorded under 770 nm LED illumination, where Cy7 absorbs light, reflecting the presence of actin. In comparing the upper and lower images, notice that both the eosin and actin staining delineate the cytoplasm of the individual cells. Also notice that eosin also darkly stains other regions that are not cellular. In light of this, the anti-actin IHC provides a better cellular counterstain in that it essentially identifies the cytoplasmic regions of all cells without the interference from connective tissue and other proteinaceous regions to which eosin binds.

### Example 5—Fluorescent Counterstain

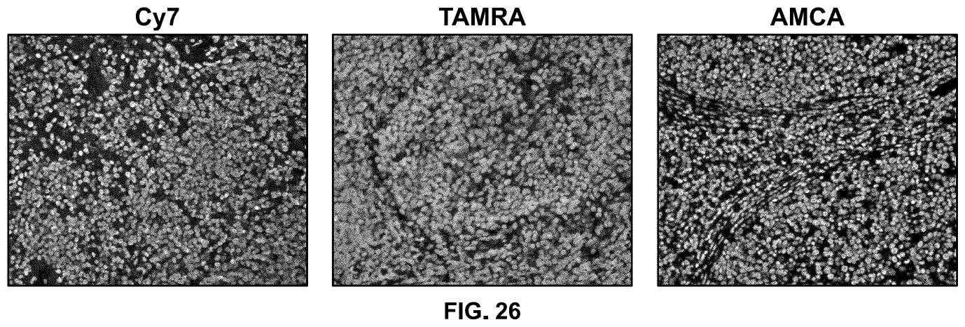

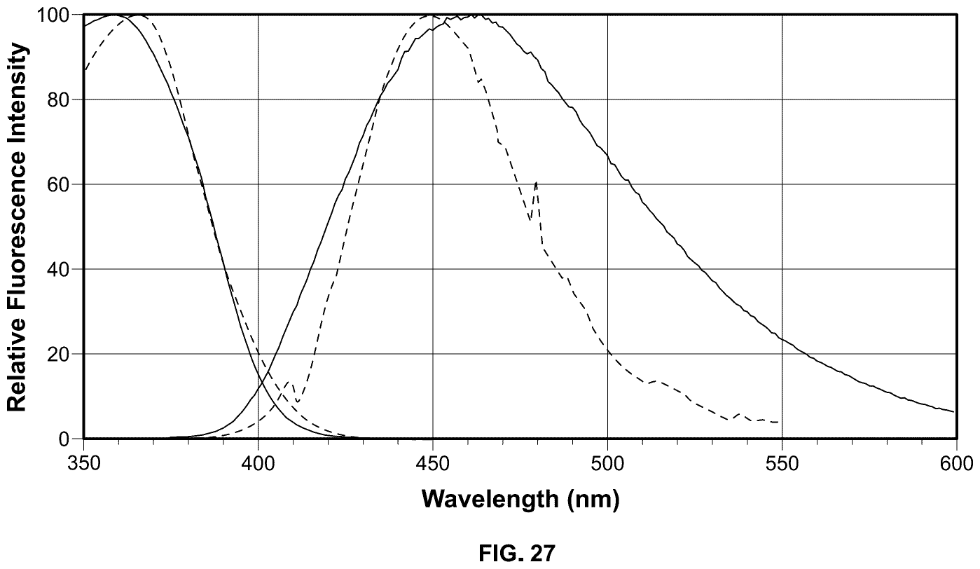

Fluorescent counterstains are important in immunofluorescence (IF) and fluorescence in situ hybridization (FISH). DAPI is widely used and is likely the most common counterstain for in situ assays, binding to DNA and staining nuclei with blue fluorescence. We examined fluorescent CDCs (fCDCs) for this same purpose, providing a choice of fluorescent colors across the spectrum, and providing a convenient route to non-nuclear counterstaining. In fact, the majority CDCs plotted in FIGS. 11 and 20 are highly fluorescent when deposited at lower concentrations than those used for chromogenic staining. FIG. 26 shows monochrome fluorescence images recorded on FFPE tonsil tissue stained with anti-ds DNA IHC using Cy7 CDC, TAMRA CDC, and AMCA CDC at 1/10 the typical chromogen concentrations. All three fCDCs provide bright and distinct staining ranging from the far red (Cy7) to the far blue (AMCA) ends of the spectrum. AMCA is particularly interesting in that it is excited and fluoresces at wavelengths similar to the common DAPI counterstain, while providing a narrower emission spectrum than DAPI. This is demonstrated in FIG. 27, which plots the excitation and emission spectra of DAPI and AMCA. The narrower AMCA emission spectrum reduces spectral cross talk with other fluorescent stains and labels used in IF and FISH, reducing potential counterstain interference in interpretation of the biomarker presence and/or expression level, and potentially permitting higher level multiplexing by allowing greater use of the spectral region near AMCA fluorescence. CDCs with other excitation and emission maxima can be selected to be spectrally distant from the florescence of biomarker staining used in particular assays.

### ADDITIONAL EMBODIMENTS

Additional Embodiment 1. A method of detecting a biomarker in morphological context within a biological sample, comprising:
   (a) labeling at least a portion of a first morphological feature of the biological sample with a first detectable moiety, wherein the labeling of the first morphological feature comprises: (i) contacting a first morphological marker characteristic of the at least the portion of the first morphological feature with a first detection probe that binds to the first morphological marker, and (ii) covalently depositing the first detectable moiety on or proximal to the first morphological marker; and,
   (b) labeling a first biomarker in the biological sample with a second detectable moiety, wherein the second detectable moiety is different from the first detectable moiety, and wherein the labeling of the first biomarker comprises: (i) contacting the first biomarker with a second detection probe that binds the first biomarker; and (ii) covalently depositing the second detectable moiety on or proximal to the first biomarker.
Additional Embodiment 2. The method of additional embodiment 1, wherein the FWHM of the first and/or second detectable moieties is less than about 200 nm.
Additional Embodiment 3. The method of additional embodiment 1, wherein the FWHM of the first and/or second detectable moieties is less than about 150 nm.
Additional Embodiment 4. The method of additional embodiment 1, wherein the first and second detectable moieties are each independently conjugated to a tyramide or a derivative thereof, a quinone methide precursor moiety or a derivative thereof, or a reactive functional group capable of participating in a click chemistry reaction; and wherein the covalent deposition of the first detectable moiety and the second detectable moiety independently comprises one of tyramide signal amplification, quinone methide chemistry, or click chemistry.
Additional Embodiment 5. The method of additional embodiment 1, wherein the absorbance maximum (λₘₐₓ) of the first detectable moiety and the absorbance maximum (λₘₐₓ) of the second detectable moiety are separated by at least about 20 nm.
Additional Embodiment 6. The method of additional embodiment 1, wherein the absorbance maximum (λₘₐₓ) of the first detectable moiety and the absorbance maximum (λₘₐₓ) of the second detectable moiety are separated by at least about 30 nm.
Additional Embodiment 7. The method of additional embodiment 1, wherein the absorbance maximum (λₘₐₓ) of the first detectable moiety and the absorbance maximum (λₘₐₓ) of the second detectable moiety are separated by at least about 40 nm.
Additional Embodiment 8. The method of additional embodiment 1, wherein the absorbance maximum (λₘₐₓ) of the first detectable moiety and the absorbance maximum (λₘₐₓ) of the second detectable moiety are separated by at least about 50 nm.
Additional Embodiment 9. The method of additional embodiment 1, wherein the first morphological marker comprises DNA.
Additional Embodiment 10. The method of additional embodiment 9, wherein the labeling of the DNA with the first detectable moiety comprises: (a) contacting the biological sample with an anti-DNA primary antibody; (b) contacting the biological sample with an anti-specifies secondary antibody specific to the anti-DNA primary antibody, wherein the anti-species antibody is conjugated directly or indirectly to at least one enzyme; and (c) contacting the biological sample with a first detectable conjugate comprising (i) the first detectable moiety, and (ii) a tyramide moiety, a quinone methide precursor moiety, or a derivative or analog of a tyramide moiety or quinone methide precursor moiety.
Additional Embodiment 11. The method of additional embodiment 9, wherein the labeling of the DNA with the first detectable moiety comprises: (a) contacting the biological sample with an anti-DNA primary antibody; (b) contacting the biological sample with an anti-specifies secondary antibody specific to the anti-DNA antibody, wherein the anti-species antibody is conjugated directly or indirectly to at least one enzyme; (c) contacting the biological sample with a first tissue reactive conjugate comprising: (i) a first member of a pair of reactive functional groups capable of participating in a click chemistry reaction, and (ii) a tyramide moiety, a quinone methide precursor moiety, or a derivative or analog of a tyramide moiety or quinone methide precursor moiety; and (d) contacting the biological sample with a detectable conjugate comprising (i) the first detectable moiety, and (ii) a second member of the pair of reactive functional groups.
Additional Embodiment 12. The method of additional embodiment 1, wherein the first morphological marker comprises a histone protein.
Additional Embodiment 13. The method of additional embodiment 12, wherein the labeling of the histone proteins with the first detectable moiety comprises: (a) contacting the biological sample with an anti-histone primary antibody; (b) contacting the biological sample with an anti-specifies secondary antibody specific to the anti-histone primary antibody, wherein the anti-species antibody is conjugated directly or indirectly to at least one enzyme; and (c) contacting the biological sample with a first detectable conjugate comprising (i) the first detectable moiety, and (ii) a tyramide moiety, a quinone methide precursor moiety, or a derivative or analog of a tyramide moiety or quinone methide precursor moiety.
Additional Embodiment 14. The method of additional embodiment 12, wherein the labeling of the histone proteins with the first detectable moiety comprises: (a) contacting the biological sample with an anti-histone primary antibody; (b) contacting the biological sample with an anti-specifies secondary antibody specific to the anti-histone antibody, wherein the anti-species antibody is conjugated directly or indirectly to at least one enzyme; (c) contacting the biological sample with a first tissue reactive conjugate comprising: (i) a first member of a pair of reactive functional groups capable of participating in a click chemistry reaction, and (ii) a tyramide moiety, a quinone methide precursor moiety, or a derivative or analog of a tyramide moiety or quinone methide precursor moiety; and (d) contacting the biological sample with a detectable conjugate comprising (i) the first detectable moiety, and (ii) a second member of the pair of reactive functional groups.
Additional Embodiment 15. The method of additional embodiment 1, wherein the first morphological marker is selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers.
Additional Embodiment 16. The method of additional embodiment 1, wherein the first biomarker is a protein biomarker. Alternatively, the method of additional embodiment 1, wherein the first biomarker is a nucleic acid biomarker.
Additional Embodiment 17. The method of additional embodiment 1, wherein the first biomarker is selected from the group consisting of PD-L1, Ki-67, CD3, CD8, CD4, CD20, CD68, p40, p63, TTF-1, ERG, ERBB2 (HER2), alpha-methylacyl-CoA racemase (AMACR), and synaptophysin.
Additional Embodiment 18. The method of additional embodiment 1, wherein the first detectable moiety comprises a coumarin core.
Additional Embodiment 19. The method of additional embodiment 18, wherein the second detectable moiety is within the visible spectrum or within the infrared spectrum.
Additional Embodiment 20. The method of additional embodiment 18, wherein the second detectable moiety is within the ultraviolet spectrum.
Additional Embodiment 21. The method of additional embodiment 20, wherein the first and second detectable moieties have absorbance maximums (λₘₐₓ) that are separated by at least 20 nm.
Additional Embodiment 22. The method of additional embodiment 1, wherein the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core.
Additional Embodiment 23. The method of additional embodiment 22, wherein the second detectable moiety is within the ultraviolet spectrum or within the infrared spectrum.
Additional Embodiment 24. The method of additional embodiment 22, wherein the second detectable moiety is within the visible spectrum.
Additional Embodiment 25. The method of additional embodiment 22, wherein the first and second detectable moieties have absorbance maximums (λₘₐₓ) that are separated by at least 20 nm.
Additional Embodiment 26. The method of additional embodiment 1, wherein the first detectable moiety comprises a heptamethine cyanine core or a croconate core.
Additional Embodiment 27. The method of additional embodiment 26, wherein the second detectable moiety is within the visible spectrum or within the ultraviolet spectrum.
Additional Embodiment 28. The method of additional embodiment 26, wherein the second detectable moiety is within the infrared spectrum.
Additional Embodiment 29. The method of additional embodiment 26, wherein the first and second detectable moieties have absorbance maximums (λₘₐₓ) that are separated by at least 20 nm.
Additional Embodiment 30. A method of detecting one or more targets within a biological sample, comprising:
   (a) labeling a first morphological marker with a first detectable moiety comprising a core selected from the group consisting of a coumarin core, a phenoxazinone core, a 4-Hydroxy-3-phenoxazinone core, a 7-amino-4-Hydroxy-3-phenoxazinone core, a thioninium core, a phenoxazine core, a phenoxathiin-3-one core, a xanthene core, a heptamethine cyanine core and a croconate core;
   (b) labeling a first biomarker with a second detectable moiety comprising a core selected from the group consisting of a coumarin core, a phenoxazinone core, a 4-Hydroxy-3-phenoxazinone core, a 7-amino-4-Hydroxy-3-phenoxazinone core, a thioninium core, a phenoxazine core, a phenoxathiin-3-one core, a xanthene core, a heptamethine cyanine core and a croconate core;
   wherein the first and second detectable moieties are different and have absorbance maximums (λₘₐₓ) which differ by at least 10 nm.
Additional Embodiment 31. The method of additional embodiment 30, wherein the first detectable moiety is within the visible spectrum.
Additional Embodiment 32. The method of additional embodiment 31, wherein the first detectable moiety is outside the visible spectrum.
Additional Embodiment 33. The method of additional embodiment 31, wherein the first detectable moiety is within the visible spectrum.
Additional Embodiment 34. The method of additional embodiment 30, wherein the first detectable moiety is within the ultraviolet spectrum.
Additional Embodiment 35. The method of additional embodiment 34, wherein the first detectable moiety is outside the ultraviolet spectrum.
Additional Embodiment 36. The method of additional embodiment 34, wherein the first detectable moiety is within the ultraviolet spectrum.
Additional Embodiment 37. The method of additional embodiment 30, wherein the first detectable moiety is within the infrared spectrum.
Additional Embodiment 38. The method of additional embodiment 37, wherein the first detectable moiety is infrared the ultraviolet spectrum.
Additional Embodiment 39. The method of additional embodiment 37, wherein the first detectable moiety is within the infrared spectrum.
Additional Embodiment 40. The method of additional embodiment 30, wherein the first biomarker is a cancer biomarker.
Additional Embodiment 41. The method of additional embodiment 30, wherein the first morphological marker comprises DNA.
Additional Embodiment 42. The method of additional embodiment 30, wherein the first morphological marker comprises histone proteins.
Additional Embodiment 43. The method of additional embodiment 30, wherein the first morphological marker is selected from the group consisting of a marker for cytosol, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers.
Additional Embodiment 44. The method of additional embodiment 31, wherein the absorbance maximums (λₘₐₓ) of the first and second detectable moieties differ by at least 20 nm.
Additional Embodiment 45. The method of additional embodiment 31, wherein the absorbance maximums (λₘₐₓ) of the first and second detectable moieties differ by at least 30 nm.
Additional Embodiment 46. The method of additional embodiment 31, further comprising labeling a second biomarker with a third detectable moiety, wherein the third detectable moiety is different than the first and second detectable moieties, and wherein the first, second, and third detectable moieties have absorbance maximums (λₘₐₓ) which differ by at least 10 nm.
Additional Embodiment 47. The method of additional embodiment 46, wherein the absorbance maximums (λₘₐₓ) of the first, second, and third detectable moieties differ by at least 20 nm.
Additional Embodiment 48. The method of additional embodiment 46, wherein the absorbance maximums (λₘₐₓ) of the first, second, and third differ by at least 30 nm.
Additional Embodiment 49. The method of additional embodiment 30, wherein the first and second detectable moieties are selected from the group consisting of:

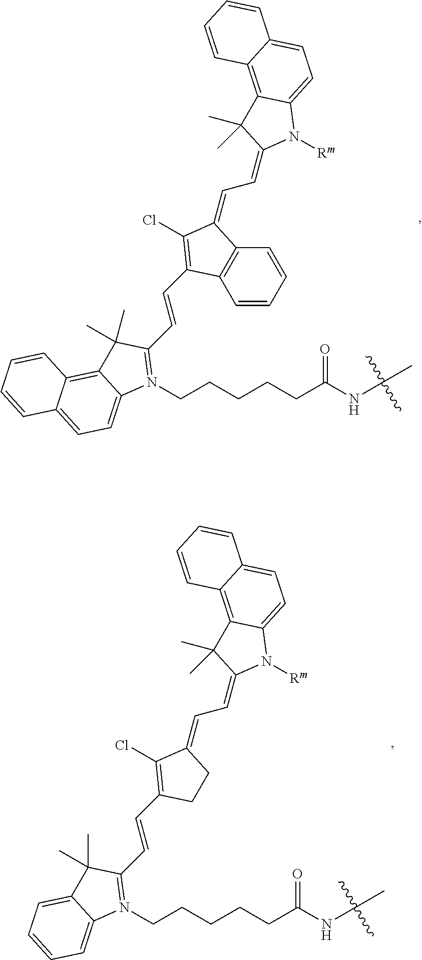

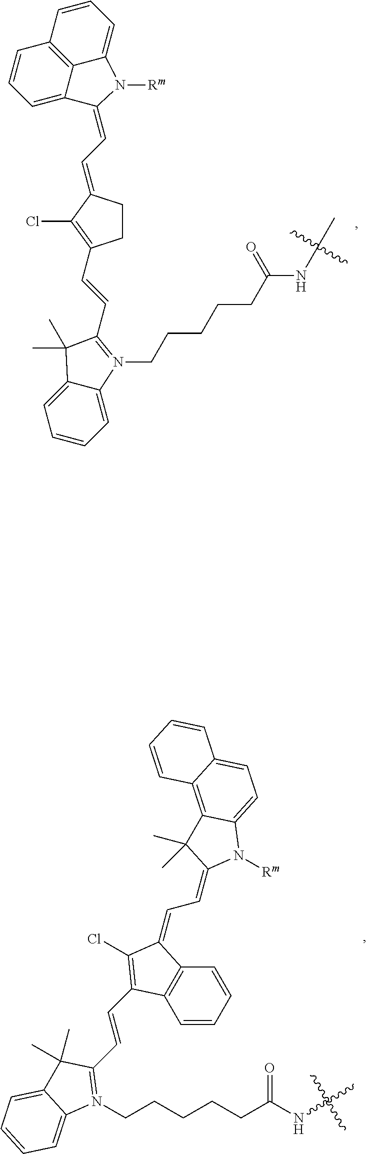

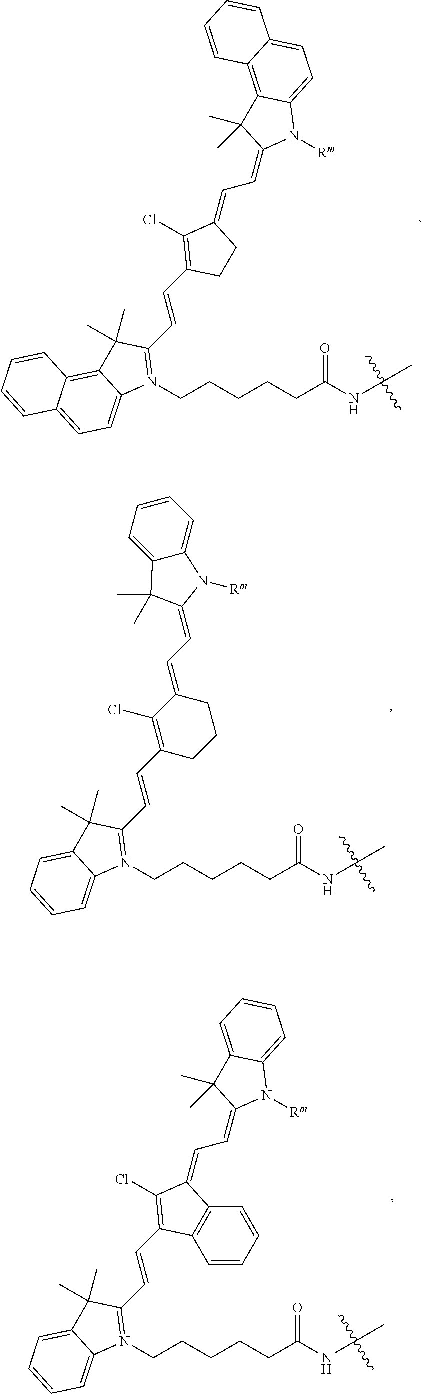

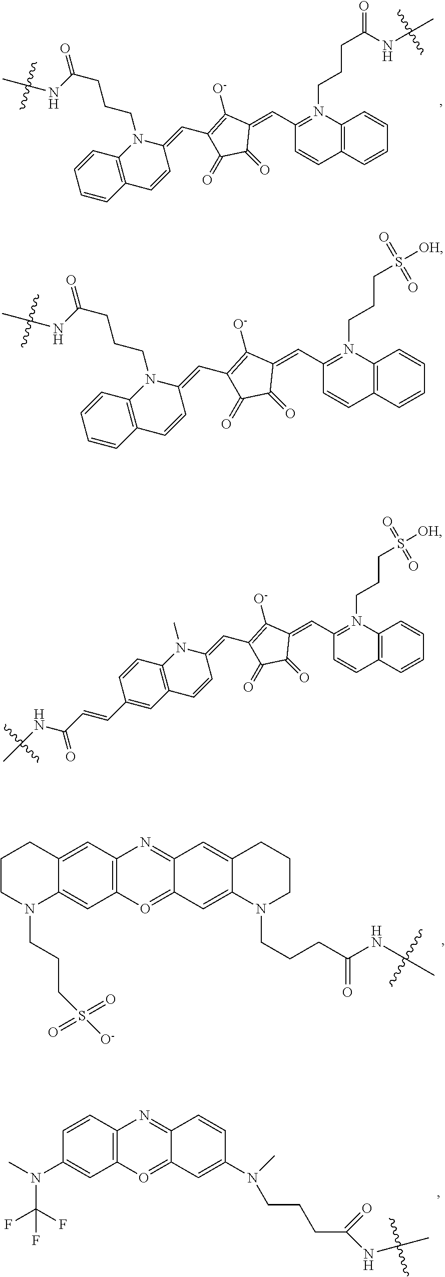

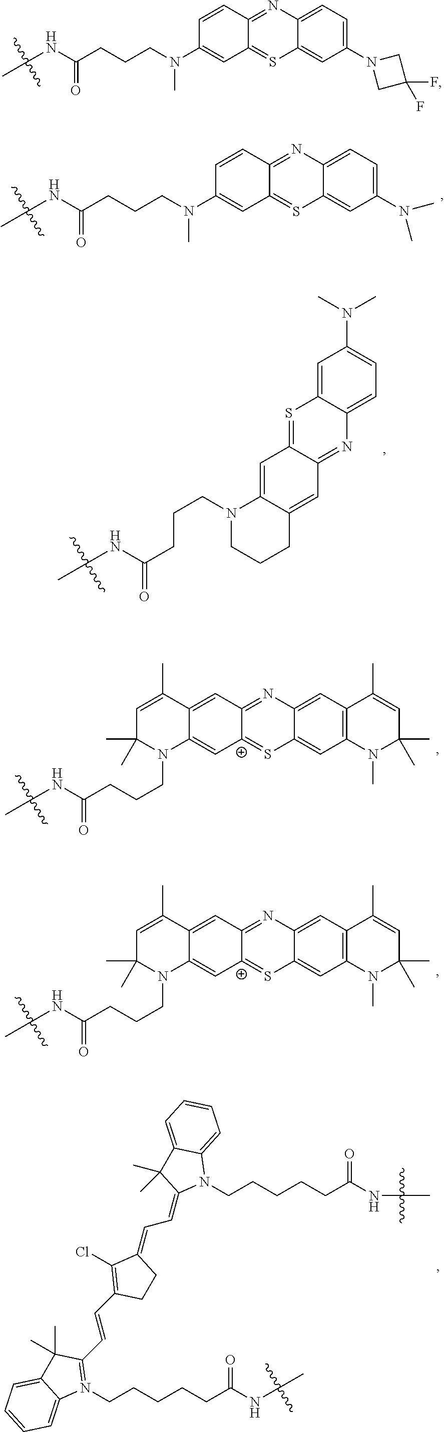

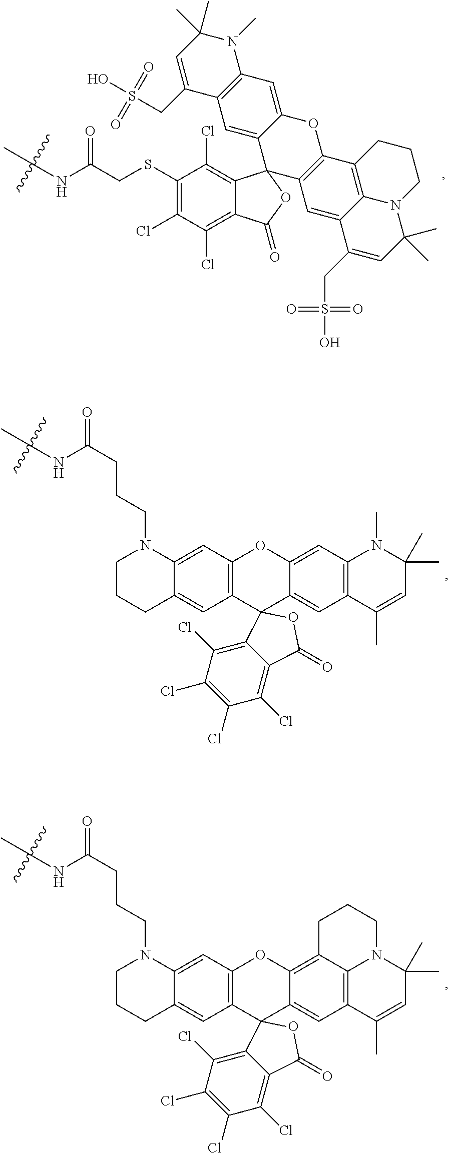 [SMILES: *NC(=O)CSc1c(Cl)c(Cl)c2c(c1Cl)C1(OC2=O)c2cc3c(cc2Oc2c1cc1c4c2CCCN4C(C)(C)C=C1CS(=O)(=O)O)N(C)C(C)(C)C=C3CS(=O)(=O)O ; *NC(=O)CCCN1CCCc2cc3c(cc21)Oc1cc2c(cc1C31OC(=O)c3c(Cl)c(Cl)c(Cl)c(Cl)c31)C(C)=CC(C)(C)N2C ; *NC(=O)CCCN1CCCc2cc3c(cc21)Oc1c(cc2c4c1CCCN4C(C)(C)C=C2C)C31OC(=O)c2c(Cl)c(Cl)c(Cl)c(Cl)c21]

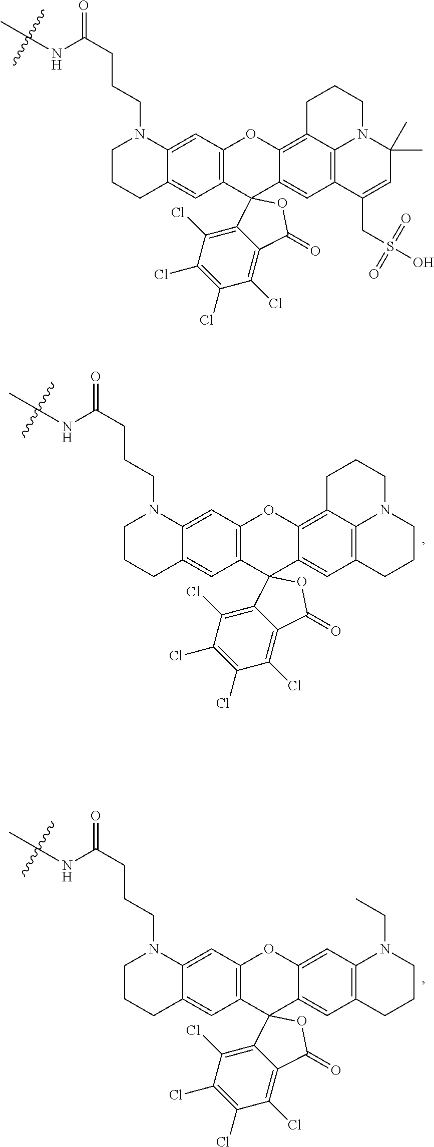 [SMILES: *NC(=O)CCCN1CCCc2cc3c(cc21)Oc1c(cc2c4c1CCCN4C(C)(C)C=C2CS(=O)(=O)O)C31OC(=O)c2c(Cl)c(Cl)c(Cl)c(Cl)c21 ; *NC(=O)CCCN1CCCc2cc3c(cc21)Oc1c(cc2c4c1CCCN4CCC2)C31OC(=O)c2c(Cl)c(Cl)c(Cl)c(Cl)c21 ; *NC(=O)CCCN1CCCc2cc3c(cc21)Oc1cc2c(cc1C31OC(=O)c3c(Cl)c(Cl)c(Cl)c(Cl)c31)CCCN2CC]

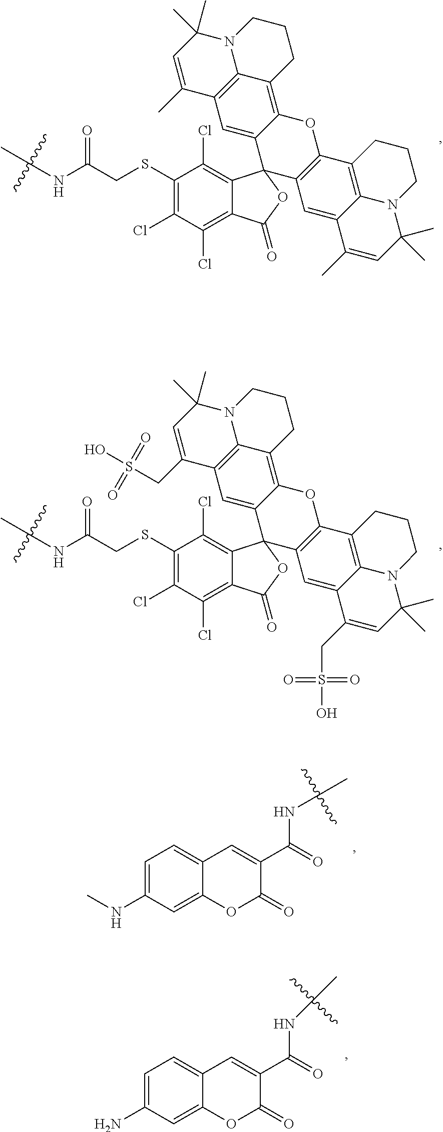

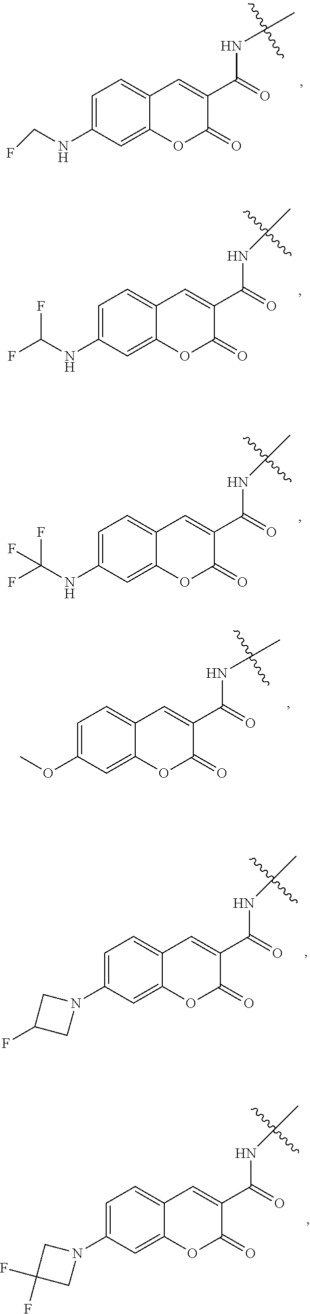 [SMILES: *NC(=O)c1cc2ccc(NCF)cc2oc1=O ; *NC(=O)c1cc2ccc(NC(F)F)cc2oc1=O ; *NC(=O)c1cc2ccc(NC(F)(F)F)cc2oc1=O ; *NC(=O)c1cc2ccc(OC)cc2oc1=O ; *NC(=O)c1cc2ccc(N3CC(F)C3)cc2oc1=O ; *NC(=O)c1cc2ccc(N3CC(F)(F)C3)cc2oc1=O]

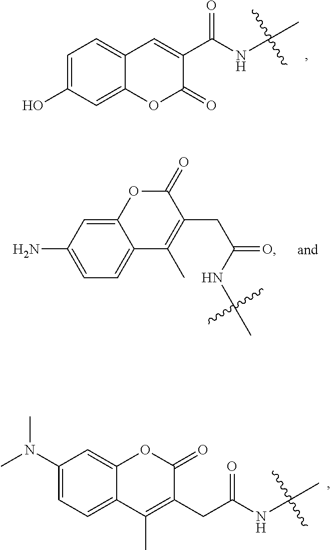 [SMILES: *NC(=O)c1cc2ccc(O)cc2oc1=O ; *NC(=O)Cc1c(C)c2ccc(N)cc2oc1=O ; *NC(=O)Cc1c(C)c2ccc(N(C)C)cc2oc1=O]

where the symbol “ [symbol] ” refers to the site in which the detectable moiety is conjugated to another moiety of a detectable conjugate.
Additional Embodiment 50. A biological sample comprising: (a) a first morphological marker labeled with a first detectable moiety; and (b) a first biomarker labeled with a second detectable moiety; wherein the first and second detectable moieties each have a first absorbance peak with FWHM of less than about 200 nm and an absorbance maximum (λₘₐₓ) between 330 nm+/−10 and 950 nm+/−10; and wherein an absorbance maximum (λₘₐₓ) of the first detectable moiety and an absorbance maximum (λₘₐₓ) of the second detectable moiety are separated by at least 20 nm.
Additional Embodiment 51. The biological sample of additional embodiment 50, wherein the separation between the absorbance maximums (λₘₐₓ) of the first and second detectable moieties is at least 30 nm.
Additional Embodiment 52. The biological sample of additional embodiment 50, wherein the separation between the absorbance maximums (λₘₐₓ) of the first and second detectable moieties is at least 45 nm.
Additional Embodiment 53. The biological sample of additional embodiment 50, wherein the separation between the absorbance maximums (λₘₐₓ) of the first and second detectable moieties is at least 60 nm.
Additional Embodiment 54. The biological sample of additional embodiment 50, wherein the first morphological marker is selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers.
Additional Embodiment 55. The biological sample of additional embodiment 50, wherein the first morphological marker is selected from the group consisting of DNA and histone proteins.
Additional Embodiment 56. The biological sample of additional embodiment 50, wherein the first detectable moiety comprises a coumarin core.
Additional Embodiment 57. The biological sample of additional embodiment 56, wherein the second detectable moiety is within the visible spectrum or within the infrared spectrum.
Additional Embodiment 58. The biological sample of additional embodiment 56, wherein the second detectable moiety is within the ultraviolet spectrum.
Additional Embodiment 59. The biological sample of additional embodiment 56, wherein the first and second detectable moieties have absorbance maximums (λₘₐₓ) that are separated by at least 20 nm.
Additional Embodiment 60. The biological sample of additional embodiment 50, wherein the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core.
Additional Embodiment 61. The biological sample of additional embodiment 60, wherein the second detectable moiety is within the ultraviolet spectrum or within the infrared spectrum.
Additional Embodiment 62. The biological sample of additional embodiment 60, the second detectable moiety is within the visible spectrum.
Additional Embodiment 63. The biological sample of additional embodiment 60, wherein the first and second detectable moieties have absorbance maximums (λₘₐₓ) that are separated by at least 20 nm.
Additional Embodiment 64. The biological sample of additional embodiment 50, wherein the first detectable moiety comprises a heptamethine cyanine core or a croconate core.
Additional Embodiment 65. The biological sample of additional embodiment 64, wherein the second detectable moiety is within the visible spectrum or within the ultraviolet spectrum.
Additional Embodiment 66. The biological sample of additional embodiment 64, wherein the second detectable moiety is within the infrared spectrum.
Additional Embodiment 67. The biological sample of additional embodiment 64, wherein the first and second detectable moieties have absorbance maximums (λₘₐₓ) that are separated by at least 20 nm.
Additional Embodiment 68. A biological sample comprising: (a) first biomarker labeled with a first detectable moiety; and (b) one of DNA or histone proteins labeled with a second detectable moiety; wherein the first and second detectable moieties each have a first absorbance peak with FWHM of less than about 200 nm and an absorbance maximum (λₘₐₓ) between 330 nm+/−10 and 950 nm+/−10; and wherein an absorbance maximum (λₘₐₓ) of the first detectable moiety and an absorbance maximum (λₘₐₓ) of the second detectable moiety are separated by at least 20 nm.
Additional Embodiment 69. The biological sample of additional embodiment 68, wherein the separation between the absorbance maximums (λₘₐₓ) of the first and second detectable moieties is at least 30 nm.
Additional Embodiment 70. The biological sample of additional embodiment 68, wherein the separation between the absorbance maximums (λₘₐₓ) of the first and second detectable moieties is at least 45 nm.
Additional Embodiment 71. The biological sample of additional embodiment 68, wherein the separation between the absorbance maximums (λₘₐₓ) of the first and second detectable moieties is at least 60 nm.
Additional Embodiment 72. The biological sample of additional embodiment 68, further comprising a second biomarker labeled with a third detectable moiety, wherein the wherein the first, second, and third detectable moieties have absorbance maximums (λₘₐₓ) which differ by at least 10 nm.
Additional Embodiment 73. The biological sample of additional embodiment 68, wherein the first and second detectable moieties are selected from the group consisting of:

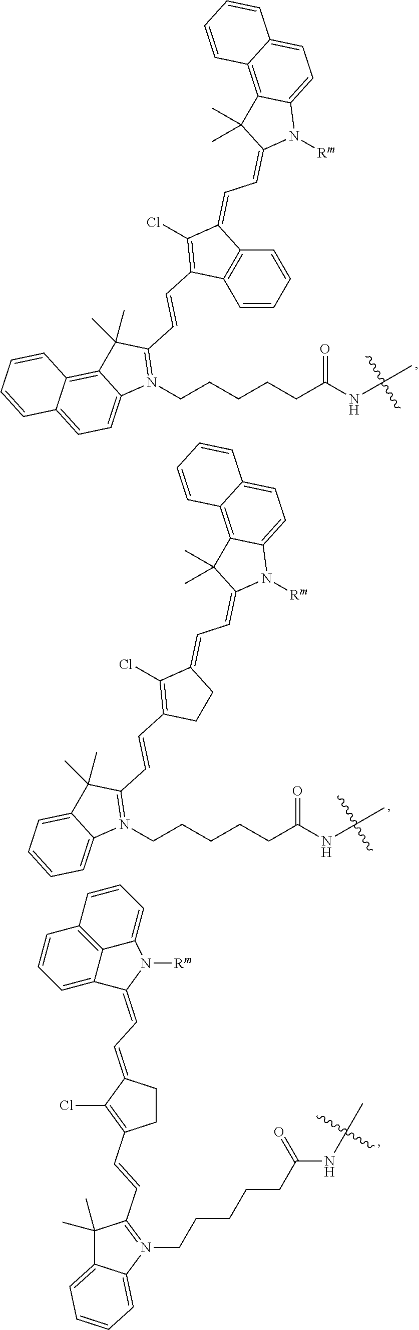

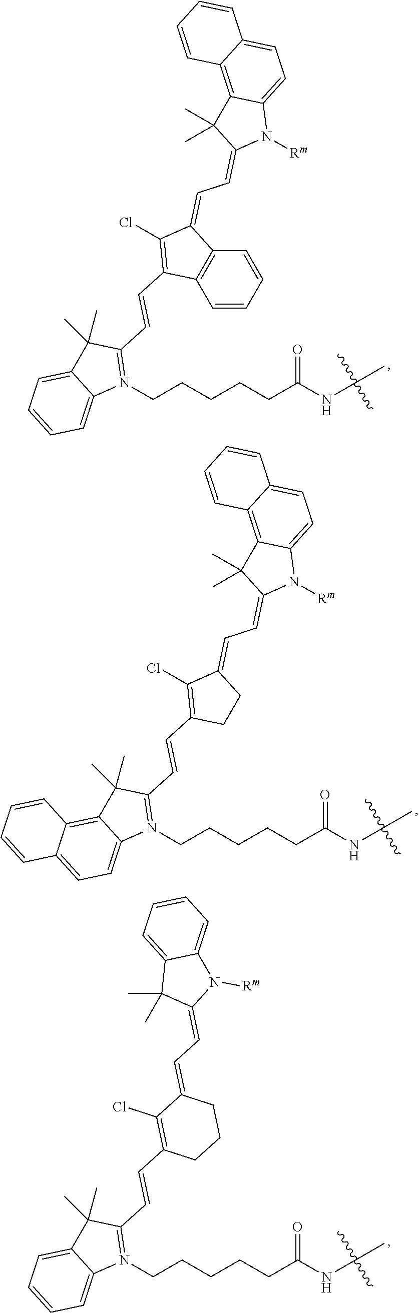

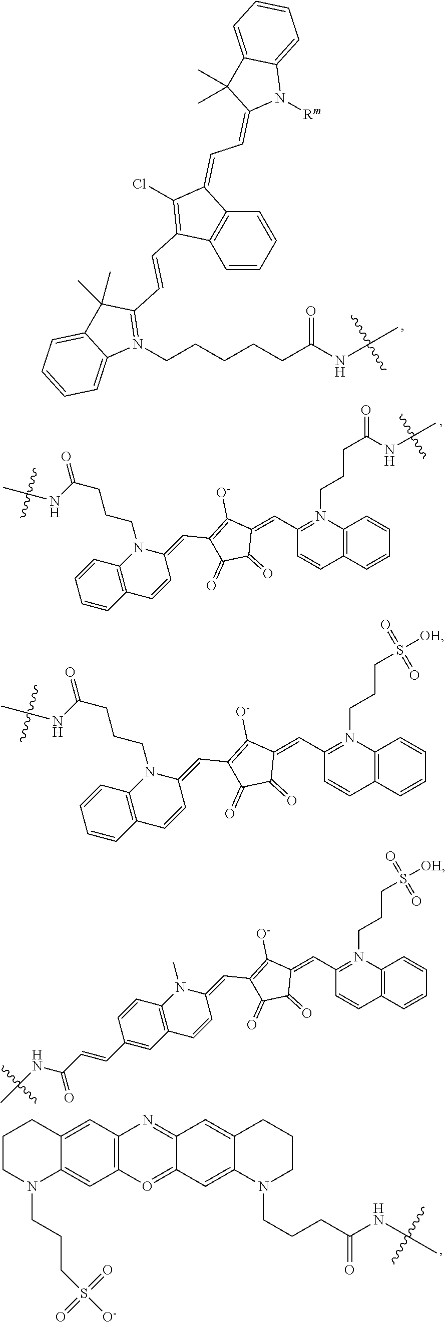

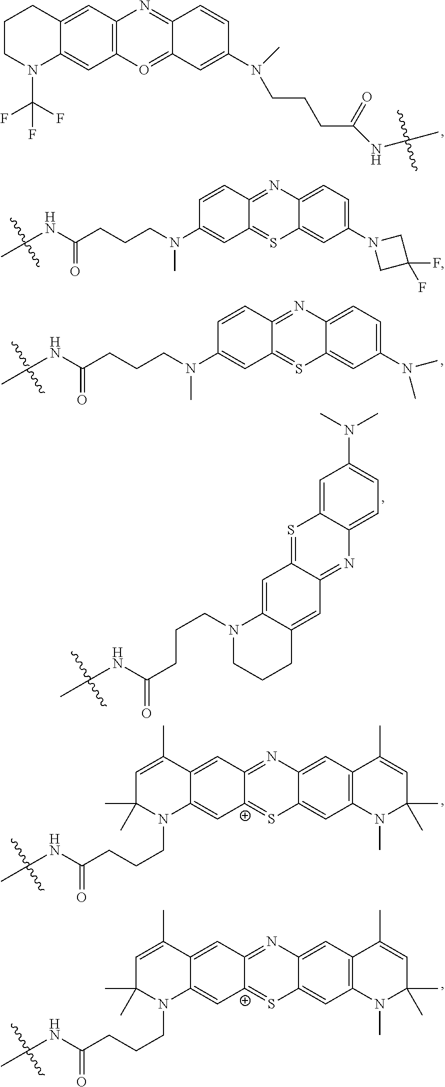

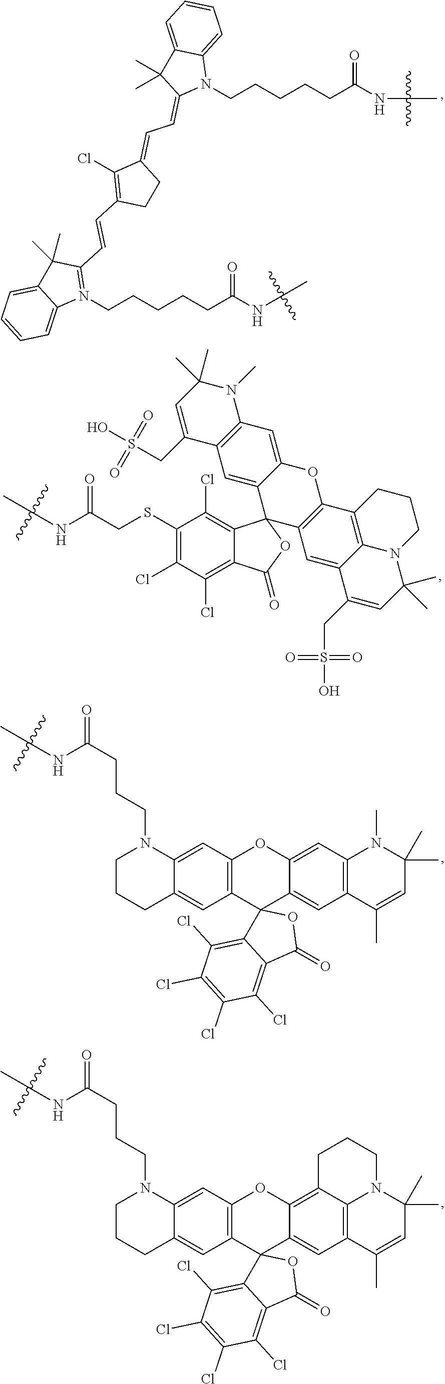

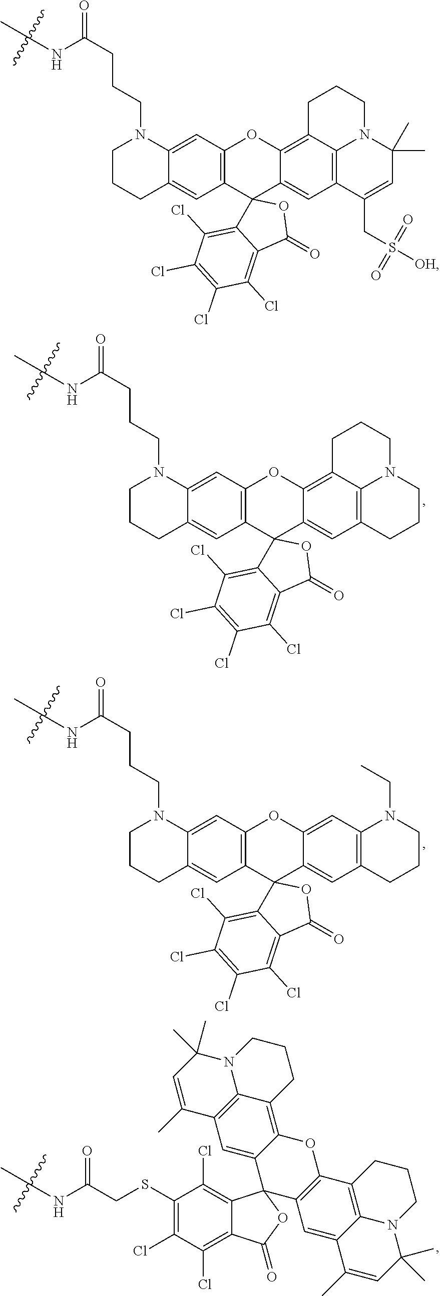

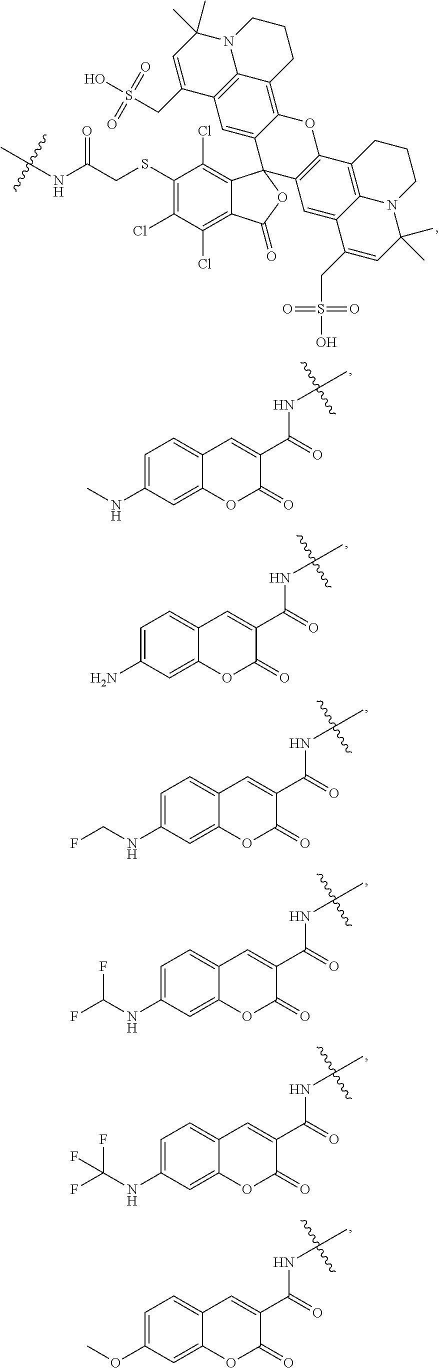

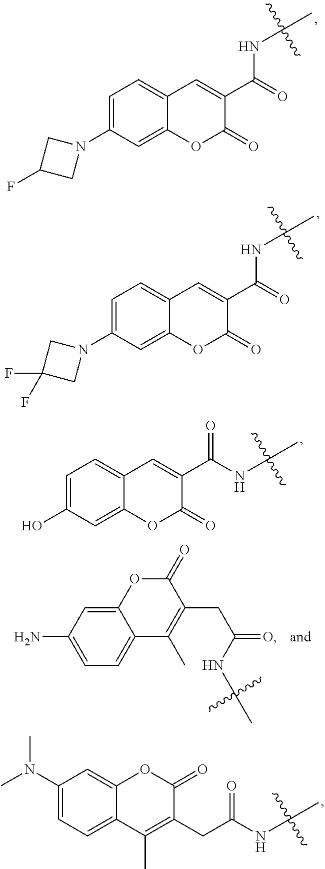

where the symbol “ [symbol] ” refers to the site in which the detectable moiety is conjugated to another moiety of a detectable conjugate.
Additional Embodiment 74. A biological sample comprising: (a) a first morphological marker labeled with a first detectable moiety; and (b) a first biomarker labeled with a second detectable moiety; wherein the first and second detectable moieties each have a first absorbance peak with FWHM of less than about 200 nm and an absorbance maximum (λₘₐₓ) between 330 nm+/−10 and 950 nm+/−1; and wherein an absorbance maximum (λₘₐₓ) of the first detectable moiety and an absorbance maximum (λₘₐₓ) of the second detectable moiety are separated by at least 20 nm; wherein the biological sample is prepared by:
   (i) contacting the biological sample with a first primary antibody specific to the first morphological marker;
   (ii) contacting the biological sample with a first secondary antibody specific to the first primary antibody, wherein the first secondary antibody is conjugated to an enzyme;
   (iii) contacting the biological sample with a first detectable conjugate comprising (a) a tyramide moiety, a quinone methide precursor moiety, or a derivative or analog of a tyramide moiety or quinone methide precursor moiety; and (b) the first detectable moiety;
   (iv) contacting the biological sample with a second primary antibody specific to the first biomarker;
   (v) contacting the biological sample with a second secondary antibody specific to the second primary antibody, wherein the second secondary antibody is conjugated to an enzyme; and
   (vi) contacting the biological sample with a second detectable conjugate comprising (a) a tyramide moiety, a quinone methide precursor moiety, or a derivative or analog of a tyramide moiety or quinone methide precursor moiety; and (b) the second detectable moiety.
Additional Embodiment 75. The biological sample of additional embodiment 74, wherein the separation between the absorbance maximums (λₘₐₓ) of the first and second detectable moieties is at least 30 nm.
Additional Embodiment 76. The biological sample of additional embodiment 74, wherein the separation between the absorbance maximums (λₘₐₓ) of the first and second detectable moieties is at least 45 nm.
Additional Embodiment 77. The biological sample of additional embodiment 74, wherein the separation between the absorbance maximums (λₘₐₓ) of the first and second detectable moieties is at least 60 nm.
Additional Embodiment 78. The biological sample of additional embodiment 74, wherein the first morphological marker is selected from the group consisting of a marker for cytosol, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers.
Additional Embodiment 79. The biological sample of additional embodiment 74, wherein the first morphological marker is selected from the group consisting of DNA and histone proteins.
Additional Embodiment 80. The biological sample of additional embodiment 74, wherein the first detectable moiety comprises a coumarin core.
Additional Embodiment 81. The biological sample of additional embodiment 80, wherein the second detectable moiety is within the visible spectrum or within the infrared spectrum.
Additional Embodiment 82. The biological sample of additional embodiment 80, wherein the second detectable moiety is within the ultraviolet spectrum.
Additional Embodiment 83. The biological sample of additional embodiment 80, wherein the first and second detectable moieties have absorbance maximums (λₘₐₓ) that are separated by at least 20 nm.
Additional Embodiment 84. The biological sample of additional embodiment 74, wherein the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core.
Additional Embodiment 85. The biological sample of additional embodiment 84, wherein the second detectable moiety is within the ultraviolet spectrum or within the infrared spectrum.
Additional Embodiment 86. The biological sample of additional embodiment 84, the second detectable moiety is within the visible spectrum.
Additional Embodiment 87. The biological sample of additional embodiment 84, wherein the first and second detectable moieties have absorbance maximums (λₘₐₓ) that are separated by at least 20 nm.
Additional Embodiment 88. The biological sample of additional embodiment 74, wherein the first detectable moiety comprises a heptamethine cyanine core or a croconate core.
Additional Embodiment 89. The biological sample of additional embodiment 88, wherein the second detectable moiety is within the visible spectrum or within the ultraviolet spectrum.
Additional Embodiment 90. The biological sample of additional embodiment 88, wherein the second detectable moiety is within the infrared spectrum.
Additional Embodiment 91. The biological sample of additional embodiment 88, wherein the first and second detectable moieties have absorbance maximums (λₘₐₓ) that are separated by at least 20 nm.
Additional Embodiment 92. A biological sample comprising: (a) a first morphological marker labeled with a first detectable moiety; and (b) a first biomarker labeled with a second detectable moiety; wherein the first and second detectable moieties each have a first absorbance peak with FWHM of less than about 200 nm and an absorbance maximum (λₘₐₓ) between 330 nm+/−10 and 950 nm+/−1; and wherein an absorbance maximum (λₘₐₓ) of the first detectable moiety and an absorbance maximum (λₘₐₓ) of the second detectable moiety are separated by at least 20 nm; wherein the biological sample is prepared by:
   (i) contacting the biological sample with a first primary antibody specific to the first morphological marker;
   (ii) contacting the biological sample with a first secondary antibody specific to the first primary antibody, wherein the first secondary antibody is conjugated to an enzyme;
   (iii) contacting the biological sample with a first tissue reactive moiety comprising (a) a tyramide moiety, a quinone methide precursor moiety, or a derivative or analog of a tyramide moiety or quinone methide precursor moiety; and (b) a first reactive functional group capable of participating in a click chemistry reaction;
   (iv) contacting the biological sample with a first detectable conjugate comprising: (a) the first detectable moiety; and (b) a second reactive functional group;
   (v) contacting the biological sample with a second primary antibody specific to the first biomarker;
   (vi) contacting the biological sample with a second secondary antibody specific to the second primary antibody, wherein the second secondary antibody is conjugated to an enzyme;
   (vii) contacting the biological sample with a second tissue reactive moiety comprising (a) a tyramide moiety, a quinone methide precursor moiety, or a derivative or analog of a tyramide moiety or quinone methide precursor moiety; and (b) a first reactive functional group capable of participating in a click chemistry reaction; (viii) contacting the biological sample with a second detectable conjugate comprising: (a) the second detectable moiety; and (b) a second reactive functional group.
Additional Embodiment 93. The biological sample of additional embodiment 92, wherein the separation between the absorbance maximums (λₘₐₓ) of the first and second detectable moieties is at least 30 nm.
Additional Embodiment 94. The biological sample of additional embodiment 92, wherein the separation between the absorbance maximums (λₘₐₓ) of the first and second detectable moieties is at least 45 nm.
Additional Embodiment 95. The biological sample of additional embodiment 92, wherein the separation between the absorbance maximums (λₘₐₓ) of the first and second detectable moieties is at least 60 nm.
Additional Embodiment 96. The biological sample of additional embodiment 92, wherein the first morphological marker is selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers.
Additional Embodiment 97. The biological sample of additional embodiment 92, wherein the first morphological marker is selected from the group consisting of DNA and histone proteins.
Additional Embodiment 98. The biological sample of additional embodiment 92, wherein the first detectable moiety comprises a coumarin core.
Additional Embodiment 99. The biological sample of additional embodiment 98, wherein the second detectable moiety is within the visible spectrum or within the infrared spectrum.
Additional Embodiment 100. The biological sample of additional embodiment 98, wherein the second detectable moiety is within the ultraviolet spectrum.
Additional Embodiment 101. The biological sample of additional embodiment 98, wherein the first and second detectable moieties have absorbance maximums (λₘₐₓ) that are separated by at least 20 nm.
Additional Embodiment 102. The biological sample of additional embodiment 92, wherein the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core.
Additional Embodiment 103. The biological sample of additional embodiment 102, wherein the second detectable moiety is within the ultraviolet spectrum or within the infrared spectrum.
Additional Embodiment 104. The biological sample of additional embodiment 102, the second detectable moiety is within the visible spectrum.
Additional Embodiment 105. The biological sample of additional embodiment 102, wherein the first and second detectable moieties have absorbance maximums (λₘₐₓ) that are separated by at least 20 nm.
Additional Embodiment 106. The biological sample of additional embodiment 92, wherein the first detectable moiety comprises a heptamethine cyanine core or a croconate core.
Additional Embodiment 107. The biological sample of additional embodiment 106, wherein the second detectable moiety is within the visible spectrum or within the ultraviolet spectrum.
Additional Embodiment 108. The biological sample of additional embodiment 106, wherein the second detectable moiety is within the infrared spectrum.
Additional Embodiment 109. A biological sample comprising: (a) a first ubiquitous marker labeled with a first detectable moiety; and (b) a first biomarker labeled with a second detectable moiety; wherein the first and second detectable moieties each have a first absorbance peak with FWHM of less than about 200 nm and an absorbance maximum (λₘₐₓ) between 330 nm+/−10 and 950 nm+/−10; and wherein an absorbance maximum (λₘₐₓ) of the first detectable moiety and an absorbance maximum (λₘₐₓ) of the second detectable moiety are separated by at least 20 nm; wherein the biological sample is prepared by:
   (i) contacting the biological sample with a first primary antibody specific to the first morphological marker;
   (ii) contacting the biological sample with a first secondary antibody specific to the first primary antibody, wherein the first secondary antibody is conjugated to an enzyme;
   (iii) contacting the biological sample with a first detectable conjugate comprising (a) a tyramide moiety, a quinone methide precursor moiety, or a derivative or analog of a tyramide moiety or quinone methide precursor moiety; and (b) the first detectable moiety;
   (iv) contacting the biological sample with a second primary antibody specific to the first biomarker;
   (v) contacting the biological sample with a second secondary antibody specific to the second primary antibody, wherein the second secondary antibody is conjugated to an enzyme;
   (vi) contacting the biological sample with a first tissue reactive moiety comprising (a) a tyramide moiety, a quinone methide precursor moiety, or a derivative or analog of a tyramide moiety or quinone methide precursor moiety; and (b) a first reactive functional group capable of participating in a click chemistry reaction;
   (vii) contacting the biological sample with a second detectable conjugate comprising: (a) the second detectable moiety; and (b) a second reactive functional group.
Additional Embodiment 110. The biological sample of additional embodiment 109, wherein the biological sample is free of hematoxylin.
Additional Embodiment 111. The biological sample of additional embodiment 109, further comprising contacting the biological sample with a third primary antibody specific to a second biomarker.
Additional Embodiment 112. The biological sample of additional embodiment 109, wherein the first and second detectable conjugates are selected from the group consisting of:

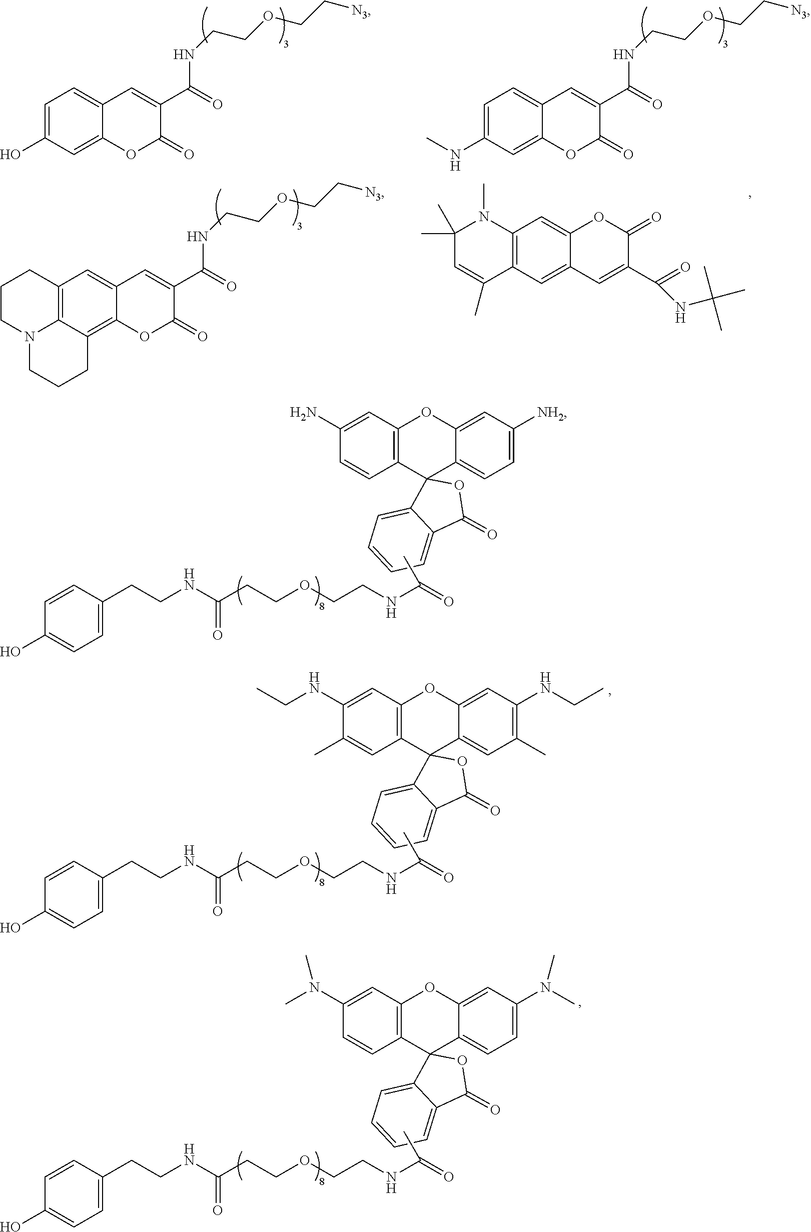

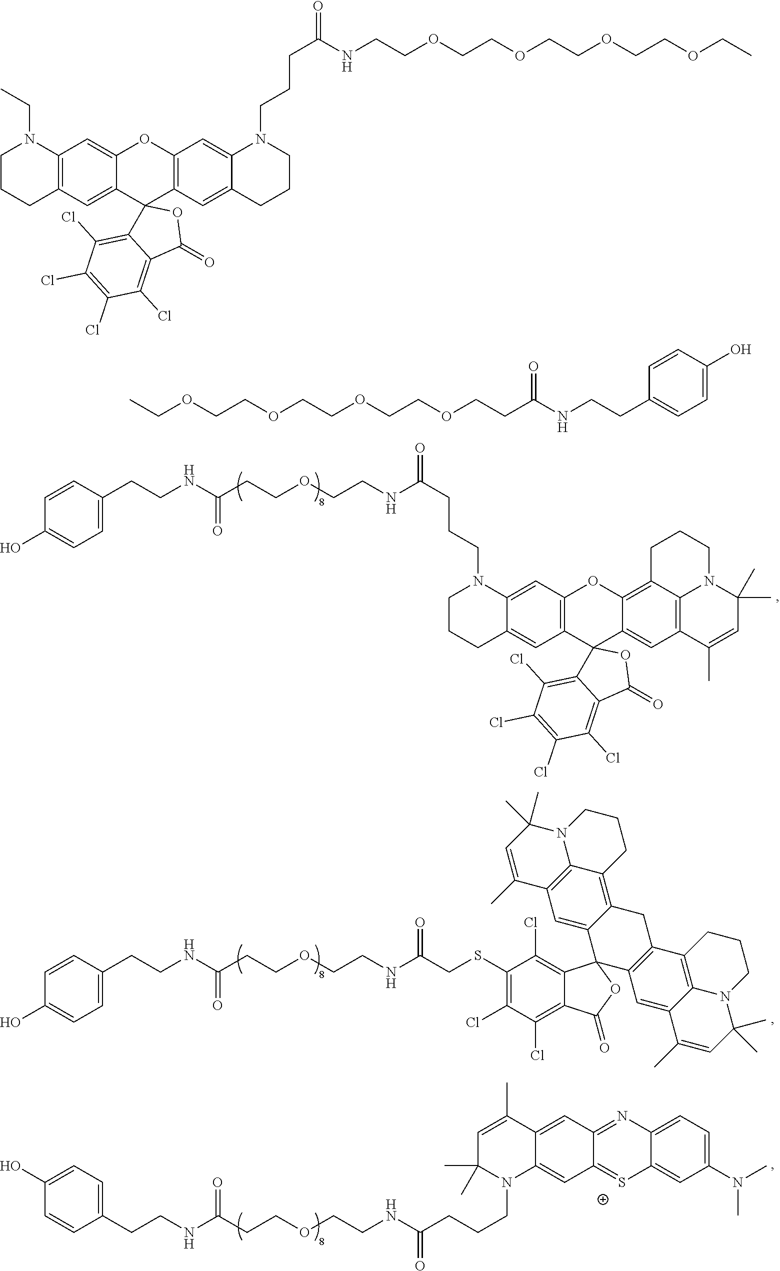

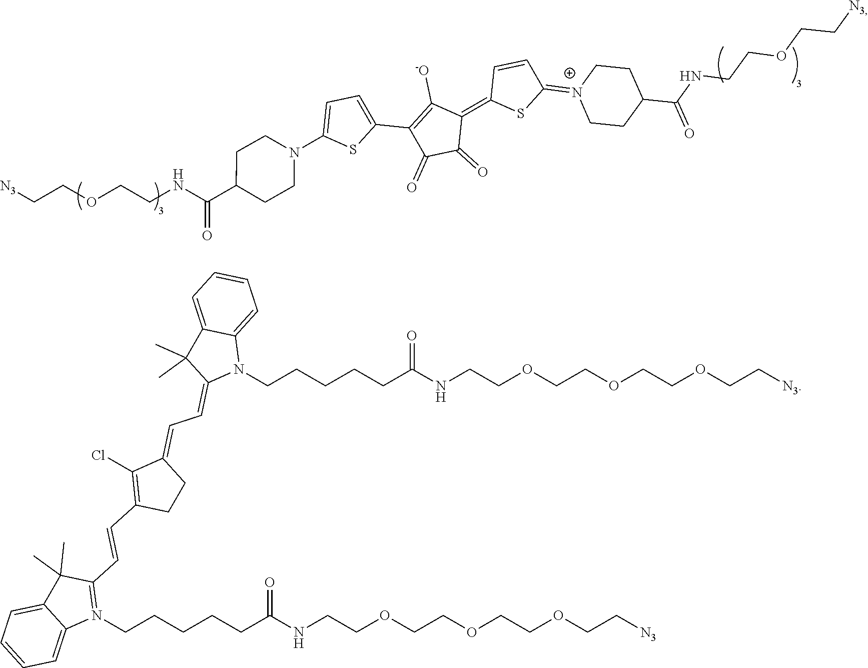

Additional Embodiment 113. A biological sample comprising: (a) a first morphological marker labeled with a first detectable moiety; and (b) a first biomarker labeled with a second detectable moiety; wherein the first and second detectable moieties each have a first absorbance peak with FWHM of less than about 200 nm and an absorbance maximum (λₘₐₓ) between 330 nm+/−10 and 950 nm+/−10; and wherein an absorbance maximum (λₘₐₓ) of the first detectable moiety and an absorbance maximum (λₘₐₓ) of the second detectable moiety are separated by at least 20 nm; wherein the biological sample is prepared by:
   (i) contacting the biological sample with a first primary antibody specific to the first morphological marker;
   (ii) contacting the biological sample with a first secondary antibody specific to the first primary antibody, wherein the first secondary antibody is conjugated to an enzyme;
   (iii) contacting the biological sample with a first tissue reactive moiety comprising (a) a tyramide moiety, a quinone methide precursor moiety, or a derivative or analog of a tyramide moiety or quinone methide precursor moiety; and (b) a first reactive functional group capable of participating in a click chemistry reaction;
   (iv) contacting the biological sample with a first detectable conjugate comprising: (a) the first detectable moiety; and (b) a second reactive functional group;
   (v) contacting the biological sample with a second primary antibody specific to the first biomarker;
   (vi) contacting the biological sample with a second secondary antibody specific to the second primary antibody, wherein the second secondary antibody is conjugated to an enzyme; and
   (vii) contacting the biological sample with a second detectable conjugate comprising (a) a tyramide moiety, a quinone methide precursor moiety, or a derivative or analog of a tyramide moiety or quinone methide precursor moiety; and (b) the second detectable moiety.
Additional Embodiment 114. The biological sample of additional embodiment 113, wherein the biological sample is free of hematoxylin.
Additional Embodiment 115. The biological sample of additional embodiment 113, further comprising contacting the biological sample with a third primary antibody specific to a second biomarker.
Additional Embodiment 116. The biological sample of additional embodiment 113, wherein the first and second detectable moieties are selected from the group consisting of:

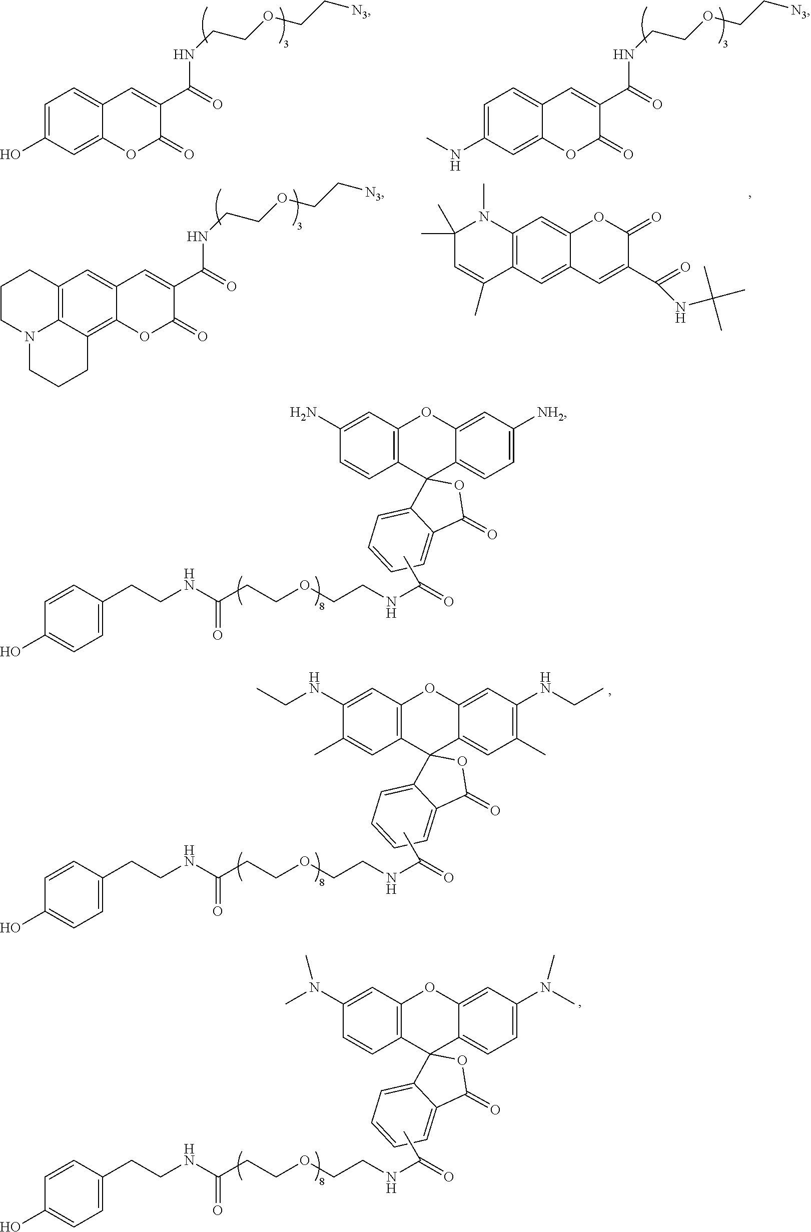

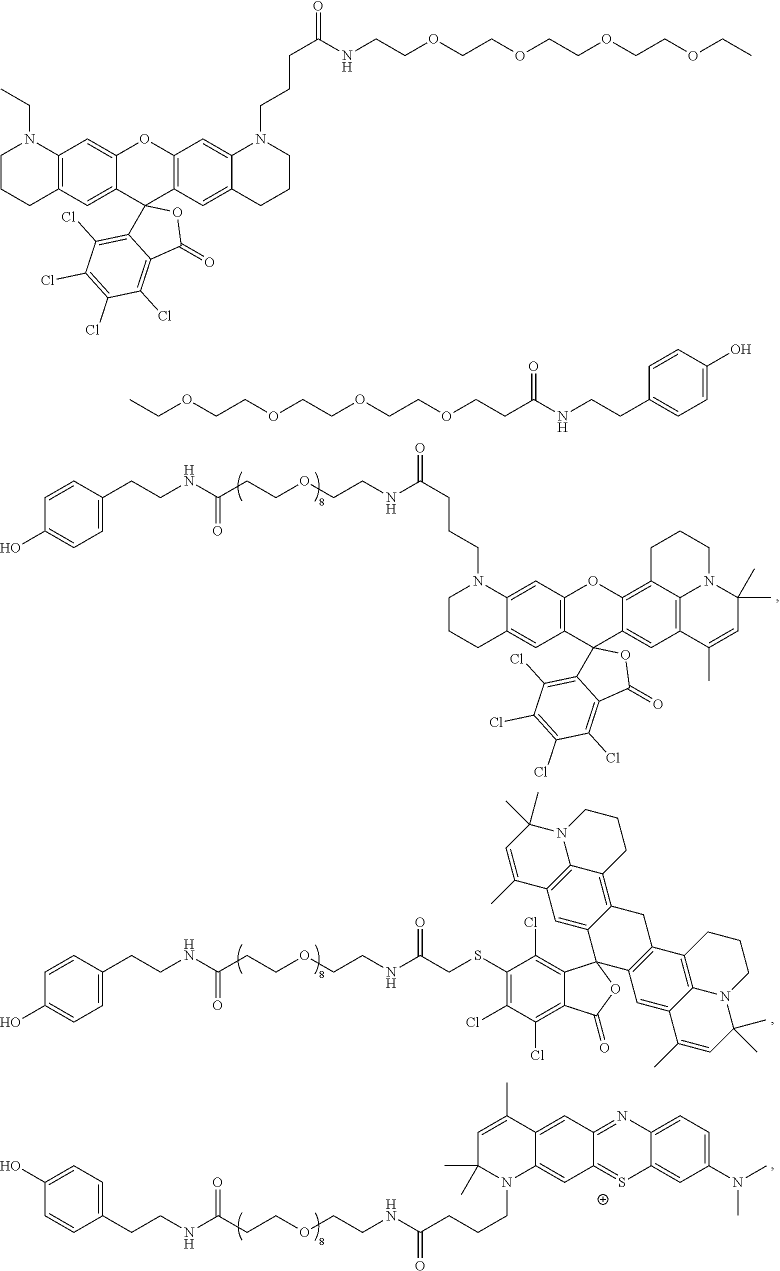

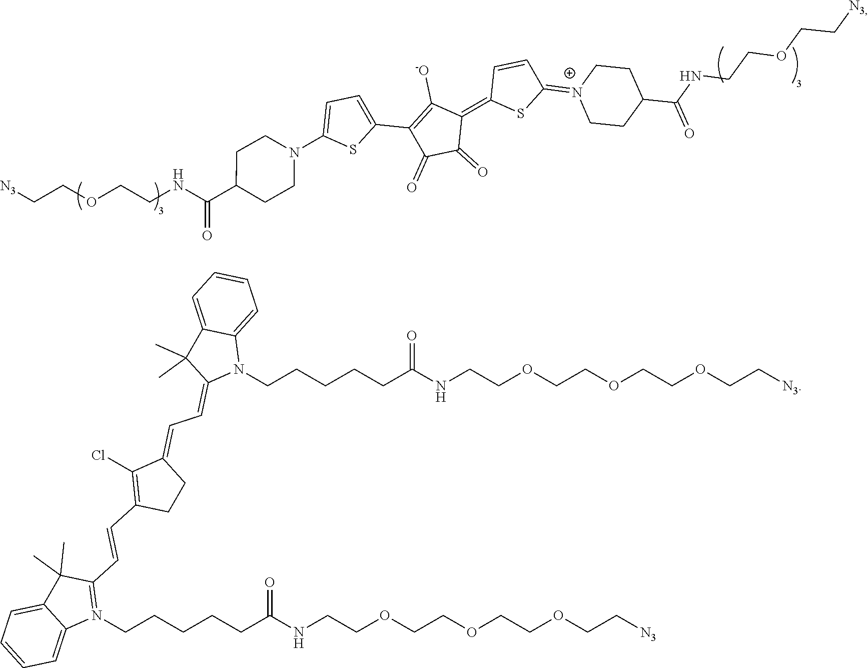

Additional Embodiment 117. A kit comprising: (a) a primary antibody specific to a first morphological marker; (b) a primary antibody specific to a first biomarker; and (c) at least two detection conjugates, wherein the at least two detection conjugates each include a different detectable moiety, wherein each detectable moiety has a first absorbance peak with FWHM of less than about 200 nm and an absorbance maximum (λₘₐₓ) between 330 nm+/−10 and 950 nm+/−10; and wherein an absorbance maximum (λₘₐₓ) of a first detectable moiety and an absorbance maximum (λₘₐₓ) of a second detectable moiety are separated by at least 20 nm.
Additional Embodiment 118. The kit of additional embodiment 117, wherein the at least two detection conjugates are selected from the group consisting of:

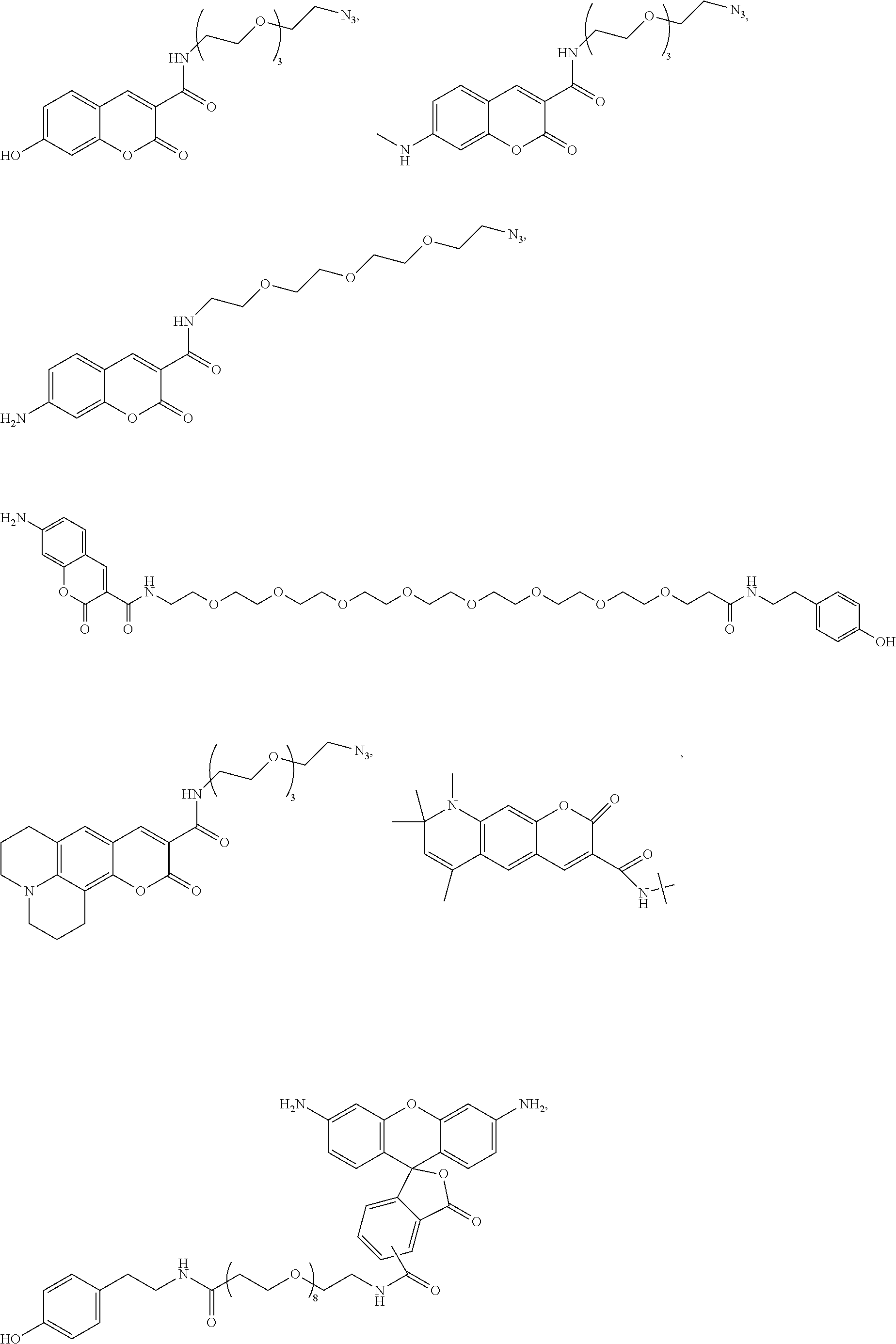

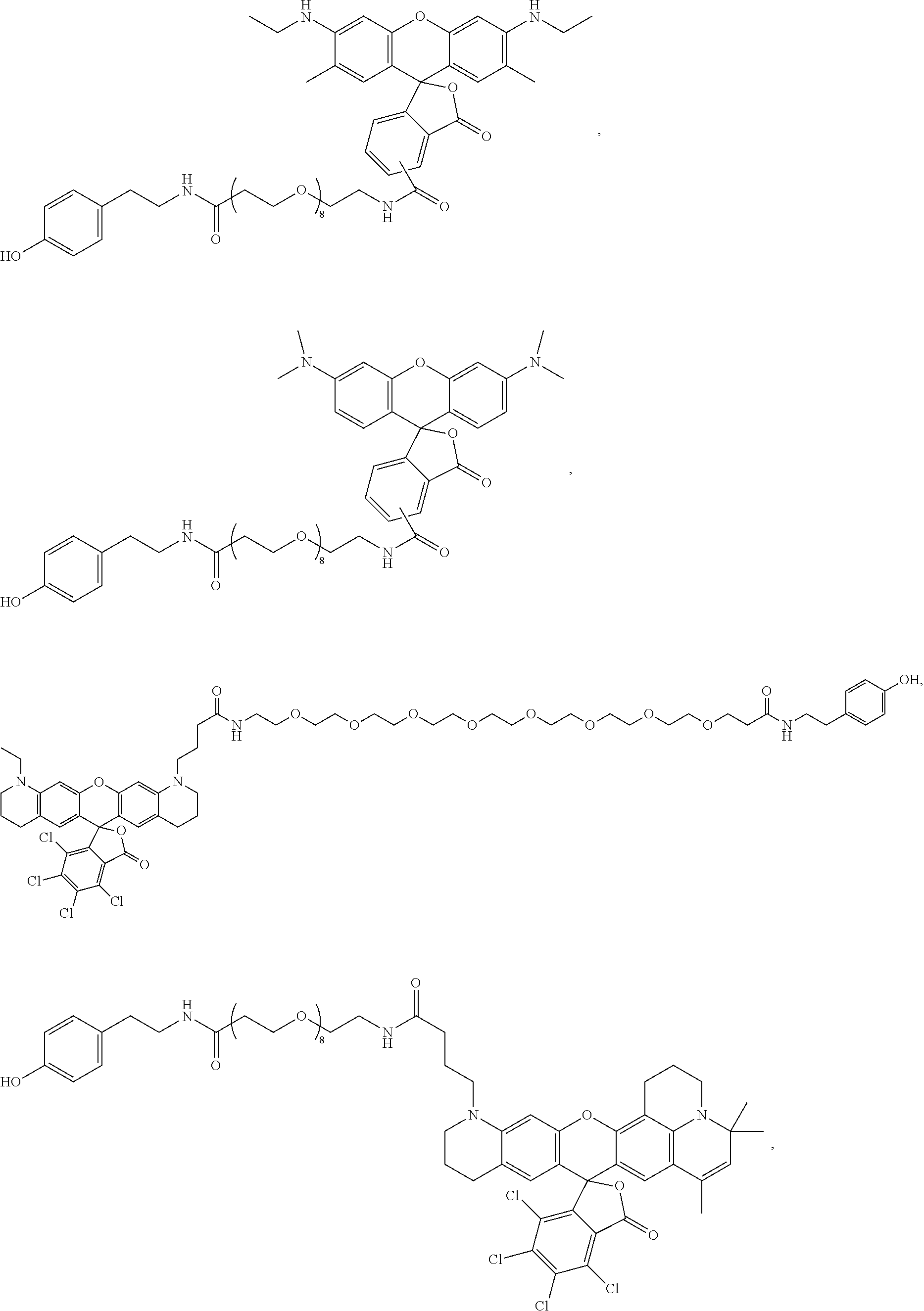

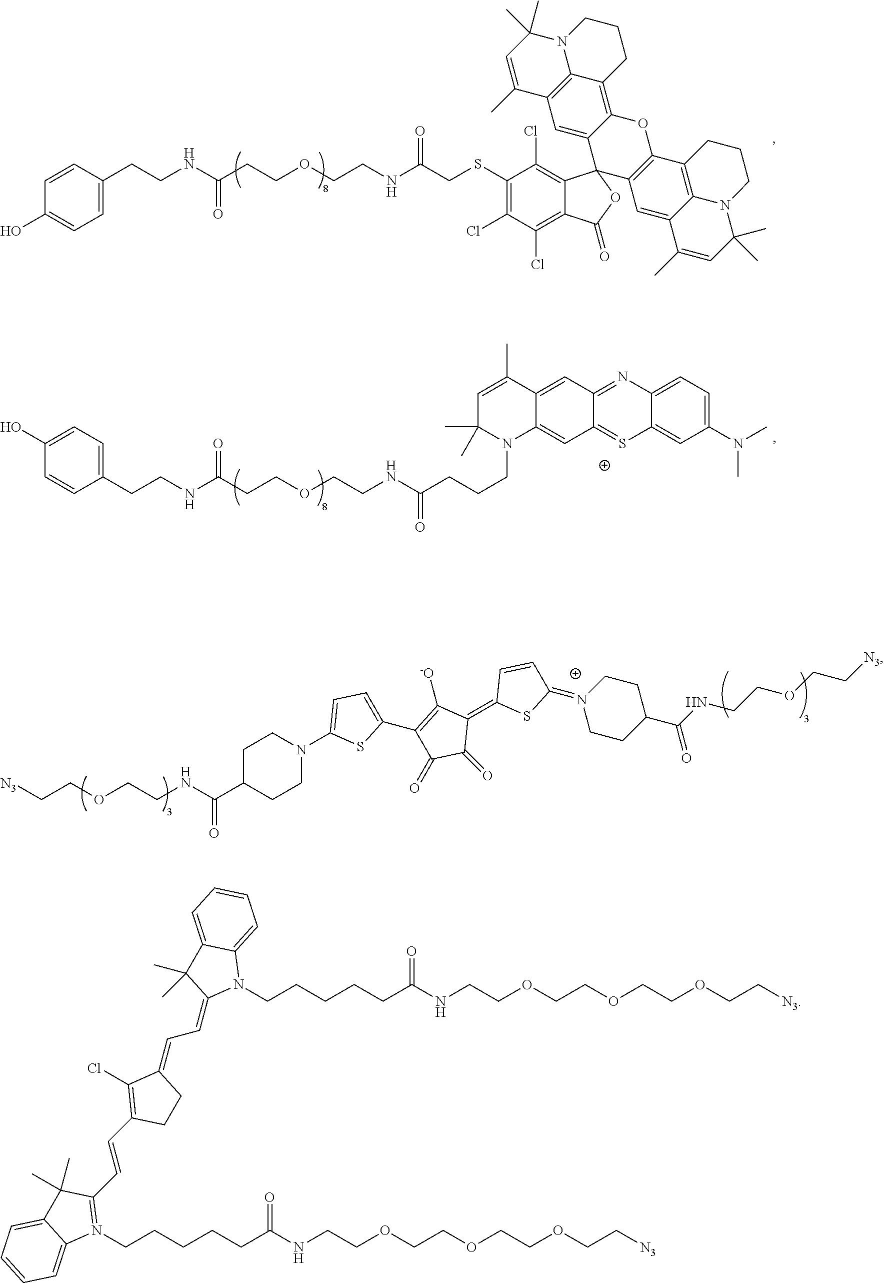

Additional Embodiment 119. A method of detecting one or more targets within a biological sample, comprising:
   (a) labeling a first morphological marker with a first detectable moiety, wherein the first detectable moiety has a first absorbance peak with FWHM of less than about 160 nm and an absorbance maximum (λₘₐₓ) between 330 nm+/−10 and 950 nm+/−10; and
   (b) labeling a first biomarker with a second detectable moiety, wherein the second detectable moiety is different than the first detectable moiety, and wherein the second detectable moiety has a first absorbance peak with FWHM of less than about 160 nm and an absorbance maximum (λₘₐₓ) between 330 nm+/−10 and 950 nm+/−10.
Additional Embodiment 120. The method of additional embodiment 119, wherein the first morphological marker is selected from the group consisting of DNA, histone proteins, markers for cytosol, markers for endoplasmic reticulum; nuclear membrane markers, markers of nucleoli or its substructures; markers for a nucleus and its substructures; markers of actin filaments, focal adhesions or their substructures; markers for centrosomes and centriolar satellites; markers for intermediate filaments or its substructures; markers for microtubule structures or substructures; markers for mitochondria; markers for localizing endoplasmic reticulum proteins across different cell lines; markers for the Golgi apparatus; markers used to localize Golgi apparatus-associated proteins across different cell lines; markers for the plasma membrane; markers for highly expressed single localizing plasma membrane proteins across different cell lines; and markers for vesicular organelles.
Additional Embodiment 121. The method of additional embodiment 119, wherein the absorbance maximum (λₘₐₓ) of the first detectable moiety and the absorbance maximum (λₘₐₓ) of the second detectable moiety are separated by at least about 20 nm.
Additional Embodiment 122. The method of additional embodiment 119, wherein the absorbance maximum (λₘₐₓ) of the first detectable moiety and the absorbance maximum (λₘₐₓ) of the second detectable moiety are separated by at least about 30 nm.
Additional Embodiment 123. The method of additional embodiment 119, wherein the absorbance maximum (λₘₐₓ) of the first detectable moiety and the absorbance maximum (λₘₐₓ) of the second detectable moiety are separated by at least about 40 nm.
Additional Embodiment 124. The method of additional embodiment 119, wherein the absorbance maximum (λₘₐₓ) of the first detectable moiety and the absorbance maximum (λₘₐₓ) of the second detectable moiety are separated by at least about 50 nm.
Additional Embodiment 125. A method of labelling at least a first biomarker in morphological context within a biological sample, comprising:
   (a) labelling a first morphological marker with a first detection probe that binds to the first morphological marker, wherein the first detection probe comprises an enzyme;
   (b) contacting the biological sample with a first anti-species antibody specific to the first detection probe, wherein the first anti-species antibody is conjugated directly or indirectly to at least one first enzyme;
   (c) contacting the biological sample with a first detectable conjugate comprising (i) a first detectable moiety, and (ii) a tyramide moiety, a quinone methide precursor moiety, or a derivative or analog of a tyramide moiety or quinone methide precursor moiety;
   (d) labeling the first biomarker in the biological sample with a second detection probe that binds the first biomarker;
   (e) contacting the biological sample with a second anti-species antibody specific to the second detection probe, wherein the second anti-species antibody is conjugated directly or indirectly to at least one second enzyme; and
   (f) contacting the biological sample with a second detectable conjugate comprising (i) a second detectable moiety, and (ii) a tyramide moiety, a quinone methide precursor moiety, or a derivative or analog of a tyramide moiety or quinone methide precursor moiety.
Additional Embodiment 126. A method of labelling at least a first biomarker in morphological context within a biological sample, comprising:
   (a) labelling a first morphological marker with a first detection probe that binds to the first morphological marker, wherein the first detection probe comprises an enzyme;
   (b) contacting the biological sample with a first anti-species antibody specific to the first detection probe, wherein the first anti-species antibody is conjugated directly or indirectly to at least one first enzyme;
   (c) contacting the biological sample with a first tissue reactive conjugate comprising: (i) a first member of a pair of reactive functional groups capable of participating in a click chemistry reaction, and (ii) a tyramide moiety, a quinone methide precursor moiety, or a derivative or analog of a tyramide moiety or quinone methide precursor moiety;
   (d) contacting the biological sample with a detectable conjugate comprising (i) a first detectable moiety, and (ii) a second member of the pair of reactive functional groups; and
   (e) labelling a first biomarker with a second detectable moiety, wherein the first and second detectable moieties are different.
Additional Embodiment 127. A method of detecting one or more targets within a biological sample, comprising:
   (a) labeling a first morphological marker with a first detectable moiety, wherein the first detectable moiety has a first absorbance peak with FWHM of less than about 160 nm; and
   (b) labeling a first biomarker with a second detectable moiety, wherein the second detectable moiety is different than the first detectable moiety, and wherein the second detectable moiety has a first absorbance peak with FWHM of less than about 160 nm; wherein an absorbance maximum (λₘₐₓ) of the first detectable moiety and an absorbance maximum (λₘₐₓ) of the second detectable moiety are separated by at least about 20 nm.
Additional Embodiment 128. The method of additional embodiment 127, wherein the absorbance maximum (λₘₐₓ) of the first detectable moiety and the absorbance maximum (λₘₐₓ) of the second detectable moiety are separated by at least about 30 nm.
Additional Embodiment 129. The method of additional embodiment 127, wherein the absorbance maximum (λₘₐₓ) of the first detectable moiety and the absorbance maximum (λₘₐₓ) of the second detectable moiety are separated by at least about 40 nm.
Additional Embodiment 130. The method of additional embodiment 127, wherein the absorbance maximum (λₘₐₓ) of the first detectable moiety and the absorbance maximum (λₘₐₓ) of the second detectable moiety are separated by at least about 50 nm.
Additional Embodiment 131. A method of detecting a biomarker in morphological context within a biological sample, comprising:
   (a) labeling at least a portion of a first morphological feature of the biological sample with a first detectable moiety, wherein the labeling of the first morphological feature comprises: (i) contacting a first morphological marker characteristic of the at least the portion of the first morphological feature with a first detection probe that binds to the first morphological marker, and (ii) covalently depositing the first detectable moiety on or proximal to the first morphological marker; and
   (b) labeling at least a portion of the first morphological feature of the biological sample with a second detectable moiety, wherein the labeling of the first morphological feature comprises: (i) contacting a second morphological marker characteristic of the at least the portion of the first morphological feature with a second detection probe that binds to the second morphological marker, and (ii) covalently depositing the second detectable moiety on or proximal to the second morphological marker; wherein the first and second detectable moieties are different. As such, in some embodiments, the same morphological feature may be labeled with two or more different detectable moieties by staining for the presence of at least two different morphological markers each characteristic of the same morphological feature. By way of example, the first morphological feature may be a cell nucleus, and the first and second morphological features may be DNA and histone proteins. In some embodiments, at least three different morphological markers characteristic of the same morphological feature are stained with different detectable moieties, including any of the detectable moieties described herein. In some embodiments, at least four different morphological markers characteristic of the same morphological feature are stained with different detectable moieties, including any of the detectable moieties described herein. In some embodiments, at least five different morphological markers characteristic of the same morphological feature are stained with different detectable moieties, including any of the detectable moieties described herein. In some embodiments, at least six different morphological markers characteristic of the same morphological feature are stained with different detectable moieties, including any of the detectable moieties described herein. In some embodiments, at least seven different morphological markers characteristic of the same morphological feature are stained with different detectable moieties, including any of the detectable moieties described herein. In some embodiments, at least eight different morphological markers characteristic of the same morphological feature are stained with different detectable moieties, including any of the detectable moieties described herein. In some embodiments, at least nine different morphological markers characteristic of the same morphological feature are stained with different detectable moieties, including any of the detectable moieties described herein. In some embodiments, at least ten different morphological markers characteristic of the same morphological feature are stained with different detectable moieties, including any of the detectable moieties described herein. In some embodiments, at least eleven different morphological markers characteristic of the same morphological feature are stained with different detectable moieties, including any of the detectable moieties described herein.
Additional Embodiment 132. The method of additional embodiment 131, wherein the FWHM of the first and/or second detectable moieties is less than about 200 nm.
Additional Embodiment 133. The method of additional embodiment 131, wherein the FWHM of the first and/or second detectable moieties is less than about 130 nm.
Additional Embodiment 134. The method of additional embodiment 131, wherein the first and second detectable moieties are each independently conjugated to a tyramide or a derivative thereof, a quinone methide precursor moiety or a derivative thereof, or a reactive functional group capable of participating in a click chemistry reaction; and wherein the covalent deposition of the first detectable moiety and the second detectable moiety independently comprises one of tyramide signal amplification, quinone methide chemistry, or click chemistry.
Additional Embodiment 135. The method of additional embodiment 131, wherein the absorbance maximum (λₘₐₓ) of the first detectable moiety and the absorbance maximum (λₘₐₓ) of the second detectable moiety are separated by at least about 20 nm.
Additional Embodiment 136. The method of additional embodiment 131, wherein the absorbance maximum (λₘₐₓ) of the first detectable moiety and the absorbance maximum (λₘₐₓ) of the second detectable moiety are separated by at least about 30 nm.
Additional Embodiment 137. The method of additional embodiment 131, wherein the absorbance maximum (λₘₐₓ) of the first detectable moiety and the absorbance maximum (λₘₐₓ) of the second detectable moiety are separated by at least about 40 nm.
Additional Embodiment 138. The method of additional embodiment 131, wherein the absorbance maximum (λₘₐₓ) of the first detectable moiety and the absorbance maximum (λₘₐₓ) of the second detectable moiety are separated by at least about 50 nm.
Additional Embodiment 139. The method of additional embodiment 131, wherein the first and second morphological markers are selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers.
Additional Embodiment 140. The method of additional embodiment 131, wherein the first detectable moiety comprises a coumarin core.
Additional Embodiment 141. The method of additional embodiment 140, wherein the second detectable moiety is within the visible spectrum or within the infrared spectrum.
Additional Embodiment 142. The method of additional embodiment 140, wherein the second detectable moiety is within the ultraviolet spectrum.
Additional Embodiment 143. The method of additional embodiment 142, wherein the first and second detectable moieties have absorbance maximums (λₘₐₓ) that are separated by at least 20 nm.
Additional Embodiment 144. The method of additional embodiment 131, wherein the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core.
Additional Embodiment 145. The method of additional embodiment 144, wherein the second detectable moiety is within the ultraviolet spectrum or within the infrared spectrum.
Additional Embodiment 146. The method of additional embodiment 144, wherein the second detectable moiety is within the visible spectrum.
Additional Embodiment 147. The method of additional embodiment 144, wherein the first and second detectable moieties have absorbance maximums (λₘₐₓ) that are separated by at least 20 nm.
Additional Embodiment 148. The method of additional embodiment 131, wherein the first detectable moiety comprises a heptamethine cyanine core or a croconate core.
Additional Embodiment 149. The method of additional embodiment 148, wherein the second detectable moiety is within the visible spectrum or within the ultraviolet spectrum.
Additional Embodiment 150. The method of additional embodiment 148, wherein the second detectable moiety is within the infrared spectrum.
Additional Embodiment 151. The method of additional embodiment 148, wherein the first and second detectable moieties have absorbance maximums (λₘₐₓ) that are separated by at least 20 nm.
Additional Embodiment 152. The method of additional embodiment 131, further comprising labeling at least a portion of the first morphological feature of the biological sample with a third detectable moiety, wherein the labeling of the first morphological feature comprises: (i) contacting a third morphological marker characteristic of the at least the portion of the first morphological feature with a third detection probe that binds to the third morphological marker, and (ii) covalently depositing the third detectable moiety on or proximal to the third morphological marker; wherein the third detectable moiety is different from the first and second detectable moieties.
Additional Embodiment 153. The method of additional embodiment 131, further comprising labeling a first biomarker in the biological sample with a third detectable moiety, wherein the third detectable moiety is different from the first detectable moiety and the second detectable moiety, and wherein the labeling of the first biomarker comprises: (i) contacting the first biomarker with a third detection probe that binds the first biomarker; and (ii) covalently depositing the third detectable moiety on or proximal to the first biomarker.
Additional Embodiment 154. A biological sample comprising: (a) a first morphological marker labeled with a first detectable moiety; and (b) a second morphological marker labeled with a second detectable moiety; wherein the first and second detectable moieties each have a first absorbance peak with FWHM of less than about 200 nm and an absorbance maximum (λₘₐₓ) between 330 nm+/−10 and 950 nm+/−10; and wherein an absorbance maximum (λₘₐₓ) of the first detectable moiety and an absorbance maximum (λₘₐₓ) of the second detectable moiety are separated by at least 20 nm. In some embodiments, the first and second morphological markers are characteristic of the same morphological feature. As such, in some embodiments, the same morphological feature may be labeled with two or more different detectable moieties by staining for the presence of at least two different morphological markers each characteristic of the same morphological feature. By way of example, the first morphological feature may be a cell nucleus, and the first and second morphological features may be DNA and histone proteins. In some embodiments, at least three different morphological markers characteristic of the same morphological feature are stained with different detectable moieties, including any of the detectable moieties described herein. In some embodiments, at least four different morphological markers characteristic of the same morphological feature are stained with different detectable moieties, including any of the detectable moieties described herein. In some embodiments, at least five different morphological markers characteristic of the same morphological feature are stained with different detectable moieties, including any of the detectable moieties described herein. In some embodiments, at least six different morphological markers characteristic of the same morphological feature are stained with different detectable moieties, including any of the detectable moieties described herein. In some embodiments, at least seven different morphological markers characteristic of the same morphological feature are stained with different detectable moieties, including any of the detectable moieties described herein. In some embodiments, at least eight different morphological markers characteristic of the same morphological feature are stained with different detectable moieties, including any of the detectable moieties described herein. In some embodiments, at least nine different morphological markers characteristic of the same morphological feature are stained with different detectable moieties, including any of the detectable moieties described herein. In some embodiments, at least ten different morphological markers characteristic of the same morphological feature are stained with different detectable moieties, including any of the detectable moieties described herein. In some embodiments, at least eleven different morphological markers characteristic of the same morphological feature are stained with different detectable moieties, including any of the detectable moieties described herein.
Additional Embodiment 155. The method of additional embodiment 154, wherein the FWHM of the first and/or second detectable moieties is less than about 200 nm.
Additional Embodiment 156. The method of additional embodiment 154, wherein the FWHM of the first and/or second detectable moieties is less than about 130 nm.
Additional Embodiment 157. The method of additional embodiment 154, wherein the first and second detectable moieties are each independently conjugated to a tyramide or a derivative thereof, a quinone methide precursor moiety or a derivative thereof, or a reactive functional group capable of participating in a click chemistry reaction; and wherein the covalent deposition of the first detectable moiety and the second detectable moiety independently comprises one of tyramide signal amplification, quinone methide chemistry, or click chemistry.
Additional Embodiment 158. The method of additional embodiment 154, wherein the absorbance maximum (λₘₐₓ) of the first detectable moiety and the absorbance maximum (λₘₐₓ) of the second detectable moiety are separated by at least about 20 nm.
Additional Embodiment 159. The method of additional embodiment 154, wherein the absorbance maximum (λₘₐₓ) of the first detectable moiety and the absorbance maximum (λₘₐₓ) of the second detectable moiety are separated by at least about 30 nm.
Additional Embodiment 160. The method of additional embodiment 154, wherein the absorbance maximum (λₘₐₓ) of the first detectable moiety and the absorbance maximum (λₘₐₓ) of the second detectable moiety are separated by at least about 40 nm.
Additional Embodiment 161. The method of additional embodiment 154, wherein the absorbance maximum (λₘₐₓ) of the first detectable moiety and the absorbance maximum (λₘₐₓ) of the second detectable moiety are separated by at least about 50 nm.
Additional Embodiment 162. The method of additional embodiment 154, wherein the first and second morphological markers are selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers.
Additional Embodiment 163. The method of additional embodiment 154, wherein the first detectable moiety comprises a coumarin core.
Additional Embodiment 164. The method of additional embodiment 163, wherein the second detectable moiety is within the visible spectrum or within the infrared spectrum.
Additional Embodiment 165. The method of additional embodiment 163, wherein the second detectable moiety is within the ultraviolet spectrum.
Additional Embodiment 166. The method of additional embodiment 163, wherein the first and second detectable moieties have absorbance maximums (λₘₐₓ) that are separated by at least 20 nm.
Additional Embodiment 167. The method of additional embodiment 154, wherein the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core.
Additional Embodiment 168. The method of additional embodiment 167, wherein the second detectable moiety is within the ultraviolet spectrum or within the infrared spectrum.
Additional Embodiment 169. The method of additional embodiment 167, wherein the second detectable moiety is within the visible spectrum.
Additional Embodiment 170. The method of additional embodiment 167, wherein the first and second detectable moieties have absorbance maximums (λₘₐₓ) that are separated by at least 20 nm.
Additional Embodiment 171. The method of additional embodiment 154, wherein the first detectable moiety comprises a heptamethine cyanine core or a croconate core.
Additional Embodiment 172. The method of additional embodiment 171, wherein the second detectable moiety is within the visible spectrum or within the ultraviolet spectrum.
Additional Embodiment 173. The method of additional embodiment 171, wherein the second detectable moiety is within the infrared spectrum.
Additional Embodiment 174. The method of additional embodiment 171, wherein the first and second detectable moieties have absorbance maximums (λₘₐₓ) that are separated by at least 20 nm.
Additional Embodiment 175. A biological sample comprising: (a) a first morphological marker labeled with a first detectable moiety; and (b) a second morphological marker labeled with a second detectable moiety; wherein the first and second detectable moieties each have a first absorbance peak with FWHM of less than about 200 nm and an absorbance maximum (λₘₐₓ) between 330 nm+/−10 and 950 nm+/−1; and wherein an absorbance maximum (λₘₐₓ) of the first detectable moiety and an absorbance maximum (λₘₐₓ) of the second detectable moiety are separated by at least 20 nm; wherein the biological sample is prepared by:
   (i) contacting the biological sample with a first primary antibody specific to the first morphological marker;
   (ii) contacting the biological sample with a first secondary antibody specific to the first primary antibody, wherein the first secondary antibody is conjugated to an enzyme;
   (iii) contacting the biological sample with a first detectable conjugate comprising (a) a tyramide moiety, a quinone methide precursor moiety, or a derivative or analog of a tyramide moiety or quinone methide precursor moiety; and (b) the first detectable moiety;
   (iv) contacting the biological sample with a second primary antibody specific to the second morphological marker;
   (v) contacting the biological sample with a second secondary antibody specific to the second primary antibody, wherein the second secondary antibody is conjugated to an enzyme; and
   (vi) contacting the biological sample with a second detectable conjugate comprising (a) a tyramide moiety, a quinone methide precursor moiety, or a derivative or analog of a tyramide moiety or quinone methide precursor moiety; and (b) the second detectable moiety.
In some embodiments, the first and second morphological markers are characteristic of the same morphological feature. As such, in some embodiments, the same morphological feature may be labeled with two or more different detectable moieties by staining for the presence of at least two different morphological markers each characteristic of the same morphological feature. By way of example, the first morphological feature may be a cell nucleus, and the first and second morphological features may be DNA and histone proteins. In some embodiments, at least three different morphological markers characteristic of the same morphological feature are stained with different detectable moieties, including any of the detectable moieties described herein. In some embodiments, at least four different morphological markers characteristic of the same morphological feature are stained with different detectable moieties, including any of the detectable moieties described herein. In some embodiments, at least five different morphological markers characteristic of the same morphological feature are stained with different detectable moieties, including any of the detectable moieties described herein. In some embodiments, at least six different morphological markers characteristic of the same morphological feature are stained with different detectable moieties, including any of the detectable moieties described herein. In some embodiments, at least seven different morphological markers characteristic of the same morphological feature are stained with different detectable moieties, including any of the detectable moieties described herein. In some embodiments, at least eight different morphological markers characteristic of the same morphological feature are stained with different detectable moieties, including any of the detectable moieties described herein. In some embodiments, at least nine different morphological markers characteristic of the same morphological feature are stained with different detectable moieties, including any of the detectable moieties described herein. In some embodiments, at least ten different morphological markers characteristic of the same morphological feature are stained with different detectable moieties, including any of the detectable moieties described herein. In some embodiments, at least eleven different morphological markers characteristic of the same morphological feature are stained with different detectable moieties, including any of the detectable moieties described herein.
Additional Embodiment 176. The method of additional embodiment 175, wherein the FWHM of the first and/or second detectable moieties is less than about 200 nm.
Additional Embodiment 177. The method of additional embodiment 175, wherein the FWHM of the first and/or second detectable moieties is less than about 130 nm.
Additional Embodiment 178. The method of additional embodiment 175, wherein the first and second detectable moieties are each independently conjugated to a tyramide or a derivative thereof, a quinone methide precursor moiety or a derivative thereof, or a reactive functional group capable of participating in a click chemistry reaction; and wherein the covalent deposition of the first detectable moiety and the second detectable moiety independently comprises one of tyramide signal amplification, quinone methide chemistry, or click chemistry.
Additional Embodiment 179. The method of additional embodiment 175, wherein the absorbance maximum (λₘₐₓ) of the first detectable moiety and the absorbance maximum (λₘₐₓ) of the second detectable moiety are separated by at least about 20 nm.
Additional Embodiment 180. The method of additional embodiment 175, wherein the absorbance maximum (λₘₐₓ) of the first detectable moiety and the absorbance maximum (λₘₐₓ) of the second detectable moiety are separated by at least about 30 nm.
Additional Embodiment 181. The method of additional embodiment 175, wherein the absorbance maximum (λₘₐₓ) of the first detectable moiety and the absorbance maximum (λₘₐₓ) of the second detectable moiety are separated by at least about 40 nm.
Additional Embodiment 182. The method of additional embodiment 175, wherein the absorbance maximum (λₘₐₓ) of the first detectable moiety and the absorbance maximum (λₘₐₓ) of the second detectable moiety are separated by at least about 50 nm.
Additional Embodiment 183. The method of additional embodiment 175, wherein the first and second morphological markers are selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers.
Additional Embodiment 184. The method of additional embodiment 175, wherein the first detectable moiety comprises a coumarin core.
Additional Embodiment 185. The method of additional embodiment 184, wherein the second detectable moiety is within the visible spectrum or within the infrared spectrum.
Additional Embodiment 186. The method of additional embodiment 184, wherein the second detectable moiety is within the ultraviolet spectrum.
Additional Embodiment 187. The method of additional embodiment 184, wherein the first and second detectable moieties have absorbance maximums (λₘₐₓ) that are separated by at least 20 nm.
Additional Embodiment 188. The method of additional embodiment 175, wherein the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core.
Additional Embodiment 189. The method of additional embodiment 188, wherein the second detectable moiety is within the ultraviolet spectrum or within the infrared spectrum.
Additional Embodiment 190. The method of additional embodiment 188, wherein the second detectable moiety is within the visible spectrum.
Additional Embodiment 191. The method of additional embodiment 188, wherein the first and second detectable moieties have absorbance maximums (λₘₐₓ) that are separated by at least 20 nm.
Additional Embodiment 192. The method of additional embodiment 175, wherein the first detectable moiety comprises a heptamethine cyanine core or a croconate core.
Additional Embodiment 193. The method of additional embodiment 192, wherein the second detectable moiety is within the visible spectrum or within the ultraviolet spectrum.
Additional Embodiment 194. The method of additional embodiment 192, wherein the second detectable moiety is within the infrared spectrum.
Additional Embodiment 195. The method of additional embodiment 192, wherein the first and second detectable moieties have absorbance maximums (λₘₐₓ) that are separated by at least 20 nm.
Additional Embodiment 196. A biological sample comprising: (a) a first morphological marker labeled with a first detectable moiety; and (b) a second morphological marker labeled with a second detectable moiety; wherein the first and second detectable moieties each have a first absorbance peak with FWHM of less than about 200 nm and an absorbance maximum (λₘₐₓ) between 330 nm+/−10 and 950 nm+/−1; and wherein an absorbance maximum (λₘₐₓ) of the first detectable moiety and an absorbance maximum (λₘₐₓ) of the second detectable moiety are separated by at least 20 nm; wherein the biological sample is prepared by:
   (i) contacting the biological sample with a first primary antibody specific to the first morphological marker;
   (ii) contacting the biological sample with a first secondary antibody specific to the first primary antibody, wherein the first secondary antibody is conjugated to an enzyme;
   (iii) contacting the biological sample with a first tissue reactive moiety comprising (a) a tyramide moiety, a quinone methide precursor moiety, or a derivative or analog of a tyramide moiety or quinone methide precursor moiety; and (b) a first reactive functional group capable of participating in a click chemistry reaction;
   (iv) contacting the biological sample with a first detectable conjugate comprising: (a) the first detectable moiety; and (b) a second reactive functional group;
   (v) contacting the biological sample with a second primary antibody specific to the second morphological marker;
   (vi) contacting the biological sample with a second secondary antibody specific to the second primary antibody, wherein the second secondary antibody is conjugated to an enzyme;
   (vii) contacting the biological sample with a second tissue reactive moiety comprising (a) a tyramide moiety, a quinone methide precursor moiety, or a derivative or analog of a tyramide moiety or quinone methide precursor moiety; and (b) a first reactive functional group capable of participating in a click chemistry reaction;
   (viii) contacting the biological sample with a second detectable conjugate comprising: (a) the second detectable moiety; and (b) a second reactive functional group.
In some embodiments, the first and second morphological markers are characteristic of the same morphological feature. As such, in some embodiments, the same morphological feature may be labeled with two or more different detectable moieties by staining for the presence of at least two different morphological markers each characteristic of the same morphological feature. By way of example, the first morphological feature may be a cell nucleus, and the first and second morphological features may be DNA and histone proteins. In some embodiments, at least three different morphological markers characteristic of the same morphological feature are stained with different detectable moieties, including any of the detectable moieties described herein. In some embodiments, at least four different morphological markers characteristic of the same morphological feature are stained with different detectable moieties, including any of the detectable moieties described herein. In some embodiments, at least five different morphological markers characteristic of the same morphological feature are stained with different detectable moieties, including any of the detectable moieties described herein. In some embodiments, at least six different morphological markers characteristic of the same morphological feature are stained with different detectable moieties, including any of the detectable moieties described herein. In some embodiments, at least seven different morphological markers characteristic of the same morphological feature are stained with different detectable moieties, including any of the detectable moieties described herein. In some embodiments, at least eight different morphological markers characteristic of the same morphological feature are stained with different detectable moieties, including any of the detectable moieties described herein. In some embodiments, at least nine different morphological markers characteristic of the same morphological feature are stained with different detectable moieties, including any of the detectable moieties described herein. In some embodiments, at least ten different morphological markers characteristic of the same morphological feature are stained with different detectable moieties, including any of the detectable moieties described herein. In some embodiments, at least eleven different morphological markers characteristic of the same morphological feature are stained with different detectable moieties, including any of the detectable moieties described herein.
Additional Embodiment 197. The method of additional embodiment 196, wherein the FWHM of the first and/or second detectable moieties is less than about 200 nm.
Additional Embodiment 198. The method of additional embodiment 196, wherein the FWHM of the first and/or second detectable moieties is less than about 130 nm.
Additional Embodiment 199. The method of additional embodiment 196, wherein the first and second detectable moieties are each independently conjugated to a tyramide or a derivative thereof, a quinone methide precursor moiety or a derivative thereof, or a reactive functional group capable of participating in a click chemistry reaction; and wherein the covalent deposition of the first detectable moiety and the second detectable moiety independently comprises one of tyramide signal amplification, quinone methide chemistry, or click chemistry.
Additional Embodiment 200. The method of additional embodiment 196, wherein the absorbance maximum (λₘₐₓ) of the first detectable moiety and the absorbance maximum (λₘₐₓ) of the second detectable moiety are separated by at least about 20 nm.
Additional Embodiment 201. The method of additional embodiment 196, wherein the absorbance maximum (λₘₐₓ) of the first detectable moiety and the absorbance maximum (λₘₐₓ) of the second detectable moiety are separated by at least about 30 nm.
Additional Embodiment 202. The method of additional embodiment 196, wherein the absorbance maximum (λₘₐₓ) of the first detectable moiety and the absorbance maximum (λₘₐₓ) of the second detectable moiety are separated by at least about 40 nm.
Additional Embodiment 203. The method of additional embodiment 196, wherein the absorbance maximum (λₘₐₓ) of the first detectable moiety and the absorbance maximum (λₘₐₓ) of the second detectable moiety are separated by at least about 50 nm.
Additional Embodiment 204. The method of additional embodiment 196, wherein the first and second morphological markers are selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers.
Additional Embodiment 205. The method of additional embodiment 196, wherein the first detectable moiety comprises a coumarin core.
Additional Embodiment 206. The method of additional embodiment 205, wherein the second detectable moiety is within the visible spectrum or within the infrared spectrum.
Additional Embodiment 207. The method of additional embodiment 205, wherein the second detectable moiety is within the ultraviolet spectrum.
Additional Embodiment 208. The method of additional embodiment 205, wherein the first and second detectable moieties have absorbance maximums (λₘₐₓ) that are separated by at least 20 nm.
Additional Embodiment 209. The method of additional embodiment 196, wherein the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core.
Additional Embodiment 210. The method of additional embodiment 209, wherein the second detectable moiety is within the ultraviolet spectrum or within the infrared spectrum.
Additional Embodiment 211. The method of additional embodiment 209, wherein the second detectable moiety is within the visible spectrum.
Additional Embodiment 212. The method of additional embodiment 209, wherein the first and second detectable moieties have absorbance maximums (λₘₐₓ) that are separated by at least 20 nm.
Additional Embodiment 213. The method of additional embodiment 196, wherein the first detectable moiety comprises a heptamethine cyanine core or a croconate core.
Additional Embodiment 214. The method of additional embodiment 213, wherein the second detectable moiety is within the visible spectrum or within the ultraviolet spectrum.
Additional Embodiment 215. The method of additional embodiment 213, wherein the second detectable moiety is within the infrared spectrum.
Additional Embodiment 216. The method of additional embodiment 213, wherein the first and second detectable moieties have absorbance maximums (λₘₐₓ) that are separated by at least 20 nm.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

Although the disclosure herein has been described with reference to particular embodiments, it is to be understood that these embodiments are merely illustrative of the principles and applications of the present disclosure. It is therefore understood that numerous modifications may be made to the illustrative embodiments and that other arrangements may be devised without departing from the spirit and scope of the present disclosure as defined by the appended claims.

1. A method of detecting a biomarker in morphological context within a biological sample, comprising:
(a) labeling at least a portion of a first morphological feature of the biological sample with a first detectable moiety, wherein the labeling of the first morphological feature comprises: (i) contacting a first morphological marker characteristic of the at least the portion of the first morphological feature with a first detection probe that binds to the first morphological marker, and (ii) covalently depositing the first detectable moiety on or proximal to the first morphological marker; and,
(b) labeling a first biomarker in the biological sample with a second detectable moiety, wherein the second detectable moiety is different from the first detectable moiety, and wherein the labeling of the first biomarker comprises: (i) contacting the first biomarker with a second detection probe that binds the first biomarker; and (ii) covalently depositing the second detectable moiety on or proximal to the first biomarker.

2. The method of claim 1, wherein the FWHM of the first and/or second detectable moieties is less than about 200 nm.

3. The method of claim 1, wherein the first and second detectable moieties are each independently conjugated to a tyramide or a derivative thereof, a quinone methide precursor moiety or a derivative thereof, or a reactive functional group capable of participating in a click chemistry reaction; and wherein the covalent deposition of the first detectable moiety and the second detectable moiety independently comprises one of tyramide signal amplification, quinone methide chemistry, or click chemistry.

4. The method of claim 1, wherein the absorbance maximum (λₘₐₓ) of the first detectable moiety and the absorbance maximum (λₘₐₓ) of the second detectable moiety are separated by at least about 20 nm.

5. The method of claim 1 wherein the first morphological marker comprises DNA.

6. The method of claim 5, wherein the labeling of the DNA with the first detectable moiety comprises: (a) contacting the biological sample with an anti-DNA primary antibody; (b) contacting the biological sample with an anti-specifies secondary antibody specific to the anti-DNA primary antibody, wherein the anti-species antibody is conjugated directly or indirectly to at least one enzyme; and (c) contacting the biological sample with a first detectable conjugate comprising (i) the first detectable moiety, and (ii) a tyramide moiety, a quinone methide precursor moiety, or a derivative or analog of a tyramide moiety or quinone methide precursor moiety.

7. The method of claim 5, wherein the labeling of the DNA with the first detectable moiety comprises: (a) contacting the biological sample with an anti-DNA primary antibody; (b) contacting the biological sample with an anti-specifies secondary antibody specific to the anti-DNA antibody, wherein the anti-species antibody is conjugated directly or indirectly to at least one enzyme; (c) contacting the biological sample with a first tissue reactive conjugate comprising: (i) a first member of a pair of reactive functional groups capable of participating in a click chemistry reaction, and (ii) a tyramide moiety, a quinone methide precursor moiety, or a derivative or analog of a tyramide moiety or quinone methide precursor moiety; and (d) contacting the biological sample with a detectable conjugate comprising (i) the first detectable moiety, and (ii) a second member of the pair of reactive functional groups.

8. The method claim 1, wherein the first morphological marker comprises a histone protein.

9. The method of claim 8, wherein the labeling of the histone proteins with the first detectable moiety comprises: (a) contacting the biological sample with an anti-histone primary antibody; (b) contacting the biological sample with an anti-specifies secondary antibody specific to the anti-histone primary antibody, wherein the anti-species antibody is conjugated directly or indirectly to at least one enzyme; and (c) contacting the biological sample with a first detectable conjugate comprising (i) the first detectable moiety, and (ii) a tyramide moiety, a quinone methide precursor moiety, or a derivative or analog of a tyramide moiety or quinone methide precursor moiety.

10. The method of claim 8, wherein the labeling of the histone proteins with the first detectable moiety comprises: (a) contacting the biological sample with an anti-histone primary antibody; (b) contacting the biological sample with an anti-specifies secondary antibody specific to the anti-histone antibody, wherein the anti-species antibody is conjugated directly or indirectly to at least one enzyme; (c) contacting the biological sample with a first tissue reactive conjugate comprising: (i) a first member of a pair of reactive functional groups capable of participating in a click chemistry reaction, and (ii) a tyramide moiety, a quinone methide precursor moiety, or a derivative or analog of a tyramide moiety or quinone methide precursor moiety; and (d) contacting the biological sample with a detectable conjugate comprising (i) the first detectable moiety, and (ii) a second member of the pair of reactive functional groups.

11. The method of claim 1, wherein the first morphological marker is selected from the group consisting of a marker for cytosol, a marker for the nucleus, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers.

12. The method of claim 1, wherein the first detectable moiety comprises a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core.

13. A method of detecting one or more targets within a biological sample, comprising:
(a) labeling a first morphological marker with a first detectable moiety comprising a core selected from the group consisting of a coumarin core, a phenoxazinone core, a 4-Hydroxy-3-phenoxazinone core, a 7-amino-4-Hydroxy-3-phenoxazinone core, a thioninium core, a phenoxazine core, a phenoxathiin-3-one core, a xanthene core, a heptamethine cyanine core and a croconate core;
(b) labeling a first biomarker with a second detectable moiety comprising a core selected from the group consisting of a coumarin core, a phenoxazinone core, a 4-Hydroxy-3-phenoxazinone core, a 7-amino-4-Hydroxy-3-phenoxazinone core, a thioninium core, a phenoxazine core, a phenoxathiin-3-one core, a xanthene core, a heptamethine cyanine core and a croconate core;
wherein the first and second detectable moieties are different and have absorbance maximums (λₘₐₓ) which differ by at least 10 nm.

14. The method of claim 13, wherein the first morphological marker comprises histone proteins.

15. The method of claim 13, wherein the first morphological marker is selected from the group consisting of a marker for cytosol, a nuclear membrane marker, a marker for nucleoli, a marker for actin filaments, a marker for centrosomes, a marker for centriolar satellites, a marker for intermediate filaments, a marker for microtubule structures, mitochondrial markers, markers for endoplasmic reticulum, Golgi apparatus markers, plasma membrane markers, and vesicular organelle markers.

16. The method of claim 13, wherein the absorbance maximums (λₘₐₓ) of the first and second detectable moieties differ by at least 30 nm.

17. The method claim 13, further comprising labeling a second biomarker with a third detectable moiety, wherein the third detectable moiety is different than the first and second detectable moieties, and wherein the first, second, and third detectable moieties have absorbance maximums (λₘₐₓ) which differ by at least 30 nm.

18. The method of claim 13, wherein the first and second detectable moieties are selected from the group consisting of: 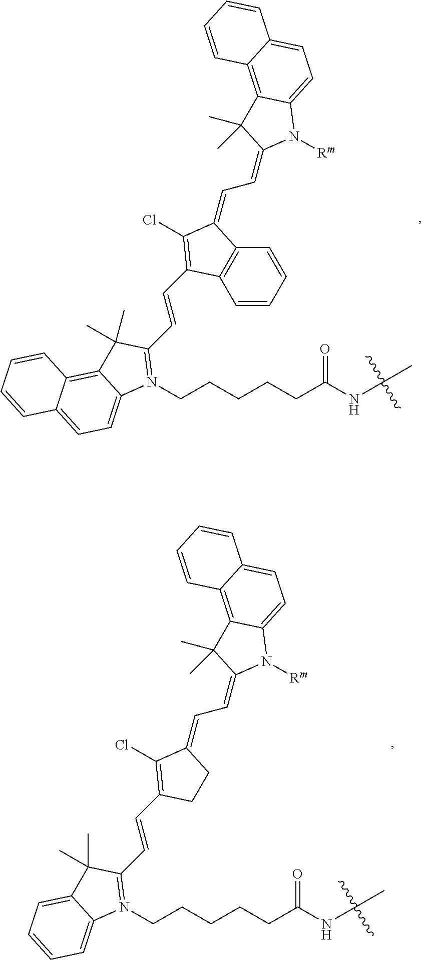 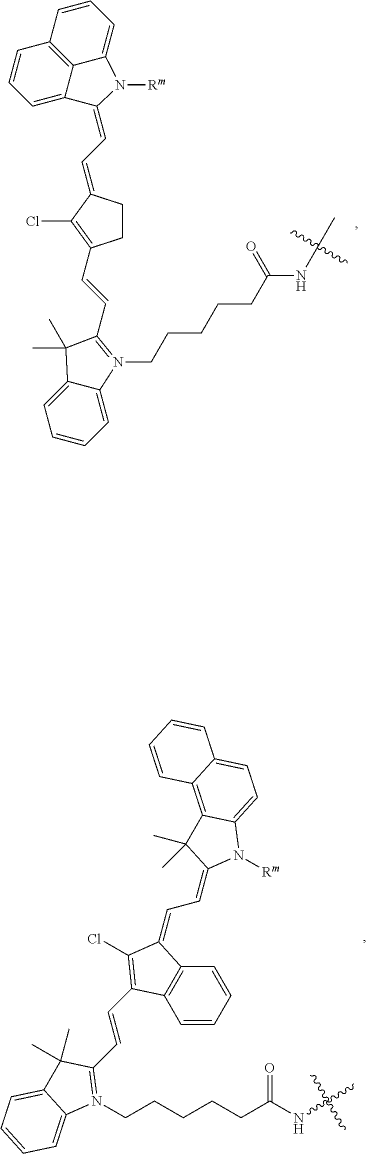 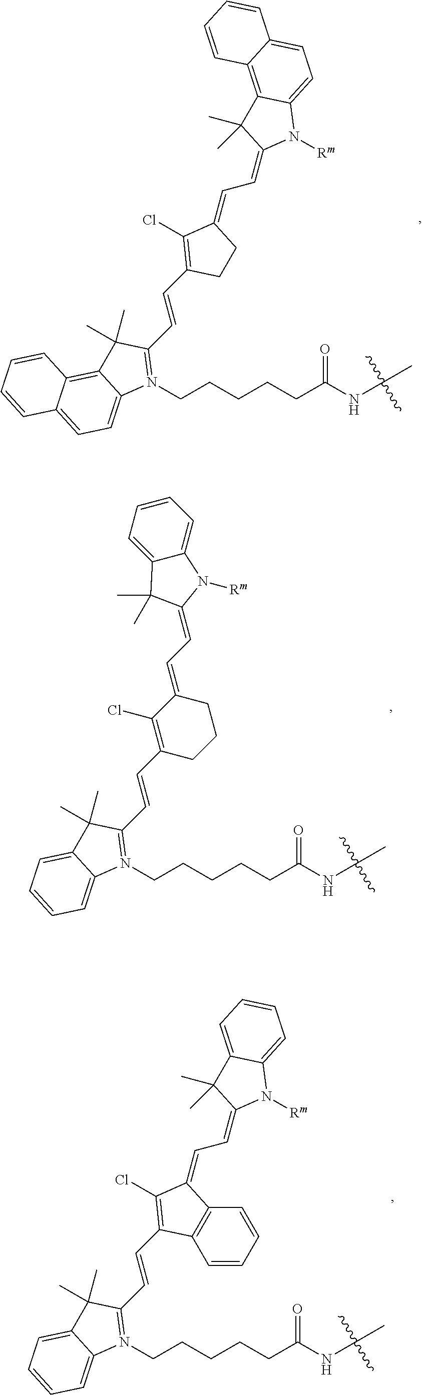 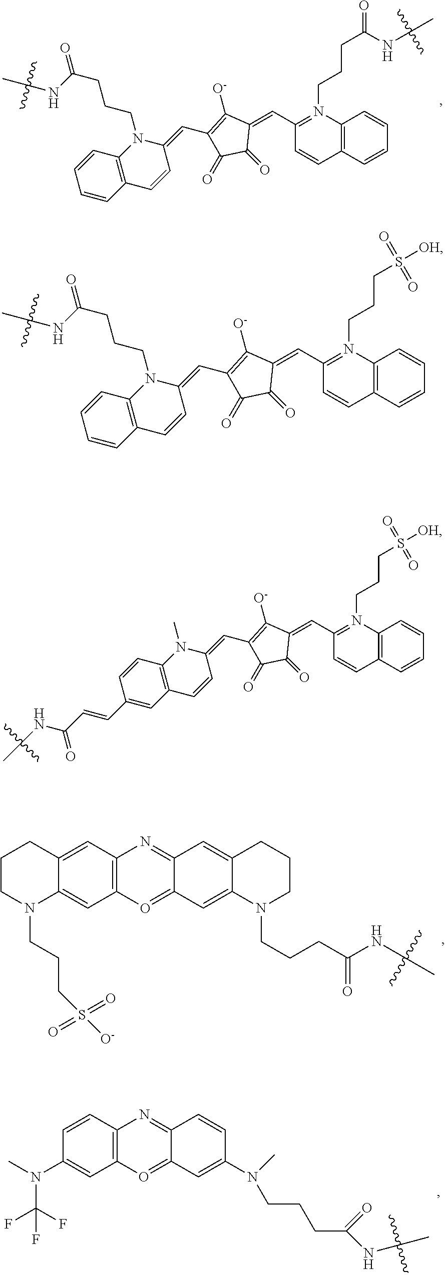 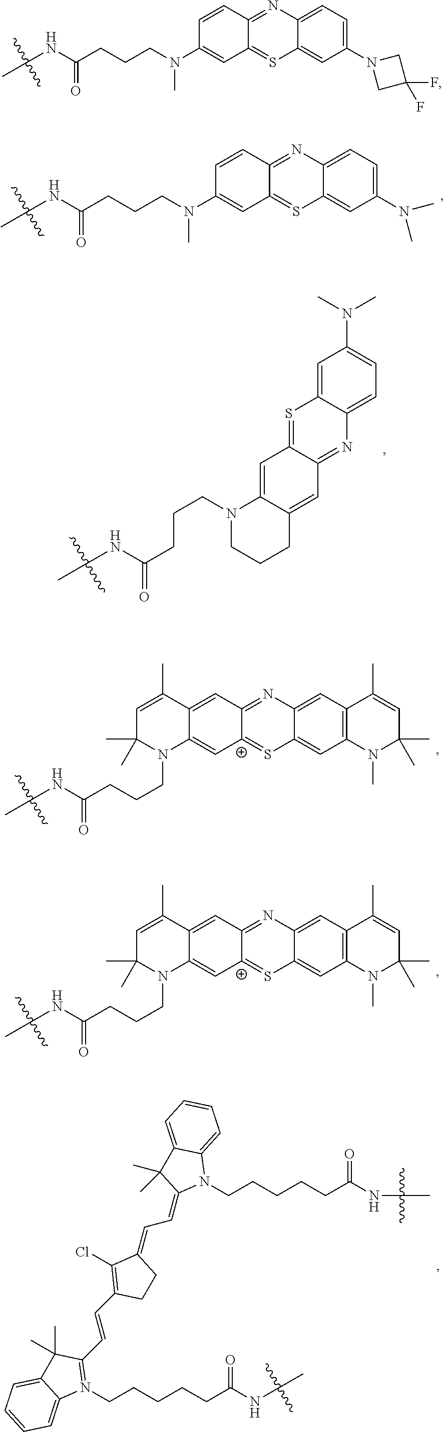 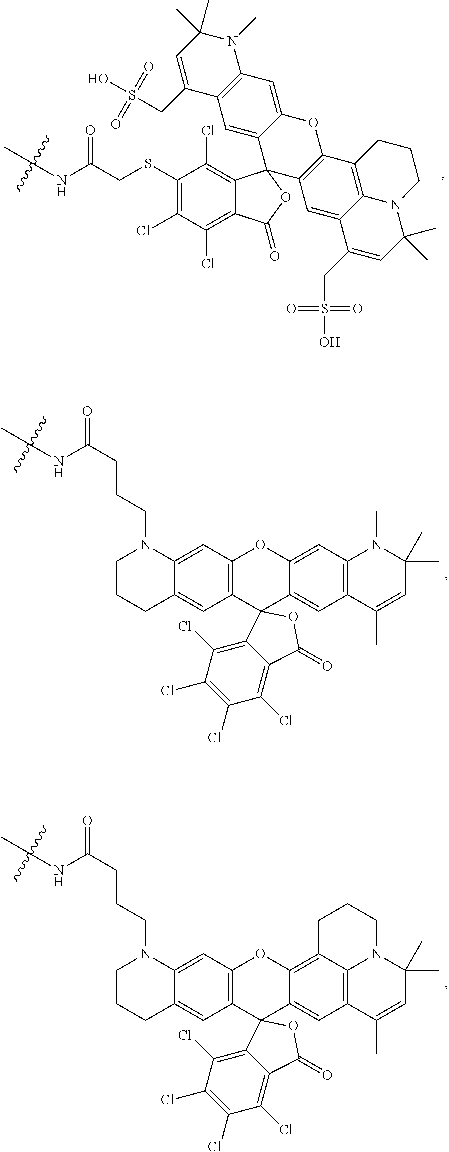 [SMILES: *NC(=O)CSc1c(Cl)c(Cl)c2c(c1Cl)C1(OC2=O)c2cc3c(cc2Oc2c1cc1c4c2CCCN4C(C)(C)C=C1CS(=O)(=O)O)N(C)C(C)(C)C=C3CS(=O)(=O)O ; *NC(=O)CCCN1CCCc2cc3c(cc21)Oc1cc2c(cc1C31OC(=O)c3c(Cl)c(Cl)c(Cl)c(Cl)c31)C(C)=CC(C)(C)N2C ; *NC(=O)CCCN1CCCc2cc3c(cc21)Oc1c(cc2c4c1CCCN4C(C)(C)C=C2C)C31OC(=O)c2c(Cl)c(Cl)c(Cl)c(Cl)c21] 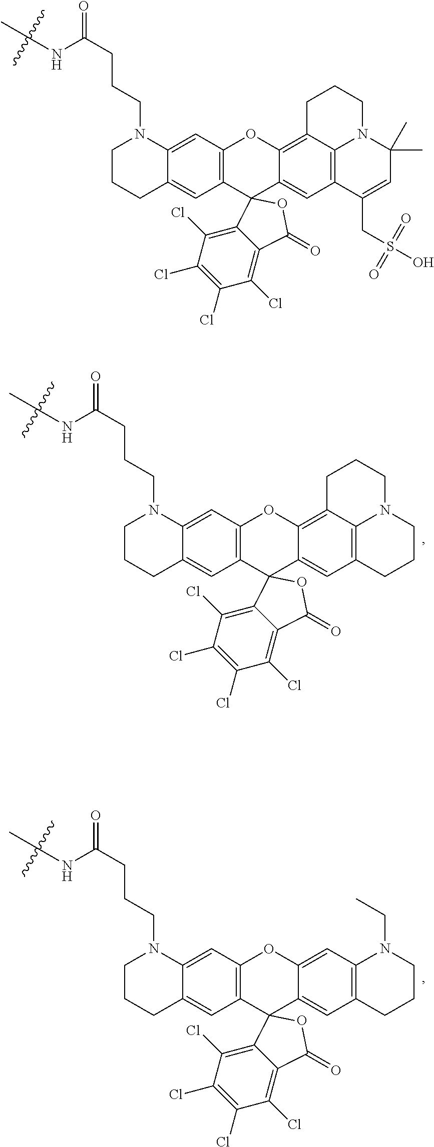 [SMILES: *NC(=O)CCCN1CCCc2cc3c(cc21)Oc1c(cc2c4c1CCCN4C(C)(C)C=C2CS(=O)(=O)O)C31OC(=O)c2c(Cl)c(Cl)c(Cl)c(Cl)c21 ; *NC(=O)CCCN1CCCc2cc3c(cc21)Oc1c(cc2c4c1CCCN4CCC2)C31OC(=O)c2c(Cl)c(Cl)c(Cl)c(Cl)c21 ; *NC(=O)CCCN1CCCc2cc3c(cc21)Oc1cc2c(cc1C31OC(=O)c3c(Cl)c(Cl)c(Cl)c(Cl)c31)CCCN2CC] 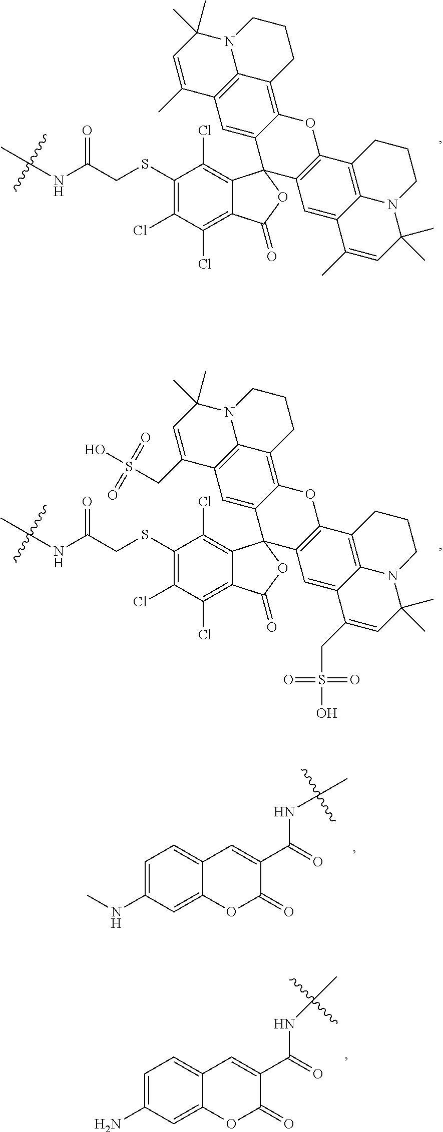 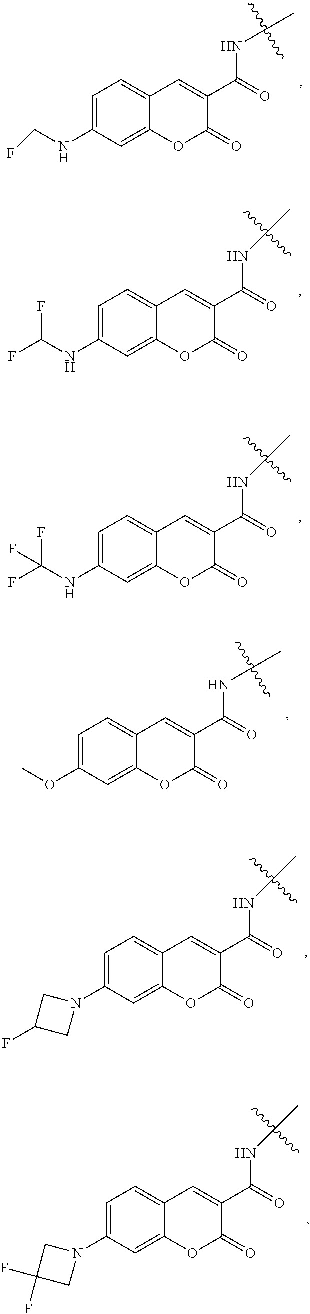 [SMILES: *NC(=O)c1cc2ccc(NCF)cc2oc1=O ; *NC(=O)c1cc2ccc(NC(F)F)cc2oc1=O ; *NC(=O)c1cc2ccc(NC(F)(F)F)cc2oc1=O ; *NC(=O)c1cc2ccc(OC)cc2oc1=O ; *NC(=O)c1cc2ccc(N3CC(F)C3)cc2oc1=O ; *NC(=O)c1cc2ccc(N3CC(F)(F)C3)cc2oc1=O] 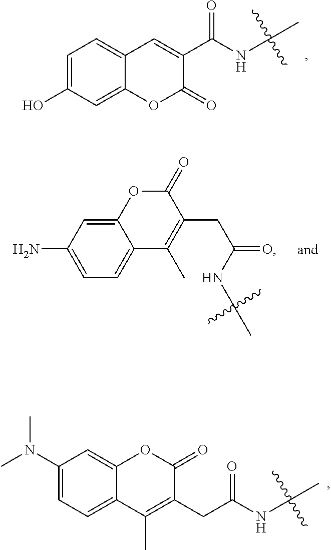 [SMILES: *NC(=O)c1cc2ccc(O)cc2oc1=O ; *NC(=O)Cc1c(C)c2ccc(N)cc2oc1=O ; *NC(=O)Cc1c(C)c2ccc(N(C)C)cc2oc1=O]
where the symbol “ [symbol] ” refers to the site in which the detectable moiety is conjugated to another moiety of a detectable conjugate.

19. A kit comprising: (a) a primary antibody specific to a first morphological marker; (b) a primary antibody specific to a first biomarker; and (c) at least two detection conjugates, wherein the at least two detection conjugates each include a different detectable moiety, wherein each detectable moiety has a first absorbance peak with FWHM of less than about 200 nm and an absorbance maximum (λₘₐₓ) between 330 nm+/−10 and 950 nm+/−10; and wherein an absorbance maximum (λₘₐₓ) of a first detectable moiety and an absorbance maximum (λₘₐₓ) of a second detectable moiety are separated by at least 20 nm.

20. The kit of claim 19, wherein the at least two detection conjugates are selected from the group consisting of: 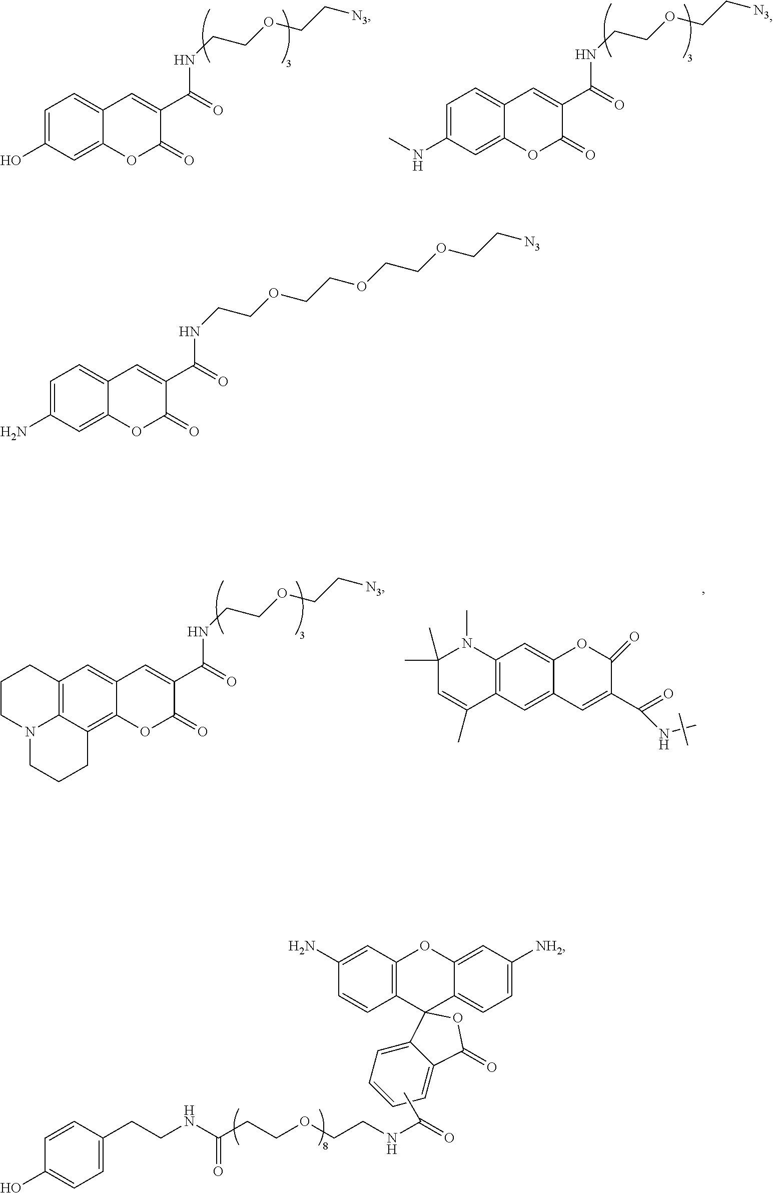 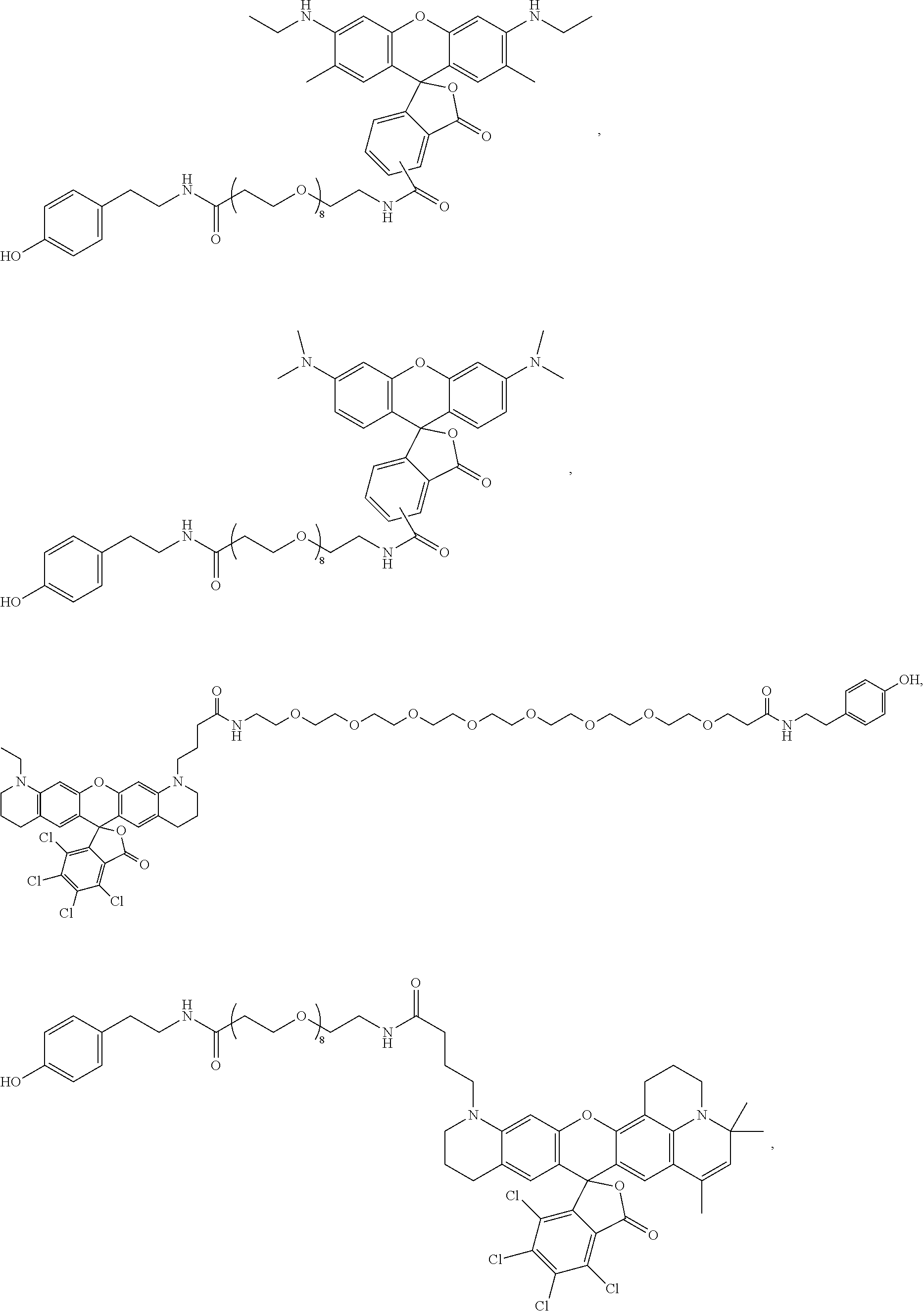 [chemical structure]
